(19) United States
(12) Patent Application Publication
(10) Pub. No.: US 2020/0216416 A1
(43) Pub. Date: Jul. 9, 2020

(54) QUINOLINE COMPOUNDS AS INHIBITORS OF TAM AND MET KINASES

(71) Applicant: Array BioPharma Inc., Boulder, CO (US)

(72) Inventors: Adam Cook, Broomfield, CO (US); Ronald Jay Hinklin, Longmont, CO (US); Oren T. McNulty, Nederland, CO (US)

(73) Assignee: Array BioPharma Inc., Boulder, CO (US)

(21) Appl. No.: 16/732,733

(22) Filed: Jan. 2, 2020

Related U.S. Application Data
(60) Provisional application No. 62/947,720, filed on Dec. 13, 2019.
(60) Provisional application No. 62/858,819, filed on Jun. 7, 2019.
(60) Provisional application No. 62/787,965, filed on Jan. 3, 2019.

(51) Int. Cl. C07D 401/14; C07D 405/14; C07D 413/14

(52) U.S. Cl. CPC C07D 401/14; A61K 45/06; C07D 413/14; C07D 405/14

(57) ABSTRACT

Provided herein are compounds of the Formula I:

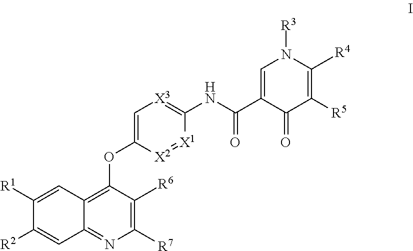

or pharmaceutically acceptable salts thereof, wherein X¹, X², X³, R¹, R², R³, R⁴, R5, ^{R6} and R⁷ are as defined herein, which are inhibitors of one or more TAM kinases and/or c-Met kinase, and are useful in the treatment and prevention of diseases which can be treated with a TAM kinase inhibitor and/or a c-Met kinase inhibitor.

### CROSS-REFERENCE TO RELATED APPLICATION

This application claims priority to U.S. Provisional application Ser. Nos. 62/787,965, filed Jan. 3, 2019, 62/858,819, filed Jun. 7, 2019 and 62/947,720 filed Dec. 13, 2019, each of which is hereby incorporated by reference in its entirety.

### BACKGROUND

Provided herein are novel inhibitors of TAM and MET kinases, pharmaceutical compositions comprising the compounds, processes for making the compounds, and the use of the compounds in therapy. More particularly, provided herein are quinoline compounds useful in the treatment and prevention of diseases which can be treated with a TAM kinase inhibitor or a MET kinase inhibitor.

Receptor tyrosine kinases (RTKs) are cell surface proteins that transmit signals from the extracellular environment to the cell cytoplasm and nucleus to regulate cellular events such as survival, growth, proliferation, differentiation, adhesion and migration.

The TAM subfamily consists of three RTKs including TYRO3, AXL and Mer (Graham et al., 2014, Nature Reviews Cancer 14, 769-785; Linger et al., 2008, Advances in Cancer Research 100, 35-83). TAM kinases are characterized by an extracellular ligand binding domain consisting of two immunoglobulin-like domains and two fibronectin type III domains. Two ligands, growth arrest specific 6 (GAS6) and protein S (PROS1), have been identified for TAM kinases. GAS6 can bind to and activate all three TAM kinases, while PROS1 is a ligand for Mer and TYRO3 (Graham et al., 2014, Nature Reviews Cancer 14, 769-785).

AXL (also known as UFO, ARK, JTK11 and TYRO7) was originally identified as a transforming gene from DNA of patients with chronic myelogenous leukemia (O'Bryan et al., 1991, Mol Cell Biol 11, 5016-5031; Graham et al., 2014, Nature Reviews Cancer 14, 769-785; Linger et al., 2008, Advances in Cancer Research 100, 35-83). GAS6 binds to AXL and induces subsequent auto-phosphorylation and activation of AXL tyrosine kinase. AXL activates several downstream signaling pathways including P13K-Akt, Raf-MAPK, PLC-PKC (Feneyrolles et al., 2014, Molecular Cancer Therapeutics 13, 2141-2148; Linger et al., 2008, Advances in Cancer Research 100, 35-83).

MER (also known as MERTK, EYK, RYK, RP38, NYK and TYRO 12) was originally identified as a phospho-protein from a lymphoblastoid expression library (Graham et al., 1995, Oncogene 10, 2349-2359; Graham et al., 2014, Nature Reviews Cancer 14, 769-785; Linger et al., 2008, Advances in Cancer Research 100, 35-83). Both GAS6 and PROSI can bind to Mer and induce the phosphorylation and activation of Mer kinase (Lew et al., 2014). Like AXL, MER activation also conveys downstream signaling pathways including P13K-Akt and Raf-MAPK (Linger et al., 2008, Advances in Cancer Research 100, 35-83).

TYRO3 (also known as DTK, SKY, RSE, BRT, TIF, ETK2) was originally identified through a PCR-based cloning study (Lai et al., Neuron 6, 691-70, 1991; Graham et al., 2014, Nature Reviews Cancer 14, 769-785; Linger et al., 2008, Advances in Cancer Research 100, 35-83). Both ligands, GAS6 and PROS1, can bind to and activate TYRO3. Although the signaling pathways downstream of TYRO3 activation are the least studied among TAM RTKs, it appears that both P13K-Akt and Raf-MAPK pathways are involved (Linger et al., 2008, Advances in Cancer Research 100, 35-83). AXL, MER and TYRO3 are found to be over-expressed in cancer cells.

The MET family includes mesenchymal-epithelial transition factor (c-Met), a single pass tyrosine kinase receptor that is expressed on the surface of various epithelial cells; its ligand is hepatocyte growth factor/scatter factor (HGF/SF) (Nakamura et al., Nature 342: 440-443, 1989). The binding of HGF to c-Met initiates a series of intracellular signals that mediate embryogenesis and wound healing in normal cells (Organ. Ther. Adv. Med. Oncol. 3(1 Supply): S7-S19, 2011). However, in cancer cells, aberrant HGF/c-Met axis activation, which is closely related to c-Met gene mutations, overexpression, and amplification, promotes tumor development and progression—e.g., by stimulating the PI3K/AKT, Ras/MAPK, JAK/STAT, SRC, and Wnt/α-catenin signal pathways (Zhang et al., Mol. Cancer 17:45, 2018; Mizuno et al., Int. J. Mol. Sci. 14:888-919, 2013). The constitutive activation of the aforementioned c-Met-dependent signaling pathways confers cancer cells with competitive growth advantage relative to normal cells and increases the likelihood of metastasis—e.g., by enabling access to blood supply and conferring the ability to dissociate from tissues (Comoglio et al., Nat. Rev. Drug Discov., 7:504-516, 2008; Birchmeier et al., Nat. Rev. Mol. Cell. Biol. 4:915-925, 2003).

Accordingly, there is a need in the art for compounds and methods of use thereof for the modulation of TAM and MET kinases in treatment of cancer.

### SUMMARY OF THE INVENTION

Provided herein is a compound of the Formula I:

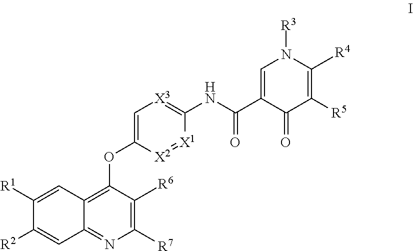

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, (R^{c}R^{d}N)C1-C6 alkyl-, hetCyc² or (hetCyc²)C1-C6 alkyl-;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

Also provided herein is a compound of Formula II

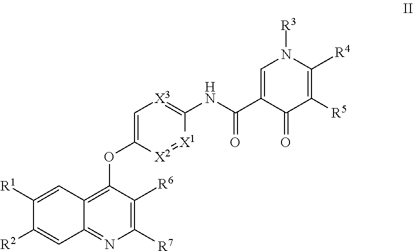

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl- or (R^{c}R^{d}N)C1-C6 alkyl-;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

Also provided herein is a compound of Formula III

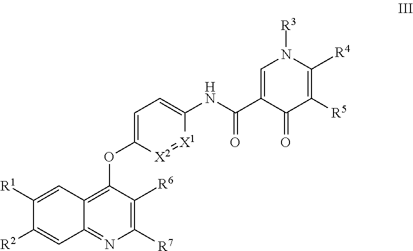

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is C1-C7 alkyl;

R⁴ is hydrogen or C1-C6 alkyl;

R⁵ is phenyl optionally substituted with one or two substituents independently selected from halogen, C1-C6 alkyl and C1-C6 alkoxy;

R⁶ is hydrogen or CN; and

R⁷ is hydrogen or C1-C3 alkyl.

Also provided herein is a compound of Formula IV

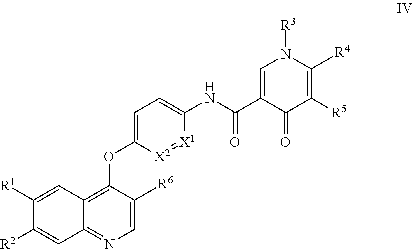

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoro C1-C6 alkoxy or halogen;

R³ is C1-C6 alkyl;

R⁴ is hydrogen or methyl;

R⁵ is phenyl optionally substituted with one or two substituents independently selected from halogen, C1-C6 alkyl and C1-C6 alkoxy; and

R⁶ is hydrogen or CN.

Also provided herein is a pharmaceutical composition comprising a compound of Formula I, II, Ill or IV or a pharmaceutically acceptable salt thereof, in admixture with a pharmaceutically acceptable diluent or carrier.

Also provided herein is a method of inhibiting cell proliferation, in vitro or in vivo, the method comprising contacting a cell with an effective amount of a compound of Formula I, II, III or IV or a pharmaceutically acceptable salt thereof, or a pharmaceutical composition thereof as defined herein.

Also provided herein is a method of treating cancer and/or inhibiting metastasis associated with a particular cancer in a patient in need of such treatment, the method comprising administering to the patient a therapeutically effective amount of a compound of Formula I, II, Ill or IV or a pharmaceutically acceptable salt thereof or a pharmaceutical composition thereof as defined herein.

Also provided herein is a compound of Formula I, II, Ill or IV or a pharmaceutically acceptable salt thereof, or a pharmaceutical composition thereof as defined herein for use in therapy.

Also provided herein is a compound of Formula I, II, Ill or IV or a pharmaceutically acceptable salt thereof or a pharmaceutical composition thereof as defined herein for use in the treatment of cancer and/or inhibiting metastasis associated with a particular cancer.

Also provided herein is a compound of Formula I, II, Ill or IV or a pharmaceutically acceptable salt thereof for use in the inhibition of TAM kinase activity.

Also provided herein is a compound of Formula I, II, Ill or IV or a pharmaceutically acceptable salt thereof or a pharmaceutical composition thereof as defined herein, for use in the treatment of a TAM-associated disease or disorder such as cancer. In some embodiments, the TAM-associated cancer is a cancer having a chromosomal translocation that results in the expression of a TMEM87B-MERTK fusion protein (e.g., amino acids 1-55 of TMEM87B and amino acids 433-1000 of MERTK) or an AXL-MBIP fusion protein.

Also provided herein is the use of a compound of Formula I, II, Ill or IV or a pharmaceutically acceptable salt thereof, as defined herein in the manufacture of a medicament for the treatment of cancer and/or inhibiting metastasis associated with a particular cancer.

Also provided herein is a use of a compound of Formula I, II, Ill or IV or a pharmaceutically acceptable salt thereof, as defined herein in the manufacture of a medicament for the inhibition of TAM kinase activity.

Also provided herein is the use of a compound of Formula I, II, Ill or IV or a pharmaceutically acceptable salt thereof, as defined herein, in the manufacture of a medicament for the treatment of a TAM-associated disease or disorder such as cancer.

Also provided herein is a pharmaceutical combination which comprises (a) a compound of Formula I, II, Ill or IV or a pharmaceutically acceptable salt thereof, and (b) an additional therapeutic agent. Also provided herein is a pharmaceutical combination which comprises (a) a compound of Formula I, II, Ill or IV or a pharmaceutically acceptable salt thereof, and (b) an additional therapeutic agent, for use in therapy. In one embodiment, the compound of Formula I, II, Ill or IV or the pharmaceutically acceptable salt thereof and the additional therapeutic agent are formulated as separate compositions or dosages for simultaneous, separate or sequential use for use in therapy, wherein the amount of the compound of Formula I, II, Ill or IV or a pharmaceutically acceptable salt thereof and of the additional therapeutic agent are together therapeutically effective. Also provided herein is a pharmaceutical composition comprising such a combination. Also provided herein is a commercial package or product comprising such a combination as a combined preparation for simultaneous, separate or sequential use.

In one embodiment, the additional therapeutic agent is an anticancer agent (e.g., any of the additional anticancer agents described herein). Accordingly, provided herein is a pharmaceutical combination for treating cancer (e.g., a TAM-associated cancer) in a patient in need thereof, which comprises (a) a compound of Formula I, II, Ill or IV or a pharmaceutically acceptable salt thereof, and (b) an additional anticancer agent (e.g., any of the additional anticancer agents described herein), wherein the compound of Formula I, II, Ill or IV or the pharmaceutically acceptable salt thereof and the additional therapeutic are formulated as separate compositions or dosages for simultaneous, separate or sequential use for the treatment of cancer, wherein the amounts of the compound of Formula I, II, Ill or IV or a pharmaceutically acceptable salt thereof and of the additional anticancer agent are together effective in treating the cancer.

Also provided herein is a pharmaceutical combination for treating cancer (e.g., a TAM-associated cancer) in a patient in need thereof, which comprises (a) a compound of Formula I, II, Ill or IV or a pharmaceutically acceptable salt thereof, and (b) an additional anticancer agent (e.g., any of the additional anticancer agents described herein), wherein the compound of Formula I, II, Ill or IV or the pharmaceutically acceptable salt thereof and the additional therapeutic are formulated as separate compositions or dosages for simultaneous, separate or sequential use for the treatment of cancer, wherein the amounts of the compound of Formula I, II, Ill or IV or a pharmaceutically acceptable salt thereof and of the additional anticancer agent are together effective in treating the cancer. Also provided herein is a pharmaceutical composition comprising such a combination. Also provided herein is the use of such a combination for the preparation of a medicament for the treatment of cancer. Also provided herein is a commercial package or product comprising such a combination as a combined preparation for simultaneous, separate or sequential use; and to a method of treatment of cancer a patient in need thereof.

Also provided are methods of treating an individual with cancer that include administering a compound of Formula I, II, Ill or IV or a pharmaceutically acceptable salt thereof, before, during, or after administration of another anticancer agent (e.g., another anticancer agent to which the subject has previously developed resistance, e.g., any of the additional anticancer agents described herein).

Also provided herein are methods of treating a patient identified or diagnosed as having a TAM-associated cancer that include administering to a patient identified or diagnosed as having a TAM-associated cancer a therapeutically effective amount of a compound of Formula I, II, Ill or IV or a pharmaceutically acceptable salt thereof, or pharmaceutical composition including a therapeutically effective amount of a compound of Formula I, II, Ill or IV or a pharmaceutically acceptable salt thereof.

Also provided herein are methods of treating a patient having a cancer that include (a) identifying the patient as having a TAM-associated cancer, and (b) administering to the patient identified as having a TAM-associated cancer a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of decreasing the risk of developing a metastasis or an additional metastasis in a patient identified or diagnosed as having a TAM-associated cancer that include administering to a patient identified or diagnosed as having a TAM-associated cancer a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of decreasing the risk of developing a metastasis or an additional metastasis in a patient having a cancer that include: (a) identifying a patient having a TAM-associated cancer, and (b) administering to the identified as having a TAM-associated cancer a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of decreasing migration and/or invasion of a cancer cell in a patient identified or diagnosed as having a TAM-associated cancer that include administering to a patient identified or diagnosed as having a TAM-associated cancer a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of decreasing migration and/or invasion of a cancer cell in a patient having a cancer that include (a) identifying the patient as having a TAM-associated cancer; and (b) administering to the patient identified as having a TAM-associated cancer a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of selecting a treatment for a patient identified or diagnosed as having a TAM-associated cancer that include selecting a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof, for a patient identified or diagnosed as having a TAM-associated cancer.

Also provided herein are methods of selecting a treatment for a patient that include (a) identifying the patient as having a TAM-associated cancer, and (b) selecting a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof, for the patient identified as having a TAM-associated cancer.

Also provided herein are methods of selecting a treatment for a patient identified or diagnosed as having a cancer that include (a) administering an additional anticancer agent to the patient, (b) after (a), detecting increased expression and/or activity of a TAM kinase in a cancer cell from the patient, and (c) after (b), selecting a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof, for the patient.

Also provided herein are methods of treating a patient identified or diagnosed as having a cancer that include: (a) administering to the patient identified or diagnosed as having a cancer one or more doses of at least one additional anticancer agent; (b) after (a), detecting an increase in the expression and/or activity of a TAM kinase in a cancer cell or an immune cell from the subject; and (c) after (b), administering to the patient a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (c) further includes administering to the patent the at least one additional anticancer agent.

Also provided are methods of treating a patient identified or diagnosed as having a cancer that include: (a) detecting an increase in the expression and/or activity of a TAM kinase in a cancer cell or an immune cell from a patient identified or diagnosed as having a cancer and previously administered one or more doses of at least one additional anticancer agent; and (b) after (a), administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (b) further includes administering to the patient the at least one additional anticancer agent.

Also provided herein are methods of treating a patient identified or diagnosed as having a cancer that has been previously administered one or more doses of at least one additional anticancer agent and has been identified as having a cancer cell or an immune cell that has increased expression and/or activity of a TAM kinase that include administering a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof, to the patient. In some embodiments of these methods, step (b) further includes administering to the patient the at least one additional anticancer agent.

Also provided herein are methods of treating a patient identified or diagnosed as having a cancer that include: (a) selecting a patient identified or diagnosed as having increased expression and/or activity of a TAM kinase in a cancer cell or an immune cell; and (b) after (a) administering to the selected patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (b) further includes administering to the patient at least one additional anticancer agent.

Also provided herein are methods of treating a patient identified or diagnosed as having a cancer that include: (a) selecting a patient identified or diagnosed as having a cancer that has been previously administered one or more doses of an additional anticancer agent and identified as having a cancer cell or an immune cell having increased expression and/or activity of a TAM kinase; and (b) after (a), administering to the selected patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (b) further includes administering to the patient at least one additional anticancer agent.

Also provided herein are methods of treating a patient identified or diagnosed as having a TAM-associated cancer that include: (a) administering to the patient identified or diagnosed as having a TAM-associated cancer one or more doses of a TAM kinase inhibitor; (b) after (a), detecting resistance of the TAM-associated cancer in the patient to the TAM kinase inhibitor; and (c) after (b), administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (c) further includes administering to the patient at least one additional anticancer agent.

Also provided herein are methods of treating a patient identified or diagnosed as having a TAM-associated cancer that include: (a) detecting resistance of the TAM-associated cancer in the patient to a TAM kinase inhibitor that was previously administered to the patient; and (b) after (a), administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. In some embodiments of these methods, step (b) further includes administering to the patient at least one additional anticancer agent.

Also provided herein are methods of treating a patient identified or diagnosed as having a TAM-associated cancer and determined to have previously developed resistance to a TAM kinase inhibitor that include administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof. Some embodiments of these methods further include administering to the patient at least one additional anticancer agent.

Also provided herein are methods of decreasing immune tolerance in a subject in need thereof that include administering to the subject a therapeutically effective amount of a compound of Formula I, II, Ill or IV or a pharmaceutically acceptable salt thereof, or a pharmaceutical composition thereof as defined herein.

Also provided herein are methods of inhibiting angiogenesis in a subject in need thereof, the method comprising administering to the subject a therapeutically effective amount of a compound of Formula I, II, Ill or IV or a pharmaceutically acceptable salt thereof, or a pharmaceutical composition thereof as defined herein.

Also provided herein are methods of suppressing resistance to a therapeutic agent in a subject in need thereof that include administering to the subject a therapeutically effective amount of (i) a compound of Formula I, II, Ill or IV or a pharmaceutically acceptable salt thereof, or any of the pharmaceutical compositions thereof described herein, and (ii) the therapeutic agent, where the therapeutic agent is selected from the group consisting of a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor, a RAF inhibitor, an Akt inhibitor, a FTL-3 inhibitor, and a MAP kinase pathway inhibitor.

Also provided herein are methods of treating a patient identified or diagnosed as having a TAM-associated cancer that include administering radiation therapy before or after administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods of treating a patient identified or diagnosed as having a TAM-associated cancer that include administering surgery before or after administering to the patient a therapeutically effective amount of a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein are methods for inhibiting a TAM kinase activity in a mammalian cell in need thereof that include contacting the mammalian cell with a compound of Formula I, II, Ill or IV or a pharmaceutically acceptable salt thereof, or a pharmaceutical composition including a therapeutically effective amount of a compound of Formula I, II, Ill or IV or a pharmaceutically acceptable salt thereof.

Also provided herein is a process for preparing a compound of Formula I, II, Ill or IV or a pharmaceutically acceptable salt thereof.

Also provided herein is a compound of Formula I, II, Ill or IV or a pharmaceutically acceptable salt thereof obtained by a process of preparing the compound as defined herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION OF THE INVENTION

Provided herein is a compound of the Formula I:

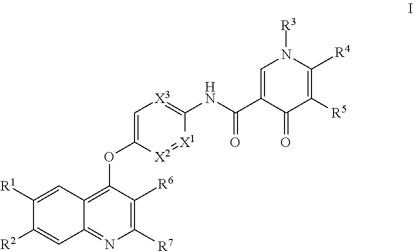

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, (R^{c}R^{d}N)C1-C6 alkyl-, hetCyc² or (hetCyc²)C1-C6 alkyl-;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

Also provided herein is a compound of the Formula II:

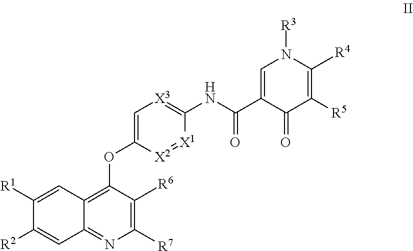

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl- or (R^{c}R^{d}N)C1-C6 alkyl-;

R^{a} and R^{b} are independently hydrogen or C1-C6 alkyl, or

R^{d} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

Also provided herein is a compound of Formula III

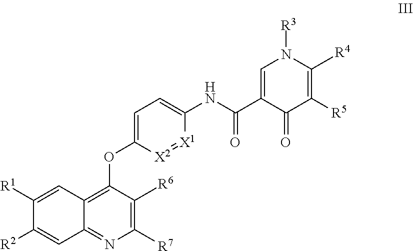

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is C1-C3 alkyl;

R⁴ is hydrogen or C1-C6 alkyl;

R⁵ is phenyl optionally substituted with one or two substituents independently selected from halogen, C1-C6 alkyl and C1-C6 alkoxy;

R⁶ is hydrogen or CN; and

R⁷ is hydrogen or C1-C3 alkyl.

Also provided herein is a compound of Formula IV

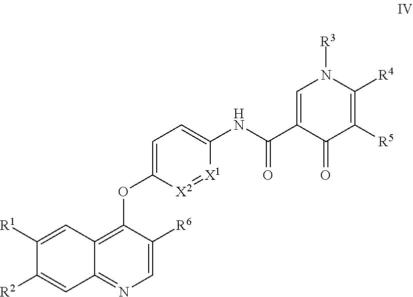

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoroC1-C6 alkoxy or halogen;

R³ is C1-C6 alkyl;

R⁴ is hydrogen or methyl;

R⁵ is phenyl optionally substituted with one or two substituents independently selected from halogen, C1-C6 alkyl and C1-C6 alkoxy; and

R⁶ is hydrogen or CN.

The terms “C1-C3 alkyl”, “C1-C6 alkyl” and “C1-C7 alkyl” as used herein refer to saturated linear or branched-chain monovalent hydrocarbon radicals of one to three, one to six or one to seven carbon atoms, respectively. Examples include, but are not limited to, methyl, ethyl, 1-propyl, isopropyl, 1-butyl, isobutyl, sec-butyl, tert-butyl, 2-methyl-2-propyl, pentyl, neopentyl, hexyl and heptan-4-yl.

The term “halogen” means —F (sometimes referred to herein as “fluoro” or “fluoros”), —CI, —Br and —I.

The term “C1-C6 alkoxy” as used herein refers to a saturated linear or branched-chain monovalent alkoxy radical of one to six carbon atoms, wherein the radical is on the oxygen atom. Examples include methoxy, ethoxy, propoxy, isopropoxy, butoxy and tert-butoxy.

The term “fluoroC1-C6 alkoxy” as used herein refers to a saturated linear or branched-chain monovalent alkoxy radical of one to six carbon atoms as defined herein, wherein the radical is on the oxygen atom, wherein 1-6 hydrogen atoms are replaced by fluoro. An example is trifluoromethoxy.

The term “(C1-C6 alkoxy)C1-C6 alkyl-” as used herein refers to saturated linear or branched-chain monovalent radicals of one to six carbon atoms, wherein one of the carbon atoms is substituted with a C1-C6 alkoxy group as defined herein. Examples include methoxymethyl (CH₃OCH₂—) and methoxyethyl (CH₃OCH₂CH₂—).

The term “hydroxyC1-C6 alkyl-” as used herein refers to a saturated linear or branched-chain monovalent alkyl radicals of one to six carbon atoms, wherein one of the carbon atoms is substituted with a hydroxyl group.

The term “R^{a}R^{b}NC(═O)C1-C6 alkyl-” as used herein refers to a saturated linear or branched-chain monovalent alkyl radicals of one to six carbon atoms, wherein one of the carbon atoms is substituted with a “R^{a}R^{b}NC(═O)— group.

The term “(R^{c}R^{d}N)C1-C6 alkyl-” as used herein refers to a saturated linear or branched-chain monovalent alkyl radicals of one to six carbon atoms, wherein one of the carbon atoms is substituted with a R^{c}R^{d}N— group.

The term “(hetCyc¹)C1-C6 alkoxy-” as used herein refers to a saturated linear or branched-chain monovalent alkoxy radical of one to six carbon atoms as defined herein, wherein one of the carbon atoms is substituted with a hetCyc¹ group as defined herein.

The term “(hetCyc²)C1-C6 alkyl-” as used herein refers to a saturated linear or branched-chain monovalent alkyl radical of one to six carbon atoms, wherein one of the carbon atoms is substituted with a hetCyc² group as defined herein.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

In some embodiments of Formula I, X¹ is N, X² is CH and X³ is CH.

In some embodiments of Formula I, X¹ is N, X² is CH and X³ is N.

In some embodiments of Formula I, X¹ is CH, X² is N and X³ is CH.

In some embodiments of Formula I, R¹ is hydrogen.

In some embodiments of Formula I, R¹ is C1-C6 alkoxy. In some such embodiments, R¹ is methoxy or ethoxy. In some such embodiments, R¹ is methoxy. In some such embodiments, R¹ is ethoxy.

In some embodiments of Formula I, R² is hydrogen.

In some embodiments of Formula I, R² is C1-C6 alkoxy. In some such embodiments, R² is methoxy or ethoxy. In some such embodiments, R² is methoxy. In some such embodiments, R² is ethoxy.

In some embodiments, R² is fluoroC1-C6 alkoxy. In some such embodiments, R² is trifluoromethoxy.

In some embodiments of Formula I, R² is halogen. In some such embodiments, R² is fluoro.

In some embodiments of Formula I, R¹ is hydrogen and R² is C1-C6 alkoxy. In some such embodiments, R¹ is hydrogen and R² is ethoxy.

In some embodiments of Formula I, R¹ is hydrogen and R² is fluoroC1-C6 alkoxy. In some such embodiments, R¹ is hydrogen and R² is trifluoromethoxy.

In some embodiments of Formula I, R¹ is hydrogen and R² is halogen.

In some embodiments of Formula I, R¹ is hydrogen and R² is (hetCyc¹)C1-C6 alkoxy-. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is hydrogen and R² is (morpholin-1-yl)propoxy.

In some embodiments of Formula I, R¹ is C1-C6 alkoxy and R² is hydrogen. In some such embodiments, R¹ is methoxy and R² is hydrogen. In some such embodiments, R¹ is ethoxy and R² is hydrogen.

In some such embodiments of Formula I, R¹ is C1-C6 alkoxy and R² is C1-C6 alkoxy. In some such embodiments, R¹ is methoxy and R² is methoxy.

In some embodiments of Formula I, R¹ is C1-C6 alkoxy and R² is halogen. In some such embodiments, R¹ is methoxy and R² is halogen. In some such embodiments, R¹ is C1-C6 alkoxy and R² is fluoro. In some such embodiments, R¹ is methoxy and R² is fluoro.

In some embodiments of Formula I, R¹ is C1-C6 alkoxy and R² is (hetCyc¹)C1-C6 alkoxy. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is methoxy and R² is (morpholin-1-yl)propoxy.

In some embodiments of Formula I, R³ is hydrogen.

In some embodiments of Formula I, R³ is C1-C7 alkyl. In some such embodiments, R³ is selected from methyl, ethyl, propyl, isopropyl, 1-isobutyl, pentan-3-yl, hetpan-4-yl and 1-isopentyl.

In some embodiments of Formula I, R³ is (C1-C6 alkoxy)C1-C6 alkyl-. In one such embodiment, R³ is (2-methoxy)ethyl.

In some embodiments of Formula I, R³ is hydroxyC1-C6 alkyl-. In one such embodiment, R³ is 2-hydroxy-2-methylpropyl.

In some embodiments of Formula I, R³ is R^{a}R^{b}NC(═O)C1-C6 alkyl-, wherein R^{a} and R^{b} are independently hydrogen or C1-C6 alkyl, or R^{a} and R^{b} together with the nitrogen atom to which they are attached form a 5-6 membered saturated heterocyclic ring having one ring nitrogen atom and optionally having a second ring heteroatom selected from O and N.

In some embodiments when R³ is R^{a}R^{b}NC(═O)C1-C6 alkyl-, R^{a} and R^{b} are independently hydrogen or C1-C6 alkyl. In some such embodiments, R³ is (CH₃)₂NC(═O)CH₂— or CH₃NHC(═O)CH₂—.

In some embodiments when R³ is R^{a}R^{b}NC(═O)C1-C6 alkyl-, R^{a} and R^{b} together with the nitrogen atom to which they are attached form a 5-6 membered saturated heterocyclic ring having one ring nitrogen atom and optionally having a second ring heteroatom selected from O and N. In some such embodiments, R³is selected from:

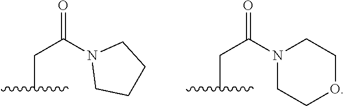 [SMILES: *CC(=O)N1CCCC1 ; *CC(=O)N1CCOCC1]

In some embodiments of Formula I, R³ is (R^{c}R^{d}N)C1-C6 alkyl-, wherein R^{c} and R^{d} are independently hydrogen or C1-C6 alkyl. In one such embodiment, R³ is dimethylaminopropyl.

In some embodiments of Formula I, R³ is hetCyc². In one such embodiment, R³ is tetrahydro-2H-pyran-4-yl.

In some embodiments of Formula I, R³ is (hetCyc²)C1-C6 alkyl, wherein hetCyc² is a 5-6 membered saturated heterocyclic ring having 1-2 ring nitrogen atoms independently selected from N and O. In one such embodiment, R³ is (tetrahydro-2H-pyran-4-yl)methyl-.

In some embodiments of Formula I, R⁴ is hydrogen.

In some embodiments of Formula I, R⁴ is C1-C6 alkyl. In some such embodiments, R⁴ is methyl or isopropyl.

In some embodiments of Formula I, R⁵ is phenyl optionally substituted with one or two substituents independently selected from halogen, C1-C6 alkyl and C1-C6 alkoxy. In some such embodiments, R⁵ is phenyl substituted with one or two substituents independently selected from halogen, C1-C6 alkyl and C1-C6 alkoxy. In some such embodiments, R⁵ is phenyl optionally substituted with one or two substituents independently selected from fluoro, chloro, methyl and methoxy. In some such embodiments, R⁵ is phenyl substituted with one or two substituents independently selected from fluoro, chloro, methyl and methoxy. In some such embodiments, R⁵ is phenyl substituted with fluoro. In some such embodiments, R⁵ is selected from the following:

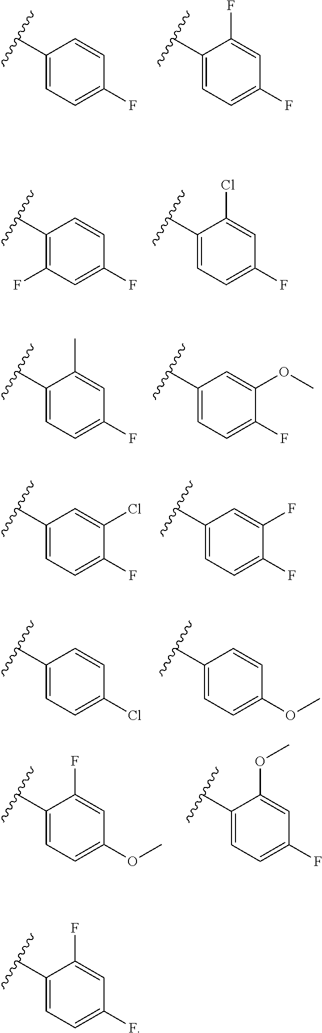

In some embodiments of Formula I, R⁶ is hydrogen.

In some embodiments of Formula I, R⁶ is CN.

In some embodiments of Formula I, R⁷ is hydrogen.

In some embodiments of Formula I, R⁷ is C1-C3 alkyl. In some such embodiments, R⁷ is methyl.

In some embodiments of Formula I, R⁶ is hydrogen and R⁷ is hydrogen.

In some embodiments of Formula I, R⁶ is hydrogen and R⁷ is C1-C3 alkyl. In some such embodiments, R⁶ is hydrogen and R⁷ is methyl.

In some embodiments of Formula I, R⁶ is CN and R⁷ is hydrogen.

In some embodiments, a compound of Formula I is selected from a compound of Examples 1-58 and pharmaceutically acceptable salts thereof.

Formula I-A

In some embodiments, the compound of Formula I has Formula I-A

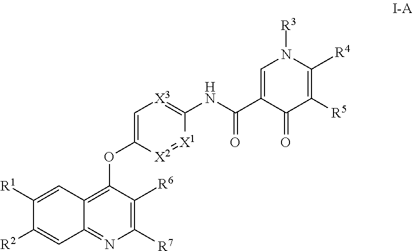

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is C1-C6 alkoxy or fluoroC1-C6 alkoxy;

R³ is hydrogen, C1-C7 alkyl, (C1-C6 alkoxy)C1-C6 alkyl-, hydroxyC-C6 alkyl-, R^{a}R^{b}NC(═O)C1-C6 alkyl-, (R^{c}R^{d}N)C1-C6 alkyl-, hetCyc² or (hetCyc²)C1-C6 alkyl;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-A, X¹ is N, X² is CH and X³ is CH.

In some embodiments of Formula I-A, X¹ is N, X² is CH and X³ is N.

In some embodiments of Formula I-A, X¹ is CH, X² is N and X³ is CH.

In some embodiments of Formula I-A, R¹ is hydrogen.

In some embodiments of Formula I-A, R¹ is C1-C6 alkoxy. In some embodiments of Formula I-A, R¹ is methoxy. In some embodiments of Formula I-A, R¹ is ethoxy.

In some embodiments of Formula I-A, R² is C1-C6 alkoxy. In some embodiments of Formula I-A, R² is methoxy. In some embodiments of Formula I-A, R² is ethoxy.

In some embodiments of Formula I-A, R¹ is C1-C6 alkoxy and R² is C1-C6 alkoxy. In some embodiments of Formula I-A, R¹ is methoxy and R² is methoxy.

In some embodiments of Formula I-A, R³ is hydrogen.

In some embodiments of Formula I-A, R³ is C1-C7 alkyl. In some embodiments of Formula I-A, R³ is methyl, ethyl, propyl, isopropyl, 1-isobutyl, pentan-3-yl, hetpan-4-yl, or 1-isopentyl.

In some embodiments of Formula I-A, R³ is (C1-C6 alkoxy)C1-C6 alkyl-.

In some embodiments of Formula I-A, R³ is hydroxyC1-C6 alkyl-.

In some embodiments of Formula I-A, R³ is R^{a}R^{b}NC(═O)C1-C6 alkyl-, wherein R^{a} and R^{b} are independently hydrogen or C1-C6 alkyl, or R^{a} and R^{b} together with the nitrogen atom to which they are attached form a 5-6 membered saturated heterocyclic ring having one ring nitrogen atom and optionally having a second ring heteroatom selected from O and N.

In some embodiments of Formula I-A, R³ is R^{a}R^{b}Nc(=0)C1-C6 alkyl-, wherein R^{a} and R^{b} are independently hydrogen or C1-C6 alkyl.

In some embodiments of Formula I-A, R³ is R^{a}R^{b}Nc(=0)C1-C6 alkyl-, wherein R^{a} and R^{b} together with the nitrogen atom to which they are attached form a 5-6 membered saturated heterocyclic ring having one ring nitrogen atom and optionally having a second ring heteroatom selected from O and N.

In some embodiments of Formula I-A, R³ is (R^{c}R^{d}N)C1-C6 alkyl-, wherein R^{c} and R^{d} are independently hydrogen or C1-C6 alkyl.

In some embodiments of Formula I-A, R³ is hetCyc².

In some embodiments of Formula I-A, R³ is (hetCyc²)C1-C6 alkyl.

In some embodiments of Formula I-A, R⁴ is hydrogen.

In some embodiments of Formula I-A, R⁴ is C1-C6 alkyl. In some embodiments of Formula I-A, R⁴ is methyl or isopropyl.

In some embodiments of Formula I-A, R⁵ is phenyl optionally substituted with one or two substituents independently selected from halogen, C1-C6 alkyl and C1-C6 alkoxy. In some embodiments of Formula I-A, R⁵ is phenyl substituted with one or two substituents independently selected from halogen, C1-C6 alkyl and C1-C6 alkoxy. In some embodiments of Formula I-A, R⁵ is phenyl optionally substituted with one or two substituents independently selected from fluoro, chloro, methyl and methoxy. In some embodiments of Formula I-A, R⁵ is phenyl substituted with one or two substituents independently selected from fluoro, chloro, methyl and methoxy. In some such embodiments, R⁵ is phenyl substituted with fluoro.

In some embodiments of Formula I-A, R⁶ is hydrogen.

In some embodiments of Formula I-A, R⁶ is CN.

In some embodiments of Formula I-A, R⁷ is hydrogen.

In some embodiments of Formula I-A, R⁷ is C1-C3 alkyl. In some embodiments, R⁷ is methyl.

In some embodiments of Formula I-A, R⁶ is hydrogen and R⁷ is hydrogen.

In some embodiments of Formula I-A, R⁶ is hydrogen and R⁷ is C1-C3 alkyl. In some embodiments, R⁶ is hydrogen and R⁷ is methyl.

In some embodiments of Formula I-A, R⁶ is CN and R⁷ is hydrogen.

Formula I-B

In some embodiments, the compound of Formula I has Formula I-B

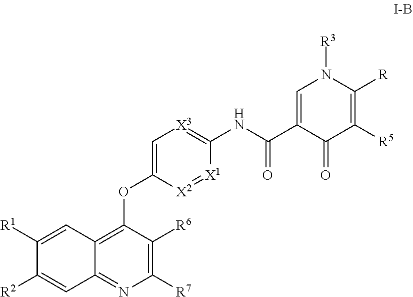

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is halogen;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, (R^{c}R^{d}N)C1-C6 alkyl-, hetCyc² or (hetCyc²)C1-C6 alkyl;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-B, X¹ is N, X² is CH and X³ is CH.

In some embodiments of Formula I-B, R¹ is C1-C6 alkoxy. In some embodiments, R¹ is methoxy.

In some embodiments of Formula I-B, R² is fluoro.

In some embodiments of Formula I-B, R¹ is C1-C6 alkoxy and R² is fluoro. In some embodiments, R¹ is methoxy.

In some embodiments of Formula I-B, R³ is C1-C7 alkyl. In some embodiments, R³ is isopropyl.

In some embodiments of Formula I-B, R² is fluoro, and R³ is C1-C7 alkyl. In some embodiments, R³ is isopropyl.

In some embodiments of Formula I-B, R¹ is C1-C6 alkoxy, R² is fluoro, and R³ is C1-C7 alkyl. In some embodiments, R¹ is methoxy. In some embodiments, R³ is isopropyl. In some embodiments, R¹ is methoxy and R³ is isopropyl.

In some embodiments of Formula I-B, R⁴ is hydrogen.

In some embodiments of Formula I-B, R¹ is C1-C6 alkoxy, R² is fluoro, R³ is C1-C7 alkyl, and R⁴ is hydrogen.

In some embodiments of Formula I-B, R⁴ is C1-C6 alkyl. In some embodiments, R⁴ is methyl

In some embodiments of Formula I-B, R² is fluoro, and R⁴ is C1-C6 alkyl. In some embodiments, R⁴ is methyl

In some embodiments of Formula I-B, R² is fluoro, R³ is C1-C7 alkyl, and R⁴ is C1-C6 alkyl. In some embodiments, R¹ is methoxy. In some embodiments, R³ is isopropyl. In some embodiments, R¹ is methoxy and R³ is isopropyl.

In some embodiments of Formula I-B, R¹ is C1-C6 alkoxy, R² is fluoro, R³ is C1-C7 alkyl, and R⁴ is C1-C6 alkyl. In some embodiments, R¹ is methoxy. In some embodiments, R³ is isopropyl. In some embodiments, R⁴ is methyl.

In some embodiments of Formula I-B, R⁵ is phenyl optionally substituted with one or two substituents independently selected from halogen, C1-C6 alkyl and C1-C6 alkoxy. In some embodiments of Formula I-B, R⁵ is phenyl substituted with one or two substituents independently selected from halogen, C1-C6 alkyl and C1-C6 alkoxy. In some embodiments of Formula I-B, R⁵ is phenyl optionally substituted with one or two substituents independently selected from fluoro, chloro, methyl and methoxy. In some embodiments of Formula I-B, R⁵ is phenyl substituted with one or two substituents independently selected from fluoro, chloro, methyl and methoxy. In some embodiments of Formula I-B, R⁵ is phenyl optionally substituted with halogen. In some embodiments of Formula I-B, R⁵ is phenyl substituted with halogen. In some embodiments of Formula I-B, R⁵ is phenyl optionally substituted with fluoro. In some embodiments of Formula I-B, R⁵ is phenyl substituted with fluoro.

In some embodiments of Formula I-B, R¹ is C1-C6 alkoxy, R² is fluoro, R³ is C1-C7 alkyl, R⁴ is C1-C6 alkyl, and R⁵ is phenyl optionally substituted with halogen. In some embodiments, R¹ is methoxy. In some embodiments, R³ is isopropyl. In some embodiments, R⁴ is methyl. In some embodiments, R⁵ is phenyl substituted with halogen.

In some embodiments of Formula I-B, R¹ is C1-C6 alkoxy, R² is fluoro, R³ is C1-C7 alkyl, R⁴ is hydrogen, and R⁵ is phenyl optionally substituted with halogen. In some embodiments, R⁵ is phenyl substituted with halogen.

In some embodiments of Formula I-B, R⁶ is hydrogen.

In some embodiments of Formula I-B, R⁷ is hydrogen.

In some embodiments of Formula I-B, R⁶ is hydrogen and R⁷ is hydrogen.

In some embodiments of Formula I-B, R¹ is C1-C6 alkoxy, R² is fluoro, R³ is C1-C7 alkyl, R⁴ is hydrogen or C1-C6 alkyl, R⁵ is phenyl optionally substituted with halogen, R⁶ is hydrogen and R⁷ is hydrogen. In some embodiments, R¹ is methoxy. In some embodiments, R³ is isopropyl. In some embodiments, R⁴ is hydrogen. In some embodiments, R⁴ is C1-C6 alkyl. In some embodiments, R⁵ is phenyl substituted with halogen. In some such embodiments, R⁵ is phenyl substituted with fluoro.

Formula I-C

In some embodiments, the compound of Formula I has Formula I-C

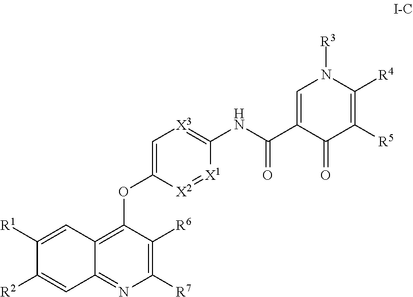

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, (R^{c}R^{d}N)C1-C6 alkyl-, hetCyc² or (hetCyc²)C1-C6 alkyl;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-C, X¹ is N, X² is CH and X³ is CH.

In some embodiments of Formula I-C, R¹ is C1-C6 alkoxy. In some embodiments of Formula I-C, R¹ is methoxy.

In some embodiments of Formula I-C, R³ is C1-C7 alkyl. In some embodiments of Formula I-C, R³ is methyl, ethyl or isopropyl.

In some embodiments of Formula I-C, R¹ is C1-C6 alkoxy and R³ is C1-C7 alkyl.

In some embodiments of Formula I-C, R³ is (R^{c}R^{d}N)C1-C6 alkyl-, wherein R^{c} and R^{d} are independently hydrogen or C1-C6 alkyl.

In some embodiments of Formula I-C, R¹ is C1-C6 alkoxy and R³ is (R^{c}R^{d}N)C1-C6 alkyl-, wherein R^{c} and R^{d} are independently hydrogen or C1-C6 alkyl.

In some embodiments of Formula I-C, R⁴ is hydrogen.

In some embodiments of Formula I-C, R⁴ is C1-C6 alkyl. In some embodiments, R⁴ is methyl.

In some embodiments of Formula I-C, R¹ is C1-C6 alkoxy, R³ is C1-C7 alkyl, and R⁴ is C1-C6 alkyl.

In some embodiments of Formula I-C, R⁵ is phenyl optionally substituted with one or two substituents independently selected from halogen, C1-C6 alkyl and C1-C6 alkoxy. In some embodiments of Formula I-C, R⁵ is phenyl substituted with one or two substituents independently selected from halogen, C1-C6 alkyl and C1-C6 alkoxy. In some embodiments, R⁵ is phenyl optionally substituted with one or two substituents independently selected from fluoro, chloro, methyl and methoxy. In some embodiments, R⁵ is phenyl substituted with one or two substituents independently selected from fluoro, chloro, methyl and methoxy. In some embodiments, R⁵ is phenyl optionally substituted with halogen. In some embodiments, R⁵ is phenyl substituted with halogen. In some embodiments, R⁵ is phenyl optionally substituted with fluoro or chloro. In some embodiments, R⁵ is phenyl substituted with fluoro or chloro. In some such embodiments, R⁵ is phenyl substituted with fluoro.

In some embodiments of Formula I-C, R¹ is C1-C6 alkoxy, R³ is C1-C7 alkyl, R⁴ is C1-C6 alkyl, and R⁵ is phenyl optionally substituted with halogen.

In some embodiments of Formula I-C, R⁶ is hydrogen.

In some embodiments of Formula I-C, R⁷ is hydrogen.

In some embodiments of Formula I-C, R⁶ is hydrogen and R⁷ is hydrogen.

In some embodiments of Formula I-C, R¹ is C1-C6 alkoxy, R³ is C1-C7 alkyl, R⁴ is C1-C6 alkyl, R⁵ is phenyl optionally substituted with halogen, R⁶ is hydrogen and R⁷ is hydrogen. In some embodiments, R⁵ is phenyl substituted with halogen. In some such embodiments, R⁵ is phenyl substituted with fluoro.

In some embodiments of Formula I-C, R¹ is C1-C6 alkoxy, R³ is (R^{c}R^{d}N)C1-C6 alkyl-, wherein R^{c} and R^{d} are independently hydrogen or C1-C6 alkyl, and R⁴ is C1-C6 alkyl.

In some embodiments of Formula I-C, R¹ is C1-C6 alkoxy, R³ is (R^{c}R^{d}N)C1-C6 alkyl-, wherein R^{c} and R^{d} are independently hydrogen or C1-C6 alkyl, R⁴ is C1-C6 alkyl, and R⁵ is phenyl optionally substituted with halogen. In some embodiments, R⁵ is phenyl substituted with halogen. In some such embodiments, R⁵ is phenyl substituted with fluoro.

In some embodiments of Formula I-C, R¹ is C1-C6 alkoxy, R³ is (R^{c}R^{d}N)C1-C6 alkyl-, wherein R^{c} and R^{d} are independently hydrogen or C1-C6 alkyl, R⁴ is C1-C6 alkyl, R⁵ is phenyl optionally substituted with halogen, R⁶ is hydrogen and R⁷ is hydrogen. In some embodiments, R⁵ is phenyl substituted with halogen.

Formula I-D

In some embodiments, the compound of Formula I has Formula I-D

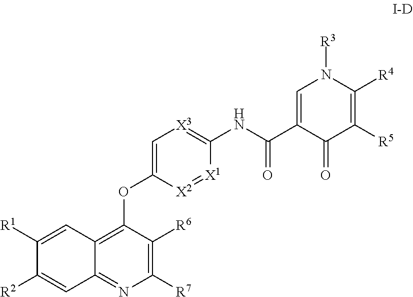

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is (hetCyc¹)C1-C6 alkoxy;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, (R^{c}R^{d}N)C1-C6 alkyl-, hetCyc² or (hetCyc²)C1-C6 alkyl;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-D, X¹ is N, X² is CH and X³ is CH.

In some embodiments of Formula I-D, R¹ is C1-C6 alkoxy. In some embodiments of Formula I-C, R¹ is methoxy.

In some embodiments of Formula I-D, R³ is C1-C7 alkyl. In some embodiments of Formula I-C, R³ is isopropyl.

In some embodiments of Formula I-D, R⁴ is hydrogen.

In some embodiments of Formula I-D, R⁵ is phenyl optionally substituted with one or two substituents independently selected from halogen, C1-C6 alkyl and C1-C6 alkoxy. In some embodiments of Formula I-D, R⁵ is phenyl substituted with one or two substituents independently selected from halogen, C1-C6 alkyl and C1-C6 alkoxy. In some embodiments of Formula I-D, R⁵ is phenyl optionally substituted with one or two substituents independently selected from fluoro, chloro, methyl and methoxy. In some embodiments of Formula I-D, R⁵ is phenyl substituted with one or two substituents independently selected from fluoro, chloro, methyl and methoxy. In some embodiments of Formula I-D, R⁵ is phenyl optionally substituted with halogen. In some embodiments of Formula I-D, R⁵ is phenyl substituted with halogen. In some embodiments of

Formula I-D, R⁵ is phenyl optionally substituted with fluoro. In some embodiments of Formula I-D, R⁵ is phenyl substituted with fluoro.

In some embodiments of Formula I-D, R⁶ is hydrogen.

In some embodiments of Formula I-D, R⁷ is hydrogen.

In some embodiments of Formula I-D, R⁶ is hydrogen and R⁷ is hydrogen.

In some embodiments of Formula I-D, R¹ is C1-C6 alkoxy, R³ is C1-C7 alkyl, R⁴ is hydrogen, R⁵ is phenyl optionally substituted with halogen, R⁶ is hydrogen and R⁷ is hydrogen. In some embodiments of Formula I-D, R⁵ is phenyl substituted with halogen.

Formula l-E

In some embodiments, the compound of Formula I has Formula I-E

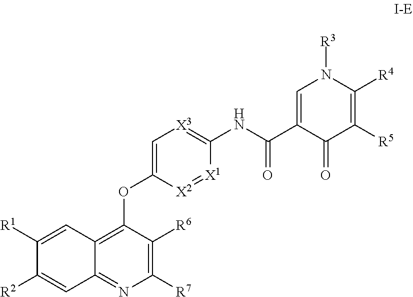

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is C1-C7 alkyl;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-E, X¹ is N, X² is CH and X³ is CH.

In some embodiments of Formula I-E, X¹ is N, X² is CH and X³ is N.

In some embodiments of Formula I-E, X¹ is CH, X² is N and X³ is CH.

In some embodiments of Formula I-E, R¹ is C1-C6 alkoxy.

In some embodiments of Formula I-E, R² is hydrogen.

In some embodiments of Formula I-E, R² is C1-C6 alkoxy.

In some embodiments of Formula I-E, R² is fluoroC1-C6 alkoxy.

In some embodiments of Formula I-E, R² is halogen.

In some embodiments of Formula I-E, R² is (hetCyc¹)C1-C6 alkoxy.

In some embodiments of Formula I-E, R³ is methyl, ethyl, propyl, isopropyl, 1-isobutyl, pentan-3-yl, hetpan-4-yl or 1-isopentyl.

In some embodiments of Formula I-E, R⁴ is hydrogen.

In some embodiments of Formula I-E, R⁴ is C1-C6 alkyl. In some embodiments of Formula I-E, R⁴ is methyl. In some embodiments of Formula I-E, R⁴ is isopropyl.

In some embodiments of Formula I-E, R⁵ is phenyl optionally substituted with one or two substituents independently selected from halogen, C1-C6 alkyl and C1-C6 alkoxy. In some embodiments of Formula I-E, R⁵ is phenyl substituted with one or two substituents independently selected from halogen, C1-C6 alkyl and C1-C6 alkoxy. In some embodiments of Formula I-E, R⁵ is phenyl optionally substituted with one or two substituents independently selected from fluoro, chloro, methyl and methoxy. In some embodiments of Formula I-E, R⁵ is phenyl substituted with one or two substituents independently selected from fluoro, chloro, methyl and methoxy. In some such embodiments, R⁵ is phenyl substituted with fluoro.

In some embodiments of Formula I-E, R⁶ is hydrogen.

In some embodiments of Formula I-E, R⁶ is CN.

In some embodiments of Formula I-E, R⁷ is hydrogen.

In some embodiments of Formula I-E, R⁷ is C1-C3 alkyl. In some embodiments, R⁷ is methyl.

In some embodiments of Formula I-E, R⁶ is hydrogen and R⁷ is hydrogen.

In some embodiments of Formula I-E, R⁶ is hydrogen and R⁷ is C1-C3 alkyl. In some embodiments, R⁶ is hydrogen and R⁷ is methyl.

In some embodiments of Formula I-E, R⁶ is CN and R⁷ is hydrogen.

In some embodiments of Formula I-E, R¹ is C1-C6 alkoxy and R² is C1-C6 alkoxy. In some embodiments, R¹ is methoxy. In some embodiments, R² is methoxy. In some embodiments, R¹ is methoxy and R² is methoxy.

In some embodiments of Formula I-E, R¹ is C1-C6 alkoxy and R² is hydrogen. In some embodiments, R¹ is methoxy. In some embodiments, R¹ is ethoxy.

In some embodiments of Formula I-E, R¹ is C1-C6 alkoxy and R² is (hetCyc¹)C1-C6 alkoxy.

In some embodiments of Formula I-E, R¹ is C1-C6 alkoxy and R² is fluoro. In some embodiments, R¹ is methoxy.

In some embodiments of Formula I-E, R¹ is hydrogen and R² is C1-C6 alkoxy. In some embodiments, R² is methoxy. In some embodiments, R² is ethoxy.

In some embodiments of Formula I-E, R¹ is hydrogen and R² is fluoroC1-C6 alkoxy. In some embodiments, R² is trifluoromethoxy.

In some embodiments of Formula I-E, X¹ is N, X² is CH, X³ is CH, R¹ is C1-C6 alkoxy and R² is hydrogen.

In some embodiments of Formula I-E, X¹ is N, X² is CH, X³ is CH, R¹ is C1-C6 alkoxy, R² is hydrogen, R⁶ is hydrogen and R⁷ is hydrogen.

In some embodiments of Formula I-E, X¹ is N, X² is CH, X³ is CH, R¹ is C1-C6 alkoxy and R² is C1-C6 alkoxy.

In some embodiments of Formula I-E, X¹ is N, X² is CH, X³ is CH, R¹ is C1-C6 alkoxy, R² is C1-C6 alkoxy, R⁶ is hydrogen and R⁷ is hydrogen.

In some embodiments of Formula I-E, X¹ is N, X² is CH, X³ is CH, R¹ is C1-C6 alkoxy, R² is C1-C6 alkoxy, R⁶ is CN and R⁷ is hydrogen.

In some embodiments of Formula I-E, X¹ is N, X² is CH, X³ is CH, R¹ is C1-C6 alkoxy, R² is C1-C6 alkoxy, R⁶ is hydrogen and R⁷ is C1-C3 alkyl.

In some embodiments of Formula I-E, X¹ is N, X² is CH, X³ is CH, R¹ is C1-C6 alkoxy, R² is C1-C6 alkoxy, R⁶ is hydrogen and R⁷ is methyl.

In some embodiments of Formula I-E, X¹ is N, X² is CH, X³ is CH, R¹ is C1-C6 alkoxy and R² is (hetCyc¹)C1-C6 alkoxy.

In some embodiments of Formula I-E, X¹ is N, X² is CH, X³ is CH, R¹ is C1-C6 alkoxy, R² is (hetCyc¹)C1-C6 alkoxy, R⁶ is hydrogen and R⁷ is hydrogen.

In some embodiments of Formula I-E, X¹ is N, X² is CH, X³ is CH, R¹ is C1-C6 alkoxy and R² is fluoro.

In some embodiments of Formula I-E, X¹ is N, X² is CH, X³ is CH, R¹ is C1-C6 alkoxy, R² is fluoro, R⁶ is hydrogen and R⁷ is hydrogen.

In some embodiments of Formula I-E, X¹ is N, X² is CH, X³ is CH, R¹ is hydrogen and R² is C1-C6 alkoxy.

In some embodiments of Formula I-E, X¹ is N, X² is CH, X³ is CH, R¹ is hydrogen, R² is C1-C6 alkoxy, In some embodiments, R⁶ is hydrogen and R⁷ is hydrogen.

In some embodiments of Formula I-E, X¹ is N, X² is CH, X³ is CH, R¹ is hydrogen and R² is fluoroC1-C6 alkoxy.

In some embodiments of Formula I-E, X¹ is N, X² is CH, X³ is CH, R¹ is hydrogen and R² is fluoroC1-C6 alkoxy, R⁶ is hydrogen and R⁷ is hydrogen.

In some embodiments of Formula I-E, X¹ is N, X² is CH, X³ is N, R¹ is C1-C6 alkoxy and R² is C1-C6 alkoxy.

In some embodiments of Formula I-E, X¹ is N, X² is CH, X³ is N, R¹ is C1-C6 alkoxy, R² is C1-C6 alkoxy, R⁶ is hydrogen and R⁷ is hydrogen.

In some embodiments of Formula I-E, X¹ is N, X² is CH, X³ is N, R¹ is C1-C6 alkoxy, and R² is C1-C6 alkoxy.

In some embodiments of Formula I-E, X¹ is N, X² is CH, X³ is N, R¹ is C1-C6 alkoxy, R² is C1-C6 alkoxy, R⁶ is hydrogen and R⁷ is hydrogen.

In some embodiments of Formula I-E, X¹ is CH, X² is N, X³ is CH, R¹ is C1-C6 alkoxy and R² is C1-C6 alkoxy.

In some embodiments of Formula I-E, X¹ is CH, X² is N, X³ is CH, R¹ is C1-C6 alkoxy, R² is C1-C6 alkoxy, R⁶ is hydrogen and R⁷ is hydrogen.

Formula I-F

In some embodiments, the compound of Formula I has Formula I-F

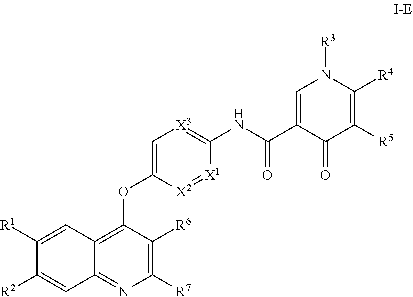

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is (C1-C6 alkoxy)C1-C6 alkyl-;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-F, X¹ is N, X² is CH and X³ is CH.

In some embodiments of Formula I-F, R¹ is C1-C6 alkoxy. In some embodiments, R¹ is methoxy.

In some embodiments of Formula I-F, R² is C1-C6 alkoxy. In some embodiments, R² is methoxy.

In some embodiments of Formula I-F, R¹ is C1-C6 alkoxy and R² is C1-C6 alkoxy.

In some embodiments of Formula I-F, R³ is methoxyethyl-.

In some embodiments of Formula I-F, R⁴ is hydrogen.

In some embodiments of Formula I-F, R⁵ is phenyl optionally substituted with one or two substituents independently selected from halogen, C1-C6 alkyl and C1-C6 alkoxy. In some embodiments of Formula I-F, R⁵ is phenyl substituted with one or two substituents independently selected from halogen, C1-C6 alkyl and C1-C6 alkoxy. In some embodiments of Formula I-F, R⁵ is phenyl optionally substituted with one or two substituents independently selected from fluoro, chloro, methyl and methoxy. In some embodiments of Formula I-F, R⁵ is phenyl substituted with one or two substituents independently selected from fluoro, chloro, methyl and methoxy. In some embodiments of Formula I-F, R⁵ is phenyl optionally substituted with halogen. In some embodiments of Formula I-F, R⁵ is phenyl substituted with halogen. In some embodiments of Formula I-F, R⁵ is phenyl optionally substituted with fluoro. In some embodiments of Formula I-F, R⁵ is phenyl substituted with fluoro.

In some embodiments of Formula I-F, R⁶ is hydrogen.

In some embodiments of Formula I-F, R⁷ is hydrogen.

In some embodiments of Formula I-F, R⁶ is hydrogen and R⁷ is hydrogen.

In some embodiments of Formula I-F, X¹ is N, X² is CH, X³ is CH, and R² is C1-C6 alkoxy.

In some embodiments of Formula I-F, X¹ is N, X² is CH, X³ is CH, R¹ is C1-C6 alkoxy and R² is C1-C6 alkoxy.

In some embodiments of Formula I-F, X¹ is N, X² is CH, X³ is CH, R¹ is C1-C6 alkoxy, R² is C1-C6 alkoxy, and R⁴ is hydrogen.

In some embodiments of Formula I-F, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl substituted with halogen.

In some embodiments of Formula I-F, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen.

Formula I-G

In some embodiments, the compound of Formula I has Formula I-G

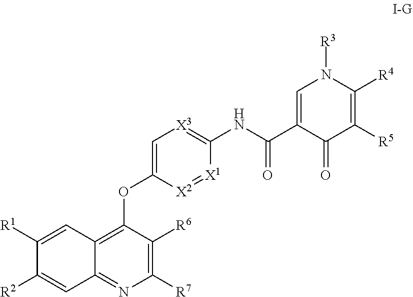

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is hydroxyC1-C6 alkyl-;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-G, X¹ is N, X² is CH and X³ is CH.

In some embodiments of Formula I-G, R¹ is C1-C6 alkoxy. In some embodiments, R¹ is methoxy.

In some embodiments of Formula I-G, R² is C1-C6 alkoxy. In some embodiments, R² is methoxy.

In some embodiments of Formula I-G, R¹ is C1-C6 alkoxy and R² is C1-C6 alkoxy.

In some embodiments of Formula I-G, R⁴ is hydrogen.

In some embodiments of Formula I-G, R⁵ is phenyl optionally substituted with one or two substituents independently selected from halogen, C1-C6 alkyl and C1-C6 alkoxy. In some embodiments of Formula I-G, R⁵ is phenyl substituted with one or two substituents independently selected from halogen, C1-C6 alkyl and C1-C6 alkoxy. In some embodiments of Formula I-G, R⁵ is phenyl optionally substituted with one or two substituents independently selected from fluoro, chloro, methyl and methoxy. In some embodiments of Formula I-G, R⁵ is phenyl substituted with one or two substituents independently selected from fluoro, chloro, methyl and methoxy. In some embodiments of Formula I-G, R⁵ is phenyl optionally substituted with one or two independently selected halogens. In some embodiments of Formula I-G, R⁵ is phenyl substituted with one or two independently selected halogens. In some embodiments of Formula I-H, R⁵ is phenyl optionally substituted with one or two independently selected halogens. In some embodiments of Formula I-H, R⁵ is phenyl substituted with one or two independently selected halogens. In some embodiments of Formula I-H, R⁵ is phenyl optionally substituted with halogen. In some embodiments of Formula I-H, R⁵ is phenyl substituted with halogen. In some embodiments, R⁵ is phenyl substituted with fluoro.

In some embodiments of Formula I-G, R⁶ is hydrogen.

In some embodiments of Formula I-G, R⁷ is hydrogen.

In some embodiments of Formula I-G, R⁶ is hydrogen and R⁷ is hydrogen.

In some embodiments of Formula I-G, X¹ is N, X² is CH, X³ is CH, and R² is C1-C6 alkoxy.

In some embodiments of Formula I-G, X¹ is N, X² is CH, X³ is CH, R¹ is C1-C6 alkoxy and R² is C1-C6 alkoxy.

In some embodiments of Formula I-G, X¹ is N, X² is CH, X³ is CH, R¹ is C1-C6 alkoxy, R² is C1-C6 alkoxy, and R⁴ is hydrogen.

In some embodiments of Formula I-G, X¹ is N, X² is CH, X³ is CH, R¹ is C1-C6 alkoxy, R² is C1-C6 alkoxy, R⁴ is hydrogen, and R⁵ is phenyl optionally substituted with halogen.

In some embodiments of Formula I-G, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen.

Formula I-H

In some embodiments, the compound of Formula I has Formula I-H

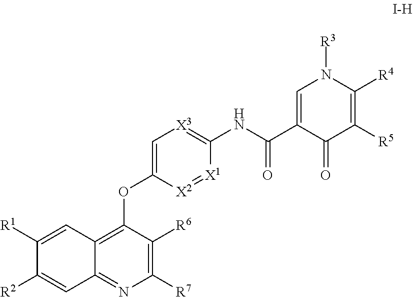

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is R^{a}R^{b}NC(═O)C1-C6 alkyl-;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-H, X¹ is N, X² is CH and X³ is CH.

In some embodiments of Formula I-H, R¹ is C1-C6 alkoxy. In some embodiments, R¹ is methoxy.

In some embodiments of Formula I-H, R² is C1-C6 alkoxy. In some embodiments, R² is methoxy.

In some embodiments of Formula I-H, R¹ is C1-C6 alkoxy and R² is C1-C6 alkoxy.

In some embodiments of Formula I-H, R⁴ is hydrogen.

In some embodiments of Formula I-H, R⁵ is phenyl optionally substituted with one or two substituents independently selected from halogen, C1-C6 alkyl and C1-C6 alkoxy. In some embodiments of Formula I-H, R⁵ is phenyl substituted with one or two substituents independently selected from halogen, C1-C6 alkyl and C1-C6 alkoxy. In some embodiments of Formula I-H, R⁵ is phenyl optionally substituted with one or two substituents independently selected from fluoro, chloro, methyl and methoxy. In some embodiments of Formula I-H, R⁵ is phenyl substituted with one or two substituents independently selected from fluoro, chloro, methyl and methoxy. In some embodiments of Formula I-H, R⁵ is phenyl optionally substituted with one or two independently selected halogens. In some embodiments of Formula I-H, R⁵ is phenyl substituted with one or two independently selected halogens. In some embodiments of Formula I-H, R⁵ is phenyl optionally substituted with one halogen. In some embodiments of Formula I-H, R⁵ is phenyl substituted with one halogen. In some such embodiments, R⁵ is phenyl substituted with fluoro.

In some embodiments of Formula I-H, R⁶ is hydrogen.

In some embodiments of Formula I-H, R⁷ is hydrogen.

In some embodiments of Formula I-H, R⁶ is hydrogen and R⁷ is hydrogen.

In some embodiments of Formula I-H, X¹ is N, X² is CH, X³ is CH, and R² is Cl-C6 alkoxy.

In some embodiments of Formula I-H, X¹ is N, X² is CH, X³ is CH, R¹ is C1-C6 alkoxy and R² is C1-C6 alkoxy.

In some embodiments of Formula I-H, X¹ is N, X² is CH, X³ is CH, R¹ is C1-C6 alkoxy, R² is C1-C6 alkoxy, and R⁴ is hydrogen.

In some embodiments of Formula I-H, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl optionally substituted with halogen.

In some embodiments of Formula I-H, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen. In some embodiments, R⁵ is phenyl substituted fluoro.

Formula I-I

In some embodiments, the compound of Formula I has Formula I-I

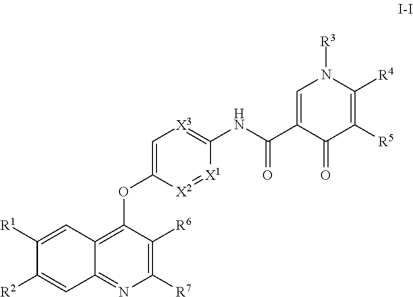

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is (R^{c}R^{d}N)C1-C6 alkyl-;

R^{c} and R^{d} are independently hydrogen or C1-C6 alkyl;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-I, X¹ is N, X² is CH and X³ is CH.

In some embodiments of Formula I-I, R¹ is C1-C6 alkoxy. In some embodiments, R¹ is methoxy.

In some embodiments of Formula I-I, R² is C1-C6 alkoxy. In some embodiments, R² is methoxy.

In some embodiments of Formula I-I, R¹ is C1-C6 alkoxy and R² is C1-C6 alkoxy.

In some embodiments of Formula I-I, R⁴ is hydrogen.

In some embodiments of Formula I-I, R⁵ is phenyl optionally substituted with one or two substituents independently selected from halogen, C1-C6 alkyl and C1-C6 alkoxy. In some embodiments of Formula I-I, R⁵ is phenyl substituted with one or two substituents independently selected from halogen, C1-C6 alkyl and C1-C6 alkoxy. In some embodiments of Formula I-I, R⁵ is phenyl optionally substituted with one or two substituents independently selected from fluoro, chloro, methyl and methoxy. In some embodiments of Formula I-I, R⁵ is phenyl substituted with one or two substituents independently selected from fluoro, chloro, methyl and methoxy. In some embodiments of Formula I-I, R⁵ is phenyl optionally substituted with one or two independently selected halogens. In some embodiments of Formula I-I, R⁵ is phenyl substituted with one or two independently selected halogens. In some embodiments of Formula I-I, R⁵ is phenyl optionally substituted with halogen. In some embodiments of Formula I-I, R⁵ is phenyl substituted with halogen. In some embodiments of Formula I-I, R⁵ is phenyl substituted with fluoro.

In some embodiments of Formula I-I, R⁶ is hydrogen.

In some embodiments of Formula I-I, R⁷ is hydrogen.

In some embodiments of Formula I-I, R⁶ is hydrogen and R⁷ is hydrogen.

In some embodiments of Formula I-I, X¹ is N, X² is CH, X³ is CH, and R² is C1-C6 alkoxy.

In some embodiments of Formula I-I, X¹ is N, X² is CH, X³ is CH, R¹ is C1-C6 alkoxy and R² is C1-C6 alkoxy.

In some embodiments of Formula I-I, X¹ is N, X² is CH, X³ is CH, R¹ is C1-C6 alkoxy, R² is C1-C6 alkoxy, and R⁴ is hydrogen.

In some embodiments of Formula I-I, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl substituted with halogen. In some embodiments, R⁵ is phenyl substituted with fluoro.

In some embodiments of Formula I-I, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen. In some embodiments, R⁵ is phenyl substituted with fluoro.

Formula I-J

In some embodiments, the compound of Formula I has Formula I-J

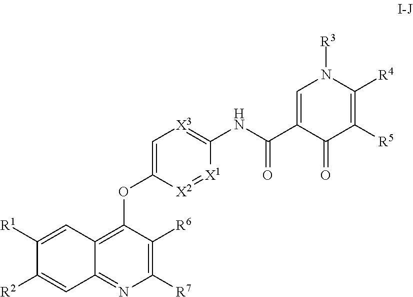

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is hetCyc²;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-J, X¹ is N, X² is CH and X³ is CH.

In some embodiments of Formula I-J, R¹ is C1-C6 alkoxy. In some embodiments, R¹ is methoxy.

In some embodiments of Formula I-J, R² is C1-C6 alkoxy. In some embodiments, R² is methoxy.

In some embodiments of Formula I-J, R¹ is C1-C6 alkoxy and R² is C1-C6 alkoxy.

In some embodiments of Formula I-J, R⁴ is hydrogen.

In some embodiments of Formula I-J, R⁵ is phenyl optionally substituted with one or two substituents independently selected from halogen, C1-C6 alkyl and C1-C6 alkoxy. In some embodiments of Formula I-J, R⁵ is phenyl substituted with one or two substituents independently selected from halogen, C1-C6 alkyl and C1-C6 alkoxy. In some embodiments of Formula I-J, R⁵ is phenyl optionally substituted with one or two substituents independently selected from fluoro, chloro, methyl and methoxy. In some embodiments of Formula I-J, R⁵ is phenyl substituted with one or two substituents independently selected from fluoro, chloro, methyl and methoxy. In some embodiments of Formula I-J, R⁵ is phenyl optionally substituted with one or two independently selected halogens. In some embodiments of Formula I-J, R⁵ is phenyl substituted with one or two independently selected halogens. In some embodiments of Formula I-J, R⁵ is phenyl optionally substituted with halogen. In some embodiments of Formula I-J, R⁵ is phenyl substituted with halogen. In some embodiments of Formula I-J, R⁵ is phenyl substituted with fluoro.

In some embodiments of Formula I-J, R⁶ is hydrogen.

In some embodiments of Formula I-J, R⁷ is hydrogen.

In some embodiments of Formula I-J, R⁶ is hydrogen and R⁷ is hydrogen.

In some embodiments of Formula I-J, X¹ is N, X² is CH, X³ is CH, and R² is C1-C6 alkoxy.

In some embodiments of Formula I-J, X¹ is N, X² is CH, X³ is CH, R¹ is C1-C6 alkoxy and R² is C1-C6 alkoxy.

In some embodiments of Formula I-J, X¹ is N, X² is CH, X³ is CH, R¹ is C1-C6 alkoxy, R² is C1-C6 alkoxy, and R⁴ is hydrogen.

In some embodiments of Formula I-J, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl substituted with halogen. In some embodiments, R⁵ is phenyl substituted with fluoro.

In some embodiments of Formula I-J, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen. In some embodiments, R⁵ is phenyl substituted with fluoro.

Formula I-K

In some embodiments, the compound of Formula I has Formula I-K

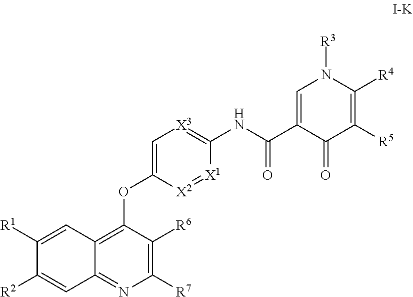

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is (hetCyc²)C1-C6 alkyl;

hetCyc² is a 5-6 membered saturated heterocyclic ring having 1-2 ring heteroatoms independently selected from N and O;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-K, X¹ is N, X² is CH and X³ is CH.

In some embodiments of Formula I-K, R¹ is C1-C6 alkoxy. In some embodiments, R¹ is methoxy.

In some embodiments of Formula I-K, R² is C1-C6 alkoxy. In some embodiments, R² is methoxy.

In some embodiments of Formula I-K, R¹ is C1-C6 alkoxy and R² is C1-C6 alkoxy.

In some embodiments of Formula I-K, R⁴ is hydrogen.

In some embodiments of Formula I-K, R⁵ is phenyl optionally substituted with one or two substituents independently selected from halogen, C1-C6 alkyl and C1-C6 alkoxy. In some embodiments of Formula I-K, R⁵ is phenyl substituted with one or two substituents independently selected from halogen, C1-C6 alkyl and C1-C6 alkoxy. In some embodiments of Formula I-K, R⁵ is phenyl optionally substituted with one or two substituents independently selected from fluoro, chloro, methyl and methoxy. In some embodiments of Formula I-K, R⁵ is phenyl substituted with one or two substituents independently selected from fluoro, chloro, methyl and methoxy. In some embodiments of Formula I-K, R⁵ is phenyl optionally substituted with one or two independently selected halogens. In some embodiments of Formula I-K, R⁵ is phenyl substituted with one or two independently selected halogens. In some embodiments of Formula I-K, R⁵ is phenyl optionally substituted with halogen. In some embodiments, R⁵ is phenyl substituted with halogen. In some embodiments, R⁵ is phenyl substituted with fluoro.

In some embodiments of Formula I-K, R⁶ is hydrogen.

In some embodiments of Formula I-K, R⁷ is hydrogen.

In some embodiments of Formula I-K, R⁶ is hydrogen and R⁷ is hydrogen.

In some embodiments of Formula I-K, X¹ is N, X² is CH, X³ is CH, and R² is C1-C6 alkoxy.

In some embodiments of Formula I-K, X¹ is N, X² is CH, X³ is CH, R¹ is C1-C6 alkoxy and R² is C1-C6 alkoxy.

In some embodiments of Formula I-K, X¹ is N, X² is CH, X³ is CH, R¹ is C1-C6 alkoxy, R² is C1-C6 alkoxy, and R⁴ is hydrogen.

In some embodiments of Formula I-K, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl substituted with halogen. In some embodiments, R⁵ is phenyl substituted with fluoro.

In some embodiments of Formula I-K, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen. In some embodiments, R⁵ is phenyl substituted with fluoro.

Formula I-L

In some embodiments, the compound of Formula I has Formula I-L

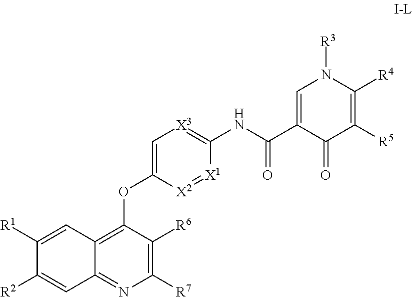

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy;

hetCyc¹ is a 5-6 membered saturated heterocyclic ring having 1-2 ring heteroatoms independently selected from N and O;

R³ is hydrogen;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula I-L, X¹ is N, X² is CH and X³ is CH.

In some embodiments of Formula I-L, R¹ is C1-C6 alkoxy. In some embodiments, R¹ is methoxy.

In some embodiments of Formula I-L, R² is C1-C6 alkoxy. In some embodiments, R² is methoxy.

In some embodiments of Formula I-L, R¹ is C1-C6 alkoxy and R² is C1-C6 alkoxy.

In some embodiments of Formula I-L, R⁴ is hydrogen.

In some embodiments of Formula I-L, R⁵ is phenyl optionally substituted with one or two substituents independently selected from halogen, C1-C6 alkyl and C1-C6 alkoxy. In some embodiments of Formula I-L, R⁵ is phenyl substituted with one or two substituents independently selected from halogen, C1-C6 alkyl and C1-C6 alkoxy. In some embodiments of Formula I-L, R⁵ is phenyl optionally substituted with one or two substituents independently selected from fluoro, chloro, methyl and methoxy. In some embodiments of Formula I-L, R⁵ is phenyl substituted with one or two substituents independently selected from fluoro, chloro, methyl and methoxy. In some embodiments of Formula I-L, R⁵ is phenyl optionally substituted with one or two independently selected halogens. In some embodiments of Formula I-L, R⁵ is phenyl substituted with one or two independently selected halogens. In some embodiments of Formula I-L, R⁵ is phenyl optionally substituted with halogen. In some embodiments, R⁵ is phenyl substituted with halogen. In some embodiments, R⁵ is phenyl substituted with fluoro.

In some embodiments of Formula I-L, R⁶ is hydrogen.

In some embodiments of Formula I-L, R⁷ is hydrogen.

In some embodiments of Formula I-L, R⁶ is hydrogen and R⁷ is hydrogen.

In some embodiments of Formula I-L, X¹ is N, X² is CH, X³ is CH, and R² is C1-C6 alkoxy.

In some embodiments of Formula I-L, X¹ is N, X² is CH, X³ is CH, R¹ is C1-C6 alkoxy and R² is C1-C6 alkoxy.

In some embodiments of Formula I-L, X¹ is N, X² is CH, X³ is CH, R¹ is C1-C6 alkoxy, R² is C1-C6 alkoxy, and R⁴ is hydrogen.

In some embodiments of Formula I-L, X¹ is N, X² is CH, X³ is CH, R¹ is C1-C6 alkoxy, R² is C1-C6 alkoxy, R⁴ is hydrogen, and R⁵ is phenyl optionally substituted with halogen. In some embodiments, R⁵ is phenyl substituted with halogen. In some embodiments, R⁵ is phenyl substituted with fluoro.

In some embodiments of Formula I-L, X¹ is N, X² is CH, X³ is CH, R¹ is C1-C6 alkoxy, R² is C1-C6 alkoxy, R⁴ is hydrogen, R⁵ is phenyl optionally substituted with halogen, R⁶ is hydrogen and R⁷ is hydrogen. In some embodiments, R⁵ is phenyl substituted with halogen. In some embodiments, R⁵ is phenyl substituted with fluoro.

In one embodiment, provided herein is a compound of Formula II

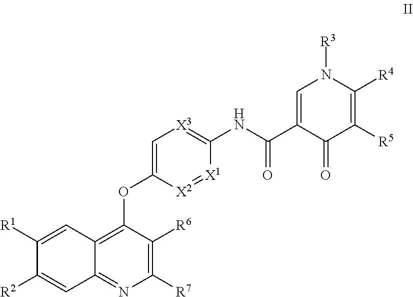

or pharmaceutically acceptable salts thereof, wherein:

X¹, X² and X³ are independently N or CH, wherein one or two of X¹, X² and X³ are N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is hydrogen, C1-C7 alkyl, (C1-C6 alkoxy)C1-C6 alkyl-, hydroxyC1-C6 alkyl-, R^{a}R^{b}Nc(═O)C1-C6 alkyl- or (R^{c}R^{d}N)C1-C6 alkyl-;

R^{a} and R^{b} are independently hydrogen or C1-C6 alkyl, or

R^{a} and R^{b} together with the nitrogen atom to which they are attached form a 5-6 membered saturated heterocyclic ring having one ring nitrogen atom and optionally having a second ring heteroatom selected from O and N;

R^{c} and R^{d} are independently hydrogen or C1-C6 alkyl;

R⁴ is hydrogen or C1-C6 alkyl;

R⁵ is phenyl optionally substituted with one to five substituents independently selected from halogen, C1-C6 alkyl and C1-C6 alkoxy;

R⁶ is hydrogen or CN; and

R⁷ is hydrogen or C1-C3 alkyl.

In some embodiments of Formula II, X¹ is N, X² is CH and X³ is CH.

In some embodiments of Formula II, X¹ is N, X² is CH and X³ is N.

In some embodiments of Formula II, X¹ is CH, X² is N and X³ is CH.

In some embodiments of Formula II, R¹ is hydrogen.

In some embodiments of Formula II, R¹ is C1-C6 alkoxy. In some such embodiments, R¹ is methoxy or ethoxy. In some such embodiments, R¹ is methoxy. In some such embodiments, R¹ is ethoxy.

In some embodiments of Formula II, R² is hydrogen.

In some embodiments of Formula II, R² is C1-C6 alkoxy. In some such embodiments, R² is methoxy or ethoxy. In some such embodiments, R² is methoxy. In some such embodiments, R² is ethoxy.

In some embodiments of Formula II, R² is fluoroC1-C6 alkoxy. In some such embodiments, R² is trifluoromethoxy.

In some embodiments of Formula II, R² is halogen. In some such embodiments, R² is fluoro.

In some embodiments of Formula II, R¹ is hydrogen and R² is C1-C6 alkoxy. In some such embodiments, R¹ is hydrogen and R² is ethoxy.

In some embodiments of Formula II, R¹ is hydrogen and R² is fluoroC1-C6 alkoxy. In some such embodiments, R¹ is hydrogen and R² is trifluoromethoxy.

In some embodiments of Formula II, R¹ is hydrogen and R² is halogen.

In some embodiments of Formula II, R¹ is hydrogen and R² is (hetCyc¹)C1-C6 alkoxy-. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is hydrogen and R² is (morpholin-1-yl)propoxy.

In some embodiments of Formula II, R¹ is C1-C6 alkoxy and R² is hydrogen. In some such embodiments, R¹ is methoxy and R² is hydrogen. In some such embodiments, R¹ is ethoxy and R² is hydrogen.

In some such embodiments of Formula II, R¹ is C1-C6 alkoxy and R² is C1-C6 alkoxy. In some such embodiments, R¹ is methoxy and R² is methoxy.

In some embodiments of Formula II, R¹ is C1-C6 alkoxy and R² is halogen. In some such embodiments, R¹ is methoxy and R² is halogen. In some such embodiments, R¹ is C1-C6 alkoxy and R² is fluoro. In some such embodiments, R¹ is methoxy and R² is fluoro.

In some embodiments of Formula II, R¹ is C1-C6 alkoxy and R² is (hetCyc¹)C1-C6 alkoxy. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is methoxy and R² is (morpholin-1-yl)propoxy.

In some embodiments of Formula II, R³ is hydrogen.

In some embodiments of Formula II, R³ is C1-C7 alkyl. In some such embodiments, R³is selected from methyl, ethyl, propyl, isopropyl, 1-isobutyl, pentan-3-yl, hetpan-4-yl and 1-isopentyl.

In some embodiments of Formula II, R³ is (C1-C6 alkoxy)C1-C6 alkyl-. In one such embodiment, R³ is (2-methoxy)ethyl.

In some embodiments of Formula II, R³ is hydroxyC1-C6 alkyl-. In one such embodiment, R³ is 2-hydroxy-2-methylpropyl.

In some embodiments of Formula II, R³ is R^{a}R^{b}NC(═O)C1-C6 alkyl-, wherein R^{a} and R^{b} are independently hydrogen or C1-C6 alkyl, or R^{a} and R^{b} together with the nitrogen atom to which they are attached form a 5-6 membered saturated heterocyclic ring having one ring nitrogen atom and optionally having a second ring heteroatom selected from O and N.

In some embodiments of Formula II when R³ is R^{a}R^{b}NC(═O)C1-C6 alkyl-, R^{a} and Rb are independently hydrogen or C1-C6 alkyl. In some such embodiments, R³ is (CH₃)₂NC(═O)CH₂— or CH₃NHC(═O)CH₂—.

In some embodiments of Formula II when R³ is R^{a}R^{b}NC(═O)C1-C6 alkyl-, R^{a} and R^{b} together with the nitrogen atom to which they are attached form a 5-6 membered saturated heterocyclic ring having one ring nitrogen atom and optionally having a second ring heteroatom selected from O and N. In some such embodiments, R³ is selected from:

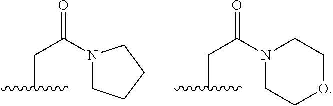 [SMILES: *CC(=O)N1CCCC1 ; *CC(=O)N1CCOCC1]

In some embodiments of Formula II, R³ is (R^{c}R^{d}N)C1-C6 alkyl-, wherein R^{c} and R^{d} are independently hydrogen or C1-C6 alkyl. In one such embodiment, R³ is dimethylaminopropyl.

In some embodiments of Formula II, R⁴ is hydrogen.

In some embodiments of Formula II, R⁴ is C1-C6 alkyl. In some such embodiments, R⁴ is methyl or isopropyl.

In some embodiments of Formula II, R⁵ is phenyl optionally substituted with one or two substituents independently selected from halogen, C1-C6 alkyl and C1-C6 alkoxy. In some such embodiments, R⁵ is phenyl substituted with one or two substituents independently selected from halogen, C1-C6 alkyl and C1-C6 alkoxy. In some such embodiments, R⁵ is phenyl optionally substituted with one or two substituents independently selected from fluoro, chloro, methyl and methoxy. In some such embodiments, R⁵ is phenyl substituted with one or two substituents independently selected from fluoro, chloro, methyl and methoxy. In some such embodiments, R⁵ is phenyl substituted with fluoro. In some such embodiments, R⁵ is selected from the following:

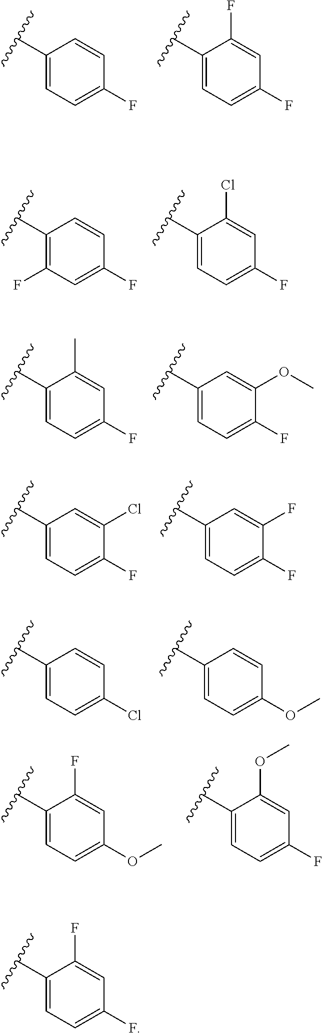

In some embodiments of Formula II, R⁶ is hydrogen.

In some embodiments of Formula II, R⁶ is CN.

In some embodiments of Formula II, R⁷ is hydrogen.

In some embodiments of Formula II, R⁷ is C1-C3 alkyl. In some such embodiments, R⁷ is methyl.

In some embodiments of Formula II, R⁶ is hydrogen and R⁷ is hydrogen.

In some embodiments of Formula II, R⁶ is hydrogen and R⁷ is C1-C3 alkyl. In some such embodiments, R⁶ is hydrogen and R⁷ is methyl.

In some embodiments of Formula II, R⁶ is CN and R⁷ is hydrogen.

Any of the aforementioned embodiments for Formula II may be combined with each other”

In some embodiments, provided herein are compounds of Formula III

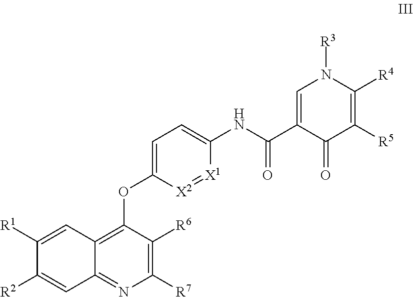

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoroC1-C6 alkoxy, halogen or (hetCyc¹)C1-C6 alkoxy-;

hetCyc¹ is a 5-6 membered saturated heterocyclic ring having 1-2 ring heteroatoms independently selected from N and O;

R³ is C1-C7 alkyl;

R⁴ is hydrogen or C1-C6 alkyl;

R⁵ is phenyl optionally substituted with one or two substituents independently selected from halogen, C1-C6 alkyl and C1-C6 alkoxy;

R⁶ is hydrogen or CN; and

R⁷ is hydrogen or C1-C3 alkyl.

In some embodiments of Formula III, X¹ is N and X² is CH.

In some embodiments of Formula III, X¹ is CH and X² is N.

In some embodiments of Formula III, R² is hydrogen.

In some embodiments of Formula III, R² is C1-C6 alkoxy. In some such embodiments, R² is methoxy or ethoxy. In some such embodiments, R² is methoxy. In some such embodiments, R² is ethoxy.

In some embodiments of Formula III, R² is fluoroC1-C6 alkoxy. In some such embodiments, R² is trifluoromethoxy.

In some embodiments of Formula III, R² is halogen. In some such embodiments, R² is fluoro.

In some embodiments of Formula III, R¹ is hydrogen and R² is C1-C6 alkoxy. In some such embodiments, R¹ is hydrogen and R² is ethoxy.

In some embodiments of Formula III, R¹ is hydrogen and R² is fluoroC1-C6 alkoxy. In some such embodiments, R¹ is hydrogen and R² is trifluoromethoxy.

In some embodiments of Formula III, R¹ is hydrogen and R² is halogen.

In some embodiments of Formula III, R¹ is hydrogen and R² is (hetCyc¹)C1-C6 alkoxy-. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is hydrogen and R² is (morpholin-1-yl)propoxy.

In some embodiments of Formula III, R¹ is C1-C6 alkoxy and R² is hydrogen. In some such embodiments, R¹ is methoxy and R² is hydrogen. In some such embodiments, R¹ is ethoxy and R² is hydrogen.

In some such embodiments of Formula III, R¹ is C1-C6 alkoxy and R² is C1-C6 alkoxy. In some such embodiments, R¹ is methoxy and R² is methoxy.

In some embodiments of Formula III, R¹ is C1-C6 alkoxy and R² is halogen. In some such embodiments, R¹ is methoxy and R² is halogen. In some such embodiments, R¹ is C1-C6 alkoxy and R² is fluoro. In some such embodiments, R¹ is methoxy and R² is fluoro.

In some embodiments of Formula III, R¹ is C1-C6 alkoxy and R² is (hetCyc¹)C1-C6 alkoxy. In some such embodiments, hetCyc¹ is morpholinyl. In some such embodiments, R² is (morpholin-1-yl)C1-C6 alkoxy. In some such embodiments, R² is (morpholin-1-yl)propoxy. In some such embodiments, R¹ is methoxy and R² is (morpholin-1-yl)propoxy.

In some embodiments of Formula III, R⁴ is hydrogen.

In some embodiments of Formula III, R⁴ is C1-C6 alkyl. In some such embodiments, R⁴ is methyl or isopropyl.

In some embodiments of Formula III, R⁵ is phenyl optionally substituted with one or two substituents independently selected from halogen, C1-C6 alkyl and C1-C6 alkoxy. In some such embodiments, R⁵ is phenyl substituted with one or two substituents independently selected from halogen, C1-C6 alkyl and C1-C6 alkoxy. In some such embodiments, R⁵ is phenyl optionally substituted with one or two substituents independently selected from fluoro, chloro, methyl and methoxy. In some such embodiments, R⁵ is phenyl substituted with one or two substituents independently selected from fluoro, chloro, methyl and methoxy. In some such embodiments, R⁵ is phenyl substituted with fluoro. In some such embodiments, R⁵ is selected from the following:

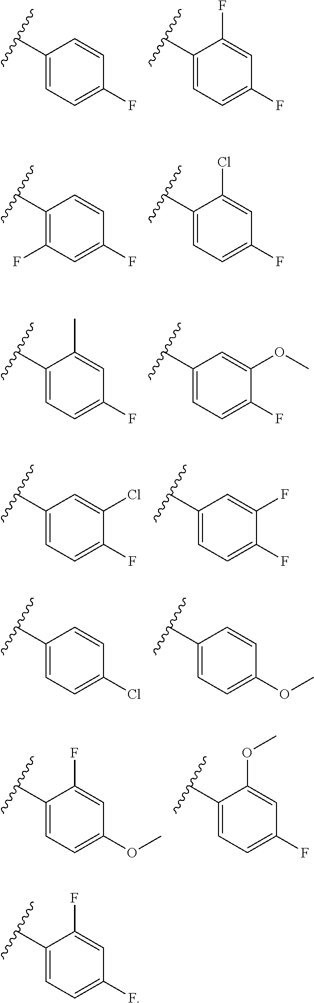

In some embodiments of Formula III, R⁶ is hydrogen.

In some embodiments of Formula III, R⁶ is CN.

In some embodiments of Formula III, R⁷ is hydrogen.

In some embodiments of Formula III, R⁷ is C1-C3 alkyl. In some such embodiments, R⁷ is methyl.

In some embodiments of Formula III, R⁶ is hydrogen and R⁷ is hydrogen.

In some embodiments of Formula III, R⁶ is hydrogen and R⁷ is C1-C3 alkyl. In some such embodiments, R⁶ is hydrogen and R⁷ is methyl.

In some embodiments of Formula III, R⁶ is CN and R⁷ is hydrogen.

Any of the aforementioned embodiments for Formula III may be combined with each other.

In some embodiments, a compound of Formula III is selected from a compound of Examples 1, 2, 3, 4, 5, 6, 10, 11, 16, 17, 18, 19, 20, 21, 27, 28, 29, 30, 31, 32, 33, 34, 35, 36, 37, 38, 39, 40, 41, 45, 48, 49, 50, 51, 52, 53, 56, 57, and 58, and pharmaceutically acceptable salts thereof.

In one embodiment, provided herein are compounds of Formula IV

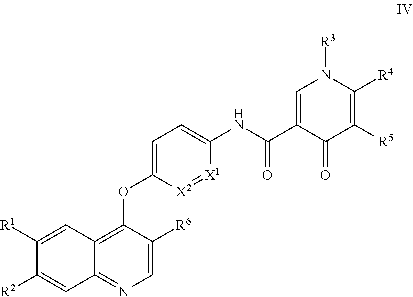

or pharmaceutically acceptable salts thereof, wherein:

X¹ is N and X² is CH, or X¹ is CH and X² is N;

R¹ is hydrogen or C1-C6 alkoxy;

R² is hydrogen, C1-C6 alkoxy, fluoroC1-C6 alkoxy or halogen;

R³ is C1-C6 alkyl;

R⁴ is hydrogen or methyl;

R⁵ is phenyl optionally substituted with one or two substituents independently selected from halogen, C1-C6 alkyl and C1-C6 alkoxy; and

R⁶ is hydrogen or CN.

In some embodiments of Formula IV, X¹ is N and X² is CH.

In some embodiments of Formula IV, X¹ is CH and X² is N.

In some embodiments of Formula IV, R¹ is hydrogen.

In some embodiments of Formula IV, R¹ is C1-C6 alkoxy. In some such embodiments, R¹ is methoxy or ethoxy. In one such embodiment, R¹ is methoxy. In one such embodiment, R² is ethoxy.

In some embodiments of Formula IV, R² is hydrogen.

In some embodiments of Formula IV, R² is C1-C6 alkoxy. In some such embodiments, R² is methoxy or ethoxy. In some such embodiments, R² is methoxy. In some such embodiments, R² is ethoxy.

In some embodiments of Formula IV, R² is fluoroC1-C6 alkoxy. In some such embodiments, R² is trifluoromethoxy.

In some embodiments of Formula IV, R² is halogen. In some such embodiments, R² is fluoro.

In some embodiments of Formula IV, R¹ is hydrogen and R² is C1-C6 alkoxy. In some such embodiments, R¹ is hydrogen and R² is ethoxy.

In some embodiments of Formula IV, R¹ is hydrogen and R² is fluoroC1-C6 alkoxy. In some such embodiments, R¹ is hydrogen and R² is trifluoromethoxy.

In some embodiments of Formula IV, R¹ is hydrogen and R² is halogen.

In some embodiments of Formula IV, R¹ is C1-C6 alkoxy and R² is hydrogen. In some such embodiments, R¹ is methoxy and R² is hydrogen. In some such embodiments, R¹ is ethoxy and R² is hydrogen.

In some such embodiments of Formula IV, R¹ is C1-C6 alkoxy and R² is C1-C6 alkoxy. In some such embodiments, R¹ is methoxy and R² is methoxy.

In some embodiments of Formula IV, R¹ is C1-C6 alkoxy and R² is halogen. In some such embodiments, R¹ is methoxy and R² is halogen. In some such embodiments, R¹ is C1-C6 alkoxy and R² is fluoro. In some such embodiments, R¹ is methoxy and R² is fluoro.

In some embodiments of Formula IV, R⁴ is hydrogen.

In some embodiments of Formula IV, R⁴ is C1-C6 alkyl. In some such embodiments, R⁴ is methyl or isopropyl.

In some embodiments of Formula IV, R⁵ is phenyl optionally substituted with one or two substituents independently selected from halogen, C1-C6 alkyl and C1-C6 alkoxy. In some such embodiments, R⁵ is phenyl substituted with one or two substituents independently selected from halogen, C1-C6 alkyl and C1-C6 alkoxy. In some such embodiments, R⁵ is phenyl optionally substituted with one or two substituents independently selected from fluoro, chloro, methyl and methoxy. In some such embodiments, R⁵ is phenyl substituted with one or two substituents independently selected from fluoro, chloro, methyl and methoxy. In some such embodiments, R⁵ is phenyl substituted with fluoro. In some such embodiments, R⁵ is selected from the following:

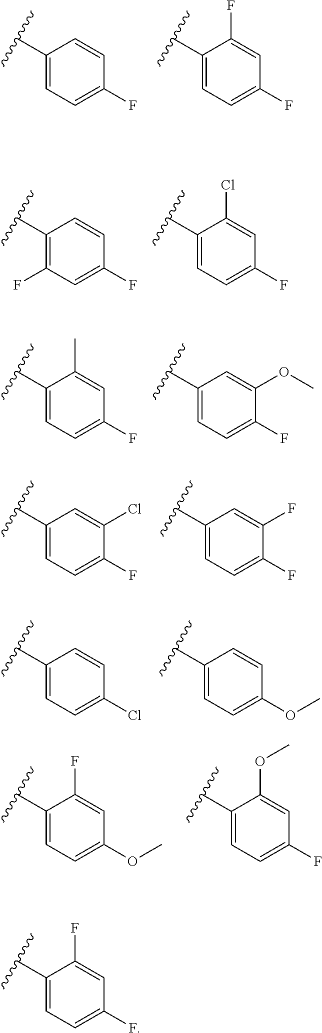

In some embodiments of Formula IV, R⁶ is hydrogen.

In some embodiments of Formula IV, R⁶ is CN.

In some embodiments of Formula IV, R⁷ is hydrogen.

In some embodiments of Formula IV, R⁷ is C1-C3 alkyl. In some such embodiments, R⁷ is methyl.

In some embodiments of Formula IV, R⁶ is hydrogen and R⁷ is hydrogen.

In some embodiments of Formula IV, R⁶ is hydrogen and R⁷ is C1-C3 alkyl. In some such embodiments, R⁶ is hydrogen and R⁷ is methyl.

In some embodiments of Formula IV, R⁶ is CN and R⁷ is hydrogen.

Any of the aforementioned embodiments for Formula IV may be combined with each other.

In one embodiment, a compound of Formula IV is selected from a compound of Example 1, 4, 5, 16, 18, 19, 21, 28, 30, 31, 33, 35, 39, 41, 48, 49, 51, 52, 57 and 58, and pharmaceutically acceptable salts thereof.

It will be appreciated that certain compounds provided herein may contain one or more centers of asymmetry and may therefore be prepared and isolated in a mixture of isomers such as a racemic mixture, or in an enantiomerically pure form.

The compounds of Formula I, II, Ill and IV may exist in the form of pharmaceutically acceptable salts such as, e.g., acid addition salts and base addition salts of the compounds of one of the formulae provided herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein. For example, the compounds of the invention that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

It will further be appreciated that the compounds of Formula I, II, Ill and IV or their salts may be isolated in the form of solvates, and accordingly that any such solvate is included within the scope of the present invention. For example, compounds of Formula I, II, Ill and IV and salts thereof can exist in unsolvated as well as solvated forms with pharmaceutically acceptable solvents such as water, ethanol, and the like.

In some embodiments, the compounds of Formula I include the compounds of Examples 1-58 and pharmaceutically acceptable salts thereof. In some embodiments, the compounds of Examples 1-58 are in the free base form.

In some embodiments, the compounds of Formula II include the compounds of Examples 1-7, 9-11 and 13-58 and pharmaceutically acceptable salts thereof. In some embodiments, the compounds of Examples 1-7, 9-11 and 13-58 are in the free base form.

In some embodiments, the compounds of Formula III include the compounds of Examples 1, 2, 3, 4, 5, 6, 10, 11, 16, 17, 18, 19, 20, 21, 27, 28, 29, 30, 31, 32, 33, 34, 35, 36, 37, 38, 39, 40, 41, 45, 48, 49, 50, 51, 52, 53, 56, 57, and 58. In some embodiments, the compounds of Examples 1, 2, 3, 4, 5, 6, 10, 11, 16, 17, 18, 19, 20, 21, 27, 28, 29, 30, 31, 32, 33, 34, 35, 36, 37, 38, 39, 40, 41, 45, 48, 49, 50, 51, 52, 53, 56, 57, and 58 are in the free base form.

In some embodiments, the compounds of Formula IV include the compounds of Examples 1, 4, 5, 16, 18, 19, 21, 28, 30, 31, 33, 35, 39, 41, 48, 49, 51, 52, 57, and 58, and pharmaceutically acceptable salts thereof. In some embodiments, the compounds of Examples 1, 4, 5, 16, 18, 19, 21, 28, 30, 31, 33, 35, 39, 41, 48, 49, 51, 52, 57, and 58 are in the free base form.

In one embodiment, the compound of Formula I is a compound of Example No. 1, 2, 3, 4, 7, 18, 19, 20, 27, 28, 29, 32, 33, 44, 46, 48, 55, 56, or 58, or a pharmaceutically acceptable salt or solvate thereof.

In one embodiment, the compound of Formula I is a compound of Example No. 1, or a pharmaceutically acceptable salt or solvate thereof.

In one embodiment, the compound of Formula I is a compound of Example No. 2, or a pharmaceutically acceptable salt or solvate thereof.

In one embodiment, the compound of Formula I is a compound of Example No. 3, or a pharmaceutically acceptable salt or solvate thereof.

In one embodiment, the compound of Formula I is a compound of Example No. 4, or a pharmaceutically acceptable salt or solvate thereof.

In one embodiment, the compound of Formula I is a compound of Example No. 7, or a pharmaceutically acceptable salt or solvate thereof.

In one embodiment, the compound of Formula I is a compound of Example No. 18, or a pharmaceutically acceptable salt or solvate thereof.

In one embodiment, the compound of Formula I is a compound of Example No. 19, or a pharmaceutically acceptable salt or solvate thereof.

In one embodiment, the compound of Formula I is a compound of Example No. 20, or a pharmaceutically acceptable salt or solvate thereof.

In one embodiment, the compound of Formula I is a compound of Example No. 27, or a pharmaceutically acceptable salt or solvate thereof.

In one embodiment, the compound of Formula I is a compound of Example No. 28, or a pharmaceutically acceptable salt or solvate thereof.

In one embodiment, the compound of Formula I is a compound of Example No. 29, or a pharmaceutically acceptable salt or solvate thereof.

In one embodiment, the compound of Formula I is a compound of Example No. 32, or a pharmaceutically acceptable salt or solvate thereof.

In one embodiment, the compound of Formula I is a compound of Example No. 33, or a pharmaceutically acceptable salt or solvate thereof.

In one embodiment, the compound of Formula I is a compound of Example No. 44, or a pharmaceutically acceptable salt or solvate thereof.

In one embodiment, the compound of Formula I is a compound of Example No. 46, or a pharmaceutically acceptable salt or solvate thereof.

In one embodiment, the compound of Formula I is a compound of Example No. 48, or a pharmaceutically acceptable salt or solvate thereof.

In one embodiment, the compound of Formula I is a compound of Example No. 55, or a pharmaceutically acceptable salt or solvate thereof.

In one embodiment, the compound of Formula I is a compound of Example No. 56, or a pharmaceutically acceptable salt or solvate thereof.

In one embodiment, the compound of Formula I is a compound of Example No. 58, or a pharmaceutically acceptable salt or solvate thereof. The term “pharmaceutically acceptable” indicates that the compound, or salt or composition thereof is compatible chemically and/or toxicologically with the other ingredients comprising a formulation and/or the patient being treated therewith.

Compounds provided herein may also contain unnatural proportions of atomic isotopes at one or more of the atoms that constitute such compounds. That is, an atom, in particular when mentioned in relation to a compound according to Formula I, II, III or IV comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O. ¹⁸O or mixtures thereof; and when fluoro is mentioned, it is understood to refer to ¹⁸F, ¹⁹F or mixtures thereof. The compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as additional anticancer agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present invention.

For illustrative purposes, Schemes 1-5 show general methods for preparing compounds provided herein as well as methods for preparing key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

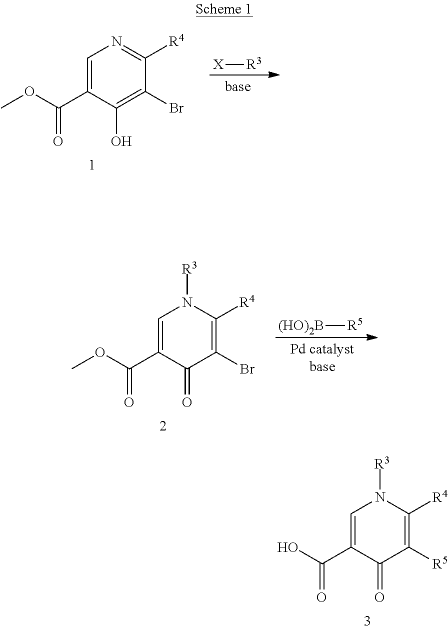

Scheme 1 shows a general scheme for the synthesis of intermediate 3, wherein R⁴ is hydrogen and R³ and R⁵ are as defined for Formula I, which is useful for preparing compounds of Formula I. Commercially available compound 1, wherein R⁴ is hydrogen, may be reacted with a reagent having the Formula X-R³ wherein X is a halogen such as iodo and R³ is C1-C7 alkyl, (C1-C6 alkoxy)C1-C6 alkyl-, hydroxyC1-C6 alkyl-, R^{a}R^{b}Nc(═O)C1-C6 alkyl-, (R^{c}R^{d}N)C1-C6 alkyl-, hetCyc² or (hetCyc²)C1-C6 alkyl-, in the presence of a base such as an alkaline carbonate base, for example cesium carbonate, to provide compound 2. Compound 2 may be reacted with a boronic acid having the Formula (HO)₂B-R⁵, wherein R⁵ is as defined for Formula I, using standard palladium-catalyzed cross-coupling reaction conditions, e.g., Suzuki coupling reaction conditions (for example, a palladium catalyst and optionally a ligand in the presence of an inorganic base, for example, Pd(PPh₃)₄ and Na₂CO₃ in dioxane at elevated temperatures), to provide compound 3.

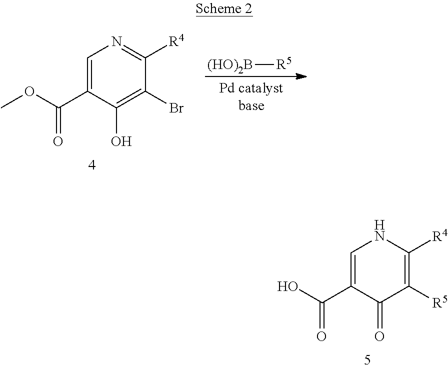

Scheme 2 shows a general scheme for the synthesis of intermediate 5, wherein R³ is hydrogen, R⁴ is hydrogen, and R⁵ is as defined for Formula I, which is useful for preparing compounds of Formula I. Commercially available compound 4, wherein R⁴ is hydrogen, may be reacted with a boronic acid having the Formula (HO)₂B—R⁵, wherein R⁵ is as defined for Formula I, using standard palladium-catalyzed cross-coupling reaction conditions, e.g., Suzuki coupling reaction conditions (for example, a palladium catalyst and optionally a ligand in the presence of an inorganic base, for example, Pd(PPh₃)₄ and Na₂CO₃ in dioxane at elevated temperatures), to provide compound 5.

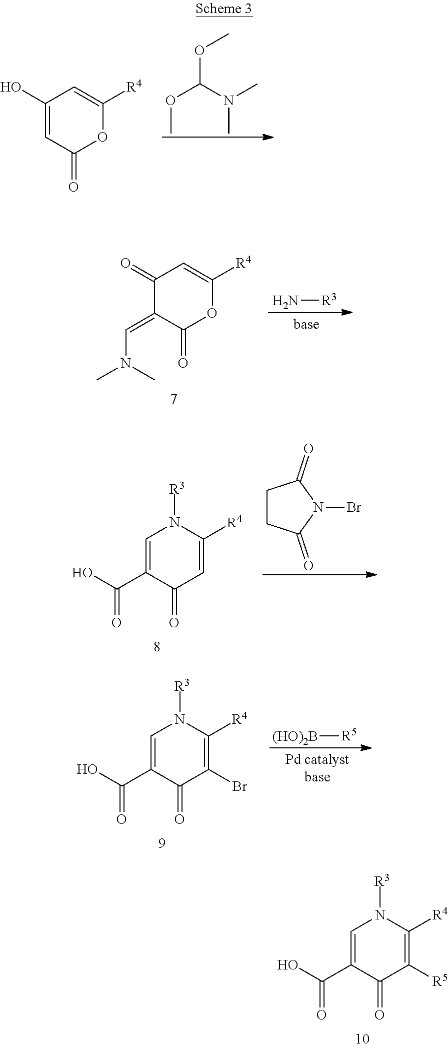

Scheme 3 shows a general scheme for the synthesis of intermediate 10, wherein R³, R⁴ and R⁵ are as defined for Formula I, which is useful for preparing compounds of Formula I. Commercially available compound 6, wherein R⁴ is as defined for Formula I, may be reacted with 1,1-dimethoxy-N,N-dimethylmethanamine to provide compound 7. Compound 7 may be reacted with an amine reagent having the Formula H₂N—R³, wherein R³ is as defined for Formula I, in the presence of a strong base such as sodium t-butoxide to provide compound 8. Compound 8 may be reacted with N-bromosuccinimide to provide compound 9. Compound 9 may be reacted with a boronic acid having the Formula (HO)₂B—R⁵, wherein R⁵ is as defined for Formula I, using standard palladium-catalyzed cross-coupling reaction conditions, e.g., Suzuki coupling reaction conditions (for example, a palladium catalyst and optionally a ligand in the presence of an inorganic base, for example, Pd(PPh₃)₄ and Na₂CO₃ in dioxane at elevated temperatures), to provide compound 10.

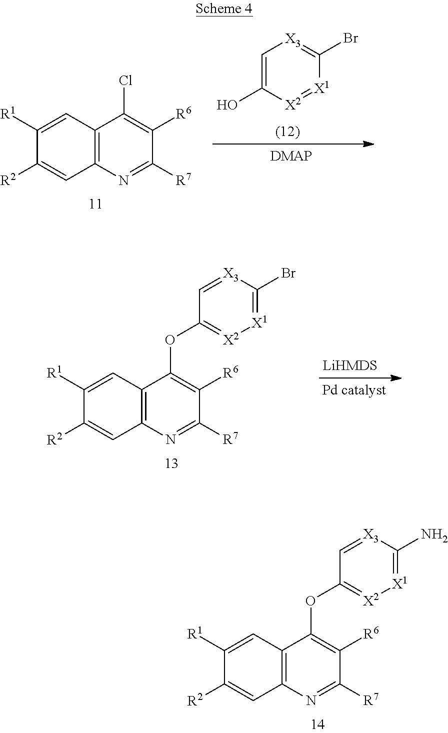

Scheme 4 shows a general scheme for the synthesis of intermediate 14, wherein R¹, R², R⁶, R⁷, X¹, X² and X³ are as defined for Formula I, which is useful for preparing compounds of Formula I. Commercially available compound 11, wherein R¹, R², R⁶, and R⁷ are as defined for Formula I, may be reacted with a commercially available reagent having the Formula 12 wherein X¹, X² and X³ are as defined for Formula I, in the presence of a catalytic amount of dimethylaminopyridine, to provide compound 13. Compound 13 may be treated with lithium hexamethyldisilazide in the presence of a palladium catalyst (e.g., Pd₂dba₃) in the presence of a ligand (e.g., X-Phos) to provide compound 14.

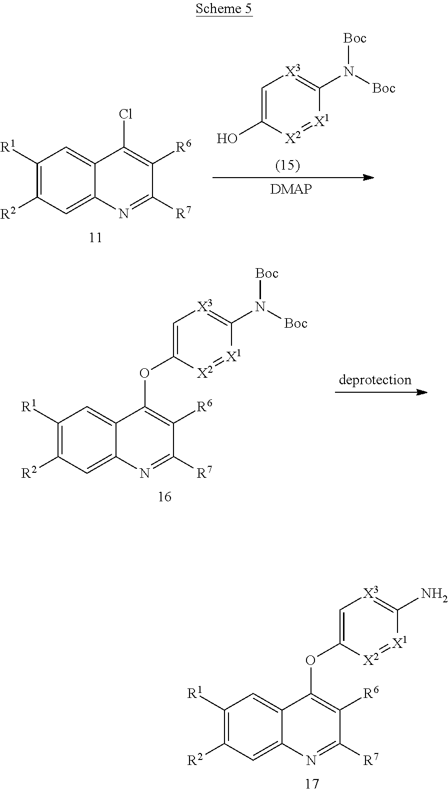

Scheme 5 shows an alternative general scheme for the synthesis of intermediate 17, wherein R¹, R², R⁶, R⁷, X¹, X² and X³ are as defined for Formula I, which is useful for preparing compounds of Formula I. Commercially available compound 11, wherein R¹, R², R⁶, and R⁷ are as defined for Formula I, may be reacted with the commercially available bis-Boc protected reagent 15, wherein X¹, X² and X³ are as defined for Formula I, at elevated temperature in the presence of a catalytic amount of dimethylaminopyridine to provide compound 16. Removal of the Boc protecting groups under standard reaction conditions (e.g., in the presence of trifluoroacetic acid) provides compound 17.

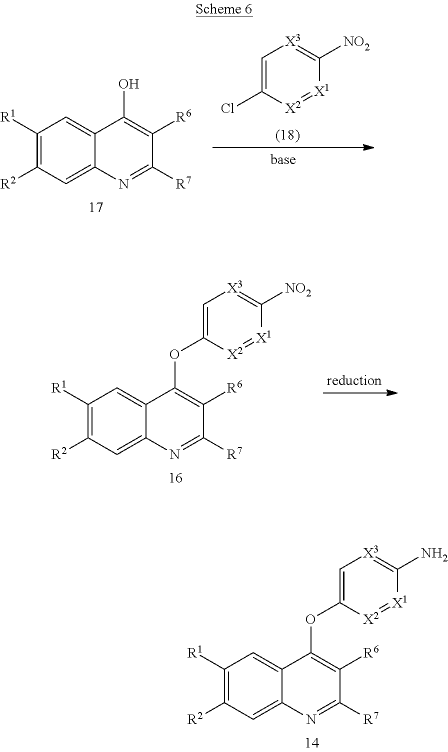

Scheme 6 shows an alternative general scheme for the synthesis of intermediate 14, wherein R¹, R², R⁶, R⁷, X¹, X² and X³ are as defined for Formula I, which is useful for preparing compounds of Formula I. Commercially available compound 11, wherein R¹, R², R⁶, and R⁷ are as defined for Formula I, may be reacted with the commercially available compound 18, wherein X′, X² and X³ are as defined for Formula I, in the presence of a base (e.g., an alkaline carbonate base, e.g., Cs₂CO₃), to provide compound 19. Reduction of the nitro group of compound 19 under standard reaction conditions (e.g., Zn and ammonium chloride) provides compound 14.

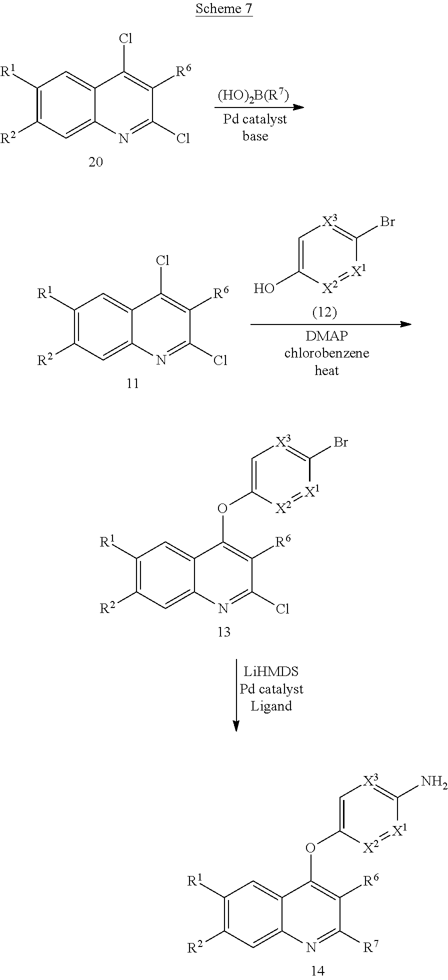

Scheme 7 shows an alternative general scheme for the synthesis of intermediate 14, wherein R¹, R², R⁶, R⁷, X¹, X² and X³ are as defined for Formula I, which is useful for preparing compounds of Formula I. Commercially available compound 11, wherein R¹, R², and R⁶ are as defined for Formula I, may be reacted with a commercially available boronic acid reagent having the Formula (HO)₂B(R⁷) wherein R⁷ is as defined for Formula I, using standard palladium-catalyzed cross-coupling reaction conditions, e.g., Suzuki coupling reaction conditions (for example, a palladium catalyst and optionally a ligand in the presence of an inorganic base, for example, Pd(PPh₃)₄ and Na₂CO₃ in dioxane at elevated temperatures), to provide compound 11. Compound 11 may be reacted with a commercially available reagent having the Formula 12 wherein X¹, X² and X³ are as defined for Formula I, in the presence of a catalytic amount of dimethylaminopyridine, to provide compound 13. Compound 13 may be treated with lithium hexamethyldisilazide in the presence of a palladium catalyst (e.g., Pd₂dba₃) in the presence of a ligand (e.g., X-Phos) to provide compound 14.

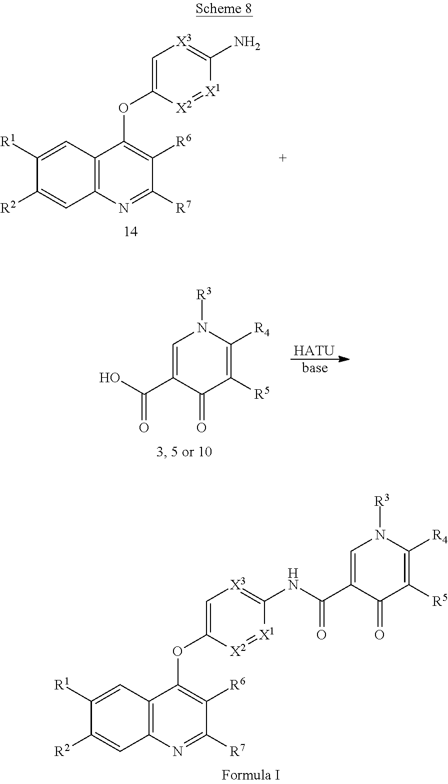

Scheme 8 shows a general scheme for the synthesis of compounds of Formula I. Compound 14, wherein R¹, R², R⁶, R⁷, X¹, X² and X³ are as defined for Formula I, prepared for example according to Scheme 4, 5, 6 or 7, may be coupled with compound 3 (wherein R⁴ is hydrogen and R³ and R⁵ are as defined for Formula I), compound 5 (wherein R³ is hydrogen, R⁴ is hydrogen, and R⁵ is as defined for Formula I) or compound 10 (wherein R³, R⁴ and R⁵ are as defined for Formula I) in the presence of 1-[bis(dimethylamino)methylene]-1H-1,2,3-triazolo[4,5-b]pyridinium 3-oxide hexafluorophosphate (HATU) and an amine base such as diisopropylethylamine, to provide a compound of Formula I.

In some embodiments, provided herein is a process for preparing a compound of Formula I, comprising:

reacting a compound having the Formula:

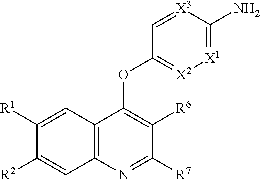

wherein R¹, R², R⁶, R⁷, X¹, X² and X³ are as defined for Formula I, with a compound having the Formula

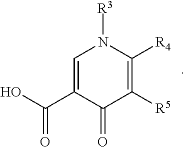

wherein R³, R⁴ and R⁵ are as defined for Formula I, in the presence of 1-[bis(dimethylamino)methylene]-1H-1,2,3-triazolo[4,5-b]pyridinium 3-oxide hexafluorophosphate and an amine base.

Compounds of Formula I or pharmaceutically acceptable salts thereof can modulate or inhibit the activity of one or more TAM kinases. The ability of compounds of Formula I to act as inhibitors of one or more TAM kinases may be demonstrated by the assays described in Examples A, B and C. IC₅₀ values are shown in Table 7.

Compounds of Formula I or pharmaceutically acceptable salts thereof can modulate or inhibit the activity of c-Met kinase. The ability of compounds of Formula Ito act as inhibitors of wild type and certain mutant c-Met kinases may be demonstrated by the assay described in Example D.

As used herein, the term “a TAM kinase” refers to one, two or all three of the TAM receptor tyrosine kinases, i.e., TYRO3, AXL and MER.

As used herein, the term “a TAM kinase inhibitor” refers to any compound exhibiting inhibition activity against one, two or all three of the TAM receptor kinases, i.e., the compounds exhibit inhibitory activity against AXL and/or MER and/or TYRO3.

As used herein, the term “a c-Met kinase inhibitor” refers to any compound exhibiting inhibitory activity against wild type and certain mutant c-Met kinases. In one embodiment, the term “a c-met kinase inhibitor” refers to any compound exhibiting inhibitory activity against wild type c-Met kinase or a mutant c-Met kinase selected from De114, D1228H, D1228N, F12001, L1195V, Y1230C, Y1230H and Y1230S.

In some embodiments, compounds of Formula I, II, III and IV or pharmaceutically acceptable salts thereof have inhibitory activity against AXL. In some embodiments, compounds of Formula I, II, III, and IV or pharmaceutically acceptable salts thereof have inhibitory activity against MER. In some embodiments, a compound of Formula I, II, III and IV has inhibitory activity against AXL and MER. In some embodiments, compounds of Formula I, II, Ill and IV or pharmaceutically acceptable salts thereof have inhibitory activity against AXL, MER and TYRO3. In some embodiments, a compound of Formula I, II, Ill and IV or pharmaceutically acceptable salts thereof has inhibitory activity against c-Met kinase. In some embodiments, a compound of Formula I, II, Ill and IV or pharmaceutically acceptable salts thereof has inhibitory activity against one or more receptor tyrosine kinases selected from AXL, MER, TYRO3, and c-Met. In some embodiments, a compound of Formula I, II, III and IV or pharmaceutically acceptable salts thereof has inhibitory activity against a c-Met kinase that does not include amino acids encoded by exon 14. In some embodiments, a compound of Formula I, II, Ill and IV or pharmaceutically acceptable salts thereof has inhibitory activity against a mutated c-Met (e.g., any of the examples of mutated c-Met proteins described herein or known in the art) (e.g., a mutation in c-Met that causes resistance to a Type 1 c-Met inhibitor).

In some embodiments, compounds of Formula I, II, Ill and IV or pharmaceutically acceptable salts thereof exhibit inhibition activity (IC₅₀) against a TAM kinase and/or c-Met of less than about 300 nM, less than about 250 nM, less than about 200 nM, less than about 150 nM, less than about 100 nM, less than about 50 nM, less than about 25 nM, less than about 10 nM, or less than about 1 nM as measured in an assay as described herein. In some embodiments, compounds of Formula I, II, Ill and IV or pharmaceutically acceptable salts thereof exhibit inhibition activity (IC₅₀) against a TAM kinase and/or c-Met of less than about 25 nM, less than about 10 nM, less than about 5 nM, or less than about 1 nM as measured in an assay as provided herein.

In some embodiments, exemplary compounds of Formula I, II, Ill and IV or pharmaceutically acceptable salts thereof exhibit inhibition activity (IC₅₀) against AXL of less than about 50 nM, less than about 25 nM, less than about 10 nM, or less than about 1 nM as measured in an assay as described herein.

In some embodiments, compounds of Formula I, II, Ill and IV or pharmaceutically acceptable salts thereof exhibit inhibition activity (IC₅₀) against MER of less than about 100 nM, less than about 75 nM, less than about 50 nM, less than about 25 nM, or less than about 10 nM, as measured in an assay as described herein.

In some embodiments, compounds of Formula I, II, Ill and IV or pharmaceutically acceptable salts thereof exhibit inhibition activity (IC₅₀) against TYRO3 of less than about 300 nM, less than about 250 nM, less than about 200 nM, less than about 100 nM, less than about 50 nM, less than about 25 nM, or less than about 10 nM, as measured in an assay as described herein.

In one embodiment, compounds of Formula I, II, Ill and IV or pharmaceutically acceptable salts thereof exhibit inhibition activity (IC₅₀) against c-Met of less than about 1000 nM, less than about 750 nM, less than about 500 nM, less than about 250 nM, less than about 200 nM, less than about 100 nM, less than about 50 nM, less about 25 nM, less than about 10 nM, or less than about 1 nM as measured in an assay as described herein.

In some embodiments, provided herein is a method for inhibiting AXL kinase, which comprises contacting the AXL kinase with compound of Formula I, II, Ill and IV or a pharmaceutically acceptable salt thereof.

In some embodiments, provided herein is a method for inhibiting MER kinase, which comprises contacting the MER kinase with compound of Formula I, or a pharmaceutically acceptable salt thereof.

In some embodiments, provided herein is a method for inhibiting TYRO3 kinase, which comprises contacting the TYRO3 kinase with compound of Formula I, or a pharmaceutically acceptable salt thereof.

In some embodiments, provided herein is a method for inhibiting c-Met kinase (e.g., any of the exemplary c-Met kinases described herein), which comprises contacting the c-Met kinase with compound of Formula I, II, Ill and IV or a pharmaceutically acceptable salt thereof.

Compounds of Formula I, II, Ill and IV or pharmaceutically acceptable salts thereof are useful in the treatment of various diseases associated with increased (e.g., at least 1%, at least 2%, at least 4%, at least 6%, at least 8%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, or at least 300%) expression, level, and/or activity of one or more of the TAM kinases and/or c-Met kinase (e.g., in a cancer cell or in an immune cell) (e.g., as compared to a control, e.g., a non-cancerous tissue or cell, or a corresponding tissue or cell from a control subject that does not have cancer). In some embodiments, compounds of Formula I, II, Ill and IV or pharmaceutically acceptable salts thereof are useful in treating or preventing proliferative disorders such as cancers. In some embodiments, tumors with an activating mutation (e.g., a point mutation or a chromosomal translocation) in a gene encoding a receptor tyrosine kinase and/or upregulation of the expression of a receptor tyrosine kinase (e.g., any of the TAM kinases or c-Met kinase described herein) may be particularly sensitive to compounds of Formula I, II, Ill and IV. In one embodiment, tumors with a mutation in a MET gene that results in exon 14 skipping during mRNA splicing are sensitive to compounds of Formula I, II, Ill and IV. In one embodiment, tumors having a mutation in a MET gene that results in expression of a c-Met protein having resistance to a Type I c-Met inhibitor are sensitive to compounds of Formula I, II, Ill and IV.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a TAM-associated disease or disorder (e.g., a TAM-associated cancer) and/or has been identified or diagnosed as having a c-Met-associated disease or disorder (e.g., a c-Met-associated cancer) (e.g., as determined using a regulatory agency-approved, e.g., FDA-approved, assay or kit). In some embodiments, the subject has been identified or diagnosed as having a cancer associated with one or more TAM kinases and/or c-met kinase (e.g., a TAK-associated cancer) (e.g., as determined using a regulatory agency-approved, e.g., FDA-approved, assay or kit). In some embodiments, the subject has a tumor that is associated with one or more TAM kinases and/or c-Met kinase (e.g., an increase in the expression, level, and/or activity of one or more TAM kinases and/or c-Met kinase in a cell (e.g., a cancer cell or an immune cell) as compared to a control, e.g., a non-cancerous tissue or a corresponding tissue from a control subject that does not have cancer) (e.g., as determined using a regulatory agency-approved assay or kit). In some embodiments, the subject is suspected of having a TAM-associated cancer and/or a c-Met-associated cancer. In some embodiments, the subject has a clinical record indicating that the subject has a tumor is associated with one or more TAM kinases (e.g., a TAM-associated cancer) and/or c-Met kinase (and optionally the clinical record indicates that the subject should be treated with any of the compositions provided herein). In some embodiments, the patient is a pediatric patient.

The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The phrase “therapeutically effective amount” means an amount of compound that, when administered to a patient in need of such treatment, is sufficient to (i) treat a TAM kinase-associated disease or disorder (e.g., a TAM-associated cancer) and/or a c-Met kinase-associated disease or disorder (e.g., a MET-associated cancer), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula I, II, Ill or IV that will correspond to such an amount will vary depending upon factors such as the particular compound, disease condition and its severity, the identity (e.g., weight) of the patient in need of treatment, but can nevertheless be routinely determined by one skilled in the art.

The term “regulatory agency” refers to a country's agency for the approval of the medical use of pharmaceutical agents with the country. For example, a non-limiting example of a regulatory agency is the U.S. Food and Drug Administration (FDA).

The term “TAM-associated disease or disorder” as used herein refers to diseases or disorders associated with or having increased expression and/or activity of one or more of the TAM kinases in a cell (e.g., a cancer cell or an immune cell) (e.g., as compared to a control, e.g., a non-cancerous tissue or cell, or a corresponding tissue or cell from a control subject that does not have cancer) and/or where activation of a TAM kinase expressed on non-cancer cells contributes to disease. Non-limiting examples of a TAM-associated disease or disorder include, for example, cancer (a TAM-associated cancer), e.g., any of the cancers described herein. In some embodiments, the disease is a cancer that overexpresses one or more TAM kinases after treatment with at least one additional anticancer agent (e.g., one or more of any of the additional anticancer agents described herein), e.g., a kinase-targeted therapeutic agent and/or a chemotherapeutic agent as described herein). In some embodiments, the disease is associated with signaling through one or more TAM kinases expressed in cells of the immune system (e.g., immune cells selected from the group of tumor-associated macrophages, natural killer (NK) cells, and subsets of tumor associated dendritic cells), wherein the expression of one or more TAM kinases in the immune cells may limit the ability of the patient's immune system to make an effective anti-tumor response.

The term “TAM-associated cancer” as used herein refers to cancers associated with or having increased expression and/or activity of one or more of the TAM kinases in a cancer cell or an immune cell (e.g., as compared to a control, e.g., a non-cancerous tissue or cell, or a corresponding tissue or cell from a control subject that does not have cancer). Non-limiting examples of a TAM-associated cancer are described herein. In some embodiments, the TAM-associated cancer is a cancer having a chromosomal translocation that results in the expression of a TMEM87B-MERTK fusion protein (e.g., amino acids 1-55 of TMEM87B and amino acids 433-1000 of MERTK) or an AXL-MBIP fusion protein. A description of an exemplary chromosomal translocation that results in the expression of a TMEM87B-MERTK fusion protein is provided in Shaver et al. (Cancer Res. 76(16):4850-4860, 2016). A description of an exemplary chromosomal translocation that results in the expression of an AXL-MBIP fusion protein is provided in Seo et al. (Genome Res. 22:2109-2119, 2012). Chromosomal translocations or the resulting expression of TMEM87B-MERTK or AXL-MBIP fusion proteins can be detected using In Situ Hybridization (e.g., Fluorescent In Situ Hybridization (FISH)). Chromosomal translocations that result in the expression of TMEM87B-MERTK or AXL-MBIP can be detected by sequencing DNA from a sample obtained from the subject (e.g., blood, plasma, urine, cerebrospinal fluid, saliva, sputum, bronchoalveolar lavage, bile, lymphatic fluid, cyst fluid, stool ascites, or a tumor biopsy obtained from the subject). Exemplary methods that can be used to sequence DNA are known in the art and include, e.g., next-generation sequencing (NGS), traditional PCR, digital PCR, and microarray analysis. Additional methods that can be used to detect chromosomal translocations that result in the expression of TMEM87B-MERTK or AXL-MBIP fusion proteins, or the expression of TMEM87B-MERTK or AXL-MBIP fusion proteins, are known in the art.

The term “c-Met-associated disease or disorder” as used herein refers to diseases or disorders associated with or having increased expression, level, and/or activity of c-Met kinase in a cell (e.g., a cancer cell or an immune cell) (e.g., as compared to a control, e.g., a non-cancerous tissue or cell, or a corresponding tissue or cell from a control subject that does not have cancer) and/or where activation of c-Met kinase expressed in non-cancer cells contributes to disease. Non-limiting examples of a c-Met-associated disease or disorder include, for example, cancer (a c-Met-associated cancer), e.g., any of the cancers described herein. In one embodiment, the disease is a cancer that overexpresses c-Met kinase after treatment with at least one additional anticancer agent (e.g., one or more of any of the additional anticancer agents described herein). In some embodiments, the disease is a cancer that has a higher protein level of c-Met kinase (e.g., due to mutation in a MET gene that results in decreased proteasome degradation of c-MET kinase in a mammalian cell). In some embodiments, the disease is a cancer that has a higher level of c-Met kinase activity due to an activating mutation in a c-Met gene (e.g., any of the activating mutations in a c-Met gene described herein) or an increase in the expression of a c-Met kinase in a mammalian cell. In some embodiments, the disease is a cancer that expresses a c-Met kinase that is resistant (e.g., to at least some extent as compared to a wildtype c-Met kinase) to a Type I c-Met inhibitor.

Receptor tyrosine kinases (RTKs) are cell surface proteins that transmit signals from the extracellular environment to the cell cytoplasm and nucleus to regulate cellular events such as survival, growth, proliferation, differentiation, adhesion, and migration. All RTKs contain an extracellular ligand binding domain and a cytoplasmic protein tyrosine kinase domain. Ligand binding leads to the dimerization of RTKs, which triggers the activation of the cytoplasmic kinase and initiates downstream signal transduction pathways. RTKs can be classified into distinct subfamilies based on their sequence similarity. The TAM receptor tyrosine kinases (TYRO3, AXL (also known as UFO) and MER) is an emerging class of innate immune checkpoints that participate in key steps of anti-tumoral immunity (Akalu, T, et al., Immunological Reviews 2017; 276:165-177). TAM kinases are characterized by an extracellular ligand binding domain consisting of two immunoglobulin-like domains and two fibronectin type III domains. Two ligands, growth arrest specific 6 (GAS6) and protein S (ProS), have been identified for TAM kinases. GAS6 can bind to and activate all three TAM kinases, while ProS is a ligand for MER and TYRO3 (Graham et al., 2014, Nature reviews Cancer 14, 769-785).

TAM kinases are ectopically expressed or over-expressed in a wide variety of cancers, including breast, colon, renal, skin, lung, liver, CNS (e.g., glioblastomas, neuroblastomas), ovarian, prostate, and thyroid malignancies, and metastatic cancers including breast cancer, lung cancer, melanoma, prostate cancer, pancreatic cancer, ovarian cancer, hepatocellular carcinoma, thyroid cancer, bladder cancer, Kaposi's sarcoma, mesothelioma, esophageal cancer, glioblastoma, colorectal cancer, cervical cancer, neuroblastoma and osteosarcoma (Graham et al., 2014, Nature Reviews Cancer 14, 769-785; and Linger et al., 2008, Oncogene 32, 3420-3431) and play important roles in tumor initiation and maintenance. When activated, AXL and MER can regulate tumor cell survival, proliferation, migration and invasion, angiogenesis, and tumor-host interactions (Schoumacher, M. et al., Curr. Oncol. Rep. 2017; 19(3);19) Accordingly, blocking TAM signaling may promote engagement of adaptive immunity and complement T-cell checkpoint blockade (Akalu, T, et al., Immunological Reviews 2017; 276:165-177). Therefore, TAM inhibition represents an attractive approach for targeting another class of oncogenic RTKs (Graham et al., 2014, Nature Reviews Cancer 14, 769-785; and Linger et al., 2008, Oncogene 32, 3420-3431).

AXL was originally identified as a transforming gene from DNA of patients with chronic myelogenous leukemia (O′Bryan et al., 1991, Molecular and Cellular Biology 11, 5016-5031). GAS6 binds to AXL and induces subsequent auto-phosphorylation and activation of AXL tyrosine kinase. AXL activates several downstream signaling pathways including P13K-AKT, RAF-MAPK, PLC-PKC (Feneyrolles et al., 2014, Molecular Cancer Therapeutics 13, 2141-2148; Linger et al., 2008, Oncogene 32, 3420-3431). Over-expression or overactivation of the AXL protein has been correlated with the promotion of multiple tumorigenic processes. High levels of AXL expression have been associates with poor prognosis in different cancers such as glioblastoma multiforme (Hutterer, M., et al., Clin. Caner Res. 2008, 14, 130-138), breast cancer (Wang, X., Cancer Res. 2013, 73, 6516-6525), lung cancer (Niederst, M. et al., Sci. Signaling, 2013, 6, re6), osteosarcoma (Han, J., Biochem. Biophys. Res. Commun. 2013, 435, 493-500), and acute myeloid leukemia (Ben-Batalla, L., et al., Blood 2013, 122, 2443-2452). AXL is over-expressed or amplified in a variety of malignancies including lung cancer, prostate cancer, colon cancer, breast cancer, melanoma, and renal cell carcinoma (Linger et al., 2008, Oncogene 32, 3420-3431), and over-expression of AXL is correlated with poor prognosis (Linger et al., 2008, Oncogene 32, 3420-3431). AXL activation promotes cancer cell survival, proliferation, angiogenesis, metastasis, and resistance to chemotherapy and targeted therapies. AXL knockdown or AXL antibody can inhibit the migration of breast cancer and NSCLC cancer in vitro, and blocked tumor growth in xenograft tumor models (Li et al., 2009, Oncogene 28, 3442-3455). In pancreatic cancer cells, inhibition of AXL decreased cell proliferation and survival (Koorstra et al., 2009, Cancer Biology & Therapy 8, 618-626). In prostate cancer, AXL inhibition decreased cell migration, invasion, and proliferation (Tai et al., 2008, Oncogene 27, 4044-4055). In triple-negative breast cancer, patients typically present a significant clinical challenge, as they do not respond to the various targeted cancer therapies due to an apparent lack of RTK activation. However, patients with triple-negative breast cancer do show some response to taxane-based chemotherapy and studies have suggested that combining anti-mitotic drugs (e.g., docetaxel) with an AXL inhibitor sensitized cancer cells to the anti-mitotic drug, and AXL in combination with an anti-mitotic drug may be an appropriate combination therapy in this disease setting (Wilson, et al., Cancer Res. 2014, 74(20), 5878-5890).

TAM kinases can contribute to therapeutic resistance by at least three mechanisms: intrinsic survival signaling in tumor cells, induction of TAM kinases as an escape mechanism for tumors that have been treated with oncogene-targeted agents, and immunosuppression in the tumor microenvironment (Graham, et al., Nature Reviews Cancer, 2014, 14, 769-785).

TAM kinases were found to promote resistance to cytotoxic chemotherapies (chemoresistance) in leukemia cells and solid tumor cells (Graham, et al., Nature Reviews Cancer, 2014, 14, 769-785). Transgenic lymphocytes ectopically expressing MER were found to be more resistant to dexamethasone than wild-type lymphocytes (Keating, A.K., et al., Oncogene, 2006, 25, 6092-6100), and stimulation of B-ALL cells with GAS6 increased resistance to cytarabine (Shiozawa, Y., et al., Neoplasia, 2010, 12, 116-127). AXL is induced in acute myeloid leukemia (AML) cells that have been treated with cytotoxic chemotherapies, and it mediates increased chemoresistance (Hong, C.C., et al., Cancer Lett., 2008, 268, 314-324). Chemotherapy-resistant chronic myeloid leukemia (CML) cell lines have upregulated levels of AXL, and shRNA-mediated knockdown of AXL increases chemosensitivity in CML cells and xenograft models (Zhao, Y., et al., Cancer Invest. 2012, 30, 287-294). Similarly, shRNA-mediated MER knock-down sensitizes B-cell acute lymphoblastic leukemia (B-ALL) and T-lineage acute lymphoblastic leukemia (T-ALL) cells to a range of chemotherapies (Linger, R.M., et al., Blood, 2013, 122, 1599-1609; Brandao, L.N., et al., Blood Cancer J., 2013, 3, e101). In solid tumors such as non-small cell lung cancer, pancreatic ductal adenocarcinoma, astrocytoma, lung adenocarcinoma, ovarian cancer, melanoma, and glioblastoma multiforme, overexpression of AXL or MER promotes chemoresistance, and shRNA-mediated inhibition sensitizes cells to treatment with cytotoxic chemotherapies (Linger, R.N., et al., Oncogene, 2013, 32, 3420-3431; Song, X., et al., Cancer, 2011, 117, 734-743; Keating, A.K., et al., Mol. Cancer Ther. 2010, 9, 1298-1307; Lay, J.D., et al., Cancer Res. 2007, 67, 3878-3887; Zhao, Y., et al., Cancer Invest, 2012, 30, 287-294; Macleod, K., Cancer Res. 2005, 65, 6789-6800; Zhu, S., et al., Proc. Natl Acad. Sci. USA, 2009, 106, 17025-17030; Wang, Y., et al., Oncogene 2013, 32, 872-882).

In contrast to chemoresistance, examples of acquired resistance for TAM kinases are currently limited to AXL. AXL is upregulated in imatinib-resistant CML and gastrointestinal stromal tumor (GIST) cell lines and tumor samples (Mahadevan, D., et al., Oncogene, 2007, 26, 3909-3919; Dufies, M., et al., Oncotarget 2011, 2, 874-885; Gioia, R., et al., Blood, 2011, 118, 2211-2221), and siRNA-mediated knockdown of AXL restored imatinib sensitivity to resistant cell lines (Dufies, M., et al.). Similarly, AXL is induced in lapatinib-resistant HER2 (also known as ERBB2)-positive breast cancer cell lines, and AXL inhibition restored lapatinib sensitivity (Liu, L., et al., Cancer Res. 2009, 69, 6871-6878). AXL has been associated with acquired resistance to epidermal growth factor receptor (EGFR) tyrosine kinase inhibitors (e.g., lapatinib and erlotinib) and therapeutic antibodies (e.g., cetuximab) in triple-negative breast cancer (Meyer, A.S. et al., Sci. Signal 2013, 6, ra66), colorectal cancer (Brand, et al., Cancer Res. 2014, 74:5152-5164), head and neck cancer (Kiles, K.M, et al., Mol. Cancer Ther. 2013, 12, 2541-2558) cell lines, and non-small cell lung cancer (Zhang, Nat. Genet. 2013, 44(8), 852-860). AXL has also been associated with acquired resistance to inhibitors targeting other kinases, including Pl3Kα inhibitors such as alpelisib (BYL719) in head and neck and esophageal squamous cell carcinomas (Elkabets, et al., Cancer Cell 2015, 27:533-546), MEK inhibitors (e.g., U0126 (1,4-Diamino-2,3-dicyano-1,4-bis(o-aminophenylmercapto)butadiene) and PD 325901 (1,4-Diamino-2,3-dicyano-1,4-bis(o-aminophenylmercapto)butadiene) in triple-negative breast cancer cell lines and melanoma cell lines (Miller, et al., Cancer Discovery 2016, 6:382-39), fibroblast growth factor (FGFR) (Ware, K. E., Oncogenesis 2013, 2, e39), anaplastic lymphoma kinase (ALK) (Kim, H. R., et al., Mol. Oncol. 2013, 7, 1093-1102) and insulin-like growth factor 1 receptor (IGF1R) (Huang, R., Cancer Res. 2010, 70, 7221-7231), and AXL inhibition has been demonstrated to overcome or delay resistance to these inhibitors. AXL is upregulated in NSCLC cell lines and xenografts that are resistant to EGFR tyrosine kinase inhibitors (erlotinib) and antibody drugs (cetuximab) (Brad, T. M., et al., Cancer Res. 2014, 74, 5152-5164; Zhang, Z., et al., Nature Genet. 2012, 44, 852-860), and it is induced in 20% of matched tumor samples taken from patients with NSCLC after development of resistance to the EGFR inhibitor erlotinib.

Regarding MER and AXL dual inhibitors, the normal roles of MER and AXL in preventing or terminating innate immune-mediated inflammation and natural killer (NK) cell responses are subverted in the tumor microenvironment. MER and AXL decrease NK cell antitumor activity, which allows increased metastases.

MER was originally identified as a phospho-protein from a lymphoblastoid expression library (Graham et al., 1995, Oncogene 10, 2349-2359). Both GAS6 and ProS can bind to MER and induce the phosphorylation and activation of MER kinase (Lew et al., 2014. eLife, 3 :e03385). Like AXL, MER activation also conveys downstream signaling pathways including P13K-Akt and Raf-MAPK (Linger et al., 2008, Oncogene 32, 3420-3431). MER is over-expressed in many cancers including multiple myeloma, gastric, prostate, breast, melanoma and rhabdomyosarcoma (Linger et al., 2008, Oncogene 32, 3420-3431). MER knockdown inhibits multiple myeloma cell growth in vitro and in xenograft models (Waizenegger et al., 2014, Leukemia, 1-9). In acute myeloid leukemia, MER knockdown induced apoptosis, decreased colony formation, and increased survival in a mouse model (Lee-Sherick et al., 2013, Oncogene 32, 5359-5368). MER inhibition increased apoptosis, decreased colony formation, increased chemo-sensitivity, and decreased tumor growth in NSCLC (Linger et al., 2013, Oncogene 32, 3420-3431). Similar effects are observed for MER knockdown in melanoma (Schlegel et al., 2013) and glioblastoma (Wang et al., 2013, Oncogene 32, 872-882).

TYRO3 was originally identified through a PCR-based cloning study (Lai and Lemke, 1991, Neuron 6, 691-704). Both ligands, GAS6 and ProS, can bind to and activate Tyro3. TYRO3 also plays a role in cancer growth and proliferation. TYRO3 is over-expressed in melanoma cells, and knockdown of TYRO3 induces apoptosis in these cells (Demarest et al., 2013, Biochemistry 52, 3102-3118).

TAM kinases have emerged as potential immune-oncology targets. The durable clinical responses to immune checkpoint blockade observed in cancer patients clearly indicate that the immune system plays a critical role in tumor initiation and maintenance. Genetic mutations from cancer cells can provide a diverse set of antigens that the immune cells can use to distinguish tumor cells from their normal counterpart. However, cancer cells have evolved multiple mechanisms to evade host immune surveillance. In fact, one hallmark of human cancer is its ability to avoid immune destruction. Cancer cells can induce an immune-suppressive microenvironment by promoting the formation of M2 tumor associated macrophages, myeloid derived suppressor cells (MDSC), and regulatory T cells. Cancer cells can also produce high levels of immune checkpoint proteins such as PD-L1 to induce T cell anergy or exhaustion. It is now clear that tumors co-opt certain immune-checkpoint pathways as a major mechanism of immune resistance (Pardoll, 2012, Cancer 12, 252-264). Antagonizing these negative regulators of T-cell function with antibodies has shown striking efficacy in clinical trials of a number of malignancies including advanced melanoma, non-small cell lung and bladder cancer. While these therapies have shown encouraging results, not all patients mount an anti-tumor response suggesting that other immune-suppressive pathways may also be important.

TAM kinases have been shown to function as checkpoints for immune activation in the tumor milieu. All TAM kinases are expressed in NK cells, and TAM kinases inhibit the antitumor activity of NK cells. LDC1267, a small molecule TAM kinase inhibitor, activates NK cells, and blocks metastasis in tumor models with different histologies (Paolino et al., 2014, Nature 507, 508-512). In addition, MER kinase decreases the activity of tumor associated macrophages through the increased secretion of immune suppressive cytokines such as ILIO and IL4, and decreased production of immune activating cytokines such as IL12 (Cook et al., 2013, The Journal of Clinical Investigation 123, 3231-3242). MER inhibition has been shown to reverse this effect. As a result, MER knockout mice are resistant to PyVmT tumor formation (Cook et al., 2013, Journal of Clinical Investigation 123, 3231-3242). The role of TAM kinases in the immune response is also supported by knockout mouse studies. TAM triple knockout mice (TKO) are viable. However, these mice displayed signs of autoimmune disease including enlarged spleen and lymph nodes, autoantibody production, swollen footpad and joints, skin lesions, and systemic lupus erythematosus (Lu and Lemke, 2001, Science 293, 306-311). This is consistent with the knockout phenotype for approved immune-oncology targets such as CTLA4 and PD-1.

Both CTLA-4 and PD-1 knockout mice showed signs of autoimmune disease, and these mice die within a few weeks after birth (Chambers et al., 1997, Immunity 7, 885-895; and Nishimura et al., 2001, Science 291, 319-322). Therefore inhibition of TAM kinases alone or in combination with other immune therapies may increase the ability of the immune system to make a therapeutically beneficial immune response against the cancer.

The MET receptor tyrosine kinases (e.g., c-Met) controls growth, invasion and metastasis in cancer cells. The c-Met is activated in human cancer by a variety of different molecular mechanisms (see, e.g., Zhang et al., Carcinogenesis 4:345-355, 2016). For example, a c-Met-associated disease or condition (e.g., a c-Met-associated cancer) include (i) mutations that alter the sequence and increase the activity of c-Met kinase; (ii) mutations in regulatory sequences controlling c-Met expression or regulators of c-Met expression that confer increased expression of c-Met; (iii) mutations that alter the c-Met polypeptide sequence to confer increased c-Met kinase half-life (e.g., a mutation in a MET gene that results in exon 14 skipping during mRNA splicing that results in an increased level of c-Met in a mammalian cell); (iv) methylation of a MET gene (see, e.g., Nones et al., Int. J. Cancer 135:1110-8, 2014); (v) methylation of long interspersed nuclear element (L1) present in the Met intron between exon 2 and exon 3 (Weber et al., Oncogene 29:5775-5784, 2010); (vi) MET gene amplification; or (vii) by simultaneous expression of receptor and ligand, which results in autocrine stimulation of cancer cells (Birchmeier et al., Nat. Rev. Mol. Cell. Biol. 4:915-925, 2003).

Exemplary mutations in a MET gene that alter the sequence of a c-Met kinase and increase the activity of c-Met kinase (e.g., as compared to wildtype c-Met kinase) include, but are not limited to those listed in Table 1.

TABLE 1
Exemplary list of mutations in a MET gene that alter the sequence
of a c-Met kinase and increase the activity of the c-Met kinase

| MET | MET | |
|---|---|---|
| Isoform 1 | Isoform 2 | |
| mutation | mutation | Reference |
| V1092I | V1110I | Schmidt et al., Oncogene 18: 2343-2350, 1999 |
| H1094L | H1112L | Schmidt et al., Oncogene 18: 2343-2350, 1999 |
| H1094R | H1112R | Schmidt et al., Cancer Research 58: |
| | | 1719-1722, 1998 |
| H1094Y | H1112Y | Schmidt et al., Oncogene 18: 2343-2350, 1999 |
| H1106D | H1124D | Schmidt et al., Oncogene 18: 2343-2350, 1999 |
| D1228H | D1246H | Bardelli et al., Proc. Natl. Acad. Sci. 95: |
| | | 14379-14383, 2002 |
| D1228N | D1246N | Bardelli et al., Proc. Natl. Acad. Sci. 95: |
| | | 14379-14383, 2002 |
| Y1230C | Y1248C | Bardelli et al., Proc. Natl. Acad. Sci. 95: |
| | | 14379-14383, 2002 |
| Y1230D | Y1248D | Schmidt et al., Oncogene 18: 2343-2350, 1999 |
| Y1230H | Y1248H | Bardelli et al., Proc. Natl. Acad. Sci. 95: |
| | | 14379-14383, 2002 |
| M1250T | M1268T | Bardelli et al., Proc. Natl. Acad. Sci. 95: |
| | | 14379-14383, 2002 |

Exemplary mutations that alter the c-Met polypeptide sequence to confer increased c-Met kinase half-life (as compared to a wildtype c-Met kinase) include, but are not limited to, the mutations listed in Table 2 that promote skipping of MET exon 14 during mRNA splicing. Other exemplary mutations that are predicted to promote skipping of MET exon 14 during mRNA splicing include, but are not limited to, those disclosed in Frampton et al., Cancer Discovery 5(8):850-9, 2015; and Heist et al., Oncologist 21(4):481-6, 2016. The portion of the c-Met protein encoded by exon 14, most prominently Y1003 in a DpYR motif, is required for efficient recruitment of the E3 ubiquitin-protein ligase CBL, which targets MET for ubiquitin-mediated degradation (Lee et al., J. Biol. Chem. 269:19457-61, 1994; Lee et al., Exp. Mol. Med. 38:565-73, 2006; Lee et al., Oncogene 33:34-43, 2014). Skipping of MET exon 14 in mRNA splicing results in a c-Met kinase that maintains the reading frame and that demonstrates increased c-Met protein stability and prolonged signaling upon HGF stimulation, leading to increased oncogenic potential (Peschard et al., Mol. Cell. 8:995-1004, 2001; Abella et al., Mol. Cell. Biol. 25:9632-45, 2005). Other exemplary mutations that alter the c-Met polypeptide sequence to confer increased c-Met kinase half-life include, but are not limited to an amino acid substitution at Y1003 (e.g., a Y1003F amino acid substitution) (Peschard et al., Mol. Cell. 8:995-1004, 2001).

TABLE 2
Exemplary list of mutations that confer
skipping of MET exon 14

| | | Altered | |
|---|---|---|---|
| | | sequence | |
| Chromo- | | (“—” | |
| somal | Reference | denotes | |
| location | sequence | deletion) | Reference |
| chr7: | AAGCTCTTT | — | Kong-Beltran |
| 116411875- | CTTTCTCTCTGTT | | et al., |
| 116411897 | | | Cancer Res. |
| | | | 66(1): |
| | | | 283-289, |
| | | | 2006 |
| chr7: | ACCGAGCTA | — | Kong-Beltran |
| 116412022- | CTTTTCCAG | | et al., |
| 116412050 | AAGGTATATT | | Cancer Res. |
| | | | 66(1): |
| | | | 283-289, |
| | | | 2006 |
| chr7: | G | T | Kong-Beltran |
| 116412043- | | | et al., |
| 116412044 | | | Cancer Res. |
| | | | 66(1): |
| | | | 283-289, |
| | | | 2006 |
| chr7: | CCCATGATA | — | Onozato |
| 116411854- | GCCGTCTTTAA | | et al., |
| 116411874 | | | J. Thorac. |
| | | | Oncol. |
| | | | 4: 5-11, |
| | | | 2009. |
| chr7: | CTTTCTCTCTG | — | Onozato |
| 116411884- | | | et al., |
| 116411895 | | | J. Thorac. |
| | | | Oncol. |
| | | | 4: 5-11, |
| | | | 2009. |
| chr7: | TTCTCTCT | — | Onozato |
| 116411886- | GTTTTAAGATC | | et al., |
| 116411905 | | | J. Thorac. |
| | | | Oncol. |
| | | | 4: 5-11, |
| | | | 2009. |
| chr7: | G | A | Onozato |
| 116412043- | | | et al., |
| 116412044 | | | J. Thorac. |
| | | | Oncol. |
| | | | 4: 5-11, |
| | | | 2009. |
| chr7: | G | T | Asaoka |
| 116412043- | | | et al., |
| 116412044 | | | Biochem. |
| | | | Biophys. |
| | | | Res. Comm. |
| | | | 394: |
| | | | 1042-6, |
| | | | 2010. |
| chr7: | CTTTCTCTCTGT | — | Jenkins |
| 116411884- | | | et al., |
| 116411896 | | | Clin. |
| | | | Lung |
| | | | Cancer |
| | | | 16: |
| | | | e101-e104, |
| | | | 2015. |
| chr7: | G | C | Waqar |
| 116412042- | | | et al., |
| 116412043 | | | J. Thorac. |
| | | | Oncol. |
| | | | 10: |
| | | | e29-31, |
| | | | 2015. |
| chr7: | G | C | Mendenhall |
| 116412042- | | | et al., |
| 116412043 | | | J. Thorac. |
| | | | Oncol. |
| | | | 10: |
| | | | e23-34, |
| | | | 2015. |

Exemplary c-Met-associated cancers include, but are not limited to those listed in Table 3.

TABLE 3
Exemplary c-Met-associated cancers exhibiting
increased expression and/or activity of c-Met

| Cancer type | Type of genetic alterations | Reference |
|---|---|---|
| Gastrointestinal cancer (GI); | MET gene amplification; | Mo et al., Chronic Dis. |
| Gastric cancer | Amino acid substitution in | Transl. Med. 3(3): 148-153, |
| | kinase domain (e.g., an | 2017; Tovar et al., Ann. |
| | amino acid substitution at | Transl. Med. 5(10): 205, |
| | position 1108, e.g., an | 2017; Asaoka et al., |
| | A1108S amino acid | Biochem. Biophys. Res. |
| | substitution); | Comm. 394: 1042-6, 2010. |
| | point mutation conferring | |
| | skipping of MET exon 14 | |
| | during mRNA splicing (e.g., | |
| | chr7:116412043-116412044, | |
| | G to T mutation) | |
| Colorectal Adenocarcinoma | Amino acid substitution at | Zenali et al., Oncoscience |
| | position 375 (e.g., a N375S | 2(5): 533-541, 2015. |
| | amino acid substitution); an | |
| | amino acid substitution at | |
| | position 1010 (e.g., a T1010I | |
| | amino acid substitution); an | |
| | amino acid substitution at | |
| | position 988 (e.g., a R988C | |
| | amino acid substitution); an | |
| | amino acid substitution at | |
| | position 1253 (e.g., a | |
| | Y1253D amino acid | |
| | substitution); and an amino | |
| | acid substitution at position | |
| | 1248 (e.g., a Y1248H amino | |
| | acid substitution) | |
| Colorectal carcinoma (CRC) | MET gene amplification; MET | Zeng et al., Cancer Lett. |
| | overexpression; | 265: 258-269, 2008; Kong- |
| | amino acid substitutions in | Beltran et al., Cancer Res. |
| | JM domain of c-Met kinase | 66: 283-9, 2006; Tovar et al., |
| | (e.g., an amino acid | Ann. Transl. Med. 5(10): 205, |
| | substitution at position 970 | 2017. |
| | (e.g., an R970C amino acid | |
| | substitution) and an amino | |
| | acid substitution at position | |
| | 992 (e.g., a T992I amino acid | |
| | substitution) | |
| Non-small cell lung cancer | Point mutation conferring | Ichimura et al., Jpn J. Cancer |
| (NSCLC) | skipping of MET exon 14 | Res. 87: 1063-1069, 1996; |
| | during mRNA splicing; | Ma et al., Cancer Res. |
| | MET gene amplification; | 63: 6272-81, 2003; Kong- |
| | amino acid substitutions in c- | Beltran et al., Cancer Res. |
| | Met kinase domain (e.g., an | 66: 283-9, 2006; Tovar et al., |
| | amino acid substitution at | 2017, Ann. Transl. Med. |
| | position 970 (e.g., a R970C | 5(10): 205, 2017 |
| | amino acid substitution), an | |
| | amino acid substitution at | |
| | position 988 (e.g., a R988C | |
| | amino acid substitution); an | |
| | amino acid substitution at | |
| | position 1010 (e.g., a T1010I | |
| | amino acid substitution); an | |
| | amino acid substitution at | |
| | position 1058 (e.g., a | |
| | S1058P amino acid | |
| | substitution)); | |
| | amino acid substitution in | |
| | the JM domain of c-Met | |
| | kinase (e.g., an amino acid | |
| | substitution at position 988 | |
| | (e.g., a R988C amino acid | |
| | substitution), an amino acid | |
| | substitution at position 1010 | |
| | (e.g., a T1010I amino acid | |
| | substitution), an amino acid | |
| | substitution at position 1058 | |
| | (e.g., a S1058P amino acid | |
| | substitution), an amino acid | |
| | substitution at position 970 | |
| | (e.g., a R970C amino acid | |
| | substitution), and an amino | |
| | acid substitution at position | |
| | 992 (e.g., a T992I amino | |
| | acid substitution)). | |
| Heptacellular carcinoma | MET overexpression; | Goyal et al., Clin. Cancer |
| (HCC) | Amino acid substitutions in | Res. 19: 2310-2318, 2013; |
| | kinase domain of c-Met (e.g., | Tovar et al., Ann. Transl. |
| | an amino acid substitution at | Med. 5(10): 205, 2017; Zenali |
| | position 1191 (e.g., a T1191I | et al., Oncoscience 2(5): 533- |
| | amino acid substitution), an | 541, 2015 |
| | amino acid substitution at | |
| | position 1262 (e.g., a J1262R | |
| | amino acid substitution), or | |
| | an amino acid substitution at | |
| | position 1268 (e.g., a | |
| | M1268T or an M1268I amino | |
| | acid substitution)); an amino | |
| | acid substitution at position | |
| | 375 (e.g., an N375S amino | |
| | acid substitution); an amino | |
| | acid substitution at position | |
| | 988 (e.g., a R988C amino | |
| | acid substitution) | |
| Hereditary papillary renal | Amino acid substitutions in | Tovar et al., Ann. Transl. |
| carcinoma (HPRC) | the kinase domain of c-Met | Med. 5(10): 205, 2017 |
| | (e.g., an amino acid | |
| | substitution at position 112 | |
| | (e.g., a H112R, a H112L, or a | |
| | H112I amino acid | |
| | substitution), an amino acid | |
| | position as position 1230 | |
| | (e.g., a Y1230C, a Y1230H, | |
| | or a Y1230D amino acid | |
| | substitution), an amino acid | |
| | substitution at position 1246 | |
| | (e.g., a D1246N amino acid | |
| | substitution), an amino acid | |
| | substitution at position 1248 | |
| | (e.g., a Y1248C amino acid | |
| | substitution), an amino acid | |
| | substitution at position 1268 | |
| | (e.g., a M1268T amino acid | |
| | substitution or a M1268I | |
| | amino acid substitution). | |
| Papillary renal carcinoma | Amino acid substitutions in | Jeffers et al., Proc. Natl. |
| | the kinase domain of c-Met | Acad. Sci. U.S.A. |
| | (e.g., those listed in Table 1) | 94(21): 11445-11450, 1997; |
| | | Schmidt et al., Nat. Genet. |
| | | 16: 68-73, 1997; Schmidt et |
| | | al., Oncogene 18: 2343-50, |
| | | 1991. |
| Melanoma | An amino acid substitution at | Zenali et al., Oncoscience |
| | position 375 (e.g., a N375S | 2(5): 533-541, 2015. |
| | amino acid substitution); an | |
| | amino acid substitution at | |
| | position 988 (e.g., a R988C | |
| | amino acid substitution); an | |
| | amino acid substitution at | |
| | position 1010 (e.g., a T1010I | |
| | amino acid substitution). | |
| Gastric adenocarcinoma | An amino acid substitution at | Zenali et al., Oncoscience |
| | position 375 (e.g., an N375S | 2(5): 533-541, 2015. |
| | amino acid substitution). | |
| Appendiceal | An amino acid substitution at | Zenali et al., Oncoscience |
| adenocarcinoma | position 375 (e.g., an N375S | 2(5): 533-541, 2015. |
| | amino acid substitution); an | |
| | amino acid substitution at | |
| | position 988 (e.g., a R988C | |
| | amino acid substitution) | |
| Duodenal adenocarcinoma | An amino acid substitution at | Zenali et al., Oncoscience |
| | position 375 (e.g., an N375S | 2(5): 533-541, 2015. |
| | amino acid substitution) | |
| Pancreatic adenocarcinoma | An amino acid substitution at | Zenali et al., Oncoscience |
| | position 375 (e.g., an N375S | 2(5): 533-541, 2015. |
| | amino acid substitution) | |
| Lung adenocarcinoma | An amino acid substitution at | Zenali et al., Oncoscience |
| | position 375 (e.g., an N375S | 2(5): 533-541, 2015. |
| | amino acid substitution); an | |
| | amino acid substitution at | |
| | position 988 (e.g., a R988C | |
| | amino acid substitution); an | |
| | amino acid substitution at | |
| | position 1010 (e.g., a T1010I | |
| | amino acid substitution) | |
| Thyroid papillary carcinoma | An amino acid substitution at | Zenali et al., Oncoscience |
| | position 375 (e.g., an N375S | 2(5): 533-541, 2015. |
| | amino acid substitution) | |
| Thyroid medullary carcinoma | An amino acid substitution at | Zenali et al., Oncoscience |
| | position 1010 (e.g., a T1010I | 2(5): 533-541, 2015. |
| | amino acid substitution) | |
| Ewing sarcoma | An amino acid substitution at | Zenali et al., Oncoscience |
| | position 1010 (e.g., a T1010I | 2(5): 533-541, 2015. |
| | amino acid substitution) | |
| Prostate adenocarcinoma | An amino acid substitution at | Zenali et al., Oncoscience |
| | position 375 (e.g., an N375S | 2(5): 533-541, 2015. |
| | amino acid substitution) | |
| Squamous cell carcinoma of | An amino acid substitution at | Zenali et al., Oncoscience |
| the head and neck and cervix | position 375 (e.g., an N375S | 2(5): 533-541, 2015. |
| | amino acid substitution); an | |
| | amino acid substitution at | |
| | position 988 (e.g., a R988C | |
| | amino acid substitution); an | |
| | amino acid substitution at | |
| | position 1010 (e.g., an | |
| | T1010I amino acid | |
| | substitution) | |
| Renal cell carcinoma | An amino acid substitution at | Zenali et al., Oncoscience |
| | position 375 (e.g., an N375S | 2(5): 533-541, 2015; Schmidt |
| | amino acid substitution); an | et al., Oncogene 18: 2343- |
| | amino acid substitution at | 2350, 1999; Scmidt et al., |
| | position 1092 (e.g., a V1092I | Cancer Research 58: 1719- |
| | amino acid substitution); an | 1722, 1998; Bardelli et al., |
| | amino acid substitution at | Proc. Natl. Acad. Sci. 95: |
| | position 1094 (e.g., a | 14379-14383, 2002.. |
| | H1094L, a H1094R, or a | |
| | H1094Y amino acid | |
| | substitution); an amino acid | |
| | substitution at position 1106 | |
| | (e.g., a H1106D amino acid | |
| | substitution); an amino acid | |
| | substitution at position 1228 | |
| | (e.g., a D1228H o ra D1228N | |
| | amino acid substitution); an | |
| | amino acid substitution at | |
| | position 1230 (e.g., a | |
| | Y1230C, a Y1230D, or a | |
| | Y1230H amino acid | |
| | substitution); an amino acid | |
| | substitution at position 1250 | |
| | (e.g., a M1250T amino acid | |
| | substitution) | |
| Pheochromocytoma and | An amino acid substitution at | Zenali et al., Oncoscience |
| composite | position 375 (e.g., an N375S | 2(5): 533-541, 2015. |
| pheochromocytoma | amino acid substitution); an | |
| | amino acid substitution at | |
| | position 988 (e.g., an R988C | |
| | amino acid substitution) | |
| Ovarian serous carcinoma | An amino acid substitution at | Zenali et al., Oncoscience |
| | position 375 (e.g., an N375S | 2(5): 533-541, 2015. |
| | amino acid substitution); an | |
| | amino acid substitution at | |
| | position 1010 (e.g., a 1010I | |
| | amino acid substitution) | |
| Ovarian clear cell carcinoma | An amino acid substitution at | Zenali et al., Oncoscience |
| | position 375 (e.g., an N375S | 2(5): 533-541, 2015. |
| | amino acid substitution) | |
| Ovarian mixed carcinoma | An amino acid substitution at | Zenali et al., Oncoscience |
| | position 1010 (e.g., a T1010I | 2(5): 533-541, 2015. |
| | amino acid substitution) | |
| Peritoneal serous carcinoma | An amino acid substitution at | Zenali et al., Oncoscience |
| | position 1010 (e.g., a T1010I | 2(5): 533-541, 2015. |
| | amino acid substitution) | |
| Breast ductal | An amino acid substitution at | Zenali et al., Oncoscience |
| adenocarcinoma | position 375 (e.g., an N375S | 2(5): 533-541, 2015. |
| | amino acid substitution); an | |
| | amino acid substitution at | |
| | position 1010 (e.g., a T1010I | |
| | amino acid substitution). | |
| Uterine leiomyosarcoma | An amino acid substitution at | Zenali et al., Oncoscience |
| | position 375 (e.g., an N375S | 2(5): 533-541, 2015. |
| | amino acid substitution); | |
| | amino acid substitution at | |
| | position 1010 (e.g., a T1010I | |
| | amino acid substitution) | |
| Uterine endometrioid | An amino acid substitution at | Zenali et al., Oncoscience |
| adenocarcinoma | position 375 (e.g., an N375S | 2(5): 533-541, 2015. |
| | amino acid substitution); an | |
| | amino acid substitution at | |
| | position 1010 (e.g., an | |
| | T1010I amino acid | |
| | substitution). | |
| Uterine malignant mixed | An amino acid substitution at | Zenali et al., Oncoscience |
| mullerian tumor | position 375 (e.g., an N375S | 2(5): 533-541, 2015. |
| | amino acid substitution) | |
| Glioblastoma | An amino acid substitution at | Zenali et al., Oncoscience |
| | position 375 (e.g., an N375S | 2(5): 533-541, 2015. |
| | amino acid substitution) | |
| Anaplastic glioma | An amino acid substitution at | Zenali et al., Oncoscience |
| | position 375 (e.g., an N375S | 2(5): 533-541, 2015. |
| | amino acid substitution) | |
| Oligodendroglioma | An amino acid substitution at | Zenali et al., Oncoscience |
| | position 1010 (e.g., an | 2(5): 533-541, 2015. |
| | T1010I amino acid | |
| | substitution) | |
| Desmoplastic small round | An amino acid substitution at | Zenali et al., Oncoscience |
| cell tumor | position 375 (e.g., an N375S | 2(5): 533-541, 2015. |
| | amino acid substitution) | |
| Squamous cell carcinoma of | An amino acid substitution at | Zenali et al., Oncoscience |
| rectum | position 375 (e.g., N375S | 2(5): 533-541, 2015. |
| | amino acid substitution) | |
| Salivary gland carcinoma | An amino acid substitution at | Zenali et al., Oncoscience |
| | position 375 (e.g., an N375S | 2(5): 533-541, 2015. |
| | amino acid substitution) | |
| Heart angiosarcoma | An amino acid substitution at | Zenali et al., Oncoscience |
| | position 375 (e.g., a N375S | 2(5): 533-541, 2015. |
| | amino acid substitution); an | |
| | amino acid substitution at | |
| | position 1010 (e.g., a T1010I | |
| | amino acid substitution) | |
| Gastrointestinal stromal | An amino acid substitution at | Zenali et al., Oncoscience |
| tumor (GIST) | position 1010 (e.g., a T1010I | 2(5): 533-541, 2015. |
| | amino acid substitution); an | |
| | amino acid substitution at | |
| | position 988 (e.g., an R988C | |
| | amino acid substitution) | |
| Invasive thymoma | An amino acid substitution at | Zenali et al., Oncoscience |
| | position 375 (e.g., an N375S | 2(5): 533-541, 2015. |
| | amino acid substitution) | |
| Spindle sarcoma | An amino acid substitution at | Zenali et al., Oncoscience |
| | position 375 (e.g., an N375S | 2(5): 533-541, 2015. |
| | amino acid substitution) | |

In some embodiments, compounds of Formula I, II, Ill or IV can be used to treat a c-Met-associated cancer expressing a c-Met kinase that is resistant (e.g., to at least some extent as compared to a wildtype c-Met kinase) to a c-Met inhibitor (e.g., a Type I c-Met inhibitor). Non-limiting examples of amino acid substitutions that result in resistance of c-Met to a c-Met inhibitor (e.g., a Type I c-Met inhibitor) include: an amino acid substitution at position 1092 (e.g., a V10921 amino acid substitution in isoform 1 of c-Met or a V11101 amino acid substitution in isoform 2 of c-Met); an amino acid substitution at position 1094 (e.g., a H1094L amino acid substitution in isoform 1 of c-Met or a H1112L amino acid substitution in isoform 2 of c-Met; an H1094Y amino acid substitution in isoform 1 of c-Met or an H1112Y amino acid substitution in isoform 2 of c-MET); an amino acid substitution at position 1155 (e.g., a V1155L amino acid substitution in isoform 1 or a V1173L amino acid substitution in isoform 2 of c-Met); an amino acid substitution at position 1163 (e.g., a G1163R amino acid substitution in isoform 1 of c-Met or a G1181R amino acid substitution in isoform 2 of c-Met); an amino acid substitution at position 1195 (e.g., an L1195F amino acid substitution in isoform 1 of c-Met or a L1213F amino acid substitution in isoform 2 of c-Met; an L1195V amino acid substitution in isoform 1 of c-Met or an L1213V amino acid substitution in isoform 2 of c-Met); an amino acid substitution at position 1200 (e.g., an F1200I amino acid substitution in isoform 1 of c-Met or an F1218I amino acid substitution in isoform 2 of c-Met); an amino acid substitution at position 1211 (e.g., an M1211L amino acid substitution in isoform 1 of c-Met or an M1229L amino acid substitution in isoform 2 of c-Met); an amino acid substitution at position 1228 (e.g., a D1228A amino acid substitution in isoform 1 of c-Met or a D1246A amino acid substitution in isoform 2 of c-Met; a D1228G amino acid substitution in isoform 1 of c-Met or a D1246G amino acid substitution in isoform 2 of c-Met; a D1228H amino acid substitution in isoform 1 of c-Met or a D1246H amino acid substitution in isoform 2 of c-Met; a D1228N amino acid substitution in isoform 1 of c-Met or a D1246N amino acid substitution in isoform 2 of c-Met; a D1228V amino acid substitution in isoform 1 of c-Met or a D1246V amino acid substitution in isoform 2 of c-Met; or a D1228Y amino acid substitution in isoform 1 of c-Met or a D1246Y amino acid substitution in isoform 2 of c-Met); an amino acid substitution at position 1230 (e.g., a Y1230C amino acid substitution in isoform 1 of c-Met or a Y1248C amino acid substitution in isoform 2 of c-Met; a Y1230H amino acid substitution in isoform 1 of c-Met or a Y1248H amino acid substitution in isoform 2 of c-Met; or a Y1230S amino acid substitution in isoform 1 of c-Met or a Y1248S amino acid substitution in isoform 2 of c-Met); or an amino acid substitution at position 1250 (e.g., a M1250T amino acid substitution in isoform 1 of c-Met or a M1268T amino acid substitution in isoform 2 of c-Met). Non-limiting examples of Type I inhibitors include crizotinib (PF-02341066), capmatinib, NVP-BVU972, AMG 337, bozitinib, glumetinib, savolitinib, and tepotinib. In some embodiments, amino acid substitutions that result in resistance of c-Met to a Type 1 c-Met inhibitor include L1195V, F12001, D1228H, D1228N, Y1230C, Y1230H, and Y1230S.

In some embodiments, compounds of Formula I, II, Ill or IV can be used to treat a c-Met-associated cancer having a chromosomal translocation that result in a fusion protein including the c-Met kinase domain, where the fusion protein has increased c-Met activity as compared to a wildtype c-Met kinase (e.g., a Met-TPR fusion protein (Rodrigues et al., Mol. Cell. Biol. 13:6711-6722, 1993) and the fusion protein/chromosomal translocation described in Cooper et al., Nature 311(5981):29-33, 1984.

Accordingly, In some embodiments, provided herein is a method for treating a TAM-associated disease or disorder (e.g., a TAM-associated cancer), a c-Met-associated disease or disorder (e.g., a c-Met-associated cancer), or both, in a patient in need thereof, the method comprising administering to the patient a therapeutically effective amount of a compound of Formula I, II, Ill or IV or a pharmaceutically acceptable salt thereof or a pharmaceutical composition thereof.

Also provided herein are methods of treating a patient identified or diagnosed as having a TAM-associated cancer, a c-Met-associated cancer, or both, that include administering to a patient identified or diagnosed as having a TAM-associated cancer, a c-Met-associated cancer, or both, a therapeutically effective amount of a compound of Formula I, II, Ill or IV or a pharmaceutically acceptable salt thereof or a pharmaceutical composition thereof.

Also provided herein are methods of treating a patient having a cancer that include: (a) identifying the patient as having a TAM-associated cancer, a c-Met-associated cancer, or both, and (b) administering to the patient identified as having a TAM-associated cancer, a c-Met-associated cancer, or both, a therapeutically effective amount of a compound of Formula I, II, Ill or IV or a pharmaceutically acceptable salt thereof or a pharmaceutical composition thereof.

Also provided herein are methods of decreasing the risk of developing a metastasis or an additional metastasis in a patient identified or diagnosed as having a TAM-associated cancer, a c-Met-associated cancer, or both, that include administering to the patient identified or diagnosed as having a TAM-associated cancer, a c-Met-associated cancer, or both, a therapeutically effective amount of a compound of Formula I, II, Ill or IV or a pharmaceutically acceptable salt thereof or a pharmaceutical composition thereof. In some embodiments, the methods result in at least a 1% reduction (e.g., at least a 2% reduction, at least a 3% reduction, at least a 4% reduction, at least a 5% reduction, at least a 6% reduction, at least a 8% reduction, at least a 10% reduction, at least a 12% reduction, at least a 14% reduction, at least a 16% reduction at least a 18% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction) in the patient's risk of developing a metastasis or an additional metastasis, e.g., as compared to a population of subjects having a similar TAM-associated cancer and/or c-Met-associated cancer but receiving a different treatment or no treatment.

Also provided are methods of decreasing the risk of developing a metastasis or an additional metastasis in a patient having a cancer that include: (a) identifying the patient as having a TAM-associated cancer, a c-Met-associated cancer, or both; and (b) administering to the patient identified as having a TAM-associated cancer, a c-Met-associated cancer, or both, a therapeutically effective amount of a compound of Formula I, II, Ill or IV or a pharmaceutically acceptable salt thereof or a pharmaceutical composition thereof. In some embodiments, the methods result in at least a 1% reduction (e.g., at least a 2% reduction, at least a 3% reduction, at least a 4% reduction, at least a 5% reduction, at least a 6% reduction, at least a 8% reduction, at least a 10% reduction, at least a 12% reduction, at least a 14% reduction, at least a 16% reduction, at least a 18% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction) in the patient's risk of developing a metastasis or an additional metastasis, e.g., as compared to a population of subjects having a similar TAM-associated cancer and/or c-Met-associated cancer, but receiving a different treatment or no treatment.

Also provided are methods of decreasing migration and/or invasion of a cancer cell in a patient identified as having a TAM-associated cancer, a c-Met-associated cancer, or both, that include administering to a patient identified or diagnosed as having a TAM-associated cancer, a c-Met-associated cancer, or both, a therapeutically effective amount of a compound of Formula I, II, Ill or IV or a pharmaceutically acceptable salt thereof or a pharmaceutical composition thereof. In some embodiments, the methods result in at least a 1% decrease (e.g., at least a 2% decrease, at least a 3% decrease, at least a 4% decrease, at least a 5% decrease, at least a 6% decrease, at least a 8% decrease, at least a 10% decrease, at least a 12% decrease, at least a 14% decrease, at least a 16% decrease, at least a 18% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease) in the migration and/or invasion of a cancer cell in the patient, e.g., as compared to the migration and/or invasion of a cancer cell or a population of cancer cells in a subject having a similar TAM-associated cancer and/or c-Met-associated cancer but receiving a different treatment or no treatment.

Also provided herein are methods of decreasing migration and/or invasion of a cancer cell in a patient having a cancer that include: (a) identifying the patient as having a TAM-associated cancer, a c-Met-associated cancer, or both; and (b) administering to the patient identified as having a TAM-associated cancer, a c-Met-associated cancer, or both, a therapeutically effective amount of a compound of Formula I, II, Ill or IV or a pharmaceutically acceptable salt thereof or a pharmaceutical composition thereof. In some embodiments, the methods result in at least a 1% decrease (e.g., at least a 2% decrease, at least a 3% decrease, at least a 4% decrease, at least a 5% decrease, at least a 6% decrease, at least a 8% decrease, at least a 10% decrease, at least a 12% decrease, at least a 14% decrease, at least a 16% decrease, at least a 18% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease) in the migration and/or invasion of a cancer cell in the patient, e.g., as compared to the migration and/or invasion of a cancer cell or a population of cancer cells in a subject having a similar TAM-associated cancer and/or a c-Met-associated cancer, but receiving a different treatment or no treatment.

Some embodiments of these methods further include administering to the patient at least one additional anticancer agent (e.g., any of the exemplary additional anticancer agents described herein or known in the art). For example, in some examples, the at least one anticancer agent or therapy can be selected from the group of: an immune checkpoint inhibitor, a kinase inhibitor, a chemotherapy, radiation and surgery.

In some embodiments of any of the methods described herein, the patient was previously treated with at least one additional anticancer agent (e.g., any of the additional anticancer agents described herein) and the previous treatment with the at least one additional anticancer agent was unsuccessful (e.g., the patient previously developed resistance to one or more of the at least one additional anticancer agent).

In some embodiments of any of the methods described herein, the at least one additional anticancer agent is selected from the group of: a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor (e.g., a type I c-Met kinase inhibitor), a RAF inhibitor, an Akt inhibitor, a FTL-3 inhibitor, and a MAP kinase pathway inhibitor.

In some embodiments of any of the methods described herein, the at least one additional anticancer agent can include a kinase inhibitor, and the patient previously developed resistance to the kinase inhibitor. In some embodiments of any of the methods described herein, the at least one anticancer agent includes a kinase inhibitor selected from the group of: bozitinib, 1-(6,7-Dihydro-5H-benzo[6,7]cyclohepta[1,2-c]pyridazin-3-yl)-N3-[7(S)-(1-pyrrolidiny0-6,7,8,9-tetrahydro-5H-benzocycloheptene-2-yl]-1H-1,2,4-triazole-3,5-diamine (BGB324), crizotinib, foretinib, (N-[4-(2-Amino-3-chloropyridin-4-yloxy)-3-fluorophenyl]-4-ethoxy-1-(4-fluorophenyl)-2-oxo-1,2-dihydropyridine-3-carboxamide (BMS-777607), amuvatinib, BMS-796302, cabozantinib, glesatinib (MGCD265), 2-(4-Fluorophenyl)-N-[3-fluoro-4-(3-phenyl-1H-pyrrolo[2,3-b]pyridin-4-yloxy)phenyl]-1,5-dimethyl-3-oxo-2,3-dihydro-1H-pyrazole-4-carboxamide (NPS-1034), N-[4-[(6,7-Dimethoxyquinolin-4-yl)oxy]-3-fluorophenyl]-4-ethoxy-1-(4-fluoro-2-methylphenyl)-1H-pyrazole-3-carboxamide hydrochloride (LDC1267), gilteritinib, [3-(2-[[3-Fluoro-4-(4-methylpiperazin-1-yl)phenyl]amino]-5-methyl-7H-pyrrolo[2,3-d]pyrimidin-4-yl)phenyl]acetonitrile (SGI-7079), dubermatinib (TP-0903), trans-4-[2-(Butylamino)-5-[4-[(4-methylpiperazin-1-yl)methyl]phenyl]-7H-pyrrolo[2,3-d]pyrimidin-7-yl]cyclohexanol (UNC2025), 3-[3-[4-(Morpholin-4-ylmethyl)-1H-pyrrol-2-ylmethylene]-2-oxo-2,3-dihydro-1H-indol-5-ylmethyl]thiazolidine-2,4-dione hydrochloride (S49076), sunitinib, 12A11, Mab173, YW327.6S2, D9, E8, merestinib, [3-(2-[[3-Fluoro-4-(4-methylpiperazin-1-yl)phenyl]amino]-5-methyl-7H-pyrrolo[2,3-d]pyrimidin-4-yl)phenyl]acetonitrile (SGI1-7079), N-[4-[(6,7-Dimethoxyquinolin-4-yl)oxy]-3-fluorophenyl]-4-ethoxy-1-(4-fluoro-2-methylphenyl)-1H-pyrazole-3-carboxamide hydrochloride, capmatinib, NVP-BVU972, AMG 337, bozitinib, glumetinib, savolitinib, and tepotinib.

In some embodiments of any of the methods described herein, the at least one additional anticancer agent includes dexamethasone, and the patient previously developed resistance to dexamethasone. In some embodiments of any of the methods described herein, the at least one additional anticancer agent includes cytarabine, and the patient previously developed resistance to cytarabine. In some embodiments of any of the methods described herein, the at least one additional anticancer agent includes imatinib, and the patient previously developed resistance to imatinib. In some embodiments of any of the methods described herein, the at least one additional anticancer agent includes lapatinib, and the patient previously developed resistance to lapatinib. In some embodiments of any of the methods described herein, the at least one additional anticancer agent includes cetuximab, and the patient previously developed resistance to cetuximab. In some embodiments of any of the methods described herein, the at least one additional anticancer agent includes erlotinib, and the patient previously developed resistance to erlotinib. In some embodiments of any of the methods described herein, the at least one additional anticancer agent includes alpelisib, and the patient previously developed resistance to alpelisib. In some embodiments of any of the methods described herein, the at least one additional anticancer agent includes cisplatin, and the patient previously developed resistance to cisplatin. In some embodiments of any of the methods described herein, the at least one additional anticancer agent includes sunitinib, and the patient previously developed resistance to sunitinib. In some embodiments of any of the methods described herein, the at least one additional anticancer agent includes metformin, and the patient previously developed resistance to metformin.

In some embodiments of any of the methods described herein, the at least one additional anticancer agent includes an anti-PD1 antibody, and the patient previously developed resistance to the anti-PD1 antibody. In some embodiments of any of the methods described herein, the at least one additional anticancer agent includes docetaxel, and the patient previously developed resistance to docetaxel. In some embodiments of any of the methods described herein, the at least one additional anticancer agent includes an EGFR inhibitor, and the patient previously developed resistance to the EGFR inhibitor.

In some embodiments of any of the methods described herein, the at least one additional anticancer agent is a Type 1 c-Met inhibitor, and the patient previously developed resistance to the c-Met inhibitor. In some embodiments of any of the methods described herein, the at least one additional Type 1 c-Met inhibitor includes crizotinib, and the patient previously developed resistance to crizotinib. In some embodiments of any of the methods described herein, the at least one additional Type 1 c-Met inhibitor includes capmatinib, and the patient previously developed resistance to capmatinib. In some embodiments of any of the methods described herein, the at least one additional Type 1 c-Met inhibitor includes NVP-BVU972, and the patient previously developed resistance to NVP-BVU972. In some embodiments of any of the methods described herein, the at least one additional Type 1 c-Met inhibitor includes AMG 337, and the patient previously developed resistance to AMG 337. In some embodiments of any of the methods described herein, the at least one additional Type 1 c-Met inhibitor includes bozitinib, and the patient previously developed resistance to bozitinib. In some embodiments of any of the methods described herein, the at least one additional Type 1 c-Met inhibitor includes glumetinib, and the patient previously developed resistance to glumetinib. In some embodiments of any of the methods described herein, the at least one additional Type 1 c-Met inhibitor includes savolitinib, and the patient previously developed resistance to savolitinib. In some embodiments of any of the methods described herein, the at least one additional Type 1 c-Met inhibitor includes tepotinib, and the patient previously developed resistance to tepotinib.

In some embodiments, the tumor developed a resistance mutation after treatment with the Type 1 c-Met inhibitor. In some embodiments, the resistance mutation in c-Met results in the expression of a c-Met protein including one or more of the following amino acid substitutions: L1195V, F12001, D1228H, D1228N, D1230C, Y1230H, and Y1230S.

Also provided herein are methods of selecting a treatment for a patient identified as having a TAM-associated cancer, a c-Met-associated cancer, or both, that include selecting a compound of Formula I, II, Ill or IV or a pharmaceutically acceptable salt thereof or a pharmaceutical composition thereof for the patient identified or diagnosed as having a TAM-associated cancer, a c-Met-associated cancer, or both. Some embodiments further comprise administering the selected compound of Formula I, II, Ill or IV or the pharmaceutically acceptable salt thereof or the pharmaceutical composition thereof to the patient.

Also provided herein are methods of selecting a treatment for a patient that include: (a) identifying the patient as having a TAM-associated cancer, a c-Met-associated cancer, or both; and (b) selecting a compound of Formula I, II, Ill or IV or a pharmaceutically acceptable salt thereof or a pharmaceutical composition thereof for the patient identified as having a TAM-associated cancer. Some embodiments further comprise administering the selected compound of Formula I, II, Ill or IV or the pharmaceutically acceptable salt thereof or the pharmaceutical composition thereof to the patient identified as having a TAM-associated cancer, a c-Met-associated cancer, or both.

In some embodiments of any of the methods described herein, the subject is identified or diagnosed as having a TAM-associated cancer (e.g., any of the TAM-associated cancers described herein, e.g., having any of the exemplary TAM mutations described herein). In some embodiments of any of the methods described herein, the subject is identified or diagnosed as having both a TAM-associated cancer (e.g., any of the TAM-associated cancers described herein, e.g., having any of the exemplary TAM mutations described herein) and a c-Met-associated cancer (e.g., any of the exemplary c-Met-associated cancers described herein, e.g., having any of the exemplary c-Met mutations described herein). In some embodiments of any of the methods described herein, the subject is identified or diagnosed as having a c-Met-associated cancer (e.g., any of the exemplary c-Met-associated cancers described herein, e.g., having any of the exemplary c-Met-associated mutations described herein).

In some embodiments of any of the methods described herein, the c-Met-associated cancer is a cancer having a mutation that increases the activity of a c-Met kinase. In some embodiments of any of the methods described herein, the mutation that increases the activity of a c-Met kinase results in one or more amino acid substitutions in the c-Met kinase. In some embodiments of any of the methods described herein, the c-Met-associated cancer is a cancer having a mutation that increases the expression of c-Met in a mammalian cell. In some embodiments of any of the methods described herein, the c-Met-associated cancer is a cancer having a mutation that confers increased half-life of c-Met kinase in a mammalian cell. In some embodiments of any of the methods described herein, the mutation that confers increased half-life of c-Met kinase in a mammalian cell is a mutation that results in c-Met exon 14 skipping during mRNA splicing. In some embodiments of any of the methods described herein, the c-Met-associated cancer is a cancer having a MET gene amplification. In some embodiments of any of the methods described herein, the c-Met-associated cancer is a c-Met-associated cancer that has resistance to a type I c-Met inhibitor.

In some embodiments of any of the methods described herein, the c-Met-associated cancer is selected from the group of: gastrointestinal cancer (GI), gastric cancer, colorectal adenocarcinoma, colorectal carcinoma (CRC), non-small cell lung cancer (NSCLC), hepatocellular carcinoma (HCC), hereditary papillary renal carcinoma (HPRC), papillary renal carcinoma, melanoma, gastric adenocarcinoma, appendiceal adenocarcinoma, duodenal adenocarcinoma, pancreatic adenocarcinoma, lung adenocarcinoma, thyroid papillary carcinoma, thyroid medullary carcinoma, Ewing sarcoma, prostate adenocarcinoma, squamous cell carcinoma of the head and neck and cervix, renal cell carcinoma, pheochromocytoma and composite pheochromocytoma, ovarian serous carcinoma, ovarian clear cell carcinoma, ovarian mixed carcinoma, peritoneal serous carcinoma, breast ductal adenocarcinoma, uterine leiomyosarcoma, uterine endometrioid adenocarcinoma, uterine malignant mixed Mullerian tumor, glioblastoma, anaplastic glioma, oligodendroglioma, desmoplastic small round cell tumor, squamous cell carcinoma of rectum, salivary gland carcinoma, heart angiosarcoma, gastrointestinal stromal tumor, invasive thymoma, and spindle sarcoma.

Also provided herein are methods of selecting a treatment for a patient identified or diagnosed as having a cancer that include: (a) administering an additional anticancer agent to the patient (e.g., any of the additional anticancer agents described herein); (b) after (a), detecting increased expression, level, and/or activity of a TAM kinase and/or c-Met kinase in a cancer cell or an immune cell from the patient; and (c) after (b), selecting a compound of Formula I, II, Ill or IV or a pharmaceutically acceptable salt thereof or a pharmaceutical composition thereof for the patient.

Also provided herein are methods of treating a patient identified or diagnosed as having a cancer that include: (a) administering to the patient identified or diagnosed as having a cancer one or more doses of at least one additional anticancer agent (e.g., at least one of any of the additional anticancer agents described herein); (b) after (a), detecting an increase in the expression, level, and/or activity of a TAM kinase and/or c-Met kinase in a cancer cell or an immune cell from the patient; and (c) after (b), administering to the patient a therapeutically effective amount of a compound of Formula I, II, Ill or IV or a pharmaceutically acceptable salt thereof or a pharmaceutical composition thereof. In some embodiments, step (c) further includes administering to the patient the at least one additional anticancer agent.

Also provided herein are methods of treating a patient identified or diagnosed as having a cancer that include: (a) detecting an increase in the expression, level and/or activity of a TAM kinase and/or c-Met kinase in a cancer cell or an immune cell from a patient identified or diagnosed as having a cancer and previously administered one or more doses of the at least on additional anticancer agent (e.g., any of the additional anticancer agents described herein); and (b) after (a), administering to the patient a therapeutically effective amount of a compound of Formula I, II, Ill or IV or a pharmaceutically acceptable salt thereof or a pharmaceutical composition thereof. In some embodiments, step (b) further includes administering to the patient the at least one additional anticancer agent.

Also provided herein are methods of treating a patient identified or diagnosed as having a cancer that has been previously administered one or more doses of at least one additional anticancer agent and has been identified as having a cancer cell or an immune cell that has increased expression, level, and/or activity of a TAM kinase and/or c-Met kinase that include administering a therapeutically effective amount of a compound of Formula I, II, Ill or IV or a pharmaceutically acceptable salt thereof or a pharmaceutical composition thereof to the patient. In some embodiments, the method further includes administering to the patient that at least one additional anticancer agent.

Also provided herein are methods of treating a patient identified or diagnosed as having a cancer that include: (a) selecting a patient identified or diagnosed as having increased expression, level, and/or activity of a TAM kinase and/or c-Met kinase in a cancer cell or an immune cell; and (b) after (a) administering to the selected patient a therapeutically effective amount of a compound of Formula I, II, Ill or IV or a pharmaceutically acceptable salt thereof or a pharmaceutical composition thereof. In some embodiments, step (b) further includes administering to the patient at least one additional anticancer agent (e.g., any of the additional anticancer agents described herein).

Also provided are methods of treating a patient identified or diagnosed as having a cancer that include: (a) selecting a patient identified or diagnosed as having a cancer that has been previously administered one or more doses of an additional anticancer agent (e.g., any of the additional anticancer agents described herein) and identified as having a cancer cell or an immune cell having increased expression and/or activity of a TAM kinase and/or c-Met kinase; and (b) after (a), administering to the selected patient a therapeutically effective amount of a compound of Formula I, II, Ill or IV or a pharmaceutically acceptable salt thereof or a pharmaceutical composition thereof. In some embodiments, step (b) further includes administering to the patient the at least one additional anticancer agent.

In some embodiments of any of the methods described herein, increased expression, level, and/or activity of a TAM kinase is detected in a cancer cell or an immune cell. In some embodiments of any of the methods described herein, the patient is identified or diagnosed as having a cancer cell or an immune cell having increased expression, level, and/or activity of a TAM kinase.

In some embodiments of any of the methods described herein, an increased expression, level, and/or activity of a TAM kinase and a c-Met kinase are detected in a cancer cell or an immune cell. In some embodiments of any of the methods described herein, the patient is identified or diagnosed as having a cancer cell or an immune cell having increased expression, level, and/or activity of a TAM kinase and a c-Met kinase.

In some embodiments of any of the methods described herein, the increased expression, level, and/or activity of a TAM kinase in a cancer cell or an immune cell results from a chromosomal translocation that results in the expression of a TREM87B-MERTK fusion protein or an AXL-MBIP fusion protein.

In some embodiments of any of the methods described herein, increased expression, level, and/or activity of a c-Met kinase is detected in a cancer cell or an immune cell. In some embodiments of any of the methods described herein, the patient is identified or diagnosed as having a cancer cell or an immune cell having increased expression, level, and/or activity of a c-Met kinase.

Also provided are methods of treating a patient identified or diagnosed as having a TAM-associated cancer that include: (a) administering to the patient identified or diagnosed as having a TAM-associated cancer one or more doses of a TAM kinase inhibitor; (b) after (a), detecting resistance of the TAM-associated cancer in the patient to the TAM kinase inhibitor; and (c) after (b), administering to the patient a therapeutically effective amount of a compound of Formula I, II, Ill or IV or a pharmaceutically acceptable salt thereof or a pharmaceutical composition thereof. In some embodiments, step (c) further includes administering to the patient at least one additional anticancer agent (e.g., any of the additional anticancer agents described herein).

Also provided are methods of treating a patient identified or diagnosed as having a TAM-associated cancer that include: (a) detecting resistance of the TAM-associated cancer in the patient to a TAM kinase inhibitor that was previously administered to the patient; and (b) after (a), administering to the patient a therapeutically effective amount of a compound of Formula I, II, Ill or IV or a pharmaceutically acceptable salt thereof or a pharmaceutical composition thereof. In some embodiments, step (b) further includes administering to the patient at least one additional anticancer agent.

Also provided herein are methods of treating a patient identified or diagnosed as having a TAM-associated cancer and determined to have a previously developed resistance to a TAM kinase inhibitor that include administering to the patient a therapeutically effective amount of a compound of Formula I, II, Ill or IV or a pharmaceutically acceptable salt thereof or a pharmaceutical composition thereof. Some embodiments of these methods further include administering to the patient at least one additional anticancer agent (e.g., any of the additional anticancer agents described herein or known in the art).

Also provided herein are methods of treating a patient identified or diagnosed as having a c-Met-associated cancer that include: (a) administering to the patient identified or diagnosed as having a c-Met-associated cancer one or more doses of a c-Met kinase inhibitor; (b) after (a), detecting resistance of the c-Met-associated cancer in the patient to the c-Met kinase inhibitor; and (c), after (b), administering to the patient a therapeutically effective amount of a compound of Formula I, II, Ill or IV or a pharmaceutically acceptable salt thereof or a pharmaceutical composition thereof. Some embodiments of these methods further include administering to the patient at least one additional anticancer agent (e.g., any of the additional anticancer agents described herein or known in the art). In one embodiment, the c-Met inhibitor administered in step (a) is a Type I c-Met inhibitor. In one embodiment, the Type 1 c-Met inhibitor is crizotinib, capmatinib, NVP-BVU972, AMG 337, bozitinib, glumetinib, savolitinib, or tepotinib.

Also provided herein are methods of treating a patient identified or diagnosed as having a c-Met-associated cancer that include: (a) detecting resistance of the c-Met-associated cancer in the patient to a c-Met kinase inhibitor that was previously administered to the patient; and (b) after (a), administering to the patient a therapeutically effective amount of a compound of Formula I, II, Ill or IV or a pharmaceutically acceptable salt thereof or a pharmaceutical composition thereof. Some embodiments of these methods, step (b) further includes administering to the patient at least one additional anticancer agent. In one embodiment, the c-Met inhibitor administered in step (a) is a Type I c-Met inhibitor. In one embodiment, the Type 1 c-Met inhibitor is crizotinib, capmatinib, NVP-BVU972, AMG 337, bozitinib, glumetinib, savolitinib, or tepotinib.

Also provided herein are methods of treating a patient identified or diagnosed as having a c-Met-associated cancer and determined to have previously developed resistance to a c-Met kinase inhibitor that include administering to the patient a therapeutically effective amount of a compound of Formula I, II, Ill or IV or a pharmaceutically acceptable salt thereof or a pharmaceutical composition thereof. Some embodiments of these methods further include administering to the patient at least one additional anticancer agent. In one embodiment, the patient developed resistance to a Type I c-Met inhibitor. In one embodiment, the Type 1 c-Met inhibitor is crizotinib, capmatinib, NVP-BVU972, AMG 337, bozitinib, glumetinib, savolitinib, or tepotinib.

In some embodiments of any of the methods described herein, the step of identifying the patient as having a TAM-associated cancer and/or c-Met-associated cancer includes performing an assay on a biopsy sample obtained from the patient. In some embodiments, the assay is selected from the group of sequencing, immunohistochemistry, enzyme-linked immunosorbent assay, and fluorescence in situ hybridization (FISH). In some embodiments, the assay is selected from the group of: denaturing gradient gel electrophoresis (DGGE), temperature gradient electrophoresis (TGGE), temperature gradient capillary electrophoresis, a single strand conformational polymorphism assay, a molecular beacon assay, a dynamic hybridization assay, a PCR-based assay and denaturing high performance liquid chromatography. Some embodiments of these methods can further include obtaining the biopsy sample from the patient.

In some embodiments of any of the methods described herein, a compound of Formula I is selected from the compounds described in Example Nos. 1-49, or pharmaceutically acceptable salts thereof. In some embodiments, a compound of Formula I is selected from i) Example Nos. 1-10; ii) Example Nos. 11-20; iii) Example Nos. 21-30; iv) Example Nos. 31-40; v) Example Nos. 41-49; Examples 50-58; or pharmaceutically acceptable salts thereof.

The compounds and methods described herein are useful for the treatment of tumors and cancers (e.g., TAM-associated cancers, and/or c-met-associated cancers). The TAM-associated cancer and/or c-Met-associated cancer treated can be a primary tumor ora metastatic tumor. In one aspect, the methods described herein are used to treat a solid TAM-associated tumor, for example, melanoma, lung cancer (including lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; urinary tract cancer; ovarian cancer or carcinoma (ovarian epithelial carcinoma or surface epithelial-stromal tumor including serous tumor, endometrioid tumor and mucinous cystadenocarcinoma); liver and bile duct carcinoma (including hepatocellular carcinoma, cholangiocarcinoma, hemangioma); esophageal carcinoma or cancer (including esophageal adenocarcinoma and squamous cell carcinoma); oral and oropharyngeal squamous cell carcinoma; salivary gland adenoid cystic carcinoma: bladder cancer; bladder carcinoma; carcinoma of the uterus (including endometrial cancer or endometrial adenocarcinoma, ocular, uterine papillary serous carcinoma, uterine clear-cell carcinoma, uterine sarcomas and leiomyosarcomas, mixed Mullerian tumors); glioma, glioblastoma, medulloblastoma, and other tumors of the CNS; kidney cancers (including renal cancer, renal cell carcinoma, clear cell carcinoma, Wilm's tumor); pituitary adenoma; cancer of the head and neck (including squamous cell carcinomas); cancer of the stomach (gastric cancers, stomach adenocarcinoma, gastrointestinal stromal tumor (GIST)); testicular cancer; germ cell tumor; neuroendocrine tumor; cervical cancer; carcinoids of the gastrointestinal tract, breast, and other organs; signet ring cell carcinoma; mesenchymal tumors including sarcomas (e.g., Kaposi's sarcoma), fibrosarcomas, hemangioma, angiomatosis, hemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomyosarcoma, skin (e.g., squamous cell skin cancer), including melanoma, cervical, retinoblastoma, head and neck cancer, pancreatic, CNS, thyroid, testicular, renal, bladder, soft tissue, adrenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, eystosarcoma phylloide, salivary cancers, thymic carcinomas, and cancers of the vagina among others.

The compounds of Formula I, II, Ill or IV or pharmaceutically acceptable salts thereof can also be used for treating lymphoma or lymphocytic or myelocytic proliferation disorder or abnormality (e.g., a TAM-associated lymphoma or lymphocytic or myelocytic proliferation disorder or abnormality). For example, the TAM-associated cancer can be a Hodgkin Lymphoma of a Non-Hodgkin Lymphoma. For example, the subject can be suffering from a TAM-associated Non-Hodgkin Lymphoma such as, but not limited to: an AIDS-Related Lymphoma; Anaplastic Large-Cell Lymphoma; Angioimmunoblastic Lymphoma; Blastic N -Cell Lymphoma; Burkitt's Lymphoma: Burkitt-like Lymphoma (Small Non-Cleaved Cell Lymphoma); Chronic Lymphocytic Leukemia/Small Lymphocytic Lymphoma: Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma: Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the subject may be suffering from a TAM-associated Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In some embodiments, the methods as described herein may be useful to treat a patient suffering from a specific TAM-associated T-cell, a B-cell, or a NK-cell based lymphoma, proliferative disorder, or abnormality. For example, the patient can be suffering from a specific TAM-associated T-cell or NK-cell lymphoma, for example, but not limited to: Peripheral T-cell lymphoma, for example, peripheral T-cell lymphoma and peripheral T-cell lymphoma not otherwise specified (PTCL-NOS); anaplastic large cell lymphoma, for example anaplastic lymphoma kinase (ALK) positive. ALK negative anaplastic large cell lymphoma, mantle cell lymphoma, or primary cutaneous anaplastic large cell lymphoma; angioimmunoblastic lymphoma; cutaneous T-cell lymphoma, for example mycosis fungoides, Sezary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma: Enteropathy-type T-cell lymphoma; Hematosplenic gamma-delta T-cell Lymphoma: Lymphoblastic Lymphoma; Nasal NK/T-cell Lymphomas; Treatment-related T-cell lymphomas; for example lymphomas that appear after solid organ or bone marrow transplantation; T-cell prolymphocyte leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the methods as described herein may be useful to treat a patient suffering from a specific TAM-associated B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mantle cell lymphoma (MCL); Burkitt lymphoma; Mediastinal large B cell lymphoma; Waldenstrom macroglobulinemia; Nodal marginal zone B cell lymphoma (NMZL); Splenic marginal zone lymphoma (SMZL); Intravascular large B-cell lymphoma; Primary effusion lymphoma; or Lymphomatoid granulomatosis; Chronic lymphocytic leukemia/small lymphocytic lymphoma; B-cell prolymphocyte leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; cell/histiocytic rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma and Burkitt lymphoma; B-cell lymphoma, unclassifiable, with features intermediate between diffuse large B-cell lymphoma and classical Hodgkin lymphoma; Nodular sclerosis classical Hodgkin lymphoma; Lymphocyte-rich classical Hodgkin lymphoma; Mixed cellularity classical Hodgkin lymphoma; or Lymphocyte-depleted classical Hodgkin lymphoma.

In some embodiments, the methods as described herein may be useful to treat a patient suffering from a TAM-associated leukemia. For example, the subject may be suffering from an acute or chronic TAM-associated leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocyte leukemia (a subtype of AML); T-cell prolymphocyte leukemia (TPLL); large granular lymphocytic leukemia; or Adult T-cell chronic leukemia; large granular lymphocytic leukemia (LGL). In some embodiments, the patient suffers from an acute myelogenous leukemia, for example an undifferentiated AML (MO); myeloblasts leukemia (MI; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblast leukemia (M7).

In some embodiments, the compounds and methods described herein are useful for treating a TAM-associated cancer in a patient, wherein the cancer overexpresses AXL, MER, or TYRO3, or a combination thereof, e.g., as compared to a control non-cancerous tissue or a control cell (e.g., from the same or a different subject). In some embodiments, the cancer overexpresses AXL. In some embodiments, the cancer overexpresses MER. In an alternative embodiment, the cancer ectopically expresses MER. In some embodiments, the TAM-associated cancer is breast, colon, renal, skin, lung (including non-small cell lung cancer), liver, gastric, CNS cancer (including glioblastoma), ovarian, pancreatic, prostate, glioblastoma multiforme, osteosarcoma, thyroid malignancies, rhabdomyosarcoma, melanoma. acute myeloid leukemia, T-cell acute lymphoid leukemia, B-cell acute lymphoid leukemia, schwannoma, and mantle cell lymphoma.

In some embodiments, the TAM-associated cancer is selected from breast, colon, renal, skin, lung (including non-small cell lung cancer), liver, gastric, CNS (including glioblastoma), ovarian, pancreatic, prostate, glioblastoma multiforme, osteosarcoma, thyroid malignancies, rhabdomyosarcoma, and melanoma.

In some embodiments, the TAM-associated cancer is selected from leukemias (including acute myeloid leukemia and chronic myeloid leukemia, B-cell myeloid leukemia (B-CLL), B-cell acute lymphoblastic leukemia, erythroid leukemia, and T-lineage acute lymphoblastic leukemia), non-small cell lung cancer, pancreatic ductal adenocarcinoma, astrocytoma, lung adenocarcinoma, ovarian cancer, melanoma, and glioblastoma multiforme.

In some embodiments, the TAM-associated cancer is selected from chronic myeloid leukemia, gastrointestinal stromal tumors (GIST), breast cancer (e.g., HER2 positive breast cancer and triple negative breast cancer), head and neck cancer, and non-small cell lung cancer.

In some embodiments of any of the methods described herein, the TAM-associated cancer is a cancer having overexpression of a TAM kinase, e.g., as compared to a non-cancerous tissue or cell in the same patient or a different subject. In some embodiments of any of the methods described herein, the TAM-associated cancer is a cancer having ectopic expression of a TAM kinase.

In some embodiments of any of the methods described herein, the TAM-associated cancer is a cancer having overexpression or ectopic expression of a TYRO3 protein. In some embodiments of any of the methods described herein, the TAM-associated cancer has one or more point mutations in a gene encoding TYRO3 that results in the expression of a TYRO3 that includes one or more amino acid substitutions. In some embodiments of any of the methods described herein, the TAM-associated cancer has a chromosomal translocation which results in the expression of a fusion protein including the kinase domain of TYRO3 and a fusion partner. Non-limiting examples of a TAM-associated cancer having overexpression or ectopic expression of TYRO3, or a mutation in a TYRO3 gene that results in the expression of TYRO3 having one or more point mutations or a TYRO3 fusion protein include: AML, multiple myeloma, lung cancer, melanoma, prostate cancer, endometrial cancer, thyroid cancer, schwannoma, pancreatic cancer, and CNS cancer. Non-limiting aspects of TAM-associated cancers having increased expression and/or activity of TYRO3 are listed in Table 4.

TABLE 4
TAM-Associated Cancers Having with Increased
Expression and/or Activity of TYRO3

| | |
|---|---|
| Melanoma | Amino acid substitutions at: Q67 and/or |
| | R462Q, and/or W708fs*5 |
| Lung Cancer | Amino acid substitution at E340 or |
| | N615K in TYRO3 |
| Pancreatic Cancer | Amino acid substitution R514Q in |
| | TYRO3 |
| Colon Cancer | Amino acid substitution G809D and/or |
| | M592I in TYRO3 |
| CNS Cancer (brain cancer) | Amino acid substitution A709T in |
| | TYRO3 |
| AML, multiple myeloma, | Overexpression or ectopic expression of |
| lung cancer, melanoma, | TYRO3 |
| prostate cancer, endometrial | |
| cancer, thyroid cancer, and | |
| schwannoma | |

Additional anticancer agents that are TYRO3 inhibitors include, e.g., 6g, merestinib (LY2801653), ASLAN002 (BMS-777607; (N-[4-(2-Amino-3-chloropyridin-4-yloxy)-3-fluorophenyl]-4-ethoxy-1-(4-fluorophenyl)-2-oxo-1,2-dihydropyridine-3-carboxamide), LDC1267 (N-[4-[(6,7-Dimethoxyquinolin-4-yl)oxy]-3-fluorophenyl]-4-ethoxy-1-(4-fluoro-2-methylphenyl)-1H-pyrazole-3-carboxamide hydrochloride, and UNC2025 (trans-4-[2-(Butylamino)-5-[4-[(4-methylpiperazin-1-yhmethyl]phenyl]-7H-pyrrolo[2,3-d]pyrimidin-7-yl]cyclohexanol).

In some embodiments of any of the methods described herein, the TAM-associated cancer is a cancer having overexpression or ectopic expression of an AXL protein. In some embodiments of any of the methods described herein, the TAM-associated cancer has one or more point mutations in a gene encoding AXL that results in the expression of an AXL that includes one or more amino acid substitutions. In some embodiments of any of the methods described herein, the TAM-associated cancer has a chromosomal translocation which results in the expression of a fusion protein including the kinase domain of AXL and a fusion partner. Non-limiting examples of a TAM-associated cancer having overexpression or ectopic expression of AXL, or a mutation in an AXL gene that results in the expression of AXL having one or more point mutations or an AXL fusion protein include: AML, CML, B-CLL, lung cancer, glioblastoma, breast cancer, colorectal cancer, gastric cancer, pancreatic cancer, esophageal cancer, melanoma, squamous cell skin cancer, prostate cancer, endometrial cancer, ovarian cancer, oral squamous cell carcinoma, thyroid cancer, bladder cancer, renal cancer, schwannoma, mesothelioma, Kaposi's sarcoma, osteosarcoma, erythroid leukemia, colon cancer, liver cancer, renal cell carcinoma, osteosarcoma, kidney cancer, PH+ CML, non-small cell lung cancer, triple-negative metastatic breast cancer, and HER2+ breast cancer. Non-limiting aspects of TAM-associated cancers having increased expression and/or activity of AXL are listed in Table 5.

TABLE 5
TAM-Associated Cancers Having with Increased
Expression and/or Activity of AXL

| | |
|---|---|
| Ovarian Cancer | Amino acid substitutions C24G and/or |
| | A358V in AXL |
| Melanoma | One or more of the amino acid substitutions |
| | of P36L, R236C, G413W, E431K, A451T, |
| | E535K, G829E, I610V, A666T, S685F, and |
| | R784Q in AXL |
| Colon Cancer | One or more of the amino acid substitutions |
| | of N43T, M580K, and L684P in AXL |
| Skin Cancer | An amino acid substitution of P238L in AXL |
| Gastric Cancer | One or more of the amino acid substitutions |
| | of V289M, R492C, S842F, and P636H |
| | in AXL |
| Lung Cancer | One or more of the amino acid substitutions |
| | of R295W, L423Q, K526N, and S599F |
| | in AXL |
| Breast Cancer | One or more of the amino acid substitutions |
| | of T343M, E745K, and S747R in AXL |
| Prostate Cancer | An amino acid substitution of R368Q in AXL |
| Pancreatic Cancer | An amino acid substitution of E484D in AXL |
| Kidney Cancer | An amino acid substitution of P742T in AXL |
| AML, CML, B-CLL, | Overexpression or ectopic expression of AXL |
| lung cancer, glioblastoma, | |
| breast cancer, colorectal | |
| cancer, gastric cancer, | |
| pancreatic cancer, | |
| esophageal cancer, | |
| melanoma, squamous | |
| cell skin cancer, prostate | |
| cancer, endometrial | |
| cancer, ovarian cancer, | |
| oral squamous cell | |
| carcinoma, thyroid | |
| cancer, bladder cancer, | |
| renal cancer, | |
| schwannoma, | |
| mesothelioma, | |
| Kaposi's sarcoma, and | |
| osteosarcoma | |

Additional anticancer agents that are AXL inhibitors include, e.g., bozitinib (SKI-606, PF-5208765, Bosulif), Bemcentinib (BGB324; R428), crizotinib (PF-2341066, Xalkon), foretinib (GSK1363089, XL880), (N-[4-(2-Amino-3-chloropyridin-4-yloxy)-3-fluorophenyl]-4-ethoxy-1-(4-fluorophenyl)-2-oxo-1,2-dihydropyridine-3-carboxamide (BMS-777607; ASLAN002), LY2801653 (merestinib), amuvatinib (MP-470), cabozantinib (XL184, BMS-907351, Cometriq), glesatinib (MGCD265), NPS-1034 (2-(4-Fluorophenyl)-N-[3-fluoro-4-(3-phenyl-1H-pyrrolo[2,3-b]pyridin-4-yloxy)phenyl]-1,5-dimethyl-3-oxo-2,3-dihydro-1H-pyrazole-4-carboxamide), LDC1267 (N-[4-[(6,7-Dimethoxyquinolin-4-yl)oxy]-3-fluorophenyl]-4-ethoxy-1-(4-fluoro-2-methylphenyl)-1H-pyrazole-3-carboxamide hydrochloride), gilteritinib (ASP2215), SGI-7079 ([3-(2-[[3-Fluoro-4-(4-methylpiperazin-1-yl)phenyl]amino]-5-methyl-7H-pyrrolo[2,3-d]pyrimidin-4-yl)phenyl]acetonitrile), dubermatinib (TP-0903), trans-4-[2-(Butylamino)-5-[4-[(4-methylpiperazin-1-yhmethyl]phenyl]-7H-pyrrolo[2,3-d]pyrimidin-7-yl]cyclohexanol (UNC2025), 3-[3-[4-(Morpholin-4-ylmethyl)-1H-pyrrol-2-ylmethylene]-2-oxo-2,3-dihydro-1H-indol-5-ylmethyl]thiazolidine-2,4-dione hydrochloride (S49076), sunitinib (SU11248, Sutent), and the monoclonal antibodies of 12A11, Mab173, YW327.652, D9, and E8.

In some embodiments of any of the methods described herein, the TAM-associated cancer is a cancer having overexpression or ectopic expression of a MER protein. In some embodiments of any of the methods described herein, the TAM-associated cancer has one or more point mutations in a gene encoding MER that results in the expression of a MER that includes one or more amino acid substitutions. In some embodiments of any of the methods described herein, the TAM-associated cancer has a chromosomal translocation which results in the expression of a fusion protein including the kinase domain of MER and a fusion partner. Non-limiting examples of a TAM-associated cancer having overexpression or ectopic expression of MER, or a mutation in a MER gene that results in the expression of MER having one or more point mutations or a MER fusion protein include: AML, ALL (B-ALL, T-ALL), lung cancer, glioma, melanoma, prostate cancer, schwannoma, mantle cell lymphoma, rhabdomyosarcoma, pancreatic cancer, breast cancer, gastric cancer, pituitary adenoma, urinary tract cancer, kidney cancer, liver cancer, colon cancer, and breast cancer. Non-limiting aspects of MER-associated cancers having increased expression and/or activity of MER are listed in Table 6.

TABLE 6
TAM-Associated Cancers Having with Increased
Expression and/or Activity of MER

| | |
|---|---|
| Melanoma | One or more amino acid substitutions of |
| | P40S, V861I, K923R, and P802S in MER |
| Lung Cancer | One or more amino acid substitutions of |
| | S159F, I431F, S905F, P672S, N718Y, |
| | and M790V in MER |
| Urinary Tract Cancer | One or more amino acid substitutions of |
| | E204K, L586F, and S626C in MER |
| Gastric Cancer | An amino acid substitutions of S428G |
| | in MER |
| Kidney Cancer | Amino acid substitutions of A446G and/or |
| | P958L in MER |
| Liver Cancer | One or more amino acid substitutions of |
| | N454S, V873I, and D983N in MER |
| Lymphoma | An amino acid substitution of W485S/C |
| | in MER |
| Colon Cancer | One or more amino acid substitutions of |
| | D990N, L688M, and R722 in MER |
| Breast Cancer | An amino acid substitution of G594R |
| | in MER |
| Head and Neck Cancer | An amino acid substitution of A708S |
| | in MER |
| AML, ALL, lung cancer, | Overexpression or ectopic expression |
| glioma, melanoma, prostate | of MER |
| cancer, schwannoma, mantle | |
| cell lymphoma, and | |
| rhabdomyosarcoma | |

Additional anticancer agents that are MER inhibitors include, e.g., foretinib, merestinib (LY2801653), (N-[4-(2-Amino-3-chloropyridin-4-yloxy)-3-fluorophenyl]-4-ethoxy-1-(4-fluorophenyl)-2-oxo-1,2-dihydropyridine-3-carboxamide (ASLAN002; BMS-777607), [3-(2-[[3-Fluoro-4-(4-methylpiperazin-1-yl)phenyl]amino]-5-methyl-7H-pyrrolo[2,3-d]pyrimidin-4-yl)phenyl]acetonitrile (SGI-7079), dubermatinib (TP-0903), trans-4-[2-(Butylamino)-5-[4-[(4-methylpiperazin-1-yl)methyl]phenyl]-7H-pyrrolo[2,3-d]pyrimidin-7-yl]cyclohexanol (UNC2025), and 3-[3-[4-(Morpholin-4-ylmethyl)-1H-pyrrol-2-ylmethylene]-2-oxo-2,3-dihydro-1H-indol-5-ylmethyl]thiazolidine-2,4-dione hydrochloride (S49076).

A subset of compounds of Formula I, i.e., compounds of Formula IV, were unexpectedly found to have low MDR1 efflux ratios relative to other compounds of Formula I, as demonstrated in Example E and Table E1, indicating that compounds of Formula IV will have increased brain penetration compared to such certain compounds of Formula I. In one embodiment, the compounds of Formula IV include the following compounds of Examples 1, 4, 5, 16, 18, 19, 21, 28, 30, 31, 33, 35, 39, 41, 48, 49, 51, 52, 57 and 58:

N-(5-((6,7-dimethoxyquinolin-4-yl)oxy)pyridin-2-yl)-5-(4-fluorophenyl)-1-isopropyl-4-oxo-1,4-dihydropyridine-3-carboxamide;

5-(2,4-difluorophenyl)-N-(5-((6,7-dimethoxyquinolin-4-yl)oxy)pyridin-2-yl)-1-isopropyl-4-oxo-1,4-dihydropyridine-3-carboxamide;

5-(4-chlorophenyl)-N-(5-(6,7-dimethoxyquinolin-4-yl)oxy)pyridin-2-yl)-1-isopropyl-4-oxo-1,4-dihydropyridine-3-carboxamide;

5-(2,4-difluorophenyl)-N-(5-(6,7-dimethoxyquinolin-4-yl)oxy)pyridin-2-yl)-4-oxo-1-(pentan-3-yl)-1,4-dihydropyridine-3-carboxamide;

5-(2-chloro-4-fluorophenyl)-N-(5(6,7-dimethoxyquinolin-4-yl)oxy)pyridin-2-yl)-1-isopropyl-4-oxo-1,4-dihydropyridine-3-carboxamide;

N-(5-((6,7-dimethoxyquinolin-4-yl)oxy)pyridin-2-yl)-5-(4-fluoro-2-methylphenyl)-1-isopropyl-4-oxo-1,4-dihydropyridine-3-carboxamide;

5-(3-chloro-4-fluorophenyl)-N-(5-((6,7-dimethoxyquinolin-4-yl)oxy)pyridin-2-yl)-1-isopropyl-4-oxo-1,4-dihydropyridine-3-carboxamide;

5-(3,4-difluorophenyl)-N-(5((6,7-dimethoxyquinolin-4-yl)oxy)pyridin-2-yl)-1-isopropyl-6-methyl-4-oxo-1,4-dihydropyridine-3-carboxamide;

N-(5((6,7-dimethoxyquinolin-4-yl)oxy)pyridin-2-yl)-1-isopropyl-5-(4-methoxyphenyl)-4-oxo-1,4-dihydropyridine-3-carboxamide;

N-(5(6,7-dimethoxyquinolin-4-yl)oxy)pyridin-2-yl)-5-(2-fluoro-4-methoxyphenyl)-1-isopropyl-4-oxo-1,4-dihydropyridine-3-carboxamide;

5-(4-fluorophenyl)-1-isopropyl-N-(5((6-methoxyquinolin-4-yl)oxy)pyridin-2-yl)-6-methyl-4-oxo-1,4-dihydropyridine-3-carboxamide;

5-(3,4-difluorophenyl)-N-(5((6,7-dimethoxyquinolin-4-yl)oxy)pyridin-2-yl)-1-isopentyl-4-oxo-1,4-dihydropyridine-3-carboxamide;

1-ethyl-5-(4-fluorophenyl)-N-(5-((6-methoxyquinolin-4-yl)oxy)pyridin-2-yl)-4-oxo-1,4-dihydropyridine-3-carboxamide;

5-(4-fluorophenyl)-N-(5-((6-methoxyquinolin-4-yl)oxy)pyridin-2-yl)-1-methyl-4-oxo-1,4-dihydropyridine-3-carboxamide;

N-(5-((7-fluoro-6-methoxyquinolin-4-yl)oxy)pyridin-2-yl)-5-(4-fluorophenyl)-1-isopropyl-6-methyl-4-oxo-1,4-dihydropyridine-3-carboxamide;

N-(5-((7-fluoro-6-methoxyquinolin-4-yl)oxy)pyridin-2-yl)-5-(4-fluorophenyl)-1-isopropyl-4-oxo-1,4-dihydropyridine-3-carboxamide;

N-(5-((6-ethoxyquinolin-4-yl)oxy)pyridin-2-yl)-5-(4-fluorophenyl)-1-isopropyl-4-oxo-1,4-dihydropyridine-3-carboxamide;

N-(5-((7-ethoxyquinolin-4-yl)oxy)pyridin-2-yl)-5-(4-fluorophenyl)-1-isopropyl-4-oxo-1,4-dihydropyridine-3-carboxamide;

N-(5-((3-cyano-6,7-dimethoxyquinolin-4-yl)oxy)pyridin-2-yl)-5-(4-fluorophenyl)-1-isopropyl-4-oxo-1,4-dihydropyridine-3-carboxamide; and

N-(6-((6,7-dimethoxyquinolin-4-yl)oxy)pyridin-3-yl)-5-(4-fluorophenyl)-1-isopropyl-4-oxo-1,4-dihydropyridine-3-carboxamide;

and pharmaceutically acceptable salts thereof.

Accordingly, in some embodiments, provided herein is a method of treating a CNS cancer in a patient in need of such treatment, the method comprising administering to the patient a therapeutically effective amount of a compound of Formula IV or a pharmaceutically acceptable salt thereof. In some such embodiments, the compound of Formula IV is selected from a compound of Examples 1, 4, 5, 16, 18, 19, 21, 28, 30, 31, 33, 35, 39, 41, 48, 49, 51, 52, 57 and 58 and pharmaceutically acceptable salts thereof.

The term “CNS cancer” or “cancer of the CNS” or as used interchangeably herein refers to a cancer (i.e., a malignant tumor) of the CNS, including cancers of the brain (also known as intracranial tumors), cancers of the spinal cord, and cancers of the meninges surrounding the brain and spinal cord.

In one embodiment, the CNS cancer is a metastatic brain cancer. The metastatic brain cancer may be the result of any cancer described herein, wherein the subject has developed at least one brain metastasis. In one embodiment, the CNS cancer is neuroblastoma with at least one brain metastasis.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Metastatic brain cancer refers to cancer in the brain, i.e., cancer which originated in a tissue other than the brain and has metastasized to the brain.

In one embodiment, the CNS cancer is a primary brain tumor. Primary brain tumors are tumors that start in the brain or spine and are known collectively as gliomas. The term “glioma” is used to describe tumors that originate in glial cells present in the CNS. According to the WHO classification of brain tumors, gliomas are graded by the cell activity and aggressiveness on a scale including Grade I (benign CNS tumors) and Grades II to IV (malignant CNS tumors):

Grade I glioma (Pilocytic astrocytoma): typically occurs in children in the cerebellum or brainstem, and occasionally in the cerebral hemispheres, and are slow growing. Grade I can occur in adults. Although they are benign (WHO grade I), the difficulty in curing this disease makes their growth malignant in behavior with high morbidity rates (Rostami, Acta Neurochir (Wien). 2017; 159(11): 2217-2221).

Grade II glioma (Low-grade gliomas): includes astrocytoma, oligodendroglioma, and mixed oligoastrocytoma. Grade II gliomas typically occur in young adults (20s-50s) and are most often found in the cerebral hemispheres. Due to the infiltrative nature of these tumors, recurrences may occur. Some grade II gliomas recur and evolve into more aggressive tumors (grade III or IV).

Grade III glioma (Malignant glioma): includes anaplastic astrocytoma, anaplastic oligodendroglioma, and anaplastic mixed oligoastrocytoma. Grade III tumors are aggressive, high-grade cancers and invade nearby brain tissue with tentacle-like projections, making complete surgical removal more difficult.

Grade IV gliomas: includes Glioblastoma multiforme (GBM) and gliosarcoma; (GBM) is a malignant glioma. GBM is the most aggressive and most common primary brain tumor. Glioblastoma multiforme usually spreads quickly and invades other parts of the brain, with tentacle-like projections, making complete surgical removal more difficult. Gliosarcoma is a malignant cancer and is defined as a glioblastoma consisting of gliomatous and sarcomatous components.

In one embodiment, the CNS cancer is a peripheral nervous system cancer. In one embodiment, the peripheral nervous system cancer is neuroblastoma.

Also provided are methods for treating a cancer (e.g., a TAM-associated cancer and/or c-Met-associated cancer) in a patient in need thereof, the method comprising: (a) determining if the cancer in the patient is a TAM-associated cancer, a c-Met-associated cancer, or both; and (b) if the cancer is determined to be a TAM-associated cancer a c-Met-associated cancer, or both, administering to the patient a therapeutically effective amount of a compound of Formula I, II, Ill or IV or a pharmaceutically acceptable salt thereof or a pharmaceutical composition thereof. Some embodiments of these methods further include administering to the subject at least one additional anticancer agent (e.g., an immunotherapy). In some embodiments, the subject was previously treated with at least one additional anticancer agent or therapy, e.g., a kinase inhibitor, an immunotherapy, chemotherapy, radiation therapy and/or surgery. In some embodiments the patient has a cancer that is resistant to the at least one additional anticancer agent. In some embodiments, the at least one additional anticancer agent does not include a compound of Formula I, II, Ill or IV.

Also provided herein is a method for treating a patient diagnosed with or identified as having a TAM-associated cancer, (e.g., any of the exemplary TAM-associated cancers disclosed herein), a c-Met-associated cancer (e.g., any of the exemplary c-Met-associated cancers disclosed herein), or both, comprising administering to the patient a therapeutically effective amount of a compound of Formula I, II, Ill or IV or a pharmaceutically acceptable salt thereof, or a pharmaceutical composition thereof as defined herein. Some embodiments of these methods further include administering to the subject at least one additional anticancer agent (e.g., an immunotherapy). In some embodiments, the subject was previously treated with at least one additional anticancer agent or therapy, e.g., a kinase inhibitor, an immunotherapy, chemotherapy, radiation therapy and or surgery. In some embodiments the patient has a cancer that is resistant to the previously administered at least one additional anticancer agent. In some embodiments, the at least one additional anticancer agent does not include a compound of Formula I, II, Ill or IV.

In some embodiments, provided herein are methods for treating a patient diagnosed with (or identified as having) a cancer (e.g., a TAM-associated cancer, a c-Met-associated cancer, or both) that include administering to the patient a therapeutically effective amount of a compound of Formula I, II, Ill or IV or a pharmaceutically acceptable salt thereof. Also provided herein are methods for treating a patient identified or diagnosed as having a TAM-associated cancer, a c-Met-associated cancer, or both, that include administering to the patient a therapeutically effective amount of a compound of Formula I, II, Ill or IV or a pharmaceutically acceptable salt thereof or a pharmaceutical composition thereof. In some embodiments, the patient that has been identified or diagnosed as having a TAM-associated cancer, a c-Met-associated cancer, or both, through the use of a regulatory agency-approved, e.g., FDA-approved test or assay for identifying abnormal (e.g., increased) expression, level, and/or activity of one or more of the TAM kinases and/or c-Met kinase, in a patient or a biopsy sample from the patient or by performing any of the non-limiting examples of assays described herein. In some embodiments, the test or assay is provided as a kit. Some embodiments of these methods further include administering to the patient at least one additional anticancer agent (e.g., an immunotherapy). In some embodiments, the patient was previously treated with at least one additional anticancer agent or therapy, e.g., a kinase inhibitor, an immunotherapy (e.g., an immune checkpoint inhibitor), chemotherapy, radiation therapy and or surgery. In some embodiments, the patient has a cancer that is resistant to the at least one additional anticancer agent. In some embodiments, the at least one additional anticancer agent does not include a compound of Formula I, II, Ill or IV.

Also provided is a compound of Formula I, II, Ill or IV or a pharmaceutically acceptable salt thereof, for use in the treatment of a cancer in a patient in need thereof or a patient identified or diagnosed as having a TAM-associated cancer (e.g., any of the TAM-associated cancers described herein), a c-Met-associated cancer (e.g., any of the c-Met-associated cancers described herein), or both. Also provided is the use of a compound of Formula I, II, Ill or IV or a pharmaceutically acceptable salt thereof for the manufacture of a medicament for treating a cancer in a patient identified or diagnosed as having a TAM-associated cancer, a c-Met-associated cancer, or both. In some embodiments, the cancer is a TAM-associated cancer. In some embodiments, the cancer is a c-Met-associated cancer. In some embodiments, the cancer is both a TAM-associated cancer and a c-Met-associated cancer. In some embodiments, a patient is identified or diagnosed as having a TAM-associated cancer, a c-Met-associated cancer, or both, through the use of a regulatory agency-approved, e.g., FDA-approved, kit for identifying abnormal (e.g., increased) expression, level, and/or activity of one or more of the TAM kinases and/or c-Met kinase, e.g., as compared to a non-cancerous tissue or cell from the same or a different subject. Some embodiments of these methods further include administering to the patient at least one additional anticancer agent (e.g., immunotherapy). In some embodiments, the subject was previously treated with at least one additional anticancer agent or therapy, e.g., an immune checkpoint inhibitor, a kinase inhibitor, an immunotherapy, chemotherapy, radiation therapy and/or surgery. In some embodiments, the patient has a cancer that is resistant to one or more of the at least one additional anticancer agents. In some embodiments, the at least one additional anticancer agent does not include a compound of Formula I, II, Ill or IV.

In some embodiments of any of the methods or uses described herein, the patient has been identified or diagnosed as having a cancer associated with or having abnormal (e.g., increased) expression, level, and/or activity of one or more of the TAM kinases and/or c-Met kinase, e.g., as compared to a non-cancerous tissue or cell in the same or a different subject. In some embodiments, provided herein are methods for treating a TAM-associated cancer, a c-Met-associated cancer, or both, in a patient in need of such treatment, the method comprising a) detecting abnormal (e.g., increased) expression and/or activity of one or more of the TAM kinases and/or c-Met kinase, e.g., as compared to a non-cancerous tissue or cell in the same or a different subject; and b) after a), administering a therapeutically effective amount of a compound of Formula I, II, Ill or IV or a pharmaceutically acceptable salt thereof. Some embodiments of these methods further include administering to the patient at least one additional anticancer agent (e.g., immunotherapy). In some embodiments, the patient was previously treated with at least one additional anticancer agent or therapy, e.g., a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

In some embodiments of any of the methods or uses described herein, the patient has a clinical record indicating that the patient has a tumor associated with or having abnormal (e.g., increased) expression, level, and/or activity of one or more of the TAM kinases and/or c-Met kinase, e.g., as compared to a non-cancerous tissue or cell in the same patient or a different subject). In some embodiments, the clinical record indicates that the patient should be treated with one or more of the compounds of Formula I, II, Ill or IV or a pharmaceutically acceptable salts thereof or compositions provided herein. Some embodiments of these methods further include administering to the patient at least one additional anticancer agent (e.g., an immunotherapy). In some embodiments, the subject was previously treated with at least one additional anticancer agent or therapy, e.g., a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

Also provided are methods of treating a patient that include administering a therapeutically effective amount of a compound of Formula I, II, Ill or IV or a pharmaceutically acceptable salt thereof to a patient having a clinical record that indicates that the patient has a cancer associated with or having abnormal (e.g., increased) expression, level and/or activity of one or more of the TAM kinases and/or c-Met kinase, e.g., as compared to a non-cancerous tissue or cell from the patient or a different subject. Also provided is the use of a compound of Formula I, II, Ill or IV or a pharmaceutically acceptable salt thereof for the manufacture of a medicament for treating a TAM-associated cancer, a c-Met-associated cancer, or both, in a patient having a clinical record that indicates that the patient has a cancer associated with or having abnormal (e.g., increased) expression, level, and/or activity of one or more of the TAM kinases and/or c-Met kinase, e.g., as compared to a non-cancerous tissue or cell from the patient or a different subject. Some embodiments of these methods and uses can further include: a step of performing an assay on a sample (e.g., a biopsy sample) obtained from the patient to determine whether the patient has abnormal (e.g., increased) expression, level, and/or activity of one or more of the TAM kinases and/or c-Met kinase (e.g., as compared to a non-cancerous tissue or cell from the patient or a different subject), and recording the information in a patient's clinical file (e.g., a computer readable medium) that the patient has been identified to have abnormal (e.g., increased) expression and/or activity of one or more of the TAM kinases and/or c-Met kinase. In some embodiments, the assay is an in vitro assay. Some embodiments of these methods further include administering to the patient at least one additional anticancer agent (e.g., an immunotherapy). In some embodiments, the subject was previously treated with at least one additional anticancer agent or therapy, e.g., a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

Also provided herein is a method of treating a patient in need thereof. The method includes performing an assay on a sample obtained from the patient to determine whether the subject has abnormal (e.g., increased) expression, level, and/or activity of one or more of the TAM kinases and/or c-Met kinase, e.g., as compared to a non-cancerous tissue or cell from the same patient or a different subject). The method also includes administering to a patient determined to have abnormal (e.g., increased) expression, level, and/or activity of one or more of the TAM kinases and/or c-Met kinase (e.g., as compared to a non-cancerous tissue or cell from the same patient or a different subject) a therapeutically effective amount of a compound of Formula I, II, Ill or IV or a pharmaceutically acceptable salt thereof. Some embodiments of these methods further include administering to the patient at least one additional anticancer agent (e.g., an immunotherapy). In some embodiments, the patient was previously treated with at least one additional anticancer agent or therapy, e.g., a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

Also provided are methods (e.g., in vitro methods) of selecting a treatment for a patient identified or diagnosed as having a TAM-associated cancer, a c-Met-associated cancer, or both. Some embodiments can further include administering the selected treatment to the patient identified or diagnosed as having a TAM-associated cancer, a c-Met-associated cancer, or both. For example, the selected treatment can include administration of a therapeutically effective amount of a compound of Formula I, II, Ill or IV or a pharmaceutically acceptable salt thereof. Some embodiments can further include a step of performing an assay on a sample (e.g., a biopsy sample) obtained from the patient to determine whether the patient has abnormal (e.g., increased) expression and/or activity of one or more of the TAM kinases and/or c-Met kinase (e.g., as compared to a non-cancerous tissue or cell from the same patient or a different subject), and identifying and diagnosing a patient determined to have abnormal (e.g., increased) expression, level, and/or activity of one or more of the TAM kinases and/or c-Met kinase, as having a TAM-associated cancer and/or c-Met-associated cancer, respectively. In some embodiments, the patient has been identified or diagnosed as having a TAM-associated cancer, a c-Met-associated cancer, or both, through the use of a regulatory agency-approved, e.g., FDA-approved, kit for identifying abnormal (e.g., increased) expression, level, and/or activity of one or more of the TAM kinases and/or c-Met kinase in a patient or a biopsy sample from the patient. In some embodiments, the TAM-associated cancer is a cancer described herein or known in the art. In some embodiments, the c-Met-associated cancer is a cancer described herein or known in the art. In some embodiments, the assay is an in vitro assay. For example, an assay that utilizes the next generation sequencing, immunohistochemistry, or break apart FISH analysis. In some embodiments, the assay is a regulatory agency-approved, e.g., FDA-approved, kit. Some embodiments of these methods further include administering to the patient at least one additional anticancer agent (e.g., an immunotherapy). Some embodiments of these methods further include administering to the subject at least one additional anticancer agent (e.g., an immunotherapy). In some embodiments, the patient was previously treated with at least one additional anticancer agent or therapy, e.g., an immune checkpoint inhibitor, a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

Also provided herein are methods of selecting a treatment for a patient, wherein the methods include a step of performing an assay on a sample obtained from the patient to determine whether the patient has abnormal (e.g., increased) expression, level, and/or activity of one or more of the TAM kinases and/or c-Met kinase, e.g., as compared to a non-cancerous tissue or cell from the patient or a different subject. Some embodiments further include administering the selected treatment to the patient identified or diagnosed as having a TAM-associated cancer, a c-Met-associated cancer, or both. For example, the selected treatment can include administration of a therapeutically effective amount of a compound of Formula I, II, Ill or IV or a pharmaceutically acceptable salt thereof to the patient identified or diagnosed as having a TAM-associated cancer. In some embodiments, the assay is an in vitro assay. Some embodiments of these methods further include administering to the patient at least one additional anticancer agent (e.g., an immunotherapy). In some embodiments, the patient was previously treated with at least one additional anticancer agent or therapy, e.g., an immune checkpoint inhibitor, a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

Also provided are methods of selecting a patient for treatment, wherein the methods include selecting, identifying, or diagnosing a patient having a TAM-associated cancer, a c-Met-associated cancer, or both, and selecting the patient for treatment including administration of a therapeutically-effective amount of a compound of Formula I, II, Ill or IV or a pharmaceutically acceptable salt thereof. In some embodiments, identifying or diagnosing a patient as having a TAM-associated cancer, a c-Met-associated cancer, or both, can include a step of performing an assay on a sample obtained from the patient to determine whether the patient has abnormal (e.g., increased) expression, level, and/or activity of one or more of the TAM kinases and/or c-Met kinase (e.g., as compared to a non-cancerous tissue or cell from the patient or a different subject), as having a TAM-associated cancer and/or c-Met-associated cancer, respectively. In some embodiments, the method of selecting a treatment can be used as a part of a clinical study that includes administration of various treatments of a TAM-associated cancer, a c-Met-associated cancer, or both. In some embodiments, the assay is an in vitro assay. Some embodiments of these methods further include administering to the subject at least one additional anticancer agent or therapy, e.g., an immune checkpoint inhibitor, a kinase inhibitor, an immunotherapy, chemotherapy, radiation therapy and/or surgery. In some embodiments, the patient has a cancer that is resistant to one or more of the at least one additional anticancer agent. In some embodiments, the at least one additional anticancer agent does not include a compound of Formula I, II, Ill or IV.

In some embodiments of any of the methods or uses described herein, an assay can be used to determine whether the patient has abnormal (e.g., increased) expression, level, and/or activity of one or more of the TAM kinases and/or c-Met kinase. In some embodiments, the sample is a biological sample or a biopsy sample (e.g., a paraffin-embedded biopsy sample) from the patient. In some embodiments, the patient is a patient suspected of having a TAM-associated cancer, a c-Met-associated cancer, or both, a patient having one or more symptoms of a TAM-associated cancer, a c-Met-associated cancer, or both, and/or a patient that has an increased risk of developing a TAM-associated cancer, a c-Met-associated cancer, or both).

In some embodiments of any the methods described herein, the compound of Formula I, II, Ill or IV or a pharmaceutically acceptable salt thereof is administered in combination with a therapeutically effective amount of at least one additional anticancer agent selected from one or more additional therapies or therapeutic agents, for example an agent that works by the same or by a different mechanism of action. In some embodiments, the compound of Formula I is selected from the compounds described in Example Nos. 1-58, or pharmaceutically acceptable salts thereof. In some embodiments, a compound of Formula I is selected from i) Example Nos. 1-10; ii) Example Nos. 11-20; iii) Example Nos. 21-30; iv) Example Nos. 31-40; v) Example Nos. 41-49; vi) Example Nos. 50-58; or pharmaceutically acceptable salts thereof.

Non-limiting examples of additional anticancer agents include immune-targeted agents including immunotherapy agents, anti-viral agents, kinase-targeted therapeutic agents, anti-viral vaccines, anti-hormonal agents, signal transduction pathway inhibitors, chemotherapeutics or other anti-cancer agents, angiogenesis inhibitors, and radiotherapy.

One or more of any of the additional anticancer agents described herein can be combined with the present compounds in a single dosage form, or the present compounds and the at least one additional anticancer agent can be administered simultaneously or sequentially as separate dosage forms.

In some embodiments, the compound of Formula I, II, Ill or IV or a pharmaceutically acceptable salt thereof is administered daily for 28 consecutive days in a 28 days cycle.

In some embodiments, compounds of Formula I, II, Ill or IV or pharmaceutically acceptable salts thereof may be combined with immune-targeted agents including immunotherapy drugs.

The term “immunotherapy agents” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy agent is an immune checkpoint inhibitor. As used herein, the term “immune checkpoint inhibitor” or “checkpoint inhibitor” refers to molecules that totally or partially reduce, inhibit, interfere with, or modulate the expression and/or activity of one or more checkpoint proteins. In some embodiments, the immunotherapy includes one or more immune checkpoint inhibitors. In some embodiments, the immune checkpoint inhibitor is a CTLA-4 inhibitor (e.g., an anti-CTLA-4 antibody), a PD-1 inhibitor (e.g., an anti-PD-1 monoclonal antibody) or a PD-L1 inhibitor (e.g., an anti-PD-LI monoclonal antibody). In some embodiments, the CTLA-4 inhibitor is ipilimumab (Yervoy®) or tremelimumab (CP-675,206). In some embodiments, the PD-1 inhibitor is pembrolizumab (Keytruda®), nivolumab (Opdivo®), or pidilizumab. In some embodiments, the anti-PD-1 monoclonal antibody is nivolumab or pembrolizumab. In some embodiments, the anti-PD-1 antibody is pembrolizumab. In some embodiments, the PD-L1 inhibitor is atezolizumab (Tecentriq®), avelumab (Bavencio®), durvalumab (Imfinzi™), MED14736, or MPDL3280A. In some embodiments, the PD-1 or PD-L1 inhibitor is a small molecule (e.g., those disclosed in US2018/305313 and WO 2018/195321). In some embodiments, the PD-L1 inhibitor is atezolizumab (Tecentriq®), avelumab (Bavencio®), or durvalumab (Imfinzi™). In some embodiments, a checkpoint inhibitor can target 4-1BB (e.g., urelumab (BMS-663513) and PF-05082566 (PF-2566)), CD27 (e.g., varlilumab (CDX-1127), CD40 (e.g., CP-870,893), OX40, TIM-3, ICOS, BTLA, A2AR, B7-H3, B7-H4, BTLA, IDO, KIR, LAG3, TIM-3, and VISTA. Additional non-limiting examples of immune checkpoint inhibitors include ulocuplumab, urelumab, PF 05082566, TRX518, varlilumab, CP 870893, PDR001MED14736, avelumab, BMS 986016, MGA271, IPH2201, emactuzumab, INCB024360, MED16469, galunisertib, BKT140, bavituximab, lirilumab, bevacizumab, MNRP1685A, lambroizumab, CC 90002, BMS-936559, and MGA271.

In some embodiments, a compound of Formula I, II, Ill or IV or pharmaceutically acceptable salt thereof is combined with an immune checkpoint inhibitor, wherein the immune checkpoint inhibitor is administered on one or more days in a 28 days cycle. In some embodiments the compound of Formula I, II, Ill or IV or a pharmaceutically acceptable salt thereof is administered daily for 28 consecutive days in a 28 days cycle.

In some embodiments, a compound of Formula I, II, Ill or IV or pharmaceutically acceptable salt thereof is combined with an immune checkpoint inhibitor, wherein the immune checkpoint inhibitor is administered one a week. In some embodiments, the immune checkpoint inhibitor is administered every two weeks. In one embodiment, the immune checkpoint inhibitor is administered every three weeks. In one embodiment, the immune checkpoint inhibitor is administered every 4 weeks. In some embodiments, the immune checkpoint inhibitor is administered on day 1 of a 28-day cycle. In some embodiments, the immune checkpoint inhibitor is administered on days 1 and 7 in a 28-day cycle. In some embodiments, the immune checkpoint inhibitor is administered in days 1, 7 and 14 in a 28-day cycle. In some embodiments, the immune checkpoint inhibitor is administered on days 1, 7, 14 and 21 in a 28-day cycle. In some embodiments, the immune checkpoint inhibitor is administered on days 1, 7, 14 and 28 in a 28-day cycle. In some embodiments the compound of Formula I, II, Ill or IV or a pharmaceutically acceptable salt thereof is administered daily for 28 consecutive days in a 28-day cycle. In some embodiments, the immune checkpoint inhibitor is administered by intravenous infusion.

In some embodiments, a compound of Formula I, II, Ill or IV or pharmaceutically acceptable salt thereof is combined with an immune checkpoint inhibitor, wherein the immune checkpoint inhibitor is administered on day 1 of cycles 1 through 13. In some embodiments the compound of Formula I, II, Ill or IV or a pharmaceutically acceptable salt thereof is administered daily for 28 consecutive days in a 28-day cycle.

In some embodiments, the immunotherapy agent is a cellular immunotherapy (e.g., adoptive T-cell therapy, dendritic cell therapy, or a natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™)

In some embodiments, the immunotherapy agent is an antibody therapy (e.g., a monoclonal antibody, a conjugated antibody). In some embodiments, the antibody therapy is bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™) cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab, or amatuximab.

In some embodiments, the immunotherapy agent is an antibody-drug conjugate. In some embodiments, the antibody-drug conjugate is gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853), or anetumab ravtansine

In some embodiments, the immunotherapy includes blinatumomab (AMG103; Blincyto®) or midostaurin (Rydapt).

In some embodiments, the immunotherapy agent includes a toxin. In some embodiments, the immunotherapy is denileukin diftitox (Ontak®).

In some embodiments, the immunotherapy agent is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy agent is an inhibitory nucleic acid-based immunotherapy agent (e.g., antisense oligonucleotides, small interfering RNAs (siRNAs), and short hairpin RNAs (shRNAs). In some embodiments, the inhibitory nucleic acid-based immunotherapy is CV9104 (see, e.g., Rausch et al. (2014) Human Vaccine Immunother. 10(11): 3146-52; and Kubler et al. (2015) J. Immunother. Cancer 3:26).

In some embodiments, the immunotherapy agent is bacillus Calmette-Guerin (BCG) therapy. In some embodiments, the immunotherapy agent is an oncolytic virus therapy. In some embodiments, the oncolytic virus therapy is talimogene alherparepvec (T-VEC; ImLygic®).

In some embodiments, the immunotherapy agent is a cancer vaccine. In some embodiments, the cancer vaccine is a human papillomavirus (HPV) vaccine. In some embodiments, the HPV vaccine is Gardasil®, Gardasil9® or Cervarix®. In some embodiments, the cancer vaccine is a hepatitis B virus (HBV) vaccine. In some embodiments, the HBV vaccine is Engerix-B®, Recombivax HBO or GI-13020 (Tarmogen®). In some embodiments, the cancer vaccine is Twinrix® or Pediarix®. In some embodiments, the cancer vaccine is BiovaxiD®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110).

In some embodiments, the immunotherapy agent is a peptide vaccine. In some embodiments, the peptide vaccine is nelipepimut-S (E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K or NEO-PV-01. In some embodiments, the cancer vaccine is a DNA-based vaccine. In some embodiments, the DNA-based vaccine is a mammaglobin-A DNA vaccine (see, e.g., Kim et al. (2016) Oncolmmunology 5(2): e1069940).

In some embodiments, immune-targeted agents are selected from aldesleukin, interferon alfa-2b, ipilimumab, lambrolizumab, nivolumab, prednisone, and sipuleucel-T.

Suitable antiviral agents contemplated for use in combination with a compound of Formula I, II, Ill or IV or a pharmaceutically acceptable salt thereof can comprise nucleoside and nucleotide reverse transcriptase inhibitors (RTIs), non-nucleoside reverse transcriptase inhibitors (NRTIs), protease inhibitors, and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (I-(ethoxy-methyl)-5-(I-methylethyl)-6-(phenylmethyl)-(2,4(IH,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside, and Yissum Project No.11607.

Compounds of Formula I, II, Ill or IV or pharmaceutically acceptable salts thereof can be used in combination with one or more other kinase inhibitors for the treatment of diseases, such as cancer, that are impacted by one or more signaling pathways.

In certain embodiments, the patient to be treated with a combination therapy described herein has not been treated with an additional anticancer agent prior to the administration the combination therapy. In certain embodiments, the patient to be treated with a combination therapy described herein has been treated with at least one additional anticancer agent prior to administration of a compound of Formula I, II, Ill or IV for use alone or in a combination therapy described herein. In certain embodiments, the patient to be treated with a compound of Formula I, II, Ill or IV as monotherapy or in a combination therapy described herein has developed drug resistance to, or has a cancer that is refractory to, at least one additional anticancer agent.

In some embodiments, compounds of Formula I, II, Ill or IV or pharmaceutically acceptable salts thereof can be combined with one or more inhibitors of the following kinases for the treatment of cancer: PIM (PIM I, PIM 2, PIM 3), IDO, AKT I, AKT2 and AKT3, TGFR, PKA, PKG, PKC, CaM-kinase, phosphorylase kinase, MEKK, ERK, MAPK, mTOR, EGFR, HER2, HER3, HER4, INS-R, IGF-1R, IR-R, PDGFaR, PDGFR, CSFIR, KIT, FLK-II, KDR/FLK-1, FLK-4, flt-1, FGFR, FGFR1, FGFR2, FGFR3, FGFR4, c-MET, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, FAK, SYK, FRK, JAK, ABL, ALK, and B-Raf.

Compounds of Formula I, II, Ill or IV or pharmaceutically acceptable salts thereof can also be used in combination with one or more additional anticancer agents, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

In some embodiments, signal transduction pathway inhibitors include kinase inhibitors of the Ras-Raf-MEK-ERK pathway (e.g., binimetinib, selumetinib, encorafenib, sorafenib, trametinib, cobimetinib, dabrafenib, and vemurafenib), kinase inhibitors of the Pl3K-AKT-mTOR-S6K pathway (e.g. everolimus, rapamycin, perifosine, temsirolimus), and other kinase inhibitors, such as baricitinib, brigatinib, capmatinib, danusertib, ibrutinib, milciclib, quercetin, regorafenib, ruxolitinib, semaxanib, ((R)-amino-N-[5,6-dihydro-2-(1-methyl-1H-pyrazol-4-yl)-6-oxo-1H-pyrrolo[4,3,2-ef][2,3]benzodiazepin-8-yl]-cyclohexaneacetamide), and TG101209 (N-tert-butyl-3-(5-methyl-2-(4-(4-methylpiperazin-1-yl)phenylamino)pyrimidin-4-ylamino)benzenesulfonamide).

A combination of a compound of Formula I, II, Ill or IV in combination with binimetinib, selumetinib, encorafenib, sorafenib, trametinib, or vemurafenib results in sensitization of tumors that are resistant to binimetinib, selumetinib, encorafenib, sorafenib, trametinib, or vemurafenib, respectively.

In one embodiment, there is provided a pharmaceutical combination which comprises (a) a compound of Formula I, II, Ill or IV or a pharmaceutically acceptable salt or solvate thereof, and (b) an additional anticancer agent selected from the group consisting of (i) binimetinib, (ii) selumetinib, (iii) encorafenib, (iv) sorafenib, (v) trametinib, and (vi) vemurafenib, each optionally in the form of a pharmaceutically acceptable salt or solvate thereof, and combinations of any thereof.

In one embodiment, there is provided a pharmaceutical combination which comprises (a) a compound of Formula I, II, Ill or IV or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Formula I, II, Ill or IV or a pharmaceutically acceptable salt or solvate thereof, and (b) binimetinib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) binimetinib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) encorafenib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) encorafenib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) selumetinib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) selumetinib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) sorafenib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) sorafenib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) trametinib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) trametinib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) vemurafenib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) vemurafenib or a pharmaceutically acceptable salt or solvate thereof.

In one embodiment, there is provided a pharmaceutical combination which comprises (a) a compound of Example No. 1, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 1, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 2, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 2, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 3, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 3, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 4, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 4, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 7, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 7, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 18, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 18, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 19, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 19, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 20, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 20, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 27, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 27, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 28, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 28, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 29, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 29, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 32, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 32, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 33, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 33, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 44, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 44, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 46, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 46, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 48, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 48, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 55, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 55, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 56, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 56, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In one embodiment, there is provided a pharmaceutical combination which comprises (a) a compound of Example No. 58, or a pharmaceutically acceptable salt or solvate thereof and (b) an additional anticancer agent selected from the group consisting of (i) binimetinib, (ii) selumetinib, (iii) encorafenib, (iv) sorafenib, (v) trametinib, (vi) vemurafenib, each optionally in the form of a pharmaceutically acceptable salt or solvate thereof, and combinations of any thereof.

In one embodiment, there is provided a pharmaceutical combination which comprises (a) a compound of Example No. 58, or a pharmaceutically acceptable salt or solvate thereof, and (b) an additional anticancer agent selected from the group consisting of (i) binimetinib and (ii) encorafenib, each optionally in the form of a pharmaceutically acceptable salt or solvate thereof, and a combination thereof.

In each of the above combinations, the compound of Formula I, II, III or IV or the pharmaceutically acceptable salt thereof and the additional anticancer agent may be formulated as separate compositions or dosages for simultaneous, separate or sequential use for the treatment of cancer, wherein the amounts of the compound of Formula I, II, Ill or IV or a pharmaceutically acceptable salt thereof and of the additional anticancer agent are together effective in treating the cancer. Also provided herein is a pharmaceutical composition comprising such a combination. Also provided herein is the use of such a combination for the preparation of a medicament for the treatment of cancer (e.g., a TAM-associated cancer or a c-Met-associated cancer). Also provided herein is a commercial package or product comprising such a combination as a combined preparation for simultaneous, separate or sequential use; and to a method of treatment of cancer a patient in need thereof.

Also provided are methods of treating an individual with cancer that include administering that include administering to a patient identified or diagnosed as having cancer (e.g., a TAM-associated cancer or a c-Met-associated cancer) a therapeutically effective amount of any of the combinations.

Also provided herein are methods of treating a patient identified or diagnosed as having a TAM-associated cancer or a c-Met-associated that include administering to a patient identified or diagnosed as having a TAM-associated cancer or a c-Met-associated a therapeutically effective amount of a therapeutically effective amount of any of the combinations.

A combination of a compound of Formula I, II, Ill or IV in combination with an EGFR inhibitor (e.g., any of the EGFR inhibitors described herein) results in effective reduction in proliferation of cancer cells having resistance to EGFR inhibitors or cancer cells having resistance to c-Met inhibitors).

In one embodiment, there is provided a pharmaceutical combination which comprises (a) a compound of Formula I, II, Ill or IV or a pharmaceutically acceptable salt or solvate thereof, and (b) an additional anticancer agent selected from the group consisting of (i) cetuximab (or a biosimilar thereof), (ii) panitumumab (or a biosimilar thereof), (iii) erlotinib, (iv) lapatinib, and (v) gefitinib, each optionally in the form of a pharmaceutically acceptable salt or solvate thereof, and combinations of any thereof.

In one embodiment, there is provided a pharmaceutical combination which comprises (a) a compound of Formula I, II, Ill or IV or a pharmaceutically acceptable salt or solvate thereof, and (b) cetuximab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) panitumumab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) erlotinib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) lapatinib or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) gefitinib or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) an additional anticancer agent selected from the group consisting of (i) cetuximab (or a biosimilar thereof), (ii) panitumumab (or a biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) cetuximab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) panitumumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) erlotinib, each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) lapatinib, each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) gefitinib, each optionally in the form of a pharmaceutically acceptable salt or solvate thereof, and combinations of any thereof.

In one embodiment, there is provided a pharmaceutical combination which comprises (a) a compound of Example No. 1, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 2, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 3, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 4, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 7, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 18, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 19, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 20, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 27, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 28, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 29, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 32, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 33, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 44, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 46, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 48, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 55, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 56, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

In one embodiment, there is provided a pharmaceutical combination which comprises (a) a compound of Example No. 58, or a pharmaceutically acceptable salt or solvate thereof and (b) an additional anticancer agent selected from the group consisting of (i) cetuximab (ora biosimilar thereof), (ii) panitumumab (ora biosimilar thereof), (iii) erlotinib, (iv) lapatinib, and (v) gefitinib each optionally in the form of a pharmaceutically acceptable salt or solvate thereof, and combinations of any thereof.

A combination of a compound of Formula I, II, Ill or IV in combination with an immune checkpoint inhibitor (e.g., any of the checkpoint inhibitors described herein, e.g., a PD-1 or a PD-L1 inhibitor) results in sensitization of tumors to immune checkpoint inhibitor therapy. For example, a compound of Formula I, II, Ill or IV in combination with an immune checkpoint inhibitor can result in one or more (e.g., two, three, four, or five) of an increase in dendritic cell-dependent antigen presentation, an increase in NK cell response, an increase in T-cell trafficking, an increase in Type 1 macrophages which results in production of immune stimulating cytokines, and an enhancement of both innate and adaptive immune response.

In one embodiment, there is provided a pharmaceutical combination which comprises (a) a compound of Formula I, II, Ill or IV or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Formula I, II, Ill or IV or a pharmaceutically acceptable salt or solvate thereof, and (b) nivolumab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) pembrolizumab (ora biosimilar thereof) ora pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) cemiplimab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) pidilizumab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) 1141PDCA-170 (ora biosimilar thereof) ora pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) atezolizumab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) avelumab (or a biosimilar thereof) or a pharmaceutically acceptable salt or solvate thereof.

In one embodiment, there is provided a pharmaceutical combination which comprises (a) a compound of Formula I, II, Ill or IV or a pharmaceutically acceptable salt or solvate thereof, and (b) durvalumab (or a biosimilar thereof) or a pharmaceutically acceptable salt or solvate thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) nivolumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) pembrolizumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) cemiplimab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) pidilizumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) 1141PDCA-170 (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) atezolizumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) avelumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt or solvate thereof, and (b) durvalumab (or a biosimilar thereof), each optionally in the form of a pharmaceutically acceptable salt or solvate thereof, and a combination thereof.

In one embodiment, there is provided a pharmaceutical combination which comprises (a) a compound of Example No. 1, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 2, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 3, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 4, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 7, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 18, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 19, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 20, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 27, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 28, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 29, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 32, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 33, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 44, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 46, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 48, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 55, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 56, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof.

In one embodiment, there is provided a pharmaceutical combination which comprises (a) a compound of Example No. 58, or a pharmaceutically acceptable salt or solvate thereof, and (b) an additional anticancer agent selected from the group consisting of (i) nivolumab (or a biosimilar thereof), (ii) pembrolizumab (or a biosimilar thereof), (iii) cemiplimab (or a biosimilar thereof), (iv) pidilizumab (or a biosimilar thereof), (v) 1141PDCA-170 (or a biosimilar thereof), (vi) atezolizumab (or a biosimilar thereof), (vii) avelumab (or a biosimilar thereof), and (viii) durvalumab (or a biosimilar thereof), each optionally in the form of a pharmaceutically acceptable salt or solvate thereof, and combinations of any thereof. Angiogenesis inhibitors may be efficacious in some tumors in combination with compounds of Formula I, II, Ill or IV or pharmaceutically acceptable salts thereof. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib.

Non-limiting examples of radiotherapy include radioiodide therapy, external-beam radiation, and radium 223 therapy.

Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art.

Accordingly, also provided herein is a method of treating cancer, comprising administering to a patient in need thereof a pharmaceutical combination for treating cancer which comprises (a) a compound of Formula I, II, Ill or IV or a pharmaceutically acceptable salt thereof, and (b) an additional anticancer agent, for simultaneous, separate or sequential use for the treatment of cancer, wherein the amounts of the compound of Formula I, II, Ill or IV or a pharmaceutically acceptable salt thereof and the additional anticancer agent are together effective in treating the cancer.

Also provided herein is a method of treating cancer, comprising administering to a patient in need thereof a pharmaceutical combination for treating cancer which comprises (a) a compound of Formula I, II, Ill or IV or a pharmaceutically acceptable salt thereof, and (b) an additional anticancer therapy, wherein the therapy is selected from radiation therapy and surgery. In some embodiments, the additional anticancer therapy is radiation therapy. In some embodiments, the additional anticancer therapy is surgery.

In some embodiments, the additional anticancer agent(s) includes any one of the above listed therapies or therapeutic agents which are standards of care in cancers wherein the cancer is a TAM-associated cancer. In some embodiments, the compound of Formula I, II, Ill or IV the additional anticancer agent is an immunotherapy agent. In some embodiments, the immunotherapy agent is a CTLA-4 inhibitor (e.g., an anti-CTLA-4 antibody), a PD-1 inhibitor (e.g., an anti-PD-1 monoclonal antibody) or a PD-L1 inhibitor (e.g., an anti-PD-LI monoclonal antibody).

In some embodiments, provided herein is a method for treating cancer, comprising administering a compound of Formula I, II, Ill or IV in combination with an immune checkpoint inhibitor. In some embodiments, the immunotherapy includes one or more immune checkpoint inhibitors (e.g., PDR001 or any of the other exemplary immune checkpoint inhibitors described herein). In some embodiments, the immune checkpoint inhibitor is a CTLA-4 inhibitor (e.g., an anti-CTLA-4 antibody), a PD-1 inhibitor (e.g., an anti-PD-1 monoclonal antibody) a PD-L1 inhibitor (e.g., an anti-PD-LI monoclonal antibody), a NOX2 inhibitor, an A2A4 inhibitor, a B7-H3 inhibitor (e.g., MGA271), a B7-H4 inhibitor (e.g., an anti-B7-H4 antibody, e.g., those described in Dangaj et al., Cancer Res. 73(15):4820-4829, 2013), an IDO inhibitor (e.g., coptisine, 1-methyl-D-tryptophan, NLG-919, indoximod, 1-DL-methyl tryptophan, or the inhibitors described in Brastianos et al., JACS 128(50:16046-16047, 2006), a TIM3 inhibitor, a LAG3 inhibitor (e.g., BMS-986016), TIGIT inhibitor, a BTLA inhibitor, a VISTA inhibitor (e.g., 1141PDCA-170), a ICOS inhibitor, a KIR inhibitor (e.g., lirilumab), a CD39 inhibitor, a SIGLEC7 inhibitor, or a SIGLEC9 inhibitor. In some embodiments, the CTLA-4 inhibitor is ipilimumab (Yervoy®), tremelimumab (CP-675,206), or the aptamers described in Santulli-Marotto et al., Cancer Res. 63(21):7483-7489, 2003. In some embodiments, the PD-1 inhibitor is pembrolizumab (Keytruda®), nivolumab (Opdivo®), cemiplimab (Libtayo®), pidilizumab, or 1141PDCA-170. In some embodiments, the anti-PD-1 monoclonal antibody is nivolumab or pembrolizumab. In some embodiments, the anti-PD-1 antibody is pembrolizumab. In some embodiments, the PD-L1 inhibitor is atezolizumab (Tecentriq®), avelumab (Bavencio®), durvalumab (Imfinzi™). In some embodiments, the PD-L1 inhibitor is atezolizumab (Tecentriq®), avelumab (Bavencio®) or durvalumab (Imfinzi™). In some embodiments, the compound of Formula I is selected from the compounds described in Example Nos. 1-58, or pharmaceutically acceptable salts thereof. In some embodiments, a compound of Formula I is selected from i) Example Nos. 1-10; ii) Example Nos. 11-20; iii) Example Nos. 21-30; iv) Example Nos. 31-40; v) Example Nos. 41-49; vi) 50-58; or pharmaceutically acceptable salts thereof. In some embodiments, provided herein is a method for treating cancer, comprising administering to a patient in need thereof a compound of Formula I, II, Ill or IV in combination with an immune checkpoint inhibitor, wherein the patient is further treated with ionizing radiation. In some embodiments, the cancer overexpresses AXL. In some embodiments, the cancer does not have a B-RAF mutation. In some embodiments, the cancer has a B-RAF mutation. In some embodiments, the cancer has a RAS mutation. In some embodiments, the cancer has an EGFR mutation. In some embodiments, the cancer overexpresses MER. In some embodiments, the cancer is lung cancer. In some embodiments, the cancer is non-small cell lung carcinoma (NSCLC). In some embodiments, the cancer is colon cancer. In some embodiments, the cancer is prostate cancer. In some embodiments, the cancer is melanoma. In some embodiments, the cancer is Acute Lymphoblastic Leukemia (ALL). In some embodiments, the cancer is Acute Myeloid Leukemia (AML).

Combination therapies as described herein may be administered without restriction on the order in which therapies are administered to a patient with a disease or disorder described herein. Thus, in some embodiments, a compound of Formula I, II, Ill or IV or pharmaceutically acceptable salt thereof can be administered prior to (e.g., 5 minutes, 15 minutes, 30 minutes, 45 minutes, 1 hour, 2 hours, 4 hours, 6 hours, 12 hours, 24 hours, 36 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 36 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent (e.g., any of the additional anticancer agents described herein) to the subject. In another embodiment, a compound of Formula I, II, Ill or IV or pharmaceutically acceptable salt thereof can be administered prior to (e.g., 5 minutes, 15 minutes, 30 minutes, 45 minutes, 1 hour, 2 hours, 4 hours, 6 hours, 12 hours, 24 hours, 36 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 36 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent (e.g., any of the anticancer agents described herein).

In some embodiments, provided herein is a method for treating cancer, comprising sensitizing said cancer to an anti-mitotic drug by administration of a compound of Formula I, II, III or IV. In some embodiments, the anti-mitotic drug is a taxane-based chemotherapeutic, such as docetaxel.

Some embodiments, compounds of Formula I, II, Ill or IV may be used in combination with other agents to treat patients who have primary or acquired resistance to at least one additional anticancer agent.

In some embodiments of methods disclosed herein for treating cancer, compounds of Formula I, II, Ill or IV may be used as monotherapy to treat patients who have developed primary or acquired resistance to at least one additional anticancer agent.

In some embodiments, compounds of Formula I, II, Ill or IV may be used to overcome resistance to at least one additional anticancer agent in a cancer. In some embodiments, a compound of Formula I, II, Ill or IV is used in combination with the at least one additional anticancer agent to which the cancer has developed resistance.

In some embodiments, compounds of Formula I, II, Ill or IV may be used to delay resistance to at least one additional anticancer agent. In some embodiments, a compound of Formula I, II, Ill or IV is used in combination with the at least one additional anticancer agent.

As used herein, the term “resistance” refers to a clinical scenario where a cancer fails to respond to a targeted therapy or immunotherapy. For example, resistance of a cancer can be observed by, e.g., a decrease in the rate of increase of tumor burden in the subject, a lack of a decrease in the tumor burden in the subject, an increase in the dosage of a therapeutic agent over time required to achieve the same therapeutic effect in a patient, and the requirement of co-administration of an additional anticancer agent to achieve the same therapeutic effect as the previous administration of the therapeutic agent as a monotherapy.

As used herein, the term “primary resistance”, also known as intrinsic resistance, refers to a clinical scenario where a cancer fails to respond to a targeted therapy or immunotherapy, that is, the cancer is resistant to a therapy without having been previously exposed to the therapy.

As used herein, the term “acquired resistance” refers to a clinical scenario in which a cancer initially responded to a targeted therapy or immunotherapy but after a period of time the cancer stops responding to the treatment (e.g., the cancer relapses and progresses).

In some embodiments of methods disclosed herein for treating cancer, compounds of Formula I, II, Ill or IV may be used as monotherapy to treat patients who have developed primary or acquired resistance to at least one additional anticancer agent.

In some embodiments of methods disclosed herein for treating cancer, compounds of Formula I, II, Ill or IV may be used as in combination with at least one additional anticancer agent to treat patients who have developed primary or acquired resistance to one or more of the at least one additional anticancer agent (e.g., a targeted therapeutic agent).

Targeted therapeutic agents include inhibitors or antibodies against EGFR, HER2, VEGFR, c-Met, Ret, IGFR1, PDGFR, FGFR1, FGFR2, FGFR3, FGFR4, TrkA, TrkB, TrkC, ROS, c-Kit, or Flt-3 and against cancer-associated fusion protein kinases such as Bcr-Abl and EML4-Alk. Inhibitors against EGFR include gefitinib, erlotinib, and nazartinib (see, e.g., U.S. Pat. No. 10,195,208 and J. Med. Chem. 59(14):6671-6689, 2016), and inhibitors against EGFR/Her2 include but are not limited to dacomitinib, afatinib, lapatinib and neratinib. Antibodies against the EGFR include but are not limited to cetuximab, panitumumab and necitumumab. Inhibitors of c-Met may be used in combination with compounds of Formula I, II, Ill or IV of pharmaceutically acceptable salts thereof. c-MET inhibitors include onartumzumab, tivantinib, and INC-280. Inhibitors against FGFRs include but not limited to AZD4547, BAY1187982, ARQ087, BGJ398, BIBF1120, TK1258, lucitanib, dovitinib, TAS-120, JNJ-42756493, and Debiol347. Inhibitors against Trks include but not limited to larotrectinib (LOXO-101), and entrectinib (RXDX-101). Inhibitors against Abl (or Bcr-Abl) include imatinib, dasatinib, nilotinib, and ponatinib and those against Alk (or EML4-ALK) include crizotinib.

In some embodiments, provided herein are methods of treating a patient having cancer who has been previously treated with a first kinase inhibitor, wherein the first kinase inhibitor is not a compound of Formula I, II, Ill or IV, comprising administering to said patient a therapeutically effective amount of a compound of Formula I, II, Ill or IV or a pharmaceutically acceptable salt thereof. In some embodiments, the patient is treated with a compound of Formula I, II, Ill or IV as a single agent. In some embodiments, the patient is treated with a combination of a compound of Formula I, II, Ill or IV and the previously administered first kinase inhibitor. In some embodiments, the patient is treated with a combination of a compound of Formula I, II, Ill or IV and the previously administered first kinase inhibitor. In some embodiments, the compound of Formula I, II, Ill or IV and the previously administered first kinase inhibitor are administered as separate dosages sequentially in any order. In some embodiments, the kinase inhibitor is an EGFR inhibitor. In some embodiments, the EGFR inhibitor is erlotinib or lapatinib. In some embodiments, the kinase inhibitor is a Pl3Kα inhibitor. In some embodiments, the Pl3Kα inhibitor is alpelisib. In some embodiments, the kinase inhibitor is a MEK inhibitor. In some embodiments, the MEK inhibitor is binimetinib, U0126, or PD 325901. In some embodiments, the kinase inhibitor is an FGFR inhibitor. In some embodiments, the kinase inhibitor is an ALK inhibitor. In some embodiments, the kinase inhibitor is an IGFR1 inhibitor. In some embodiments, the cancer is breast cancer (e.g., triple negative breast cancer), head and neck cancer (e.g., squamous cell head and neck cancer), non-small cell lung cancer, colorectal cancer, esophageal squamous cell carcinoma, or melanoma.

In some embodiments, provided herein are methods of treating a patient having cancer who has been previously treated with an EGFR antibody, comprising administering to said patient a therapeutically effective amount of a compound of Formula I, II, Ill or IV or a pharmaceutically acceptable salt thereof. In some embodiments, the patient is treated with a compound of Formula I, II, Ill or IV as a single agent. In some embodiments, the patient is treated with a combination of a compound of Formula I, II, Ill or IV and the previously administered EGFR antibody. In some embodiments, the compound of Formula I, II, Ill or IV and the previously administered EGFR antibody are administered as separate dosages sequentially in any order. In some embodiments, the EGFR antibody is cetuximab. In some embodiments, the cancer is breast cancer, head and neck cancer, or non-small cell lung cancer

In some embodiments, provided herein are methods of treating a patient having cancer who has been previously treated with a first kinase inhibitor, wherein the first kinase inhibitor is not a compound of Formula I, II, Ill or IV, comprising (a) determining that said cancer overexpresses a TAM kinase and/or c-Met kinase (e.g., as compared to a non-cancerous tissue or a cell in the patient or a different subject), and (b) after (a), administering to said patient a therapeutically effective amount of a compound of Formula I, II, Ill or IV or a pharmaceutically acceptable salt thereof. In some embodiments, the step of determining if the cancer overexpresses a TAM kinase and/or c-Met kinase includes a step of performing an assay on a sample obtained from the patient to determine whether the patient has abnormal (e.g., increased) expression, level, and/or activity of one or more of the TAM kinases and/or c-Met kinase (e.g., as compared to a non-cancerous tissue or cell in the patient or a different subject), e.g., AXL and/or MER and/or TYRO3 and/or c-Met. In some embodiments, the cancer that was previously treated with the first kinase inhibitor overexpresses AXL. In some embodiments, the cancer that was previously treated with the first kinase inhibitor overexpresses MER. In one embodiment, the cancer that was previously treated with the first kinase inhibitor overexpresses TYRO3. In one embodiment, the cancer that was previously treated with the first kinase inhibitor overexpresses c-Met kinase. In some embodiments, the method further comprises obtaining a sample from the patient. In some embodiments, the sample is a biopsy sample. In some embodiments, the assay is selected from the group consisting of sequencing, immunohistochemistry, enzyme-linked immunosorbent assay, and fluorescence in situ hybridization (FISH). In some embodiments, the first kinase inhibitor is an EGFR inhibitor. In some embodiments, the EGFR inhibitor is erlotinib or lapatinib. In some embodiments, the first kinase inhibitor is a Pl3Kα inhibitor. In some embodiments, the Pl3Kα inhibitor is alpelisib. In some embodiments, the first kinase inhibitor is a MEK inhibitor. In some embodiments, the MEK inhibitor is binimetinib, U0126, or PD 325901. In some embodiments, the first kinase inhibitor is an FGFR inhibitor. In some embodiments, the first kinase inhibitor is an ALK inhibitor. In some embodiments, the first kinase inhibitor is an IGFR1 inhibitor. In some embodiments, the cancer is breast cancer (e.g., triple negative breast cancer), head and neck cancer (e.g., squamous cell head and neck cancer), non-small cell lung cancer, colorectal cancer, esophageal squamous cell carcinoma, or melanoma. In some embodiments, the patient is treated with a compound of Formula I, II, Ill or IV as a single agent. In some embodiments, the patient is treated with a combination of a compound of Formula I, II, Ill or IV and the first kinase inhibitor. In some embodiments, the compound of Formula I, II, Ill or IV and the previously prescribed kinase inhibitor are administered as separate dosages sequentially in any order.

In some embodiments, provided herein is a method of treating a subject having cancer, wherein the method comprises (a) determining that a cancer cell in a sample obtained from a subject having a cancer and previously administered one or more doses of a first kinase inhibitor, wherein the first kinase inhibitor is not a compound of Formula I, II, Ill or IV, overexpresses one or more TAM kinases and/or c-Met kinase (e.g., as compared to a non-cancerous tissue or cell in the subject or a different subject); and (b) administering a compound of Formula I, II, Ill or IV or a pharmaceutically acceptable salt or solvate thereof as a monotherapy or in conjunction with the previously administered first kinase inhibitor to the subject. In some embodiments, the cancer that was previously treated with the first kinase inhibitor overexpresses AXL. In some embodiments, the cancer that was previously treated with the first kinase inhibitor overexpresses MER. In one embodiment, the cancer that was previously treated with the first kinase inhibitor overexpresses TYRO3. In one embodiment, the cancer that was previously treated with the first kinase inhibitor overexpresses c-Met kinase. In some embodiments, the first kinase inhibitor is an EGFR inhibitor. In some embodiments, the EGFR inhibitor is erlotinib or lapatinib. In some embodiments, the first kinase inhibitor is a Pl3Kα inhibitor. In some embodiments, the Pl3Kα inhibitor is alpelisib. In some embodiments, the first kinase inhibitor is a MEK inhibitor. In some embodiments, the MEK inhibitor is binimetinib, U0126, or PD 325901. In some embodiments, the first kinase inhibitor is an FGFR inhibitor. In some embodiments, the first kinase inhibitor is an ALK inhibitor. In some embodiments, the first kinase inhibitor is an IGFR1 inhibitor. In some embodiments, the cancer is breast cancer (e.g., triple negative breast cancer), head and neck cancer (e.g., squamous cell head and neck cancer), non-small cell lung cancer, colorectal cancer, esophageal squamous cell carcinoma, or melanoma. In some embodiments, the patient is treated with a compound of Formula I, II, Ill or IV as a single agent. In some embodiments, the patient is treated with a combination of a compound of Formula I, II, Ill or IV and the first kinase inhibitor. In some embodiments, the compound of Formula I, II, Ill or IV and the previously prescribed kinase inhibitor are administered as separate dosages sequentially in any order.

In some embodiments of methods disclosed herein for treating cancer, compounds of Formula I, II, Ill or IV may be used as monotherapy to treat patients who have developed primary or acquired resistance to chemotherapy.

In some embodiments of methods disclosed herein for treating cancer, compounds of Formula I, II, Ill or IV may be used as in combination with a chemotherapeutic agent to treat patients who have developed primary or acquired resistance to the chemotherapeutic agent.

In some embodiments, provided herein are methods of treating a patient having cancer who has been previously treated with a chemotherapeutic, comprising administering to said patient a therapeutically effective amount of a compound of Formula I, II, Ill or IV or a pharmaceutically acceptable salt thereof. In some embodiments, the patient is treated with a compound of Formula I, II, Ill or IV as a single agent. In some embodiments, the patient is treated with a combination of a compound of Formula I, II, Ill or IV and the previously administered chemotherapeutic. In some embodiments, the chemotherapeutic is selected from taxane-based chemotherapies (e.g., docetaxol), dexamethasone, and cytarabine. In some embodiments, the patient is treated with a compound of Formula I, II, Ill or IV as a single agent. In some embodiments, the patient is treated with a compound of Formula I, II, Ill or IV in combination with the previously administered chemotherapeutic. In some embodiments, the compound of Formula I, II, Ill or IV and the previously administered chemotherapeutic are administered as separate dosages sequentially in any order. In some embodiments, the cancer is selected from leukemias (including acute myeloid leukemia and chronic myeloid leukemia, B-cell acute lymphoblastic leukemia, and T-lineage acute lymphoblastic leukemia), non-small cell lung cancer, pancreatic ductal adenocarcinoma, astrocytoma, lung adenocarcinoma, ovarian cancer, melanoma, and glioblastoma multiforme.

In some embodiments, provided herein are methods of treating a patient having cancer who has been previously treated with a chemotherapeutic, comprising (a) determining that said cancer overexpresses a TAM kinase and/or c-Met kinase (e.g., as compared to a non-cancerous tissue or cell in the patient or a different subject), and (b) after (a), administering to said patient a therapeutically effective amount of a compound of Formula I, II, Ill or IV or a pharmaceutically acceptable salt thereof. In some embodiments, the step of determining if the cancer overexpresses a TAM kinase and/or c-Met kinase includes a step of performing an assay on a sample obtained from the patient to determine whether the patient has abnormal expression, level, and/or activity of one or more of the TAM kinases and/or c-Met kinase, e.g., AXL and/or MER and/or TYRO3 and/or c-Met kinase. In some embodiments, the method further comprises obtaining a sample from the patient. In some embodiments, the sample is a biopsy sample. In some embodiments, the assay is selected from the group consisting of sequencing, immunohistochemistry, enzyme-linked immunosorbent assay, and fluorescence in situ hybridization (FISH). In some embodiments, the patient is treated with a compound of Formula I, II, Ill or IV as a single agent. In some embodiments, the patient is treated with a combination of a compound of Formula I, II, Ill or IV and the previously administered chemotherapeutic. In some embodiments, the chemotherapeutic is selected from taxane-based chemotherapies (e.g., docetaxel), dexamethasone, and cytarabine. In some embodiments, the patient is treated with a compound of Formula I, II, Ill or IV as a single agent. In some embodiments, the patient is treated with a compound of Formula I, II, Ill or IV in combination with the previously administered chemotherapeutic. In some embodiments, the compound of Formula I, II, Ill or IV and the previously administered chemotherapeutic are administered as separate dosages sequentially in any order. In some embodiments, the cancer is selected from leukemias (including acute myeloid leukemia and chronic myeloid leukemia, B-cell acute lymphoblastic leukemia, and T-lineage acute lymphoblastic leukemia), non-small cell lung cancer, pancreatic ductal adenocarcinoma, astrocytoma, lung adenocarcinoma, ovarian cancer, melanoma, and glioblastoma multiforme.

In some embodiments, provided herein is a method of treating a subject having cancer, wherein the method comprises (a) determining that a cancer cell in a sample obtained from a subject having a cancer and previously administered one or more doses of a chemotherapeutic, overexpresses one or more TAM kinases and/or c-Met kinase (e.g., as compared to a non-cancerous tissue or cell in the subject or a different subject); and (b) administering a compound of Formula I, II, Ill or IV or a pharmaceutically acceptable salt or solvate thereof as a monotherapy or in conjunction with the previously administered chemotherapeutic or a different chemotherapeutic. In some embodiments, the cancer that was previously treated with the chemotherapeutic overexpresses AXL. In some embodiments, the cancer that was previously treated with the chemotherapeutic overexpresses MER. In one embodiment, the cancer that was previously treated with the chemotherapeutic overexpresses TYRO3. In one embodiment, the cancer that was previously treated with the chemotherapeutic overexpresses c-Met kinase. In some embodiments, the patient is treated with a compound of Formula I, II, Ill or IV as a single agent. In some embodiments, the patient is treated with a combination of a compound of Formula I, II, Ill or IV and the previously administered chemotherapeutic. In some embodiments, the chemotherapeutic is selected from taxane-based chemotherapies (e.g., docetaxel), dexamethasone, and cytarabine. In some embodiments, the patient is treated with a compound of Formula I, II, Ill or IV as a single agent. In some embodiments, the patient is treated with a compound of Formula I, II, Ill or IV in combination with the previously administered chemotherapeutic. In some embodiments, the compound of Formula I, II, Ill or IV and the previously administered chemotherapeutic are administered as separate dosages sequentially in any order. In some embodiments, the cancer is selected from leukemias (including acute myeloid leukemia and chronic myeloid leukemia, B-cell acute lymphoblastic leukemia, and T-lineage acute lymphoblastic leukemia), non-small cell lung cancer, pancreatic ductal adenocarcinoma, astrocytoma, lung adenocarcinoma, ovarian cancer, melanoma, and glioblastoma multiforme.

Also provided herein is (i) a pharmaceutical combination for treating a cancer in a patient in need thereof, which comprises (a) a compound of Formula I, II, Ill or IV or a pharmaceutically acceptable salt thereof, and (b) at least one additional anticancer agent (e.g., any of the exemplary additional anticancer agents described herein or known in the art), for simultaneous, separate or sequential use for the treatment of cancer, wherein the amounts of the compound of Formula I, II, Ill or IV or pharmaceutically acceptable salt thereof and of the additional anticancer agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a patient in need thereof. In some embodiments the patient is a human. In some embodiments, the cancer is a TAM-associated cancer.

The term “pharmaceutical combination,” as used herein, refers to a pharmaceutical therapy resulting from the mixing or combining of more than one active ingredient and includes both fixed and non-fixed combinations of the active ingredients. The term “fixed combination” means that a compound of Formula I, II, Ill or IV or a pharmaceutically acceptable salt thereof and at least one additional anticancer agent (e.g., a chemotherapeutic agent), are both administered to a patient simultaneously in the form of a single composition or dosage. The term “non-fixed combination” means that a compound of Formula I, II, Ill or IV or a pharmaceutically acceptable salt thereof and at least one additional anticancer agent (e.g., chemotherapeutic agent) are formulated as separate compositions or dosages such that they may be administered to a patient in need thereof simultaneously, separately or sequentially with variable intervening time limits, wherein such administration provides effective levels of the two or more compounds in the body of the patient. These also apply to cocktail therapies, e.g. the administration of three or more active ingredients

Accordingly, also provided herein is a method of treating a cancer, comprising administering to a patient in need thereof a pharmaceutical combination for treating cancer which comprises (a) a compound of Formula I, II, Ill or IV or pharmaceutically acceptable salt thereof, and (b) an additional anticancer agent for simultaneous, separate or sequential use for the treatment of cancer, wherein the amounts of the compound of Formula I, II, Ill or IV or pharmaceutically acceptable salt thereof and the additional anticancer agent are together effective in treating the cancer. In some embodiments, the compound of Formula I, II, Ill or IV or pharmaceutically acceptable salt thereof, and the additional anticancer agent are administered simultaneously as separate dosages. In some embodiments, the compound of Formula I, II, Ill or IV or pharmaceutically acceptable salt thereof, and the additional anticancer agent are administered as separate dosages sequentially in any order, in jointly therapeutically effective amounts, e.g. in daily or intermittently dosages. In some embodiments, the compound of Formula I, II, Ill or IV or pharmaceutically acceptable salt thereof, and the additional anticancer agent are administered simultaneously as a combined dosage.

Accordingly, also provided herein are methods for inhibiting, preventing, aiding in the prevention, or decreasing the symptoms of metastasis of a cancer in a patient in need thereof, the method comprising administering to the patient a therapeutically effective amount of a compound of Formula I, II, Ill or IV or a pharmaceutically acceptable salt thereof or a pharmaceutical composition thereof. Such methods can be used in the treatment of one or more of the cancers described herein. In some embodiments, the cancer is a TAM-associated cancer, a c-Met-associated cancer, or both. In some embodiments, the compound of Formula I, II, Ill or IV or a pharmaceutically acceptable salt thereof is used in combination with an additional anticancer agent, including an immunotherapy.

Also provided are methods of decreasing the risk of developing a metastasis or an additional metastasis in a patient having a TAM-associated cancer, a c-Met-associated cancer, or both, that include: selecting, identifying, or diagnosing a patient as having a TAM-associated cancer, a c-Met-associated cancer, or both, and administering a therapeutically effective amount of a compound of Formula I, II, Ill or IV or a pharmaceutically acceptable salt thereof to the patient selected, identified, or diagnosed as having a TAM-associated cancer, a c-Met-associated cancer, or both. Also provided are methods of decreasing the risk of developing a metastasis or an additional metastasis in a patient having a TAM-associated cancer, a c-Met-associated cancer or both, that includes administering a therapeutically effective amount of a compound of Formula I, II, Ill or IV or a pharmaceutically acceptable salt or solvent thereof to a patient having a TAM-associated cancer, a c-Met-associated cancer, or both. The decrease in the risk of developing a metastasis or an additional metastasis in a patient having a TAM-associated cancer, a c-Met-associated cancer, or both can be compared to the risk of developing a metastasis or an additional metastasis in the patient prior to treatment, or as compared to a patient or a population of patients having a similar or the same TAM-associated cancer, c-Met-associated cancer, or both, that has received no treatment or a different treatment.

The phrase “risk of developing a metastasis” means the risk that a subject or patient having a primary tumor will develop an additional tumor (e.g., a solid tumor) at a site distant from a primary tumor in a subject or patient over a set period of time, where the additional tumor includes the same or similar cancer cells as the primary tumor. Methods for reducing the risk of developing a metastasis in a subject or patient having a cancer are described herein.

The phrase “risk of developing additional metastases” means the risk that a subject or patient having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Also provided is a method for inhibiting TAM kinase activity and/or inhibiting c-Met kinase activity in a cell (e.g., a mammalian cell), comprising contacting the cell with a compound of Formula I, II, Ill or IV or a pharmaceutically acceptable salt thereof, or a pharmaceutical composition thereof. In some embodiments, the contacting is in vitro. In some embodiments, the contacting is in vivo. In some embodiments, the contacting is in vivo, wherein the method comprises administering an effective amount of a compound of Formula I, II, Ill or IV or a pharmaceutically acceptable salt thereof to a subject having a cell having TAM kinase activity and/or c-Met kinase activity. In some embodiments, the cell is a cancer cell (e.g., a human cancer cell). In some embodiments, the cancer cell is any cancer as described herein. In some embodiments, the cancer cell is a TAM-associated cancer cell. In some embodiments, the cancer cell is a c-Met-associated cancer cell. In some embodiments, the cancer cell is both a TAM-associated cancer cell and a c-Met-associated cancer cell.

In some embodiments, the mammalian cell is in vitro. In some embodiments, the mammalian cell is in vivo. In some embodiments, the mammalian cell is ex vivo.

Also provided herein is a method of inhibiting cell proliferation, in vitro or in vivo, the method comprising contacting a cell with an effective amount of a compound of Formula I, II, III or IV or a pharmaceutically acceptable salt thereof, or a pharmaceutical composition thereof as defined herein.

Also provided herein are methods of decreasing immune tolerance in a subject in need thereof that include administering to the subject a therapeutically effective amount of a compound of Formula I, II, Ill or IV or a pharmaceutically acceptable salt thereof, or a pharmaceutical composition thereof as defined herein. As used herein, the term “immune tolerance” refers to a decrease (e.g., a 1% to about 99% decrease, or any of the subranges of this range described herein) in one or more of: the processing of tumor-associated antigens by antigen-presenting cells (e.g., dendritic cells), presentation of antigens to tumor antigen-specific T cells, activation and proliferation of tumor antigen-specific T cells, and maintenance of the T-cell response in a subject (e.g., in a solid tumor in a subject), e.g., as compared to a control (e.g., a corresponding level in a similar subject that does not have a cancer)). In some embodiments of these methods, the subject has been identified or diagnosed as having a cancer (e.g., a TAM-associated cancer (e.g., any of the exemplary TAM-associated cancers described herein), a c-Met-associated cancer (e.g., any of the exemplary c-Met-associated cancers described herein), or both). In some examples, a decrease in immune tolerance in a subject can be detected by observing an about 1% to about 99% (e.g., about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 99%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 10%, about 10% to about 99%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%) decrease in the level of myeloid-derived suppressor cells (MDSCs) (e.g., cells characterized by expression of CD33, CD14, and low levels of HLA DR) in the subject (e.g., in a sample comprising blood or a biopsy sample obtained from the subject) (e.g., as compared to the level of MDSCs in the subject prior to administration of treatment (e.g., prior to administration of any of the compounds of Formula I, II, Ill or IV or any of the pharmaceutical compositions described herein).

In some examples, a decrease in immune tolerance in a subject can be detected by observing an about 1% to about 99% (or any of the subranges of this range described herein) decrease in the level of Treg cells (e.g., cells characterized by expression of CD4, FOXP3, and CD25) in the subject (e.g., in a sample comprising blood or a biopsy sample obtained from the subject) (e.g., as compared to the level of Tregs in the subject prior to administration of treatment (e.g., prior to administration of any of the compounds of Formula I, II, Ill or IV or any of the pharmaceutical compositions described herein).

In some examples, a decrease in immune tolerance in a subject can be detected by observing an about 1% to about 99% (or any of the subranges of this range described herein) decrease in the level of dendritic cells with reduced expression of CD80/CD86 in the subject (e.g., in a sample comprising blood or a biopsy sample obtained from the subject) (e.g., as compared to the level of dendritic cells with reduced expression of CD80/CD86 in the subject prior to administration of treatment (e.g., prior to administration of any of the compounds of Formula I, II, III or IV or any of the pharmaceutical compositions described herein). Exemplary methods for detecting the levels of MDSCs, Tregs, and dendritic cells with reduced expression of CD80/CD86 include, fluorescence-assisted cell sorting and immunofluorescence microscopy.

Also provided herein are methods of inhibiting angiogenesis in a subject in need thereof, the method comprising administering to the subject a therapeutically effective amount of a compound of Formula I, II, Ill or IV or a pharmaceutically acceptable salt thereof, or a pharmaceutical composition thereof as defined herein. In some embodiments, the angiogenesis is tumor angiogenesis and the subject has been identified or diagnosed as having a cancer (e.g., a TAM-associated cancer, a c-Met-associated cancer, or both). In some embodiments, these methods result in a decrease (e.g., a 1% to about 99% decrease, or any of the subranges of this range described herein) in the rate of development of new blood vessels (e.g., as compared to the rate of development of new blood vessels in a similar subject administered a placebo or a different treatment over a similar period of time). Exemplary methods for detecting the formation of new blood vessels include Doppler ultrasound (e.g., Color Dopler Flow Imaging), Ultrasound-Guided Diffus Optical Tomography, MRI, perfusion CT (also called functional multi-detector row CT (f-MDCT)), positron emission tomography (PET), dynamic MRI, dynamic susceptibility contrast enhanced MRI (DSC-MRI), and T1-weighted dynamic MRI (DCE-MRI). Non-limiting methods that can be used to detect the formation of new blood vessels (angiogenesis) are described in Jeswani et al., Cancer Imaging 5(1):131-138, 2005.

Also provided herein are methods of suppressing (e.g., decreasing, e.g., a 1% to about 99% decrease, or any of the subranges of this range described herein) resistance to a therapeutic agent in a subject in need thereof that include administering to the subject a therapeutically effective amount of (i) a compound of Formula I, II, Ill or IV or a pharmaceutically acceptable salt thereof, or any of the pharmaceutical compositions thereof described herein, and (ii) the therapeutic agent, where the therapeutic agent is selected from the group consisting of a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor, a RAF inhibitor, an Akt inhibitor, a FTL-3 inhibitor, and a MAP kinase pathway inhibitor. In some examples of these methods, the c-Met inhibitor is a Type 1 c-Met inhibitor, e.g., crizotinib, capmatinib, NVP-BVU972, AMG 337, bozitinib, glumetinib, savolitinib, or tepotinib. In some examples of these methods, the compound of Formula I, II, Ill or IV or a pharmaceutically acceptable salt thereof, or a pharmaceutical composition thereof, and the therapeutic agent, are administered to the subject at substantially the same time. In some embodiments of these methods, the compound of Formula I, II, Ill or IV or a pharmaceutically acceptable salt thereof, or a pharmaceutical composition thereof, and the therapeutic agent, are formulated in a single dosage form. In some embodiments of these methods, (i) the compound of Formula I, II, Ill or IV or a pharmaceutically salt thereof, or any of the pharmaceutical compositions thereof described herein is administered to the subject prior to administration of (ii) the therapeutic agent to the subject. In some embodiments of these methods, (ii) the therapeutic agent is administered to the subject prior to administration of (i) the compound of Formula I, II, Ill or IV or a pharmaceutically salt thereof, or any of the pharmaceutical compositions thereof described herein.

In some embodiments of these methods, the compound of Formula I, II, Ill or IV or a pharmaceutically acceptable salt thereof, or a pharmaceutical composition thereof, is administered to the subject prior to administration of the therapeutic agent to the subject. In some embodiments of these methods, the therapeutic agent is administered to the subject prior to administration of the compound of Formula I, II, Ill or IV or a pharmaceutically acceptable salt thereof, or a pharmaceutical composition thereof, to the subject.

As used herein, the term “resistance to a therapeutic agent” refers to a reduced or decreased level of sensitivity to treatment with a therapeutic agent (e.g., a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor (e.g., a Type 1 c-Met kinase inhibitor, e.g., crizotinib, capmatinib, and NVP-BVU972), a RAF inhibitor, an Akt inhibitor, a FTL-3 inhibitor, and a MAP kinase pathway inhibitor) in a subject (e.g., as compared to a similar subject or as compared to the level of sensitivity to the therapeutic agent at an earlier time point). For example, resistance to an therapeutic agent in a subject can be observed by a physician, e.g., by observing the requirement of an increasing dosage amounts of a therapeutic agent over time in order to achieve the same therapeutic effect in a subject, observing the requirement for an increased number of doses and/or an increased frequency of doses of a therapeutic agent over time in order to achieve the same therapeutic effect in a subject, a decrease in the observed therapeutic response to treatment with the same dosage of a therapeutic agent over time, or an observed progression of disease or disease relapse in a subject administered a therapeutic agent.

When employed as pharmaceuticals, the compounds of Formula I, II, Ill or IV can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, a compound of Formula I, II, Ill or IV is formulated as a tablet. In some embodiments, a compound of Formula I, II, Ill or IV is formulated as a capsule. In some embodiments, a compound of Formula I, II, Ill or IV is administered orally. In some embodiments, a compound of Formula I, II, Ill or IV is administered orally once a day. In some embodiments, a compound of Formula I, II, Ill or IV is administered orally twice a day.

Also provided herein are pharmaceutical compositions which contain, as the active ingredient, a compound of Formula I, II, Ill or IV or a pharmaceutically acceptable salt thereof, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is formulated as a tablet or capsule.

The compositions comprising a compound of Formula I, II, Ill or IV or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other patients, each unit containing a predetermined quantity of active material (i.e., a compound for Formula I, II, Ill or IV as provided herein) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### EXAMPLES

The following examples illustrate the invention.

### Biological Examples

### Example A

AXL Enzyme Assay

Compounds disclosed herein were screened for their ability to inhibit AXL kinase using Invitrogen's LanthaScreen™Eu Kinase Binding technology. His-tagged recombinant human AXL cytoplasmic domain was incubated with 20 nM Alexa-Fluor® Tracer 236 (PR9078A), 2 nM biotinylated anti-His (Cat. No. M4408), and 2 nM europium-labeled Streptavidin (Cat. No. PV5899) along with test compound in a buffer consisting of 25 mM HEPES, pH 7.4, 10 mM MgCl₂, 0.01% Triton X-100, and 2% DMSO. Compounds were typically prepared in a threefold serial dilution in DMSO and added to the assay to give the appropriate final concentration. After a 60-minute incubation at 22° C., the reaction was measured using a PerkinElmer EnVision multimode plate reader via TR-FRET dual wavelength detection, and the percent of control (POC) was calculated using a ratiometric emission factor. 100 POC was determined using no test compounds and 0 POC was determined using a concentration of control compound that completely inhibits the enzyme. The POC values are fit to a 4 parameter logistic curve and the IC50 value is point where the curve crosses 50 POC.

### Example B

MER Enzyme Assay

Compounds disclosed herein were screened for their ability to inhibit AXL kinase using Invitrogen's LanthaScreen^{™}Eu Kinase Binding technology. His-tagged recombinant human MER cytoplasmic domain (5 nM) was incubated with 20 nM Alexa-Fluor® Tracer 236 (PR9078A), 2 nM biotinylated anti-His (Cat. No. M4408), and 2 nM europium-labeled Streptavidin (Cat. No. PV5899) along with test compound in a buffer consisting of 25 mM HEPES, pH 7.4, 10 mM MgCl ₂, 0.01% Triton X-100, and 2% DMSO. Compounds were typically prepared in a threefold serial dilution in DMSO and added to the assay to give the appropriate final concentration. After a 60-minute incubation at 22° C., the reaction was measured using a PerkinElmer EnVision multimode plate reader via TR-FRET dual wavelength detection, and the percent of control (POC) was calculated using a ratiometric emission factor. 100 POC was determined using no test compounds and 0 POC was determined using a concentration of control compound that completely inhibits the enzyme. The POC values are fit to a 4 parameter logistic curve and the IC₅₀ value is point where the curve crosses 50 POC.

### Example C

TYRO3 Enzyme Assay

Compounds disclosed herein were screened for their ability to inhibit TYRO3 kinase using Invitrogen's LanthaScreen™ Eu Kinase Binding technology. GST-tagged recombinant human TYRO3 kinase domain from Carna (5 nM; Cat. No. PR7480A) was incubated with 20 nM Alexa-Fluor® Tracer 236 (PR9078A) and 2 nM Europium-anti-GST (Cat. No. A15116) along with test compound in a buffer consisting of 25 mM HEPES, pH 7.4, 10 mM MgCl₂, 0.01% Triton X-100, and 2% DMSO. Compounds are typically prepared in a threefold serial dilution in DMSO and added to the assay to give the appropriate final concentration. After a 60-minute incubation at 22° C., the reaction was measured using a PerkinElmer EnVision multimode plate reader via TR-FRET dual wavelength detection, and the percent of control (POC) calculated using a ratiometric emission factor. 100 POC was determined using no test compounds and 0 POC was determined using a concentration of control compound that completely inhibits the enzyme. The POC values were fit to a 4 parameter logistic curve and the IC50 value is point where the curve crosses 50 POC.

The averaged IC₅₀′s of compounds tested in the assay of Examples A, B and C are shown in Table 7.

TABLE 7

| | Axl enzyme | Mer enzyme | Tyro3 enzyme |
|---|---|---|---|
| Ex. No. | IC₅₀ (nM) | IC₅₀ (nM) | IC₅₀ (nM) |
| 1 | 1 | 3 | 5 |
| 2 | 1 | 3 | 6 |
| 3 | 1 | 3 | 5 |
| 4 | 1 | 2 | 5 |
| 5 | 2 | 4 | 12 |
| 6 | 2 | 5 | 11 |
| 7 | 2 | 3 | 7 |
| 8 | 2 | 4 | 15 |
| 9 | 2 | 3 | 13 |
| 10 | 4 | 8 | 15 |
| 11 | 5 | 16 | 33 |
| 12 | 2 | 4 | 23 |
| 13 | 2 | 5 | 22 |
| 14 | 2 | 12 | 129 |
| 15 | 2 | 29 | 292 |
| 16 | 4 | 9 | 11 |
| 17 | 28 | 52 | 76 |
| 18 | 2 | 2 | 4 |
| 19 | 2 | 2 | 4 |
| 20 | 3 | 3 | 6 |
| 21 | 10 | 10 | 26 |
| 22 | 2 | 3 | 14 |
| 23 | 1 | 3 | 14 |
| 24 | 1 | 5 | 54 |
| 25 | 2 | 5 | 54 |
| 26 | 7 | 5 | 15 |
| 27 | 1 | 2 | 3 |
| 28 | 1 | 3 | 7 |
| 29 | 1 | 3 | 6 |
| 30 | 2 | 5 | 20 |
| 31 | 1 | 5 | 23 |
| 32 | 2 | 3 | 9 |
| 33 | 1 | 5 | 10 |
| 34 | 2 | 9 | 14 |
| 35 | 6 | 15 | 30 |
| 36 | 3 | 8 | 14 |
| 37 | 5 | 16 | 30 |
| 38 | 3 | 6 | 18 |
| 39 | 3 | 16 | 83 |
| 40 | 6 | 19 | 19 |
| 41 | 5 | 33 | 141 |
| 42 | 2 | 9 | 16 |
| 43 | 2 | 5 | 11 |
| 44 | 2 | 3 | 4 |
| 45 | 5 | 27 | 91 |
| 46 | 1 | 2 | 5 |
| 47 | 1 | 7 | 56 |
| 48 | 2 | 5 | 10 |
| 49 | 1 | 5 | 58 |
| 50 | 16 | 79 | 470 |
| 51 | 5 | 14 | 112 |
| 52 | 4 | 11 | 18 |
| 53 | 448 | 950 | >1000 |
| 54 | 5 | 10 | 21 |
| 55 | 2 | 5 | 8 |
| 56 | 3 | 6 | 10 |
| 57 | 12 | 29 | 255 |
| 58 | 1 | 2 | 6 |

### Example D

c-Met Enzyme Assay

The affinity of compound binding to wild type and mutant human MET kinases was measured using Invitrogen's LanthaScreen™ Eu Kinase Binding technology. Briefly, GST-tagged recombinant human MET kinase domain from Signal Chem (see Table 8 below for concentration in assay) was incubated with 50 nM Alexa-Fluor® Tracer 236 (Invitrogen Cat No. PR9078A) and 2 nM Europium-anti-GST (Invitrogen Cat. No. A15116) along with test compound in a buffer consisting of 25 mM HEPES, pH 7.4, 10 mM MgCl2, 0.01% Triton X-100, 1mM DTT, and 2% DMSO. Compounds were typically prepared in a three-fold serial dilution in DMSO and added to the assay to give the appropriate final concentration. After a 60-minute incubation at 22° C., the reaction was measured using a PerkinElmer EnVision multimode plate reader via TR-FRET dual wavelength detection, and the percent of control (POC) calculated using a ratiometric emission factor. 100 POC was determined using no test compounds and 0 POC is determined using a concentration of control compound that completely inhibits the enzyme. The POC values were fit to a 4 parameter logistic curve and the IC₅₀ value is point where the curve crosses 50 POC.

TABLE 8
Concentration of Wild Type and Mutant
MET kinases in binding assay

| | | | | Enzyme |
|---|---|---|---|---|
| Met | | | | Concentration |
| Mutant | | Catalog | MET Amino | in Binding |
| Enzyme | Source | Number | Acids | Assay (nM) |
| del Ex14 | SignalChem | M52-12PG | 956-1390 (end) | 5 |
| L1195V | SignalChem | NP-18-156G | 956-1390 (end) | 10 |
| F1200I | SignalChem | M52-12GG | 956-1390 (end) | 2 |
| D1228H | SignalChem | M52-12HG | 956-1390 (end) | 2 |
| D1228N | SignalChem | M52-12IG | 956-1390 (end) | 2 |
| Y1230C | SignalChem | M52-12KG | 956-1390 (end) | 2 |
| Y1230H | SignalChem | M52-12MG | 956-1390 (end) | 5 |
| Y1230S | SignalChem | NP18-157G | 956-1390 (end) | 8 |
| MET (wt) | SignalChem | M52-18G | 956-1390 (end) | 10 |

The averaged IC₅₀′s of compounds tested in the assay of Example D are shown in Table 9.

TABLE 9

| | SigChem | | | | | | | | |
|---|---|---|---|---|---|---|---|---|---|
| | WT | Del14 | D1228 | D1228 | F1200I | L1195V | Y1230 | Y1230 | Y1230 |
| Ex. | IC₅₀ | IC₅₀ | H IC₅₀ | N IC₅₀ | IC₅₀ | IC₅₀ | C IC₅₀ | H IC₅₀ | S IC₅₀ |
| No. | (nM) | (nM) | (nM) | (nM) | (nM) | (nM) | (nM) | (nM) | (nM) |
| 1 | 2.4 | 2.6 | 1.8 | 3.4 | 2.5 | 7.1 | 1.5 | 2.9 | 1.3 |
| 2 | 6.4 | 5.3 | 16.6 | 29.3 | 6.8 | 87.1 | 9.5 | 18.2 | 9.8 |
| 3 | 3.2 | 3.0 | 4.7 | 7.4 | 5.0 | 15.3 | 4.2 | 6.4 | 4.7 |
| 4 | 2.1 | 1.7 | 3.2 | 6.5 | 3.0 | 21.9 | 2.8 | 4.9 | 2.2 |
| 5 | 6.4 | 6.4 | 9.6 | 16.1 | 5.7 | 9.8 | 8.1 | 16.6 | 5.4 |
| 6 | 8.8 | 7.8 | 12.1 | 24.2 | 8.1 | 62.1 | 12.4 | 24.2 | 11.9 |
| 7 | 3.1 | 2.6 | 2.0 | 6.4 | 3.7 | 7.3 | 2.9 | 5.4 | 2.1 |
| 8 | 4.1 | 3.9 | 7.2 | 10.7 | 4.9 | 29.1 | 7.4 | 8.6 | 4.3 |
| 9 | 2.9 | 5.6 | 10.0 | 15.8 | 6.4 | 47.7 | 6.2 | 13.1 | 9.1 |
| 10 | 3.6 | 3.5 | 3.7 | 4.1 | 4.2 | 9.7 | 3.1 | 6.1 | 2.8 |
| 11 | 7.8 | 7.7 | 6.5 | 9.4 | 6.9 | 21.4 | 6.5 | 9.3 | 4.9 |
| 12 | 5.4 | 5.6 | 8.4 | 9.7 | 7.0 | 33.0 | 5.5 | 5.4 | 4.6 |
| 13 | 3.4 | 4.9 | 4.4 | 11.6 | 4.8 | 83.8 | 3.3 | 5.7 | 6.0 |
| 14 | 7.7 | 15.9 | 35.4 | 30.6 | 12.4 | 186.7 | 5.5 | 22.3 | 16.3 |
| 15 | 33.8 | 29.5 | 99.1 | 73.7 | 24.4 | 61.4 | 66.8 | 37.5 | 53.3 |
| 16 | 6.1 | 4.6 | 5.4 | 8.1 | 6.8 | 34.6 | 6.1 | 7.7 | 6.0 |
| 17 | 17.8 | 17.1 | 13.3 | 25.0 | 23.4 | 82.6 | 15.5 | 26.9 | 11.4 |
| 18 | 2.7 | 3.2 | 2.4 | 4.7 | 4.3 | 21.4 | 2.4 | 4.6 | 2.7 |
| 19 | 1.8 | 1.9 | 1.6 | 3.6 | 2.8 | 10.0 | 2.0 | 2.7 | 1.8 |
| 20 | 5.2 | 3.8 | 9.9 | 10.4 | 5.1 | 58.1 | 7.7 | 9.9 | 6.2 |
| 21 | 13.5 | 13.6 | 18.7 | 31.0 | 16.2 | 77.7 | 20.4 | 51.3 | 29.2 |
| 22 | 1.8 | 3.1 | 4.2 | 6.5 | 2.5 | 17.7 | 2.0 | 4.3 | 2.2 |
| 23 | 2.8 | 2.9 | 11.5 | 13.5 | 4.3 | 39.6 | 14.5 | 7.8 | 3.8 |
| 24 | 7.8 | 10.5 | 15.2 | 36.7 | 11.2 | 92.0 | 14.3 | 22.7 | 16.5 |
| 25 | 8.2 | 9.5 | 22.3 | 37.8 | 11.5 | 18.7 | 10.4 | 21.2 | 8.0 |
| 26 | 6.0 | 5.2 | 12.5 | 19.2 | 6.4 | 5000.0 | 6.5 | 17.0 | 9.1 |
| 27 | 1.8 | 1.1 | 1.1 | 2.0 | 1.6 | 2.9 | 1.5 | 2.3 | 1.1 |
| 28 | 2.0 | 1.9 | 1.4 | 3.6 | 2.4 | 4.0 | 1.7 | 7.8 | 1.8 |
| 29 | 4.3 | 2.5 | 2.4 | 3.1 | 3.2 | 5.1 | 2.6 | 4.2 | 1.5 |
| 30 | 39.7 | 50.3 | 57.4 | 100.1 | 59.1 | 39.9 | 83.5 | 103.1 | 89.2 |
| 31 | 41.0 | 44.7 | 55.7 | 62.7 | 46.5 | 59.1 | 69.3 | 98.2 | 72.1 |
| 32 | 3.0 | 3.2 | 10.0 | 17.4 | 4.2 | 44.8 | 6.7 | 12.1 | 7.1 |
| 33 | 3.9 | 4.9 | 5.2 | 13.3 | 4.6 | 20.2 | 2.7 | 11.2 | 5.0 |
| 34 | 4.0 | 5.7 | 5.5 | 7.2 | 7.4 | 11.2 | 4.2 | 5.9 | 4.1 |
| 35 | 13.8 | 14.0 | 8.3 | 16.2 | 11.5 | 34.3 | 10.5 | 18.3 | 8.5 |
| 36 | 2.4 | 2.4 | 1.8 | 4.8 | 3.2 | 14.4 | 2.8 | 4.4 | 2.3 |
| 37 | 10.0 | 12.2 | 10.5 | 16.8 | 16.4 | 23.2 | 9.3 | 16.8 | 10.2 |
| 38 | N/A | N/A | N/A | N/A | N/A | N/A | N/A | N/A | N/A |
| 39 | 29.4 | 25.6 | 66.3 | 178.7 | 31.6 | 507.8 | 47.0 | 208.7 | 55.3 |
| 40 | N/A | N/A | N/A | N/A | N/A | N/A | N/A | N/A | N/A |
| 41 | 116.7 | 108.0 | 257.8 | 645.7 | 105.0 | 2220.9 | 173.1 | 328.1 | 225.5 |
| 42 | 3.2 | 1.9 | 2.4 | 7.0 | 3.4 | 8.2 | 2.4 | 6.7 | 2.7 |
| 43 | 1.4 | 2.5 | 1.6 | 2.7 | 2.0 | 14.2 | 1.4 | 2.9 | 1.1 |
| 44 | 2.2 | 2.1 | 1.9 | 3.2 | 4.2 | 18.9 | 1.7 | 2.7 | 2.1 |
| 45 | 4.4 | 5.7 | 11.1 | 13.4 | 8.3 | 41.4 | 4.4 | 9.9 | 6.2 |
| 46 | 3.0 | 2.8 | 3.2 | 5.4 | 3.4 | 21.9 | 1.7 | 3.4 | 1.7 |
| 47 | 10.2 | 14.3 | 31.1 | 29.7 | 8.5 | 413.6 | 8.8 | 29.4 | 18.9 |
| 48 | 6.8 | 4.9 | 11.9 | 14.6 | 5.4 | 31.7 | 9.4 | 15.8 | 8.8 |
| 49 | 27.0 | 22.0 | 64.3 | 129.8 | 27.8 | 459.3 | 39.0 | 112.1 | 67.1 |
| 50 | 59.2 | 90.4 | 222.8 | 444.5 | 104.3 | 1033.5 | 198.7 | 396.8 | 165.6 |
| 51 | 22.2 | 29.4 | 59.5 | 178.1 | 35.0 | 494.8 | 61.8 | 200.4 | 44.0 |
| 52 | 14.7 | 10.3 | 14.5 | 26.3 | 20.1 | 73.6 | 13.1 | 32.6 | 14.3 |
| 53 | 3067.1 | 5000.0 | 4222.5 | 5000.0 | 3692.9 | 5000.0 | 3312.4 | 1666.7 | 3707.0 |
| 54 | 4.7 | 3.8 | 3.8 | 8.3 | 6.7 | 14.7 | 4.3 | 7.2 | 3.2 |
| 55 | 2.8 | 3.1 | 2.2 | 3.4 | 3.4 | 5.1 | 2.2 | 2.8 | 2.6 |
| 56 | 3.9 | 4.5 | 1.9 | 4.3 | 5.0 | 5.3 | 3.2 | 5.2 | 3.0 |
| 57 | 296.7 | 304.7 | 531.6 | 1058.1 | 346.0 | 5000.0 | 525.8 | 788.9 | 513.8 |
| 58 | 2.7 | 3.1 | 3.5 | 7.7 | 3.5 | 35.0 | 2.2 | 5.3 | 2.7 |

### Example E

MDR1 LLC-PK1 permeability assay

MDR1 transfected LLC-PK1 cells were cultured and plated according to manufacturer's recommendations with the exception that the passage media contained only 2% fetal bovine serum to extend passage time out to seven days.

Both positive and negative controls were used to assess functionality of P-gp efflux in the assay. Stock solutions for assay controls and the test article were prepared in DMSO for final test concentrations of 10 and 1 μM, respectively. Final organic concentration in the assay was 1%. All dosing solutions contained 10 μM lucifer yellow to monitor LLC-PK1 cell monolayer integrity.

For the apical to basolateral determination (A to B), 75 μL of the test article in transport buffer were added to the apical side of the individual transwells and 250 μL of basolateral media, without compound or lucifer yellow, were added to each well. For the basolateral to apical determination (B to A), 250 μL of test article in transport buffer were added to each well and 75 μL transport buffer, without compound or lucifer yellow, were added to each transwell. All tests were performed in triplicate, and each compound was tested for both apical to basolateral and basolateral to apical transport. The plates were incubated for 2 hours on a Lab-Line Instruments Titer Orbital Shaker (VWR, West Chester, Pa.) at 50 rpm and 37° C. with 5% CO₂. All culture plates were removed from the incubator and 50 μL of media were removed from the apical and basolateral portion of each well and added to 150 μL of 1 μM labetalol in 2:1 acetonitrile (acetonitrile): H₂O, v/v.

The plates were read using a Molecular Devices (Sunnyvale, Calif.) Gemini Fluorometer to evaluate the lucifer yellow concentrations at excitation/emission wavelengths of 425/535 nm. These values were accepted when found to be below 2% for apical to basolateral and 5% basolateral to apical flux across the MDR1-transfected LLC-PK1-transfected cell monolayers. The plates were sealed and the contents of each well analyzed by LC-MS/MS. The compound concentrations were determined from the ratio of the peak areas of the compound to the internal standard (labetalol) in comparison to the dosing solution.

LC-MS analysis

The LC-MS/MS system was comprised of an HTS-PAL autosampler (Leap Technologies, Carrboro, N.C.), an HP1200 HPLC (Agilent, Palo Alto, Calif.), and a MDS Sciex 4000 Q Trap system (Applied Biosystems, Foster City, Calif.). Chromatographic separation of the analyte and internal standard was achieved at room temperature using a C18 column (Kinetics®, 50×300 mm, 2.6 μm particle size, Phenomenex, Torrance, Calif.) in conjunction with gradient conditions using mobile phases A (water containing 1% isopropyl alcohol and 0.1% formic acid) and B (0.1% formic acid in acetonitrile). The total run time, including re-equilibration, for a single injection was 1.2 minutes. Mass spectrometric detection of the analytes was accomplished using the ion spray positive mode. Analyte responses were measured by multiple reaction monitoring (MRM) of transitions unique to each compound (the protonated precursor ion and selected product ions for each test article and m/z 329 to m/z 162 for labetalol, the internal standard).

The permeability coefficient (Pₐₚₚ) is calculated from the following equation:

Pₐₚₚ=[((C_{d}*V*(1×10⁶))/(t*0.12 cm²*C)]

where C_{d}, V, t and C₀ are the detected concentration (μM), the volume on the dosing side (mL), the incubation time (s) and the initial dosing concentration (μM), respectively. The calculations for Pₐₚₚ were made for each replicate and then averaged. Permeability coefficients for compounds of Formula I are provided in Table E1. In this assay, a compound is defined has having high permeability if the permeability is greater than 8×10⁻⁶ cm/sec, a compound is defined has having medium permeability if the permeability is from 2×10⁻⁶ cm/sec to 8×10⁻⁶ cm/sec, and a compound is defined has having low permeability if the permeability is less than 2×10⁻⁶ cm/sec.

An efflux ratio was calculated from the mean apical to basolateral (A-B) Pₐₚₚ data and basolateral to apical (B-A) Papp data:

Efflux ratio=Pₐₚₚ(B−A)/Pₐₚₚ(A−B)

Efflux ratios for representative compounds of Formula I when tested in this assay are shown in Table E1. Compounds of Formula IV (Examples 1, 4, 5, 16, 18, 19, 21, 28, 30, 31, 33, 35, 39, 41, 48, 49, 51, 52, 57 and 58) show a trend towards lower efflux ratios (i.e., efflux ratios 3.5) in the MDR1 assay compared to certain compounds of Formula I, indicating that compounds of Formula IV will have increased brain penetration compared to such certain compounds of Formula I.

TABLE E1

| | | | MDR1 |
|---|---|---|---|
| Ex. | | Permeability | Pe |
| No. | Structure | (*10-6 cm/s) | Ratio |
| 1 | 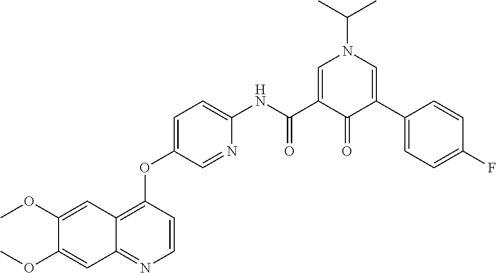 [SMILES: COc1cc2nccc(Oc3ccc(NC(=O)c4cn(C(C)C)cc(-c5ccc(F)cc5)c4=O)nc3)c2cc1OC] | 10 | 3.2 |
| 2 | 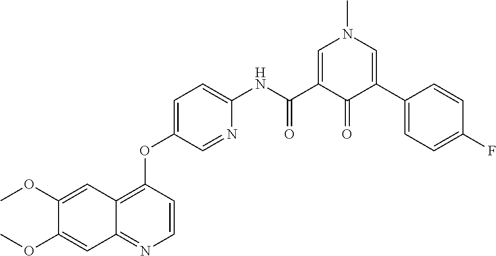 [SMILES: COc1cc2nccc(Oc3ccc(NC(=O)c4cn(C)cc(-c5ccc(F)cc5)c4=O)nc3)c2cc1OC] | 15 | 8.3 |
| 3 | 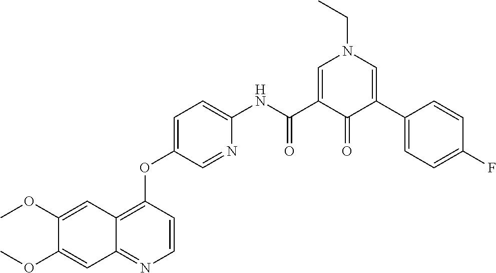 [SMILES: CCn1cc(C(=O)Nc2ccc(Oc3ccnc4cc(OC)c(OC)cc34)cn2)c(=O)c(-c2ccc(F)cc2)c1] | 10 | 4.3 |
| 4 | 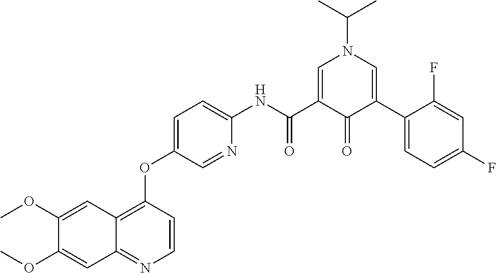 [SMILES: COc1cc2nccc(Oc3ccc(NC(=O)c4cn(C(C)C)cc(-c5ccc(F)cc5F)c4=O)nc3)c2cc1OC] | 10 | 1.5 |
| 5 | 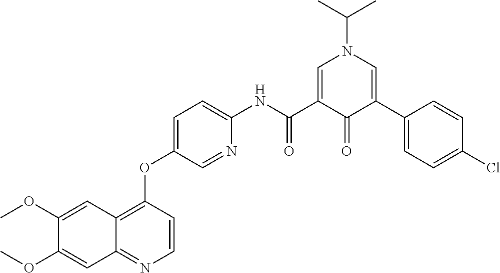 [SMILES: COc1cc2nccc(Oc3ccc(NC(=O)c4cn(C(C)C)cc(-c5ccc(Cl)cc5)c4=O)nc3)c2cc1OC] | 5 | 2.6 |
| 6 | 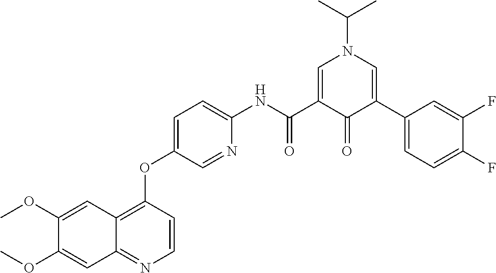 [SMILES: COc1cc2nccc(Oc3ccc(NC(=O)c4cn(C(C)C)cc(-c5ccc(F)c(F)c5)c4=O)nc3)c2cc1OC] | 7 | 4.9 |
| 7 | 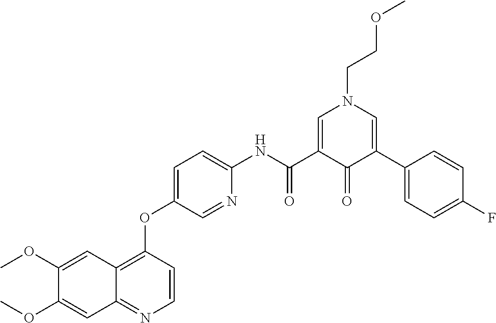 [SMILES: COCCn1cc(C(=O)Nc2ccc(Oc3ccnc4cc(OC)c(OC)cc34)cn2)c(=O)c(-c2ccc(F)cc2)c1] | 11 | 8.2 |
| 8 | 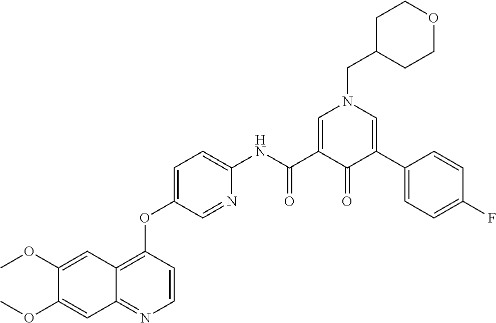 [SMILES: COc1cc2nccc(Oc3ccc(NC(=O)c4cn(CC5CCOCC5)cc(-c5ccc(F)cc5)c4=O)nc3)c2cc1OC] | 9 | 10 |
| 9 | 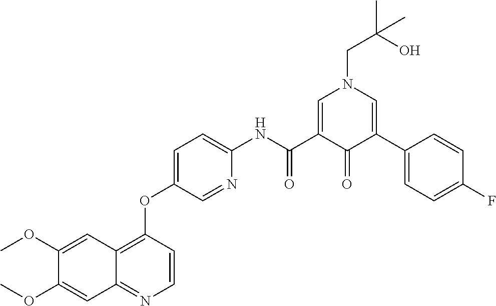 [SMILES: COc1cc2nccc(Oc3ccc(NC(=O)c4cn(CC(C)(C)O)cc(-c5ccc(F)cc5)c4=O)nc3)c2cc1OC] | 14 | 26 |
| 10 | 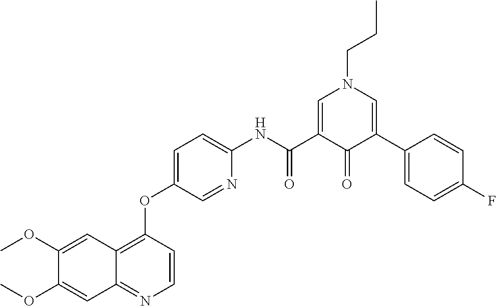 [SMILES: CCCn1cc(C(=O)Nc2ccc(Oc3ccnc4cc(OC)c(OC)cc34)cn2)c(=O)c(-c2ccc(F)cc2)c1] | 7 | 2.7 |
| 11 | 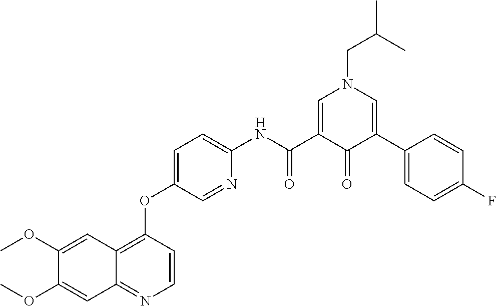 [SMILES: COc1cc2nccc(Oc3ccc(NC(=O)c4cn(CC(C)C)cc(-c5ccc(F)cc5)c4=O)nc3)c2cc1OC] | — | 31 |
| 12 | 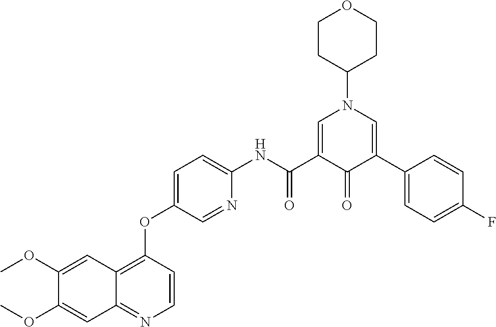 [SMILES: COc1cc2nccc(Oc3ccc(NC(=O)c4cn(C5CCOCC5)cc(-c5ccc(F)cc5)c4=O)nc3)c2cc1OC] | 13 | 15 |
| 13 | 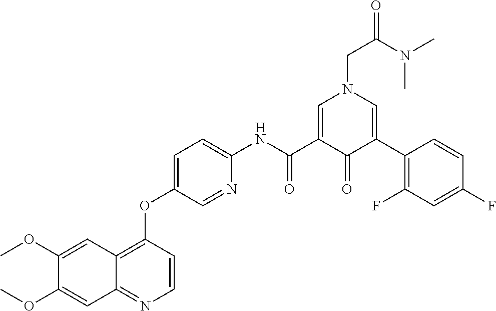 [SMILES: COc1cc2nccc(Oc3ccc(NC(=O)c4cn(CC(=O)N(C)C)cc(-c5ccc(F)cc5F)c4=O)nc3)c2cc1OC] | 6 | 112 |
| 14 | 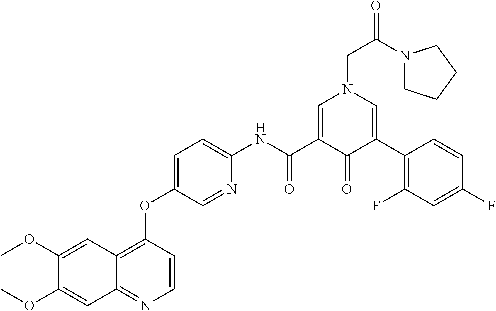 [SMILES: COc1cc2nccc(Oc3ccc(NC(=O)c4cn(CC(=O)N5CCCC5)cc(-c5ccc(F)cc5F)c4=O)nc3)c2cc1OC] | 10 | 125 |
| 15 | 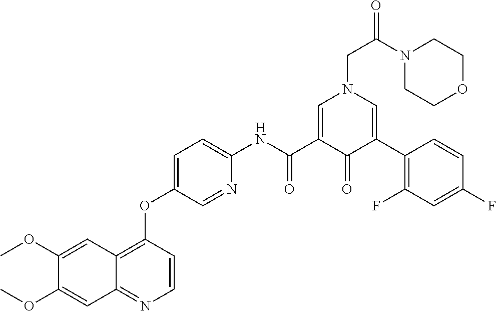 [SMILES: COc1cc2nccc(Oc3ccc(NC(=O)c4cn(CC(=O)N5CCOCC5)cc(-c5ccc(F)cc5F)c4=O)nc3)c2cc1OC] | 2 | 135 |
| 16 | 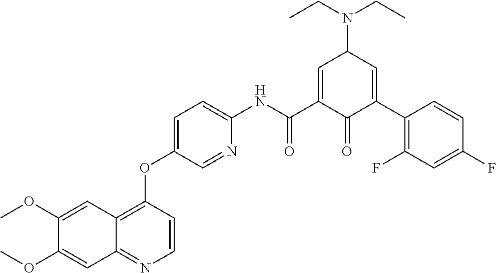 [SMILES: CCN(CC)C1C=C(C(=O)Nc2ccc(Oc3ccnc4cc(OC)c(OC)cc34)cn2)C(=O)C(c2ccc(F)cc2F)=C1] | 4 | 2.0 |
| 17 | 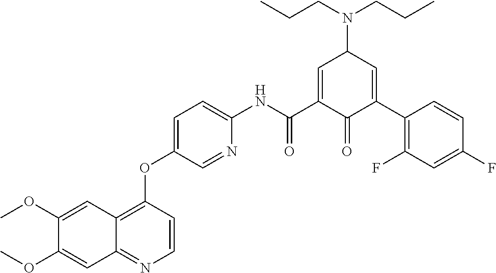 [SMILES: CCCN(CCC)C1C=C(C(=O)Nc2ccc(Oc3ccnc4cc(OC)c(OC)cc34)cn2)C(=O)C(c2ccc(F)cc2F)=C1] | <1 | 24 |
| 18 | 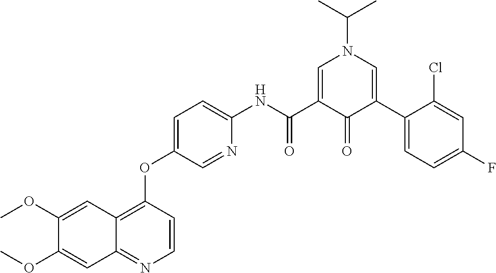 [SMILES: COc1cc2nccc(Oc3ccc(NC(=O)c4cn(C(C)C)cc(-c5ccc(F)cc5Cl)c4=O)nc3)c2cc1OC] | 11 | 2.1 |
| 19 | 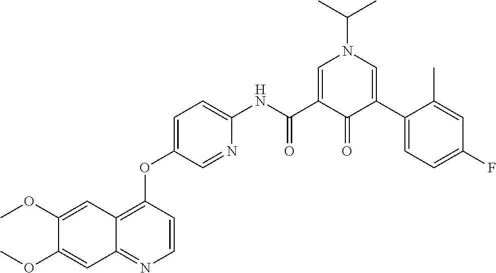 [SMILES: COc1cc2nccc(Oc3ccc(NC(=O)c4cn(C(C)C)cc(-c5ccc(F)cc5C)c4=O)nc3)c2cc1OC] | 11 | 2.8 |
| 20 | 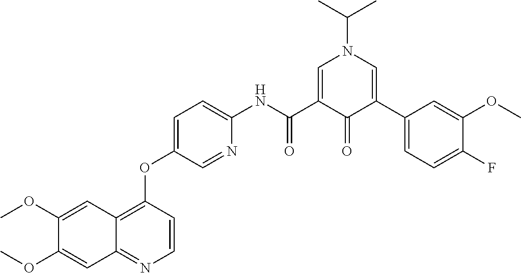 | 9 | 3.6 |
| 21 | 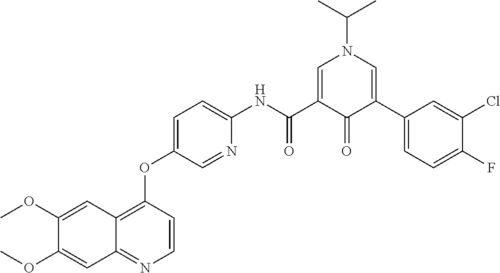 [SMILES: COc1cc2nccc(Oc3ccc(NC(=O)c4cn(C(C)C)cc(-c5ccc(F)c(Cl)c5)c4=O)nc3)c2cc1OC] | 1.2 | 1.3 |
| 22 | 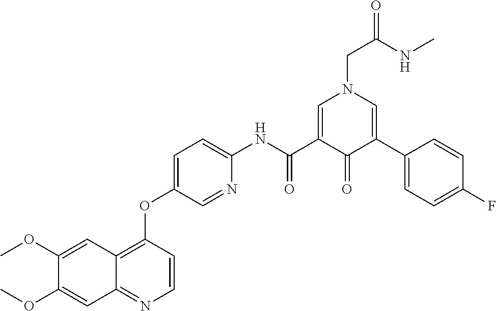 [SMILES: CNC(=O)Cn1cc(C(=O)Nc2ccc(Oc3ccnc4cc(OC)c(OC)cc34)cn2)c(=O)c(-c2ccc(F)cc2)c1] | 2 | 80 |
| 23 | 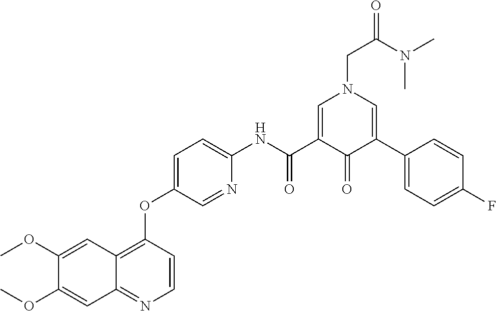 [SMILES: COc1cc2nccc(Oc3ccc(NC(=O)c4cn(CC(=O)N(C)C)cc(-c5ccc(F)cc5)c4=O)nc3)c2cc1OC] | 6 | 105 |
| 24 | 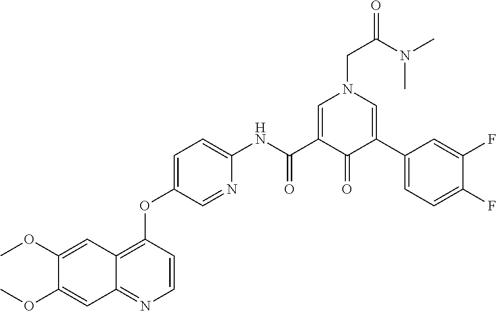 [SMILES: COc1cc2nccc(Oc3ccc(NC(=O)c4cn(CC(=O)N(C)C)cc(-c5ccc(F)c(F)c5)c4=O)nc3)c2cc1OC] | 9.5 | 210 |
| 25 | 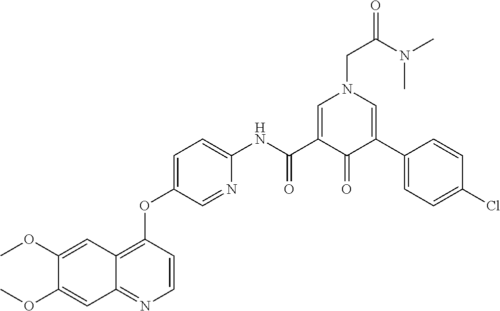 [SMILES: COc1cc2nccc(Oc3ccc(NC(=O)c4cn(CC(=O)N(C)C)cc(-c5ccc(Cl)cc5)c4=O)nc3)c2cc1OC] | 6.4 | 136 |
| 26 | 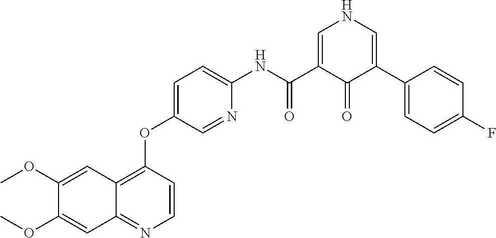 [SMILES: COc1cc2nccc(Oc3ccc(NC(=O)c4c[nH]cc(-c5ccc(F)cc5)c4=O)nc3)c2cc1OC] | 3 | 44 |
| 27 | 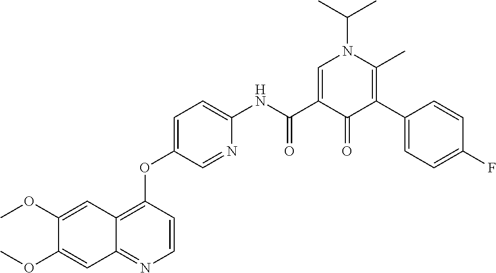 [SMILES: COc1cc2nccc(Oc3ccc(NC(=O)c4cn(C(C)C)c(C)c(-c5ccc(F)cc5)c4=O)nc3)c2cc1OC] | 16 | 5.5 |
| 28 | 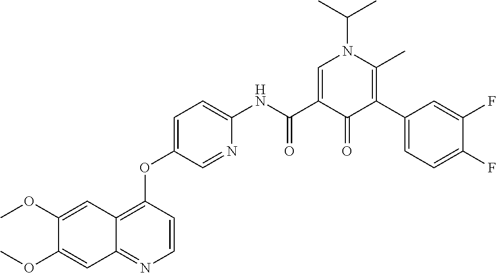 [SMILES: COc1cc2nccc(Oc3ccc(NC(=O)c4cn(C(C)C)c(C)c(-c5ccc(F)c(F)c5)c4=O)nc3)c2cc1OC] | 15 | 2.8 |
| 29 | 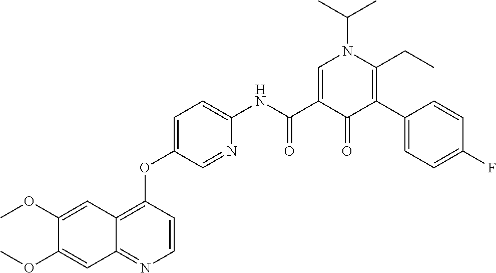 [SMILES: CCc1c(-c2ccc(F)cc2)c(=O)c(C(=O)Nc2ccc(Oc3ccnc4cc(OC)c(OC)cc34)cn2)cn1C(C)C] | 10 | 3.6 |
| 30 | 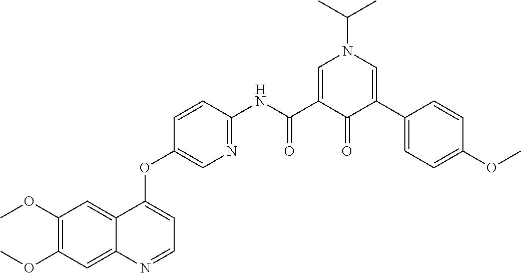 | 7 | 3.1 |
| 31 | 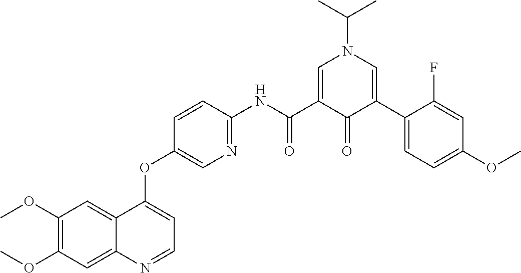 | 11 | 2.0 |
| 32 | 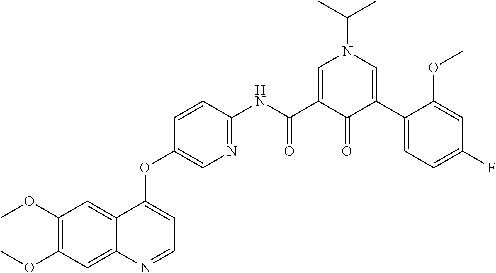 [SMILES: COc1cc2nccc(Oc3ccc(NC(=O)c4cn(C(C)C)cc(-c5ccc(F)cc5OC)c4=O)nc3)c2cc1OC] | 14 | 7.1 |
| 33 | 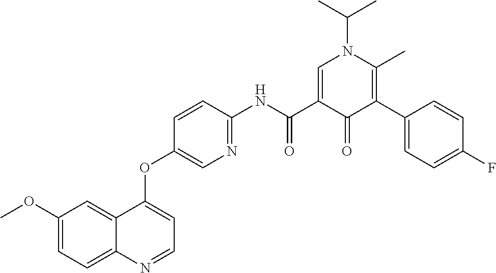 [SMILES: COc1ccc2nccc(Oc3ccc(NC(=O)c4cn(C(C)C)c(C)c(-c5ccc(F)cc5)c4=O)nc3)c2c1] | 10 | 2.0 |
| 34 | 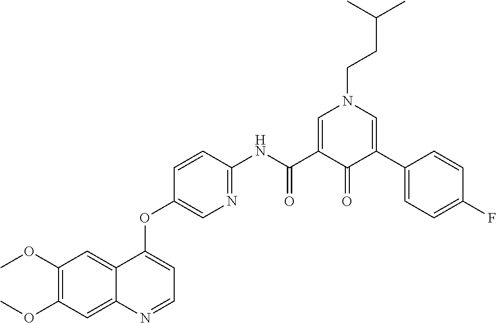 [SMILES: COc1cc2nccc(Oc3ccc(NC(=O)c4cn(CCC(C)C)cc(-c5ccc(F)cc5)c4=O)nc3)c2cc1OC] | <1 | 5.3 |
| 35 | 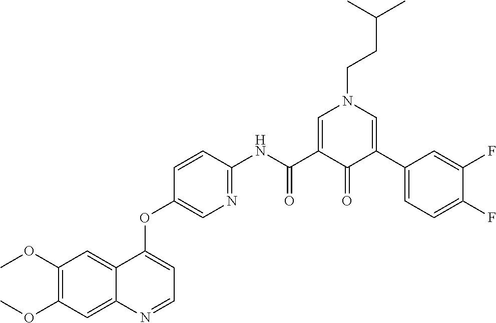 [SMILES: COc1cc2nccc(Oc3ccc(NC(=O)c4cn(CCC(C)C)cc(-c5ccc(F)c(F)c5)c4=O)nc3)c2cc1OC] | <1 | 3.5 |
| 36 | 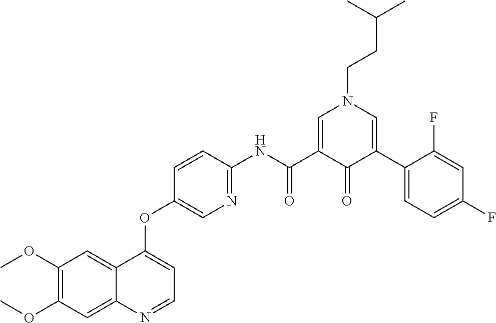 [SMILES: COc1cc2nccc(Oc3ccc(NC(=O)c4cn(CCC(C)C)cc(-c5ccc(F)cc5F)c4=O)nc3)c2cc1OC] | 1 | 4.3 |
| 37 | 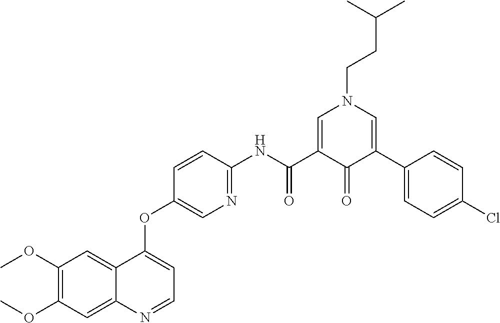 [SMILES: COc1cc2nccc(Oc3ccc(NC(=O)c4cn(CCC(C)C)cc(-c5ccc(Cl)cc5)c4=O)nc3)c2cc1OC] | <1 | 13 |
| 38 | 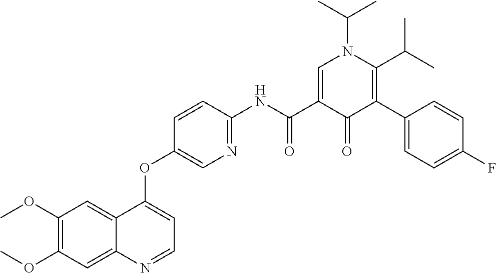 [SMILES: COc1cc2nccc(Oc3ccc(NC(=O)c4cn(C(C)C)c(C(C)C)c(-c5ccc(F)cc5)c4=O)nc3)c2cc1OC] | 25 | — |
| 39 | 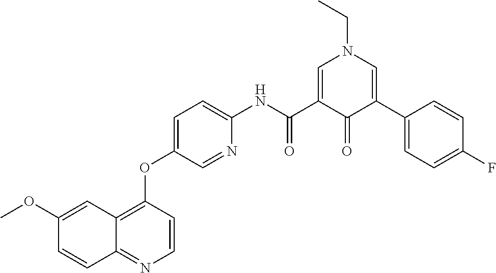 [SMILES: CCn1cc(C(=O)Nc2ccc(Oc3ccnc4ccc(OC)cc34)cn2)c(=O)c(-c2ccc(F)cc2)c1] | 7 | 0.9 |
| 40 | 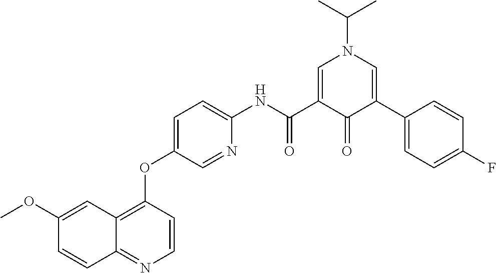 [SMILES: COc1ccc2nccc(Oc3ccc(NC(=O)c4cn(C(C)C)cc(-c5ccc(F)cc5)c4=O)nc3)c2c1] | — | — |
| 41 | 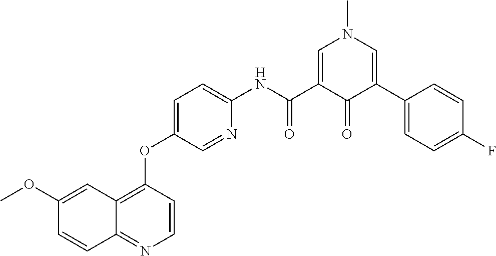 [SMILES: COc1ccc2nccc(Oc3ccc(NC(=O)c4cn(C)cc(-c5ccc(F)cc5)c4=O)nc3)c2c1] | 7 | 2.5 |
| 42 | 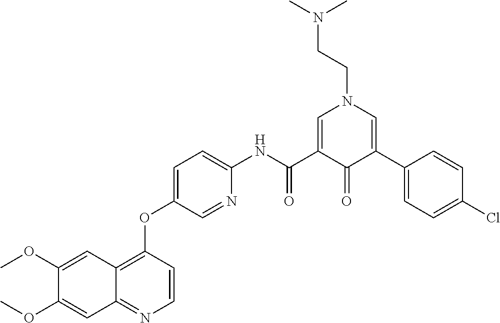 [SMILES: COc1cc2nccc(Oc3ccc(NC(=O)c4cn(CCN(C)C)cc(-c5ccc(Cl)cc5)c4=O)nc3)c2cc1OC] | — | 12 |
| 43 | 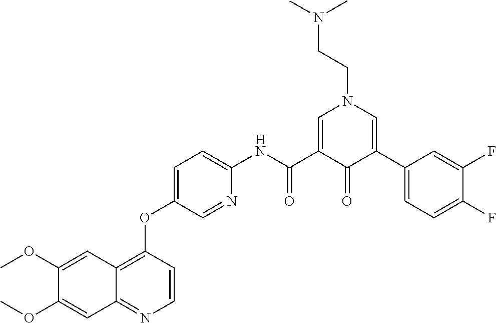 [SMILES: COc1cc2nccc(Oc3ccc(NC(=O)c4cn(CCN(C)C)cc(-c5ccc(F)c(F)c5)c4=O)nc3)c2cc1OC] | — | 13 |
| 44 | 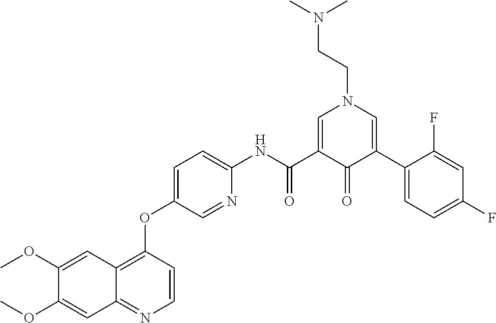 [SMILES: COc1cc2nccc(Oc3ccc(NC(=O)c4cn(CCN(C)C)cc(-c5ccc(F)cc5F)c4=O)nc3)c2cc1OC] | 2 | 11 |
| 45 | 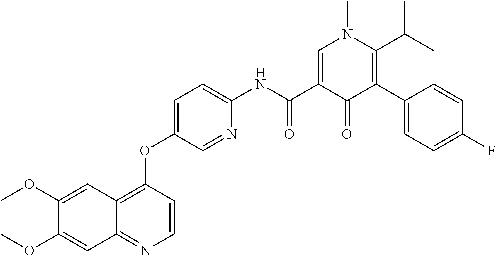 [SMILES: COc1cc2nccc(Oc3ccc(NC(=O)c4cn(C)c(C(C)C)c(-c5ccc(F)cc5)c4=O)nc3)c2cc1OC] | 5 | 16 |
| 46 | 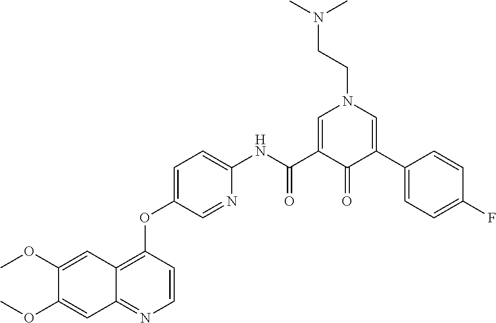 [SMILES: COc1cc2nccc(Oc3ccc(NC(=O)c4cn(CCN(C)C)cc(-c5ccc(F)cc5)c4=O)nc3)c2cc1OC] | 2 | 13 |
| 47 | 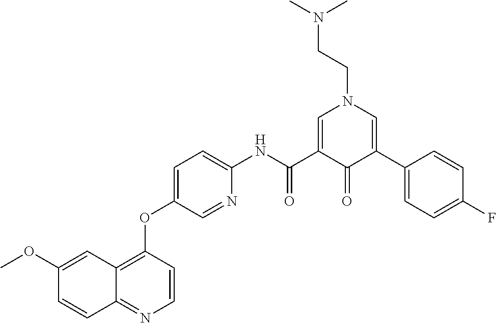 [SMILES: COc1ccc2nccc(Oc3ccc(NC(=O)c4cn(CCN(C)C)cc(-c5ccc(F)cc5)c4=O)nc3)c2c1] | — | 9.3 |
| 48 | 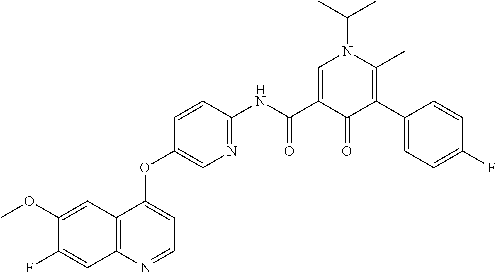 [SMILES: COc1cc2c(Oc3ccc(NC(=O)c4cn(C(C)C)c(C)c(-c5ccc(F)cc5)c4=O)nc3)ccnc2cc1F] | 15 | 1.6 |
| 49 | 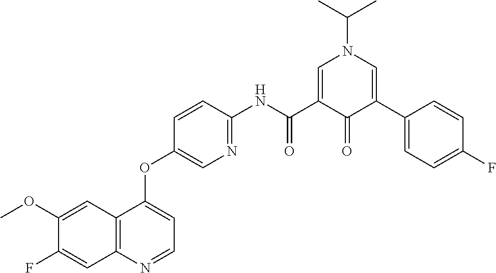 [SMILES: COc1cc2c(Oc3ccc(NC(=O)c4cn(C(C)C)cc(-c5ccc(F)cc5)c4=O)nc3)ccnc2cc1F] | 4 | 0.99 |
| 50 | 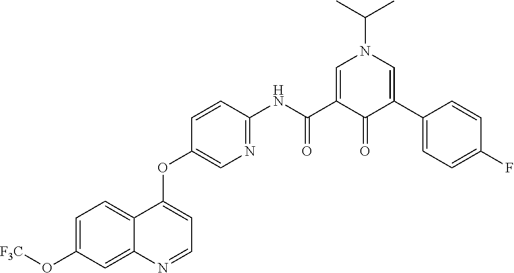 [SMILES: CC(C)n1cc(C(=O)Nc2ccc(Oc3ccnc4cc(OC(F)(F)F)ccc34)cn2)c(=O)c(-c2ccc(F)cc2)c1] | <1 | 4.7 |
| 51 | 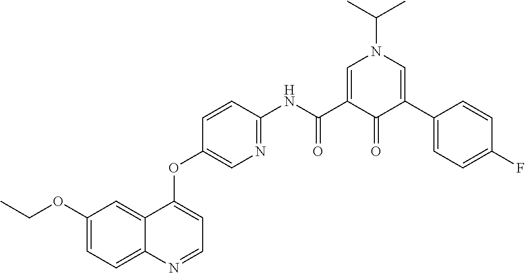 [SMILES: CCOc1ccc2nccc(Oc3ccc(NC(=O)c4cn(C(C)C)cc(-c5ccc(F)cc5)c4=O)nc3)c2c1] | 3 | 2.0 |
| 52 | 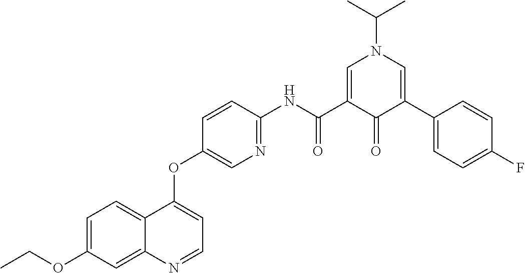 [SMILES: CCOc1ccc2c(Oc3ccc(NC(=O)c4cn(C(C)C)cc(-c5ccc(F)cc5)c4=O)nc3)ccnc2c1] | 2 | 1.8 |
| 53 | 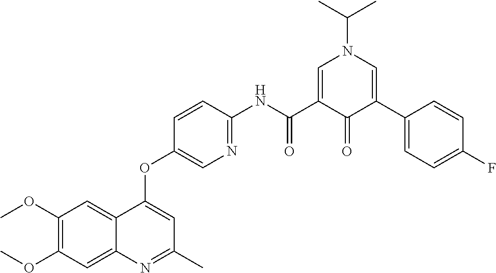 [SMILES: COc1cc2nc(C)cc(Oc3ccc(NC(=O)c4cn(C(C)C)cc(-c5ccc(F)cc5)c4=O)nc3)c2cc1OC] | 5 | 3.4 |
| 54 | 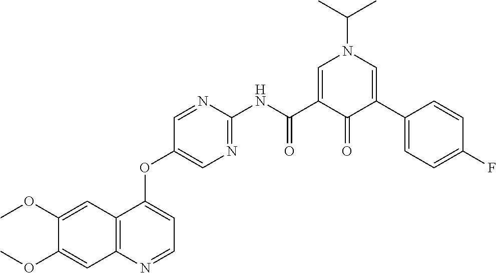 [SMILES: COc1cc2nccc(Oc3cnc(NC(=O)c4cn(C(C)C)cc(-c5ccc(F)cc5)c4=O)nc3)c2cc1OC] | 12 | 114 |
| 55 | 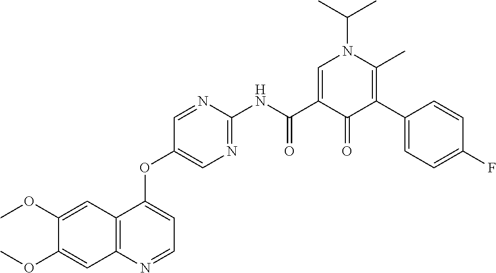 [SMILES: COc1cc2nccc(Oc3cnc(NC(=O)c4cn(C(C)C)c(C)c(-c5ccc(F)cc5)c4=O)nc3)c2cc1OC] | 8 | 144 |
| 56 | 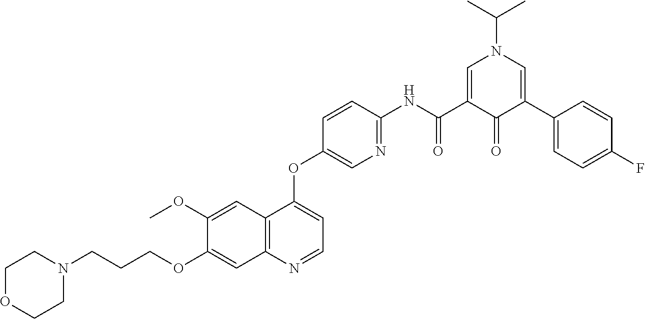 [SMILES: COc1cc2c(Oc3ccc(NC(=O)c4cn(C(C)C)cc(-c5ccc(F)cc5)c4=O)nc3)ccnc2cc1OCCCN1CCOCC1] | <1 | 20 |
| 57 | 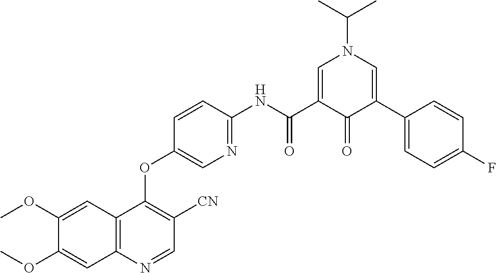 [SMILES: COc1cc2ncc(C#N)c(Oc3ccc(NC(=O)c4cn(C(C)C)cc(-c5ccc(F)cc5)c4=O)nc3)c2cc1OC] | 15 | 2.5 |
| 58 | 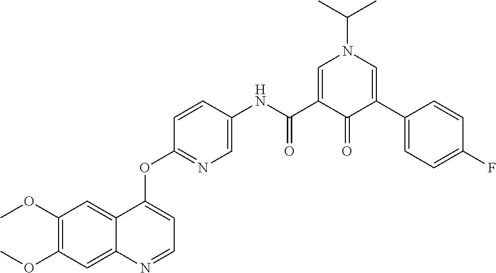 [SMILES: COc1cc2nccc(Oc3ccc(NC(=O)c4cn(C(C)C)cc(-c5ccc(F)cc5)c4=O)cn3)c2cc1OC] | 10 | 3 |

N/A = Not available

### Synthetic Examples

Synthesis of Synthetic Intermediates

Preparation 1

### 5-(4-fluorophenyl)-1-methyl-4-oxo-1,4-dihydropyridine-3-carboxylic acid

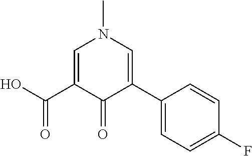 [SMILES: Cn1cc(C(=O)O)c(=O)c(-c2ccc(F)cc2)c1]

Step A: Methyl 5-bromo-4-hydroxynicotinate (100 mg, 0.431 mmol) and Cs₂CO₃ (211 mg, 0.646 mmol) were diluted with DMF (2 mL), placed under nitrogen and heated to 75° C. for 10 min. The reaction mixture was allowed to cool to room temperature. Mel (40.4 ΞL, 0.646 mmol) was added and the reaction mixture was stirred for 3 h. The reaction mixture was diluted with water and extracted with DCM/IPA (3:1) four times. The organic layers were combined, dried over MgSO₄, filtered and concentrated. The residue was purified over silica gel (1-10% methanol in DCM with 1% NI-1₄0H) to afford methyl 5-bromo-1-methyl-4-oxo-1,4-dihydropyridine-3-carboxylate (92 mg, 0.374 mmol, 86.8% yield).

Step B: Methyl 5-bromo-1-methyl-4-oxo-1,4-dihydropyridine-3-carboxylate (92 mg, 0.37 mmol), (4-fluorophenyl)boronic acid (105 mg, 0.75 mmol) and Pd(PPh₃)₄ (22 mg, 0.019 mmol) were combined and diluted with dioxane (1 mL) followed by the addition of Na₂CO₃ (561 pL, 1.1 mmol, 2.0 M). The reaction mixture was purged with argon, sealed and heated to 90° C. overnight. The reaction mixture was allowed to cool to room temperature, diluted with water and the pH was adjusted to 2 with 1N HCI. The mixture was extracted with three times with DCM/IPA (3:1). The organic layers were combined, dried over MgSO₄, filtered and concentrated. The product was triturated with diethyl ether to afford 5-(4-fluorophenyl)-1-methyl-4-oxo-1,4-dihydropyridine-3-carboxylic acid.

Preparation 2

### 5-(4-fluorophenyl)-1-isopropyl-4-oxo-1,4-dihydropyridine-3-carboxylic acid

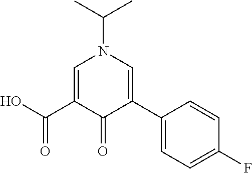 [SMILES: CC(C)n1cc(C(=O)O)c(=O)c(-c2ccc(F)cc2)c1]

Step A: Methyl 5-bromo-4-hydroxynicotinate (1.13 g, 4.87 mmol) and Cs₂CO₃ (1.90 g, 5.84 mmol) were diluted with DMF (20 mL), placed under nitrogen and heated to 75° C. for 10 minutes. The reaction was allowed to cool and 2-bromopropane (0.686 mL, 7.30 mmol) was added. The reaction was heated to 55° C. and stirred for 12 h. The reaction was cooled and diluted with water. The material was extracted with DCM/IPA (3/1) three times. The organic layers were combined, dried over MgSO₄, filtered and concentrated. The residue was purified over silica gel (20-80% ethyl acetate in hexanes) to afford methyl 5-bromo-1-isopropyl-4-oxo-1,4-dihydropyridine-3-carboxylate (1.1 g, 4.01 mmol, 82.4% yield).

Step B: Methyl 5-bromo-1-isopropyl-4-oxo-1,4-dihydropyridine-3-carboxylate (3.5 g, 12.8 mmol) was dissolved in dioxane (65 mL) and (4-fluorophenyl)boronic acid (2.32 g, 16.6 mmol) and 2M Na₂CO₃ (12.8 ml, 25.5 mmol) were added and nitrogen bubbled through for 5 min. Pd(Ph₃P)₄ (0.738 g, 0.638 mmol) was added and the reaction heated to 90° C. overnight. The reaction was cooled and poured into 1N NaOH (50 mL). 50 mL EtOAc added and shaken for 5 min and phases separated. The organic layer was extracted again with 1N NaOH (50 mL). The aqueous layers were combined and acidified with conc. HCI to pH 1. Extracted with EtOAc (x2), dried over Na₂SO₄, filtered and concentrated. The residue was purified over silica gel (100% EtOAc) to afford 5-(4-fluorophenyl)-1-isopropyl-4-oxo-1,4-dihydropyridine-3-carboxylic acid (2.54 g, 9.23 mmol, 72.3% yield) as a white foam.

Following the procedure in Preparation 1, the following synthetic intermediates were also prepared:

| Preparation | Structure | Name |
|---|---|---|
| 3 | 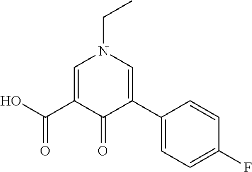 [SMILES: CCn1cc(C(=O)O)c(=O)c(-c2ccc(F)cc2)c1] | 1-ethyl-5-(4-fluorophenyl)-4- oxo-1,4-dihydropyridine-3- carboxylic acid |
| 4 | 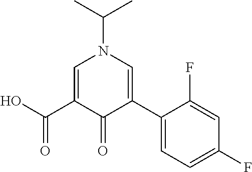 [SMILES: CC(C)n1cc(C(=O)O)c(=O)c(-c2ccc(F)cc2F)c1] | 5-(2,4-difluorophenyl)-1- isopropyl-4-oxo-1,4- dihydropyridine-3-carboxylic acid |
| 5 | 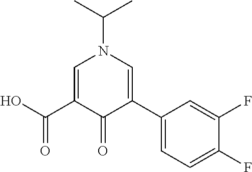 [SMILES: CC(C)n1cc(C(=O)O)c(=O)c(-c2ccc(F)c(F)c2)c1] | 5-(3,4-difluorophenyl)-1- isopropyl-4-oxo-1,4- dihydropyridine-3-carboxylic acid |
| 6 | 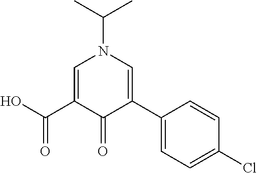 [SMILES: CC(C)n1cc(C(=O)O)c(=O)c(-c2ccc(Cl)cc2)c1] | 5-(4-chlorophenyl)-1-isopropyl- 4-oxo-1,4-dihydropyridine-3- carboxylic acid |
| 7 | 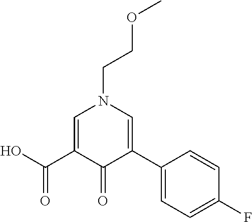 [SMILES: COCCn1cc(C(=O)O)c(=O)c(-c2ccc(F)cc2)c1] | 5-(4-fluorophenyl)-1-(2- methoxyethyl)-4-oxo-1,4- dihydropyridine-3-carboxylic acid |
| 8 | 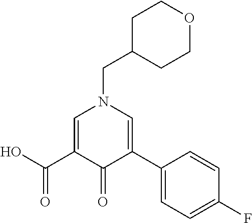 [SMILES: O=C(O)c1cn(CC2CCOCC2)cc(-c2ccc(F)cc2)c1=O] | 5-(4-fluorophenyl)-4-oxo-1- ((tetrahydro-2H-pyran-4- yl)methyl)-1,4-dihydropyridine- 3-carboxylic acid |
| 9 | 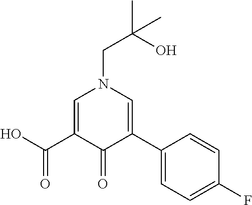 [SMILES: CC(C)(O)Cn1cc(C(=O)O)c(=O)c(-c2ccc(F)cc2)c1] | 5-(4-fluorophenyl)-1-(2- hydroxy-2-methylpropyl)-4- oxo-1,4-dihydropyridine-3- carboxylic acid |
| 10 | 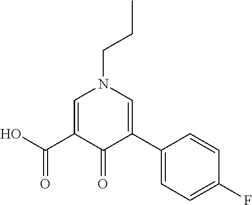 [SMILES: CCCn1cc(C(=O)O)c(=O)c(-c2ccc(F)cc2)c1] | 5-(4-fluorophenyl)-4-oxo-1- propyl-1,4-dihydropyridine-3- carboxylic acid |
| 11 | 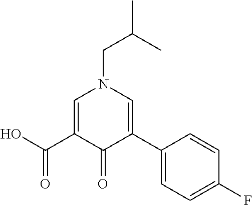 [SMILES: CC(C)Cn1cc(C(=O)O)c(=O)c(-c2ccc(F)cc2)c1] | 5-(4-fluorophenyl)-1-isobutyl-4- oxo-1,4-dihydropyridine-3- carboxylic acid |
| 12 | 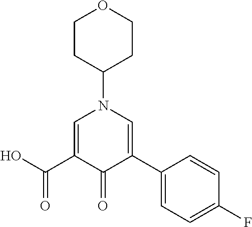 [SMILES: O=C(O)c1cn(C2CCOCC2)cc(-c2ccc(F)cc2)c1=O] | 5-(4-fluorophenyl)-4-oxo-1- (tetrahydro-2H-pyran-4-yl)-1,4- dihydropyridine-3-carboxylic acid |
| 13 | 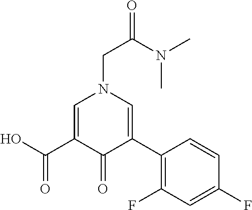 [SMILES: CN(C)C(=O)Cn1cc(C(=O)O)c(=O)c(-c2ccc(F)cc2F)c1] | 5-(2,4-difluorophenyl)-1-(2- (dimethylamino)-2-oxoethyl)-4- oxo-1,4-dihydropyridine-3- carboxylic acid |
| 14 | 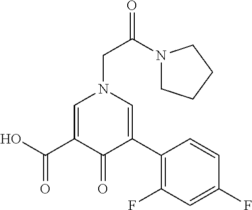 [SMILES: O=C(O)c1cn(CC(=O)N2CCCC2)cc(-c2ccc(F)cc2F)c1=O] | 5-(2,4-difluorophenyl)-4-oxo-1- (2-oxo-2-(pyrrolidin-1-yl)ethyl)- 1,4-dihydropyridine-3- carboxylic acid |
| 15 | 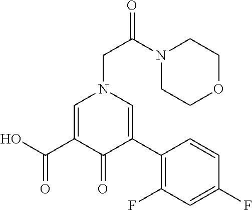 [SMILES: O=C(O)c1cn(CC(=O)N2CCOCC2)cc(-c2ccc(F)cc2F)c1=O] | 5-(2,4-difluorophenyl)-1-(2- morpholino-2-oxoethyl)-4-oxo- 1,4-dihydropyridine-3- carboxylic acid |
| 16 | 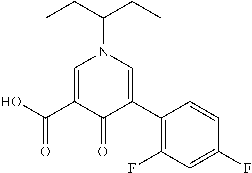 [SMILES: CCC(CC)n1cc(C(=O)O)c(=O)c(-c2ccc(F)cc2F)c1] | 5-(2,4-difluorophenyl)-4-oxo-1- (pentan-3-yl)-1,4- dihydropyridine-3-carboxylic acid |
| 17 | 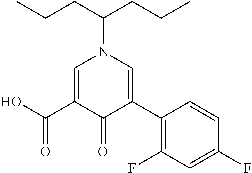 [SMILES: CCCC(CCC)n1cc(C(=O)O)c(=O)c(-c2ccc(F)cc2F)c1] | 5-(2,4-difluorophenyl)-1- (heptan-4-yl)-4-oxo-1,4- dihydropyridine-3-carboxylic acid |
| 18 | 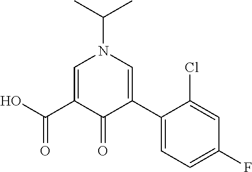 [SMILES: CC(C)n1cc(C(=O)O)c(=O)c(-c2ccc(F)cc2Cl)c1] | 5-(2-chloro-4-fluorophenyl)-1- isopropyl-4-oxo-1,4- dihydropyridine-3-carboxylic acid |
| 19 | 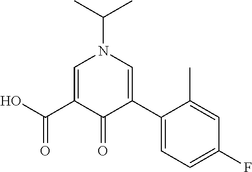 [SMILES: Cc1cc(F)ccc1-c1cn(C(C)C)cc(C(=O)O)c1=O] | 5-(4-fluoro-2-methylphenyl)-1- isopropyl-4-oxo-1,4- dihydropyridine-3-carboxylic acid |
| 20 | 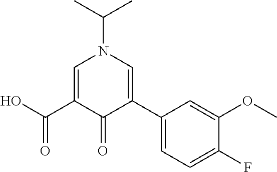 [SMILES: COc1cc(-c2cn(C(C)C)cc(C(=O)O)c2=O)ccc1F] | 5-(4-fluoro-3-methoxyphenyl)- 1-isopropyl-4-oxo-1,4- dihydropyridine-3-carboxylic acid |
| 21 | 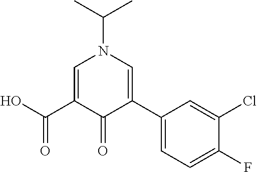 [SMILES: CC(C)n1cc(C(=O)O)c(=O)c(-c2ccc(F)c(Cl)c2)c1] | 5-(3-chloro-4-fluorophenyl)-1- isopropyl-4-oxo-1,4- dihydropyridine-3-carboxylic acid |
| 22 | 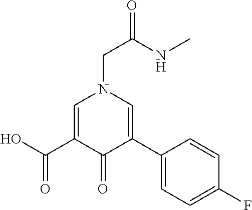 [SMILES: CNC(=O)Cn1cc(C(=O)O)c(=O)c(-c2ccc(F)cc2)c1] | 5-(4-fluorophenyl)-1-(2- (methylamino)-2-oxoethyl)-4- oxo-1,4-dihydropyridine-3- carboxylic acid |
| 23 | 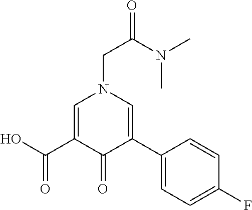 [SMILES: CN(C)C(=O)Cn1cc(C(=O)O)c(=O)c(-c2ccc(F)cc2)c1] | 1-(2-(dimethylamino)-2- oxoethyl)-5-(4-fluorophenyl)-4- oxo-1,4-dihydropyridine-3- carboxylic acid |
| 24 | 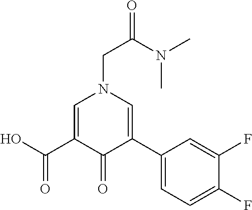 [SMILES: CN(C)C(=O)Cn1cc(C(=O)O)c(=O)c(-c2ccc(F)c(F)c2)c1] | 5-(3,4-difluorophenyl)-1-(2- (dimethylamino)-2-oxoethyl)-4- oxo-1,4-dihydropyridine-3- carboxylic acid |
| 25 | 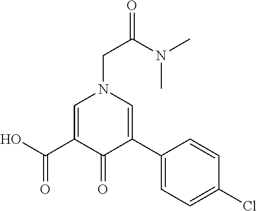 [SMILES: CN(C)C(=O)Cn1cc(C(=O)O)c(=O)c(-c2ccc(Cl)cc2)c1] | 5-(4-chlorophenyl)-1-(2- (dimethylamino)-2-oxoethyl)-4- oxo-1,4-dihydropyridine-3- carboxylic acid |
| 26 | 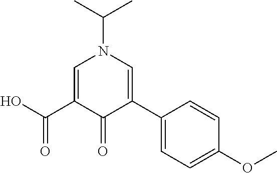 [SMILES: COc1ccc(-c2cn(C(C)C)cc(C(=O)O)c2=O)cc1] | 1-isopropyl-5-(4- methoxyphenyl)-4-oxo-1,4- dihydropyridine-3-carboxylic acid |
| 27 | 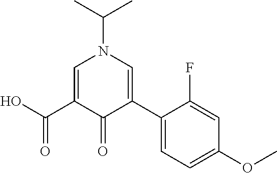 [SMILES: COc1ccc(-c2cn(C(C)C)cc(C(=O)O)c2=O)c(F)c1] | 5-(2-fluoro-4-methoxyphenyl)- 1-isopropyl-4-oxo-1,4- dihydropyridine-3-carboxylic acid |
| 28 | 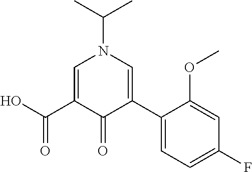 [SMILES: COc1cc(F)ccc1-c1cn(C(C)C)cc(C(=O)O)c1=O] | 5-(4-fluoro-2-methoxyphenyl)- 1-isopropyl-4-oxo-1,4- dihydropyridine-3-carboxylic acid |
| 29 | 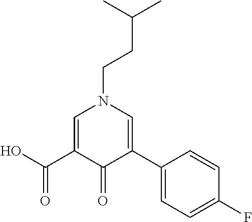 [SMILES: CC(C)CCn1cc(C(=O)O)c(=O)c(-c2ccc(F)cc2)c1] | 5-(4-fluorophenyl)-1-isopentyl- 4-oxo-1,4-dihydropyridine-3- carboxylic acid |
| 30 | 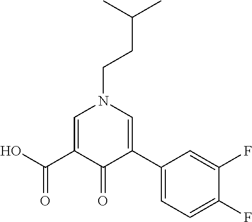 [SMILES: CC(C)CCn1cc(C(=O)O)c(=O)c(-c2ccc(F)c(F)c2)c1] | 5-(3,4-difluorophenyl)-1- isopentyl-4-oxo-1,4- dihydropyridine-3-carboxylic acid |
| 31 | 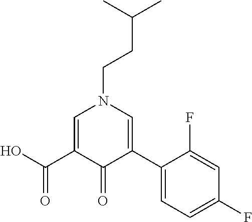 [SMILES: CC(C)CCn1cc(C(=O)O)c(=O)c(-c2ccc(F)cc2F)c1] | 5-(2,4-difluorophenyl)-1- isopentyl-4-oxo-1,4- dihydropyridine-3-carboxylic acid |
| 32 | 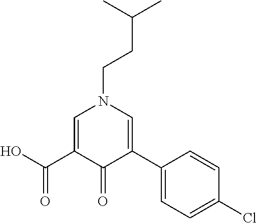 [SMILES: CC(C)CCn1cc(C(=O)O)c(=O)c(-c2ccc(Cl)cc2)c1] | 5-(4-chlorophenyl)-1-isopentyl- 4-oxo-1,4-dihydropyridine-3- carboxylic acid |
| 33 | 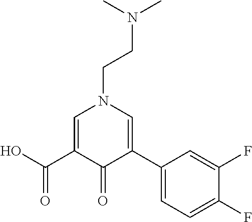 [SMILES: CN(C)CCn1cc(C(=O)O)c(=O)c(-c2ccc(F)c(F)c2)c1] | 5-(3,4-difluorophenyl)-1-(2- (dimethylamino)ethyl)-4-oxo- 1,4-dihydropyridine-3- carboxylic acid |
| 34 | 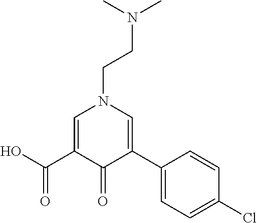 [SMILES: CN(C)CCn1cc(C(=O)O)c(=O)c(-c2ccc(Cl)cc2)c1] | 5-(4-chlorophenyl)-1-(2- (dimethylamino)ethyl)-4-oxo- 1,4-dihydropyridine-3- carboxylic acid |
| 35 | 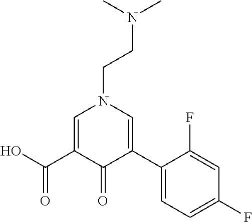 [SMILES: CN(C)CCn1cc(C(=O)O)c(=O)c(-c2ccc(F)cc2F)c1] | 5-(2,4-difluorophenyl)-1-(2- (dimethylamino)ethyl)-4-oxo- 1,4-dihydropyridine-3- carboxylic acid |

Preparation 36

### 5-(4-fluorophenyl)-4-oxo-1,4-dihydropyridine-3-carboxylic acid

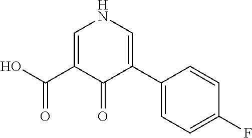 [SMILES: O=C(O)c1c[nH]cc(-c2ccc(F)cc2)c1=O]

Methyl 5-bromo-4-oxo-1,4-dihydropyridine-3-carboxylate (200 mg, 0.862 mmol), (4-fluorophenyl)boronic acid (193 mg, 1.38 mmol) and Pd(PPh₃)₄ (29.9 mg, 0.0259 mmol) were diluted with dioxane (2 mL) followed by the addition of 2.0 M Na₂CO₃ (1077 ΞL, 2.15 mmol). The reaction mixture was purged with argon, sealed and heated to 90° C. overnight. The reaction mixture was allowed to cool, diluted with DCM and water. The pH of the water was adjusted with 1N HCI to a pH of 2. The layers were separated, and the water layer was extracted again with DCM. The organic layers were combined, dried over MgSO₄, filtered and concentrated to afford 5-(4-fluorophenyl)-4-oxo-1,4-dihydropyridine-3-carboxylic acid (150 mg, 0.643 mmol, 74.6 yield).

Preparation 37

### 5-(4-fluorophenyl)-1-isopropyl-6-methyl-4-oxo-1,4-dihydropyridine-3-carboxylic acid

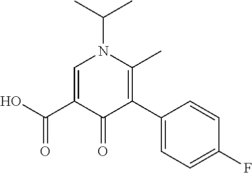 [SMILES: Cc1c(-c2ccc(F)cc2)c(=O)c(C(=O)O)cn1C(C)C]

Step A: To a solution of 4-hydroxy-6-methyl-2-pyrone (2.0 g, 15.9 mmol) in toluene (5.29 mL, 15.9 mmol) was added 1,1-dimethoxy-N,N-dimethylmethanamine (2.31 mL, 17.4 mmol) and the reaction mixture was stirred at room temperature for 40 h. The reaction mixture was concentrated, toluene added and the reaction mixture was concentrated to afford crude 3-((dimethylamino)methylene)-6-methyl-2H-pyran-2,4(3H)-dione (3.06 g, 16.9 mmol, 106% yield), which was used in the next step without further purification.

Step B: To a solution of 3-((dimethylamino)methylene)-6-methyl-2H-pyran-2,4(3H)-dione (3.06 g, 16.9 mmol) in EtOH (16.9 mL, 16.9 mmol) was added sodium tert-butoxide (2.43 g, 25.3 mmol) and then propan-2-amine (2.07 mL, 25.3 mmol). The mixture was heated to 90° C. for 18 hours and then concentrated. The residue was partitioned between water (50 mL) and DCM (50 mL). The aqueous phase was extracted with DCM (2×25 mL). The pH of the aqueous phase was adjusted to 2 with 2M HCI and the organic phase was extracted with DCM (3×25 mL). The organic layers were combined and washed with 1N NaOH (25 mL) and brine (10 mL), dried over Na₂SO₄, filtered and concentrated in vacuo to afford crude 1-isopropyl-6-methyl-4-oxo-1,4-dihydropyridine-3-carboxylic acid (1.08 g, 5.53 mmol, 32.8% yield) as a red solid.

Step C: To a solution of 1-isopropyl-6-methyl-4-oxo-1,4-dihydropyridine-3-carboxylic acid (1.08 g, 5.53 mmol) in DCE (27.7 mL, 5.53 mmol) was added N-bromosuccinimide (1.48 g, 8.30 mmol) and the reaction mixture was stirred overnight. The reaction was not complete, so an additional 1.5 eq. of NBS was added and the reaction mixture was allowed to stir for 1 h. DCM (50 mL) and 1 N NaOH (25 mL) were added and the organic phase was separated. The organic phase was extracted with 1N NaOH (2 x 15 mL) and then the aqueous layers were combined and extracted with DCM (25 mL). The aqueous phase was acidified with 2N HCI to a pH 2 and the organic phase was extracted with EtOAc (3×25 mL). The organic layers were combined, washed with brine (25 mL), dried over Na₂SO₄, filtered and concentrated in vacuo. The resulting solid was purified over silica gel (1-10% MeOH in DCM) to afford 5-bromo-1-isopropyl-6-methyl-4-oxo-1,4-dihydropyridine-3-carboxylic acid (0.250 g, 0.912 mmol, 16.5 yield) as a slightly red solid.

Step D: 5-Bromo-1-isopropyl-6-methyl-4-oxo-1,4-dihydropyridine-3-carboxylic acid (75 mg, 0.27 mmol), (4-fluorophenyl)boronic acid (77 mg, 0.55 mmol) and Pd(PPh₃)₄ (16 mg, 0.014 mmol) were diluted with 1,4-dioxane (1368 μl, 0.27 mmol), followed by the addition of 2.0 M Na₂CO₃ (410 μL, 0.82 mmol). The reaction mixture was purged with argon, sealed and heated to 90° C. overnight. The reaction mixture was partitioned between DCM and water. The pH of the aqueous phase was adjusted with 1N NaOH to a pH of 12. The layers were separated and the aqueous layer was extracted again with DCM (2×15 mL). EtOAc was added to the aqueous phase, and then the pH was adjusted with 1N HCI to a pH of 2 (a white solid crashed out of the aqueous phase). The layers were separated and the organic layer was extracted with EtOAc (2×15 mL). The organic layers were combined, dried over Na₂SO₄, filtered and concentrated to afford crude 5-(4-fluorophenyl)-1-isopropyl-6-methyl-4-oxo-1,4-dihydropyridine-3-carboxylic acid (0.063 g, 0.19 mmol, 70% yield).

Following the procedure in preparation 37, the following compounds were also synthesized.

| Preparation | Structure | Name |
|---|---|---|
| 38 | 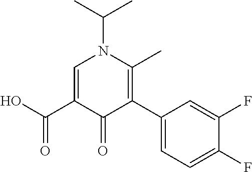 [SMILES: Cc1c(-c2ccc(F)c(F)c2)c(=O)c(C(=O)O)cn1C(C)C] | 5-(3,4-difluorophenyl)-1- isopropyl-6-methyl-4-oxo-1,4- dihydropyridine-3-carboxylic acid |
| 39 | 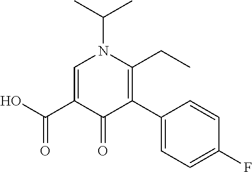 [SMILES: CCc1c(-c2ccc(F)cc2)c(=O)c(C(=O)O)cn1C(C)C] | 6-ethyl-5-(4-fluorophenyl)-1- isopropyl-4-oxo-1,4- dihydropyridine-3-carboxylic acid |
| 40 | 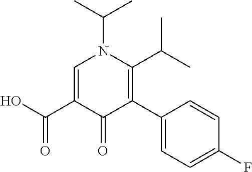 [SMILES: CC(C)c1c(-c2ccc(F)cc2)c(=O)c(C(=O)O)cn1C(C)C] | 5-(4-fluorophenyl)-1,6- diisopropyl-4-oxo-1,4- dihydropyridine-3-carboxylic acid |
| 41 | 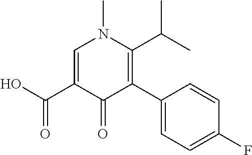 [SMILES: CC(C)c1c(-c2ccc(F)cc2)c(=O)c(C(=O)O)cn1C] | 5-(4-fluorophenyl)-6- isopropyl-1-methyl-4-oxo-1,4- dihydropyridine-3-carboxylic acid |

Preparation 42

### 5-((6-methoxyquinolin-4-yl)oxy)pyridin-2-amine

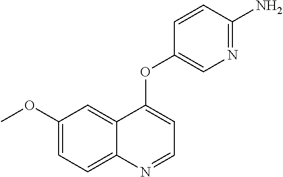 [SMILES: COc1ccc2nccc(Oc3ccc(N)nc3)c2c1]

Step A: 4-Chloro-6-methoxyquinoline (150 mg, 0.775 mmol) was diluted with chlorobenzene followed by the addition of 2-bromo-5-hydroxypyridine (148 mg, 0.852 mmol) and DMAP (9.46 mg, 0.0775 mmol). The reaction mixture was placed under nitrogen and heated to 110° C. After stirring for 12 hours, the reaction mixture was allowed to cool to room temperature, then diluted with ethyl acetate and water. Some material crashed out of solution and was removed by filtration. The organic layer was isolated, dried over MgSO₄, filtered and concentrated. The solids that crashed out of solution and the material from the concentrated organic layer were combined and purified on silica gel (2-8% methanol/DCM) to afford 4-((6-bromopyridin-3-yl)oxy)-6-methoxyquinoline (150 mg, 0.453 mmol, 58.5% yield).

Step B: 4-((6-Bromopyridin-3-yl)oxy)-6-methoxyquinoline (150 mg, 0.453 mmol) was diluted with THF (2 mL) followed by the addition of tris(dibenzylideneacetone)dipalladium(0) chloroform adduct (23.4 mg, 0.0226 mmol), 2-(dicyclohexylphosphino)biphenyl (15.9 mg, 0.0453 mmol) and 1M LiHMDS in THF (1132 μL, 1.13 mmol). The reaction mixture was purged with argon, sealed and heated to 80° C. for 12 h. The reaction mixture was allowed to cool to room temperature and then poured into water, extracted with twice with DCM, dried over MgSO₄, filtered and concentrated. The residue was purified on silica gel (1-10% methanol/DCM) to afford 5-((6-methoxyquinolin-4-yl)oxy)pyridin-2-amine (90 mg, 0.337 mmol, 74.3% yield).

Following the procedure in Preparation 42, the following compounds were also synthesized:

| Preparation | Structure | Name |
|---|---|---|
| 43 | 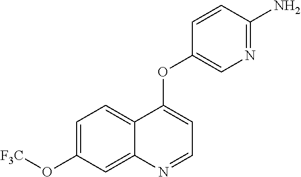 [SMILES: Nc1ccc(Oc2ccnc3cc(OC(F)(F)F)ccc23)cn1] | 4-((6-bromopyridin-3-yl)oxy)- 7-(trifluoromethoxy)quinoline |
| 44 | 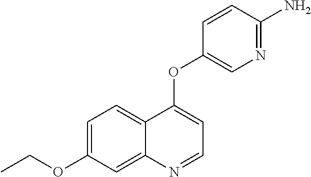 [SMILES: CCOc1ccc2c(Oc3ccc(N)nc3)ccnc2c1] | 5-((7-ethoxyquinolin-4- yl)oxy)pyridin-2-amine |
| 45 | 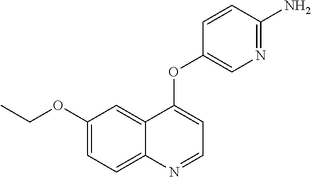 [SMILES: CCOc1ccc2nccc(Oc3ccc(N)nc3)c2c1] | 5-((6-ethoxyquinolin-4- yl)oxy)pyridin-2-amine |
| 46 | 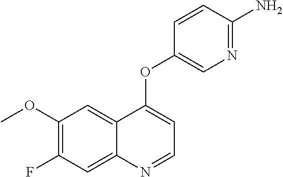 [SMILES: COc1cc2c(Oc3ccc(N)nc3)ccnc2cc1F] | 5-((7-fluoro-6- methoxyquinolin-4- yl)oxy)pyridin-2-amine |

Preparation 47

### 5-((6,7-dimethoxyquinolin-4-yl)oxy)pyrimidin-2-amine

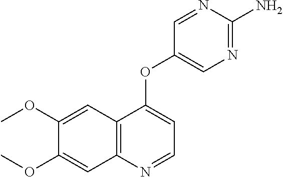 [SMILES: COc1cc2nccc(Oc3cnc(N)nc3)c2cc1OC]

Step A: A mixture of 4-chloro-6,7-dimethoxyquinoline (0.250 g, 1.12 mmol), 2-(5-hydroxy-2-pyrimidinyl)imidodicarbonic acid 1,3-bis(1,1-dimethylethyl) ester (0.383 g, 1.23 mmol) and DMAP (0.0068 g, 0.056 mmol) was suspended in chlorobenzene (2.8 mL, 1.12 mmol), and the reaction mixture was heated to 100° C. overnight. The reaction mixture was cooled to room temperature, diluted with water (25 mL) and DCM (25 mL) and then filtered. The organic layer was separated, and the aqueous layer was extracted with DCM (2×15 mL). The combined organic layers were washed with brine (15 mL), dried over Na₂SO₄, filtered and concentrated in vacuo to obtain imidodicarbonic acid,2-[5-(6,7-dimethoxyquinolin-4-yl)oxy)-2-pyrimidinyl]-1,3-bis(1,1-dimethylethyl)ester (0.557 g, 1.12 mmol, 100.0% yield).

Step B: Imidodicarbonic acid,2-[5-(6,7-dimethoxyquinolin-4-yl)oxy)-2-pyrimiodinyl]-1,3-bis(1,1-dimethylethyl)ester (557 mg, 1.12 mmol) was dissolved in DCM (2.8 mL) TFA (2.8 mL) was added and the reaction stirred at room temperature for 1 h. The reaction was concentrated and partitioned between EtOAc and aqueous NaHCO₃. The organic layer was concentrated and the residue was purified over silica gel to afford 5-((6,7-dimethoxyquinolin-4-yl)oxy)pyrimidin-2-amine (130 mg, 39% yield).

Preparation 48

### 5-((6-methoxy-7-(3-morpholinopropoxy)quinolin-4-yl)oxy)pyridin-2-amine

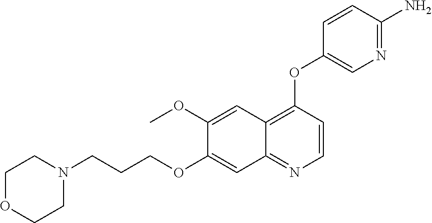 [SMILES: COc1cc2c(Oc3ccc(N)nc3)ccnc2cc1OCCCN1CCOCC1]

Step A: To a mixture of 4-(3-((4-chloro-6-methoxyquinolin-7-yl)oxy)propyl)morpholine (0.509 g, 1.50 mmol), 2-bromo-5-hydroxypyridine (0.288 g, 1.65 mmol) and DMAP (0.00918 g, 0.0751 mmol) was added chlorobenzene (3.7 mL) and the reaction mixture was heated at 110° C. for 3 days. The mixture reaction was cooled to room temperature, diluted with water (50 mL) and extracted with DCM (3×50 mL). The organic layers were combined, washed with brine (15 mL), dried over Na₂SO₄, filtered and concentrated in vacuo to obtain crude 4-(3-(4-(6-bromopyridin-3-yl)oxy)-6-methoxyquinolin-7-yl)oxy)propyl)morpholine (0.350 g, 0.738 mmol, 49.1% yield) as a slightly yellow oil.

Step B: To a solution of 4-(3-((4-((6-bromopyridin-3-yl)oxy)-6-methoxyquinolin-7-yl)oxy)propyl)morpholine (0.300 g, 0.632 mmol) in THF (1.2 mL) was added 1M LHMDS (1.90 mL, 1.90 mmol) in THF, followed by tris(dibenzylideneacetone)dipalladium(0) (0.0290 g, 0.0316 mmol) and 2-(dicyclohexylphosphino)biphenyl (0.0222 g, 0.0632 mmol) under an Ar atmosphere. The reaction mixture was heated to 80° C. for 2.5 h. The reaction mixture was cooled to room temperature and quenched with 2M HCI (2 mL) and stirred for 1 hour. Solid Na₂CO₃ was added slowly to adjust the pH to >8. The mixture was filtered and extracted with DCM (3×50 mL). The combined organic layers were concentrated and the residue was purified over silica gel (1-10% MeOH in DCM) to afford 5-((6-methoxy-7-(3-morpholinopropoxy)quinolin-4-yhoxy)pyridin-2-amine (0.106 g, 0.258 mmol, 40.8% yield).

Preparation 49

### 4-((6-aminopyridin-3-yl)oxy)-6,7-dimethoxyquinoline-3-carbonitrile

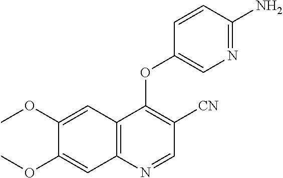 [SMILES: COc1cc2ncc(C#N)c(Oc3ccc(N)nc3)c2cc1OC]

Step A: To a mixture of 4-chloro-6,7-dimethoxyquinoline-3-carbonitrile (0.500 g, 2.01 mmol), 2-bromo-5-hydroxypyridine (0.385 g, 2.21 mmol) and DMAP (0.0123 g, 0.101 mmol) was added chlorobenzene (4.0 mL). The reaction mixture was heated to 110° C. overnight. The reaction mixture was cooled and 50 mL cold water was added. The reaction mixture was filtered, and the isolated solids were slurried in water (50 mL) and the pH was adjusted to 12 with 1N NaOH. The mixture was stirred at for 15 min, filtered and dried to afford 4-((6-bromopyridin-3-yl)oxy)-6,7-dimethoxyquinoline-3-carbonitrile (0.130 g, 0.337 mmol, 16.7% yield).

Step B: A mixture of 4-((6-bromopyridin-3-yl)oxy)-6,7-dimethoxyquinoline-3-carbonitrile (0.088 g, 0.23 mmol), tert-butyl carbamate (0.080 g, 0.68 mmol), Pd₂(dba)₃ (0.021 g, 0.023 mmol), XPHOS (0.022 g, 0.046 mmol) and Cs₂CO₃ (0.15 g, 0.46 mmol) in dioxane (1.1 mL)was heated to 90° C. for 1 h. The reaction mixture was filtered and the solids were washed with DCM (50 mL). The combined filtrate was reduced in vacuo and the resulting oil was suspended in 5 mL DCM. TFA (5 mL) was added and the mixture was stirred for 1 h. The mixture was reduced in vacuo and then suspended in 25 mL DCM, washed with saturated NaHCO₃ (3 x 50 mL), brine (25 mL). The organic layer was dried over Na₂SO₄, filtered and concentrated in vacuo. The residue was purified over silica gel (1-10% MeOH in DCM) to afford 4-((6-aminopyridin-3-yl)oxy)-6,7-dimethoxyquinoline-3-carbonitrile (0.032 g, 0.099 mmol, 44% yield) as a white solid.

Preparation 50

### 6-((6,7-dimethoxyquinolin-4-yl)oxy)pyridin-3-amine

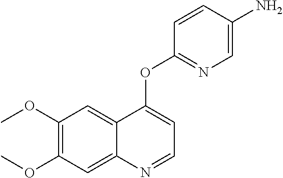 [SMILES: COc1cc2nccc(Oc3ccc(N)cn3)c2cc1OC]

Step A: 2-Chloro-5-nitropyridine (680 mg, 4.29 mmol), 6,7-dimethoxyquinolin-4-ol (800 mg, 3.90 mmol) and Cs₂CO₃ (1397 mg, 4.29 mmol) were diluted with ACN (10 mL). The reaction was placed under nitrogen and stirred for 14 hours. The reaction was filtered and rinsed with minimal acetonitrile. The filtrate was concentrated and the residue was purified on silica gel (30-100% ethyl acetate in hexanes) to afford 6,7-dimethoxy-4-((5-nitropyridin-2-yl)oxy)quinoline (300 mg, 0.917 mmol, 23.5% yield).

Step B: 6,7-Dimethoxy-4-((5-nitropyridin-2-yl)oxy)quinoline (46 mg, 0.14 mmol) was diluted with THF (1 mL) followed by the addition of zinc (46 mg, 0.70 mmol). Saturated ammonium chloride (500 μL) was added and the reaction mixture was stirred for 1 hour. The reaction mixture was diluted with ethyl acetate and saturated sodium carbonate. The layers were separated and the organic layer was dried over MgSO4, filtered and concentrated to afford 6-((6,7-dimethoxyquinolin-4-yl)oxy)pyridin-3-amine (30 mg, 0.10 mmol, 72% yield).

Preparation 51

### 5-((6,7-dimethoxy-2-methylquinolin-4-yl)oxy)pyridin-2-amine

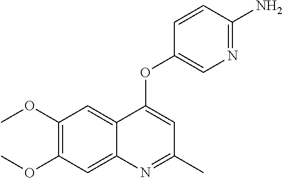 [SMILES: COc1cc2nc(C)cc(Oc3ccc(N)nc3)c2cc1OC]

Step A: A mixture of 2,4-Dichloro-6,7-dimethoxyquinoline (0.500 g, 1.94 mmol), methylboronic acid (0.116 g, 1.94 mmol) and Pd(PPh₃)₄ (0.112 g, 0.0969 mmol) was diluted with dioxane (9.7 mL), followed by the addition of 2M Na₂CO₃ (2.91 mL, 5.81 mmol). The vessel was sealed and the reaction mixture was heated to 100° C. overnight. Additional methylboronic acid (1 equiv) was added and the reaction mixture was allowed to stir over a second night at 100° C. The reaction mixture was allowed to cool and added to cold water (50 mL). The resulting solid was suspended in DCM (50 mL), filtered and concentrated in vacuo. The crude product was purified over silica gel (0-100% EtOAc in hexanes) to afford 4-chloro-6,7-dimethoxy-2-methylquinoline (0.178 g, 0.749 mmol, 38.7% yield).

Step B: A mixture of 4-chloro-6,7-dimethoxy-2-methylquinoline (0.144 g, 0.606 mmol), 2-bromo-5-hydroxypyridine (0.116 g, 0.666 mmol) and DMAP (0.00370 g, 0.0303 mmol) in chlorobenzene (1.2 mL), was heated to 110° C. overnight. The mixture was cooled and partitioned between water (50 mL) and DCM (3×50 mL). The organic layers were combined, washed with brine (15 mL), dried over Na₂SO₄, filtered and concentrated in vacuo to obtain crude 4-((6-bromopyridin-3-yl)oxy)-6,7-dimethoxy-2-methylquinoline (0.220 g, 0.586 mmol, 96.8 yield).

Step C: To a solution of 4-((6-bromopyridin-3-yl)oxy)-6,7-dimethoxy-2-methylquinoline (0.220 g, 0.586 mmol) in THF (1.2 mL) was added 1M LHMDS (1.76 mL, 1.76 mmol) in THF, followed by tris(dibenzylideneacetone)dipalladium (0) (0.0268 g, 0.0293 mmol) and 2-(dicyclohexylphosphino)biphenyl (0.0205 g, 0.0586 mmol) under Ar. The reaction mixture was heated to 80° C. for 1 h. The reaction mixture was cooled to room temperature and 2M HCI (10 mL) was added and the reaction mixture was stirred for 1 h. The reaction mixture was neutralized with slow addition of solid Na₂CO₃ and then filtered. The filtrate was washed with DCM (3×50 mL). The combined organic layers were concentrated and the residue was purified over silica gel (1-10% MeOH in DCM) to afford 5-((6,7-dimethoxy-2-methylquinolin-4-yl)oxy)pyridin-2-amine (0.102 g, 0.328 mmol, 55.9% yield).

Preparation of Synthetic Examples

### Example 1

### N-(5-(6,7-dimethoxyquinolin-4-yl)oxy)pyridin-2-yl)-5-(4-fluorophenyl)-1-isopropyl-4-oxo-1,4-dihydropyridine-3-carboxamide

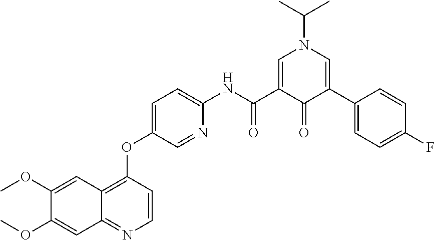 [SMILES: COc1cc2nccc(Oc3ccc(NC(=O)c4cn(C(C)C)cc(-c5ccc(F)cc5)c4=O)nc3)c2cc1OC]

5-(4-Fluoropheny0-1-isopropyl-4-oxo-1,4-dihydropyridine-3-carboxylic acid (Preparation 2; 25 mg, 0.092 mmol), HATU (45 mg, 0.12 mmol) and 5-[(6,7-Dimethoxy-4-quinolinyl)oxy]-2-pyridinamine (APAC Pharmaceutical, Cat. # 663886; 25 mg, 0.084 mmol) were diluted with DMF (500 μL) followed by the addition of DIEA (44 ΞL, 0.25 mmol). After stirring for 12 hours, the reaction mixture was diluted with water and extracted twice with ethyl acetate. The organic layers were combined, washed with water and brine, dried over MgSO₄, filtered and concentrated. The residue was purified over silica gel eluting with 10% methanol/DCM (1% NH₄OH) to afford N-(5-(6,7-dimethoxyquinolin-4-yl)oxy)pyridin-2-yl)-5-(4-fluorophenyl)-1-isopropyl-4-oxo-1,4-dihydropyridine-3-carboxamide (20 mg, 0.036 mmol, 43% yield). Mass spectrum: m/z=555.2 (M+H). ¹H NMR (CDCl₃) δ 13.33 (s, 1H), 8.73 (d, 1H), 8.68 (d, 1H), 8.45 (dd, 1H), 8.31 (dd, 1H), 7.73 (s, 1H), 7.65-7.54 (m, 5H), 7.14 (m, 2H), 6.54 (d, 1H), 4.33 (m, 1H), 4.06 (s, 3H), 4.05 (s, 3H), 1.62 (d, 6H).

### Example 2

### N-(5-(6,7-dimethoxyquinolin-4-yl)oxy)pyridin-2-yl)-5-(4-fluorophenyl)-1-methyl-4-oxo-1,4-dihydropyridine-3-carboxamide

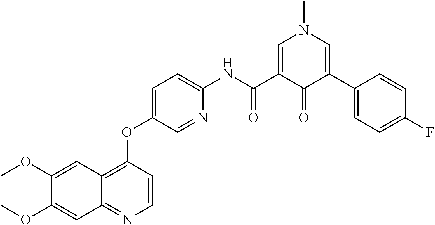 [SMILES: COc1cc2nccc(Oc3ccc(NC(=O)c4cn(C)cc(-c5ccc(F)cc5)c4=O)nc3)c2cc1OC]

5-[(6,7-Dimethoxy-4-quinolinyl)oxy]-2-pyridinamine (25 mg, 0.084 mmol), HATU (45 mg, 0.12 mmol) and 5-(4-fluorophenyl)-1-methyl-4-oxo-1,4-dihydropyridine-3-carboxylic acid (Preparation 1; 23 mg, 0.092 mmol) were combined and diluted with DMF (500 μL) followed by the addition of DIEA (44 ΞL, 0.25 mmol). After stirring for 12 h, the reaction mixture was diluted with water and extracted twice with ethyl acetate. The organic layers were combined, and then washed with water and brine. The organic layer was dried over MgSO₄, filtered and concentrated. The residue was purified over silica gel (10% methanol/DCM (1% NH₄OH)) to afford N-(5-((6,7-dimethoxyquinolin-4-y0oxy)pyridin-2-yl)-5-(4-fluorophenyl)-1-methyl-4-oxo-1,4-dihydropyridine-3-carboxamide (32 mg, 0.061 mmol, 72% yield). Mass spectrum: m/z=527.2 (M+H). ¹H NMR (CDCl₃) δ 13.23 (s, 1H), 8.57 (d, 1H), 8.51 (d, 1H), 8.43 (dd, 1H), 8.30 (dd, 1H), 7.62 (m, 2H), 7.58-7.53 (m, 2H), 7.50 (d, 1H), 7.45 (s, 1H), 7.13 (m, 2H), 6.49 (d, 1H), 4.06 (s, 6H), 3.90 (s, 3H)

### Example 3

### N-(5(6,7-dimethoxyquinolin-4-yl)oxy)pyridin-2-yl)-1-ethyl-5-(4-fluorophenyl)-4-oxo-1,4-dihydropyridine-3-carboxamide

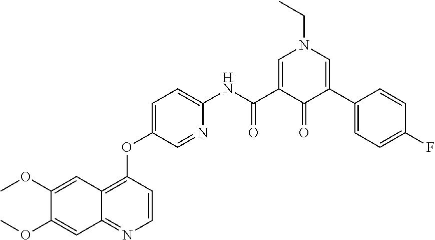 [SMILES: CCn1cc(C(=O)Nc2ccc(Oc3ccnc4cc(OC)c(OC)cc34)cn2)c(=O)c(-c2ccc(F)cc2)c1]

5-[(6,7-Dimethoxy-4-quinolinyl)oxy]-2-pyridinamine (16 mg, 0.054 mmol), 1-ethyl-5-(4-fluorophenyl)-4-oxo-1,4-dihydropyridine-3-carboxylic acid (Preparation 3; 15 mg, 0.059 mmol) and HATU (29 mg, 0.075 mmol) were combined and diluted with DMF (500 μL) followed by the addition of DIEA (28 ΞL, 0.16 mmol). After stirring for 3 h, the reaction mixture was diluted with ethyl acetate and washed with water and brine. The organic layer was dried over MgSO₄, filtered and concentrated. The resulting crude material was purified over silica gel (10% methanol/DCM (1% NH₄OH)) to afford N-(5(6,7-dimethoxyquinolin-4-yl)oxy)pyridin-2-yl)-1-ethyl-5-(4-fluorophenyl)-4-oxo-1,4-dihydropyridine-3-carboxamide (16 mg, 0.030 mmol, 55 yield). Mass spectrum: m/z=541.2 (M+H). ¹H NMR (CDCl₃) δ 13.27 (s, 1H), 8.62 (d, 1H), 8.53 (d, 1H), 8.43 (dd, 1H), 8.31 (dd, 1H), 7.63 (m, 2H), 7.57-7.52 (m, 3H), 7.46 (s, 1H), 7.14 (m, 2H), 6.48 (d, 1H), 4.09 (q, 2H), 4.06 (s, 2H), 4.05 (s, 2H), 1.60 (t, 3H).

### Example 4

### 5-(2,4-difluorophenyl)-N-(5(6,7-dimethoxyquinolin-4-yl)oxy)pyridin-2-yl)-1-isopropyl-4-oxo-1,4-dihydropyridine-3-carboxamide

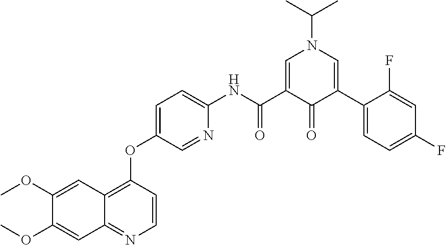 [SMILES: COc1cc2nccc(Oc3ccc(NC(=O)c4cn(C(C)C)cc(-c5ccc(F)cc5F)c4=O)nc3)c2cc1OC]

5-(2,4-DifluorophenyI)-1-isopropyl-4-oxo-1,4-dihydropyridine-3-carboxylic acid (Preparation 4; 27 mg, 0.092 mmol), HATU (45 mg, 0.12 mmol) and 5-[(6,7-Dimethoxy-4-quinolinyl)oxy]-2-pyridinamine (25 mg, 0.084 mmol) were diluted with DMF (500 μL) followed by the addition of DIEA (44 ΞL, 0.25 mmol). After stirring for 12 h, the reaction mixture was diluted with water and extracted twice with ethyl acetate. The organic layers were combined, washed with water and brine. The combined organic layers were dried over MgSO₄, filtered and concentrated. The resulting crude material was purified over silica gel (10% methanol/DCM (1% NH₄OH)) to afford 5-(2,4-difluorophenyl)-N-(5-((6,7-dimethoxyquinolin-4-yl)oxy)pyridin-2-yl)-1-isopropyl-4-oxo-1,4-dihydropyridine-3-carboxamide (22 mg, 0.038 mmol, 46% yield). Mass spectrum: m/z=573.2 (M+H). ¹H NMR (CDCl₃) δ 13.18 (s, 1H), 8.70 (d, 1H), 8.52 (d, 1H), 8.43 (dd, 1H), 8.30 (dd, 1H), 7.71-7.64 (m, 2H), 7.57-7.53 (m, 2H), 7.45 (s, 1H), 7.01-6.90 (m, 2H), 6.48 (d, 1H), 4.34 (m, 1H), 4.06 (s, 6H), 1.61 (d, 6H).

Following the procedures above, the compounds of Examples 5-53 were also synthesized.

### Example 5

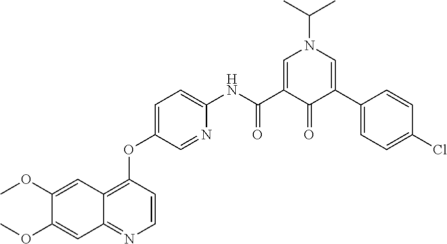 [SMILES: COc1cc2nccc(Oc3ccc(NC(=O)c4cn(C(C)C)cc(-c5ccc(Cl)cc5)c4=O)nc3)c2cc1OC]

### 5-(4-chlorophenyl)-N-(5-((6,7-dimethoxyquinolin-4-yl)oxy)pyridin-2-yl)-1-isopropyl-4-oxo-1,4-dihydropyridine-3-carboxamide

Mass spectrum: m/z=571.2 (M+H). ¹H NMR (CDCl₃) δ 13.27 (s, 1H), 8.68 (d, J=2.5 Hz, 1H), 8.52 (d, J=5.3 Hz, 1H), 8.44 (dd, J=9.0, 0.6 Hz, 1H), 8.31 (dd, J=2.9, 0.6 Hz, 1H), 7.62-7.53 (m, 5H), 7.46-7.40 (m, 3H), 6.48 (d, J=5.3 Hz, 1H), 4.33 (m, 1H), 4.06 (s, 3H), 4.05 (s, 3H), 1.62 (d, J=6.8 Hz, 6H).

### Example 6

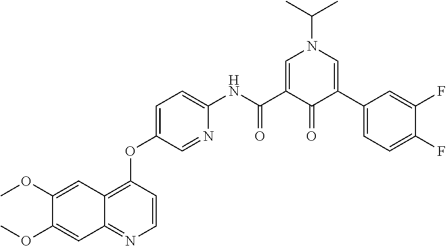 [SMILES: COc1cc2nccc(Oc3ccc(NC(=O)c4cn(C(C)C)cc(-c5ccc(F)c(F)c5)c4=O)nc3)c2cc1OC]

5-(3,4-difluorophenyl)-N-(5(6,7-dimethoxyquinolin-4-yl)oxy)pyridin-2-yl)-1-isopropyl-4-oxo-1,4-dihydropyridine-3-carboxamide

Mass spectrum: m/z=573.2 (M+H). ¹H NMR (CDCl₃) δ 13.24 (s, 1H), 8.69 (d, J=2.5 Hz, 1H), 8.52 (d, J=5.5 Hz, 1H), 8.44 (dd, J=9.0, 0.6 Hz, 1H), 8.32 (dd, J=2.9, 0.6 Hz, 1H), 7.62-7.54 (m, 3H), 7.46 (s, 1H), 7.35 (m, 1H), 7.22, (m, 1H), 6.49 (d, J=5.3 Hz, 1H), 4.34 (m, 1H), 4.06 (s, 6H), 1.62 (d, J=6.8 Hz, 6H).

### Example 7

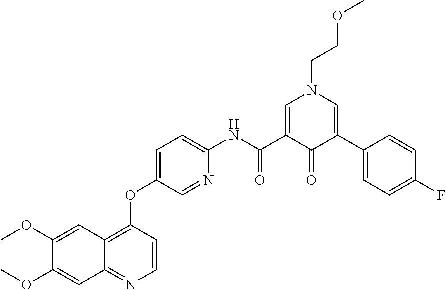 [SMILES: COCCn1cc(C(=O)Nc2ccc(Oc3ccnc4cc(OC)c(OC)cc34)cn2)c(=O)c(-c2ccc(F)cc2)c1]

N-(5((6,7-dimethoxyquinolin-4-yl)oxy)pyridin-2-yl)-5-(4-fluorophenyl)-1-(2-methoxyethyl)-4-oxo-1,4-dihydropyridine-3-carboxamide

Mass spectrum: m/z=571.2 (M+H). ¹H NMR (CDCl₃) δ 13.26 (s, 1H), 8.61 (d, J=2.3 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.66-7.53 (m, 5H), 7.43 (s, 1H), 7.13 (m, 2H), 6.47 (d, J=5.3 Hz, 1H), 4.14 (t, J=4.7 Hz, 2H), 4.06 (s, 3H), 4.05 (s, 3H), 3.76 (t, J=4.7 Hz, 2H), 3.40 (s, 3H).

### Example 8

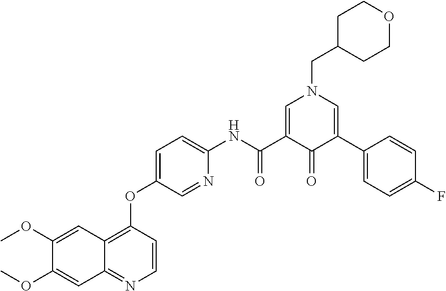 [SMILES: COc1cc2nccc(Oc3ccc(NC(=O)c4cn(CC5CCOCC5)cc(-c5ccc(F)cc5)c4=O)nc3)c2cc1OC]

### N-(5-((6,7-dimethoxyquinolin-4-yl)oxy)pyridin-2-yl)-5-(4-fluorophenyl)-4-oxo-1-((tetrahydro-2H-pyran-4-yl)methyl)-1,4-dihydropyridine-3-carboxamide

Mass spectrum: m/z=611.3 (M+H). ¹H NMR (CDCl₃) δ 13.21 (s, 1H), 8.55 (d, J=2.5 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.65-7.60 (m, 2H), 7.58-7.53 (m, 2H), 7.48 (d, J=2.3 Hz, 1H), 7.43 (s, 1H), 7.14 (m, 2H), 6.47 (d, J=5.3 Hz, 1H), 4.06 (s, 6H), 4.03 (m, 2H), 3.88 (d, J=7.2 Hz, 2H), 3.40 (td, J=11.7, 2.0 Hz, 2H), 2.12 (m, 1H), 1.62 (m, 2H), 1.51-1.40 (m, 2H).

### Example 9

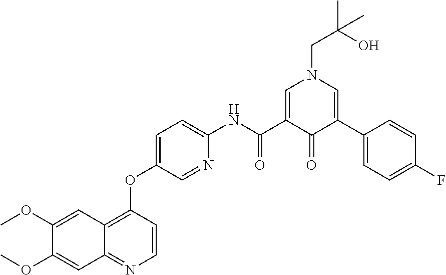 [SMILES: COc1cc2nccc(Oc3ccc(NC(=O)c4cn(CC(C)(C)O)cc(-c5ccc(F)cc5)c4=O)nc3)c2cc1OC]

### N-(5-((6,7-dimethoxyquinolin-4-yl)oxy)pyridin-2-yl)-5-(4-fluorophenyl)-1-(2-hydroxy-2-methylpropyl)-4-oxo-1,4-dihydropyridine-3-carboxamide

Mass spectrum: m/z=585.2 (M+H). ¹H NMR (CDCl₃) δ 13.27 (s, 1H), 8.59 (d, J=2.3 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.68 (d, J=2.3 Hz, 1H), 7.64 (m, 2H), 7.57-7.53 (m, 2H), 7.45 (s, 1H), 7.13 (m, 2H), 6.48 (d, J=5.3 Hz, 1H), 4.06 (s, 3H), 4.05 (s, 3H), 3.94 (s, 2H), 1.37 (s, 6H).

### Example 10

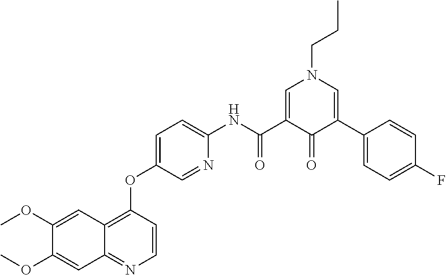 [SMILES: CCCn1cc(C(=O)Nc2ccc(Oc3ccnc4cc(OC)c(OC)cc34)cn2)c(=O)c(-c2ccc(F)cc2)c1]

### N-(5((6,7-dimethoxyquinolin-4-yl)oxy)pyridin-2-yl)-5-(4-fluorophenyl)-4-oxo-1-propyl-1,4-dihydropyridine-3-carboxamide

Mass spectrum: m/z=555.2 (M+H). ¹H NMR (CDCl₃) δ 13.26 (s, 1H), 8.59 (d, J=2.5 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.66-7.60 (m, 2H), 7.57-7.53 (m, 2H), 7.51 (d, J=2.5 Hz, 1H), 7.43 (s, 1H), 7.14 (m, 2H), 6.47 (d, J=5.3 Hz, 1H), 4.06 (s, 3H), 4.05 (s, 3H), 3.98 (t, J=7.0 Hz, 2H), 1.97 (sextet, J=7.0 Hz, 2H), 1.05 (t, J=7.2 Hz, 3H).

### Example 11

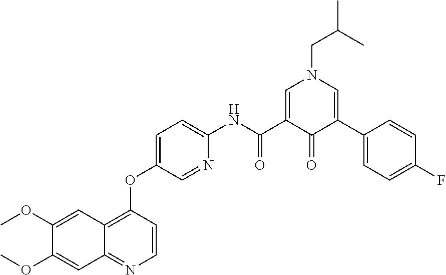 [SMILES: COc1cc2nccc(Oc3ccc(NC(=O)c4cn(CC(C)C)cc(-c5ccc(F)cc5)c4=O)nc3)c2cc1OC]

### N-(5-((6,7-dimethoxyquinolin-4-yl)oxy)pyridin-2-yl)-5-(4-fluorophenyl)-1-isobutyl-4-oxo-1,4-dihydropyridine-3-carboxamide

Mass spectrum: m/z=569.2 (M+H). ¹H NMR (CDCl₃) δ 13.26 (s, 1H), 8.55 (d, J=2.5 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.66-7.60 (m, 2H), 7.57-7.53 (m, 2H), 7.48 (d, J=2.3 Hz, 1H), 7.43 (s, 1H), 7.14 (m, 2H), 6.47 (d, J=5.1 Hz, 1H), 4.06 (s, 3H), 4.05 (s, 3H), 3.80 (d, J=7.2 Hz, 2H), 2.20 (m, 1H), 1.05 (d, J=6.7 Hz, 6H).

### Example 12

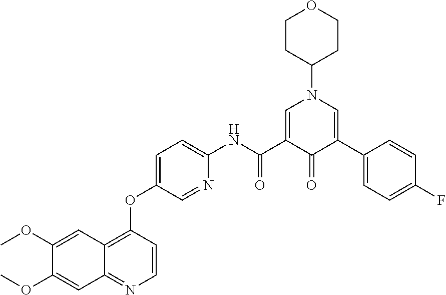 [SMILES: COc1cc2nccc(Oc3ccc(NC(=O)c4cn(C5CCOCC5)cc(-c5ccc(F)cc5)c4=O)nc3)c2cc1OC]

### N-(5((6,7-dimethoxyquinolin-4-yl)oxy)pyridin-2-yl)-5-(4-fluorophenyl)-4-oxo-1-(tetrahydro-2H-pyran-4-yl)- 1,4-dihydropyridine-3-carboxamide

Mass spectrum: m/z=597.2 (M+H). ¹H NMR (d₆-DMSO) δ 13.47 (s, 1H), 8.80 (m, 2H), 8.49 (m, 2H), 8.31 (d, J=2.3 Hz, 1H), 7.98 (dd, J=9.0, 2.9 Hz, 1H), 7.80-7.74 (m, 3H), 7.54 (s, 1H), 7.29 (m, 2H), 6.99 (d, J=6.3 Hz, 1H), 4.56 (m, 1H), 4.06-4.00 (m, 8H), 3.45 (m, 2H), 2.13 (m, 2H), 2.01 (m, 2H).

Example 13

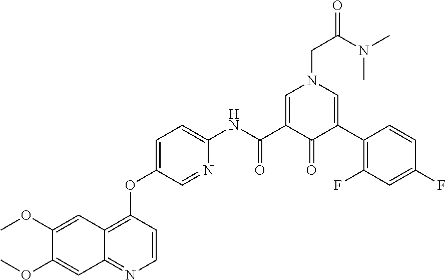 [SMILES: COc1cc2nccc(Oc3ccc(NC(=O)c4cn(CC(=O)N(C)C)cc(-c5ccc(F)cc5F)c4=O)nc3)c2cc1OC]

5-(2,4-difluorophenyl)-N-(5((6,7-dimethoxyquinolin-4-yl)oxy)pyridin-2-yl)-1-(2-(dimethylamino)-2-oxoethyl)-4-oxo-1,4-dihydropyridine-3-carboxamide

Mass spectrum: m/z=616.2 (M+H). ¹H NMR (CDCl₃) δ 13.05 (s, 1H), 8.51 (d, J=5.3 Hz, 1H), 8.49 (d, J=2.5 Hz, 1H), 8.41 (dd, J=9.0, 0.6 Hz, 1H), 8.29 (dd, J=2.9, 0.6 Hz, 1H), 7.65 (m, 1H), 7.56 (s, 1H), 7.55-7.51 (m, 2H), 7.44 (s, 1H), 6.93 (m, 2H), 6.47 (d, J=5.3 Hz, 1H), 4.80 (s, 2H), 4.06 (s, 3H), 4.05 (s, 3H), 3.12 (s, 3H), 3.05 (s, 3H).

### Example 14

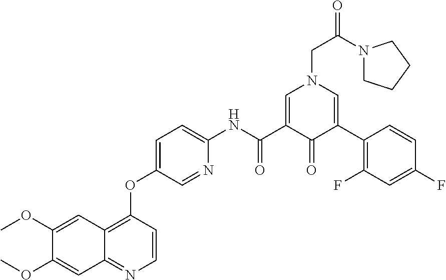 [SMILES: COc1cc2nccc(Oc3ccc(NC(=O)c4cn(CC(=O)N5CCCC5)cc(-c5ccc(F)cc5F)c4=O)nc3)c2cc1OC]

### 5-(2,4-difluorophenyl)-N-(5-((6,7-dimethoxyquinolin-4-yl)oxy)pyridin-2-yl)-4-oxo-1-(2-oxo-2-(pyrrolidin-1-yhethyl)-1,4-dihydropyridine-3-carboxamide

Mass spectrum: m/z=642.2 (M+H). ¹H NMR (CDCl₃) δ 13.06 (s, 1H), 8.51 (m, 2H), 8.41 (dd, J=9.0, 0.6 Hz, 1H), 8.29 (dd, J=2.9, 0.6 Hz, 1H), 7.65 (m, 1H), 7.57 (dd, J=2.5, 1.4 Hz, 1H), 7.55 (s, 1H), 7.54 (dd, J=9.0, 2.9 Hz, 1H), 7.45 (s, 1H), 6.93 (m, 2H), 6.47 (d, J=5.3 Hz, 1H), 4.71 (s, 2H), 4.06 (s, 3H), 4.05 (s, 3H), 3.56 (t, J=6.8 Hz, 2H), 3.50 (t, J=6.8 Hz, 2H), 2.10 (m, 2H), 1.95 (m, 2H).

### Example 15

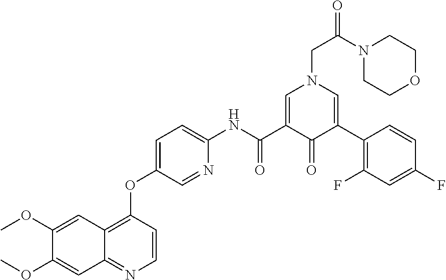 [SMILES: COc1cc2nccc(Oc3ccc(NC(=O)c4cn(CC(=O)N5CCOCC5)cc(-c5ccc(F)cc5F)c4=O)nc3)c2cc1OC]

### 5-(2,4-difluorophenyl)-N-(5-((6,7-dimethoxyquinolin-4-yl)oxy)pyridin-2-yl)-1-(2-morpholino-2-oxoethyl)-4-oxo-1,4-dihydropyridine-3-carboxamide

Mass spectrum: m/z=658.2 (M+H). ¹H NMR (CDCl₃) δ 13.03 (s, 1H), 8.51 (d, J=5.3 Hz, 1H), 8.49 (d, J=2.5 Hz, 1H), 8.40 (dd, J=9.0, 0.6 Hz, 1H), 8.29 (dd, J=2.9, 0.6 Hz, 1H), 7.65 (m, 1H), 7.55 (s, 1H), 7.55-7.50 (m, 2H), 7.45 (s, 1H), 6.93 (m, 2H), 6.43 (d, J=5.3 Hz, 1H), 4.80 (s, 2H), 4.06 (s, 3H), 4.05 (s, 3H), 3.81-3.73 (m, 4H), 3.68 (m, 2H), 3.49 (m, 2H).

### Example 16

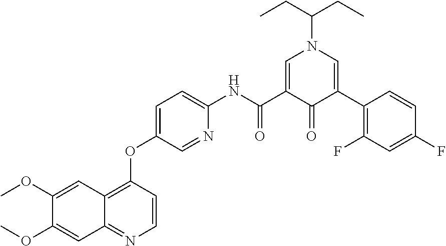 [SMILES: CCC(CC)n1cc(C(=O)Nc2ccc(Oc3ccnc4cc(OC)c(OC)cc34)cn2)c(=O)c(-c2ccc(F)cc2F)c1]

### 5-(2,4-difluorophenyl)-N-(5-((6,7-dimethoxyquinolin-4yl)oxy)pyridin-2-yl)-4-oxo-1-(pentan-3-yl)-1,4-dihydropyridine-3-carboxamide

Mass spectrum: m/z=601.2 (M+H). ¹H NMR (CDCl₃) δ 13.21 (s, 1H), 8.60 (d, J=2.5 Hz, 1H), 8.52 (d, J=5.3 Hz, 1H), 8.43 (dd, J=9.0, 0.6 Hz, 1H), 8.30 (dd, J=2.9, 0.6 Hz, 1H), 7.72 (td, J=8.6, 6.7 Hz, 1H), 7.59-7.53 (m, 3H), 7.44 (s, 1H), 7.01-6.90 (m, 2H), 6.48 (d, J=5.5 Hz, 1H), 4.06 (s, 6H), 3.70 (m, 1H), 2.01-1.79 (m, 4H), 0.95 (t, J=7.4 Hz, 6H).

### Example 17

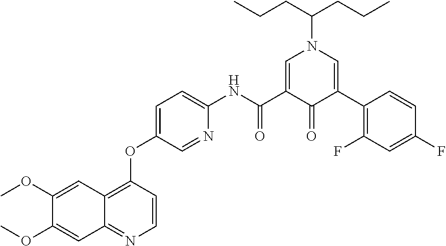 [SMILES: CCCC(CCC)n1cc(C(=O)Nc2ccc(Oc3ccnc4cc(OC)c(OC)cc34)cn2)c(=O)c(-c2ccc(F)cc2F)c1]

### 5-(2,4-difluorophenyl)-N-(5-((6,7-dimethoxyquinolin-4yl)oxy)pyridin-2-yl)-1-(heptan-4-yl)-4-oxo-1,4-dihydropyridine-3-carboxamide

Mass spectrum: m/z=629.3 (M+H). ¹H NMR (CDCl₃) δ 13.21 (s, 1H), 8.59 (d, J=2.5 Hz, 1H), 8.52 (d, J=5.3 Hz, 1H), 8.43 (dd, J=9.0, 0.6 Hz, 1H), 8.30 (dd, J=2.9, 0.6 Hz, 1H), 7.75-7.64 (m, 2H), 7.59-7.52 (m, 3H), 7.49-7.43 (m, 2H), 7.01-6.89 (m, 2H), 6.48 (d, J=5.3 Hz, 1H), 4.06 (s, 6H), 3.88 (pentet, J=7.2 Hz, 1H), 1.83 (q, J=7.6 Hz, 4H), 1.39-1.25 (m, 4H), 0.96 (t, J=7.4 Hz, 6H).

### Example 18

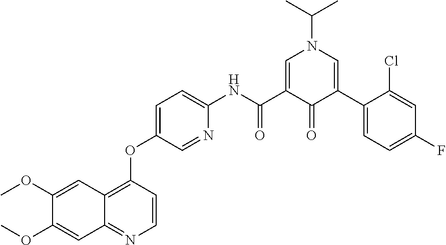 [SMILES: COc1cc2nccc(Oc3ccc(NC(=O)c4cn(C(C)C)cc(-c5ccc(F)cc5Cl)c4=O)nc3)c2cc1OC]

### 5-(2-chloro-4-fluorophenyl)-N-(5-((6,7-dimethoxyquinolin-4-yl)oxy)pyridin-2-yl)-1-isopropyl-4-oxo-1,4-dihydropyridine-3-carboxamide

Mass spectrum: m/z=589.2 (M+H). ¹H NMR (CDCl₃) δ 13.11 (s, 1H), 8.71 (d, J=2.5 Hz, 1H), 8.51 (d, J=5.3 Hz, 1H), 8.41 (dd, J=9.0, 0.6 Hz, 1H), 8.28 (dd, J=2.9, 0.6 Hz, 1H), 7.60 (d, J=2.3 Hz, 1H), 7.56-7.52 (m, 2H), 7.45 (dd, J=8.6, 6.3 Hz, 1H), 7.43 (s, 1H), 7.26 (m, 1H), 7.08 (m, 1H), 6.46 (d, J=5.1 Hz, 1H), 4.32 (m, 1H), 4.05 (s, 6H), 1.61 (d, J=6.8 Hz, 6H)

### Example 19

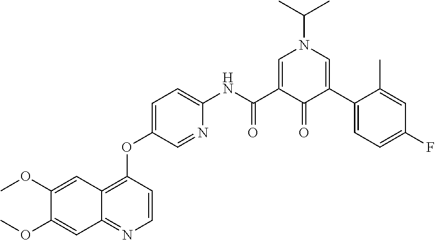 [SMILES: COc1cc2nccc(Oc3ccc(NC(=O)c4cn(C(C)C)cc(-c5ccc(F)cc5C)c4=O)nc3)c2cc1OC]

### N-(5-((6,7-dimethoxyquinolin-4yl)oxy)pyridin-2-yl)-5-(4-fluoro-2-methylphenyl)-1-isopropyl-4-oxo-1,4-dihydropyridine-3-carboxamide

Mass spectrum: m/z=569.2 (M+H). ¹H NMR (CDCl₃) δ 13.21 (s, 1H), 8.72 (d, J=2.5 Hz, 1H), 8.51 (d, J=5.3 Hz, 1H), 8.42 (dd, J=9.0, 0.6 Hz, 1H), 8.28 (dd, J=2.9, 0.6 Hz, 1H), 7.56-7.52 (m, 2H), 7.47 (d, J=2.5 Hz, 1H), 7.43 (s, 1H), 7.14 (dd, J=8.4, 5.9 Hz, 1H), 7.01 (m, 1H), 6.95 (m, 1H), 6.46 (d, J=5.3 Hz, 1H), 4.31 (m, 1H), 4.06 (s, 3H), 4.05 (s, 3H), 2.26 (s, 3H), 1.61 (d, J=6.7 Hz, 6H).

### Example 20

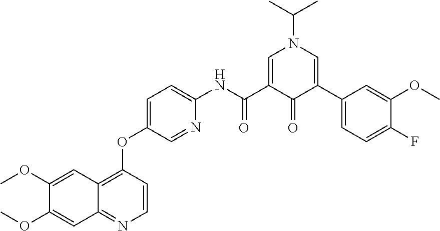

### N-(5-((6,7-dimethoxyquinolin-4yl)oxy)pyridin-2-yl)-5-(4-fluoro-3-methoxyphenyl)-1-isopropyl-4-oxo-1,4-dihydropyridine-3-carboxamide

Mass spectrum: m/z=585.2 (M+H). ¹H NMR (CDCl₃) δ 13.28 (s, 1H), 8.69 (d, J=2.5 Hz, 1H), 8.51 (d, J=5.3 Hz, 1H), 8.45 (dd, J=9.0, 0.6 Hz, 1H), 8.32 (dd, J=2.9, 0.6 Hz, 1H), 7.60-7.54 (m, 3H), 7.46-7.42 (m, 2H), 7.14 (dd, J=11.2, 8.4 Hz, 1H), 7.02 (m, 1H), 6.48 (d, J=5.3 Hz, 1H), 4.33 (m, 1H), 4.06 (s, 6H), 3.96 (s, 3H), 1.62 (d, J=6.7 Hz, 6H).

### Example 21

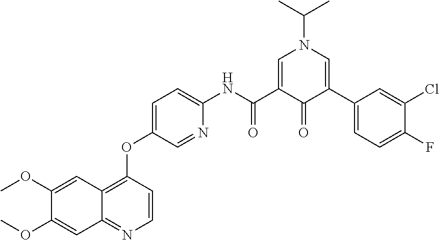 [SMILES: COc1cc2nccc(Oc3ccc(NC(=O)c4cn(C(C)C)cc(-c5ccc(F)c(Cl)c5)c4=O)nc3)c2cc1OC]

### 5-(3-chloro-4-fluorophenyl)-N-(5-((6,7-dimethoxyquinolin-4-yl)oxy)pyridin-2-yl)-1-isopropyl-4-oxo-1,4-dihydropyridine-3-carboxamide

Mass spectrum: m/z=589.2 (M+H). ¹H NMR (CDCl₃) δ 13.23 (s, 1H), 8.69 (d, J=2.5 Hz, 1H), 8.52 (d, J=5.3 Hz, 1H), 8.43 (dd, J=9.0, 0.6 Hz, 1H), 8.32 (dd, J=2.9, 0.6 Hz, 1H), 7.74 (dd, J=7.0, 2.2 Hz, 1H), 7.59 (d, J=2.5 Hz, 1H), 7.58-7.51 (m, 3H), 7.44 (s, 1H), 7.22 (8, J=8.6 Hz, 1H), 6.48 (d, J=5.3 Hz, 1H), 4.33 (m, 1H), 4.06 (s, 3H), 4.05 (s, 3H), 1.62 (d, J=6.7 Hz, 6H). [001265] Example 22

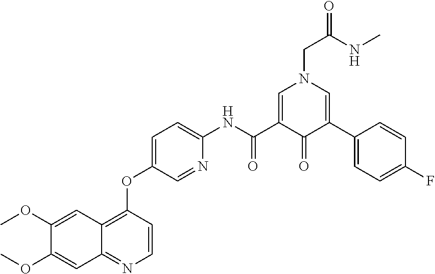 [SMILES: CNC(=O)Cn1cc(C(=O)Nc2ccc(Oc3ccnc4cc(OC)c(OC)cc34)cn2)c(=O)c(-c2ccc(F)cc2)c1]

### N-(5-((6,7-dimethoxyquinolin-4yl)oxy)pyridin-2-yl)-5-(4-fluorophenyl)-1-(2-(methylamino)-2-oxoethy0-4-oxo-1,4-dihydropyridine-3-carboxamide

Mass spectrum: m/z=584.2 (M+H). ¹H NMR (CDCl₃) δ 13.13 (s, 1H), 8.57 (d, J=2.3 Hz, 1H), 8.49 (d, J=5.1 Hz, 1H), 8.29 (d, J=9.0 Hz, 1H), 8.26 (d, J=2.7 Hz, 1H), 7.60-7.50 (m, 5H), 7.42 (s, 1H), 7.09 (m, 2H), 6.90 (m, 1H), 6.78 (m, 1H), 6.68 (br s, 1H), 6.46 (d, J=5.3 Hz, 1H), 4.68 (s, 2H), 4.05 (s, 3H), 4.02 (s, 3H), 2.81 (d, J=4.7 Hz, 3H).

### Example 23

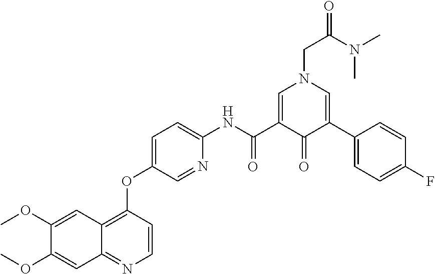 [SMILES: COc1cc2nccc(Oc3ccc(NC(=O)c4cn(CC(=O)N(C)C)cc(-c5ccc(F)cc5)c4=O)nc3)c2cc1OC]

### N-(5-((6,7-dimethoxyquinolin-4yl)oxy)pyridin-2-yl)-1-(2-(dimethylamino)-2-oxoethyl)-5-(4-fluorophenyl)-4-oxo-1,4-dihydropyridine-3-carboxamide

Mass spectrum: m/z=598.2 (M+H). ¹H NMR (CDCl₃) δ 13.17 (s, 1H), 8.51 (d, J=5.3 Hz, 1H), 8.47 (d, J=2.5 Hz, 1H), 8.41 (dd, J=9.0, 0.6 Hz 1H), 8.30 (dd, J=2.9, 0.6 Hz, 1H), 7.62 (m, 2H), 7.56 (s, 1H), 7.53 (dd, J=8.8, 2.7 Hz, 1H), 7.46 (d, J=2.5 Hz, 1H), 7.43 (s, 1H), 7.11 (m, 2H), 6.48 (d, J=5.3 Hz, 1H), 4.79 (s, 2H), 4.06 (s, 3H), 4.05 (s, 3H), 3.12 (s, 3H), 3.06 (s, 3H).

### Example 24

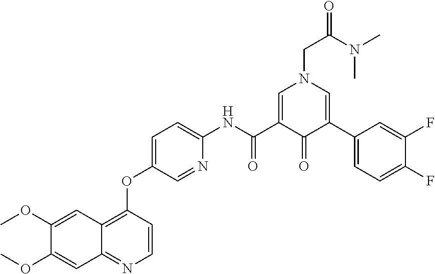 [SMILES: COc1cc2nccc(Oc3ccc(NC(=O)c4cn(CC(=O)N(C)C)cc(-c5ccc(F)c(F)c5)c4=O)nc3)c2cc1OC]

### 5-(3,4-difluorophenyl)-N-(5-((6,7-dimethoxyquinolin-4-yl)oxy)pyridin-2-yl)-1-(2-(dimethylamino)-2-oxoethyl)-4-oxo-1,4-dihydropyridine-3-carboxamide

Mass spectrum: m/z=616.2 (M+H). ¹H NMR (CDCl₃) δ 13.11 (s, 1H), 8.52 (d, J=5.3 Hz, 1H), 8.47 (d, J=2.5 Hz, 1H), 8.41 (dd, J=9.0, 0.6 Hz 1H), 8.31 (dd, J=2.9, 0.6 Hz, 1H), 7.61-7.52 (m, 3H), 7.48 (d, J=2.5 Hz, 1H), 7.44 (s, 1H), 7.35 (m, 1H), 7.20 (dt, J=10.2, 8.4 Hz, 1H), 6.48 (d, J=5.3 Hz, 1H), 4.80 (s, 2H), 4.06 (s, 3H), 4.05 (s, 3H), 3.13 (s, 3H), 3.06 (s, 3H)

Example 25

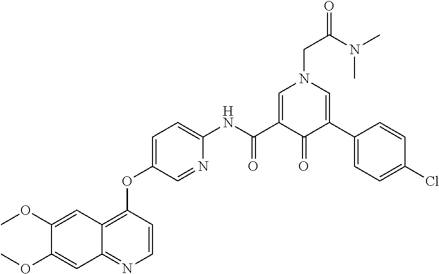 [SMILES: COc1cc2nccc(Oc3ccc(NC(=O)c4cn(CC(=O)N(C)C)cc(-c5ccc(Cl)cc5)c4=O)nc3)c2cc1OC]

### 5-(4-chlorophenyl)-N-(5-((6,7-dimethoxyquinolin-4-yhoxy)pyridin-2-yl)-1-(2-(dimethylamino)-2-oxoethyl)-4-oxo-1,4-dihydropyridine-3-carboxamide

Mass spectrum: m/z=614.1 (M+H). ¹H NMR (CDCl₃) δ 13.15 (s, 1H), 8.52 (d, J=5.3 Hz, 1H), 8.47 (d, J=2.5 Hz, 1H), 8.41 (dd, J=9.0, 0.6 Hz 1H), 8.30 (dd, J=2.9, 0.6 Hz, 1H), 7.59 (m, 2H), 7.56 (s, 1H), 7.54 (dd, J=8.8, 2.7 Hz, 1H), 7.47 (d, J=2.5 Hz, 1H), 7.45 (s, 1H), 7.39 (m, 2H), 6.48 (d, J=5.5 Hz, 1H), 4.79 (s, 2H), 4.06 (s, 3H), 4.05 (s, 3H), 3.12 (s, 3H), 3.06 (s, 3H).

Example 26

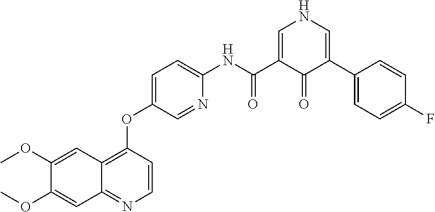 [SMILES: COc1cc2nccc(Oc3ccc(NC(=O)c4c[nH]cc(-c5ccc(F)cc5)c4=O)nc3)c2cc1OC]

### N-(5-((6,7-dimethoxyquinolin-4yl)oxy)pyridin-2-yl)-5-(4-fluorophenyl)-4-oxo-1,4-dihydropyridine-3-carboxamide

Mass spectrum: m/z=513.2 (M+H). ¹H NMR (CDCl₃) δ 8.62 (d, J=1.6 Hz, 1H), 8.50 (d, J=5.3 Hz, 1H), 8.42 (d, J=9.0 Hz, 1H), 8.31 (d, J=2.5 Hz, 1H), 7.65-7.53 (m, 6H), 7.43 (s, 1H), 7.13 (m, 2H), 6.49 (d, J=5.3 Hz, 1H), 4.79 (s, 2H), 4.06 (s, 6H).

### Example 27

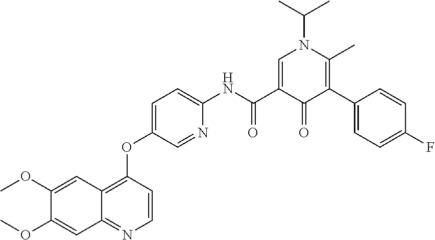 [SMILES: COc1cc2nccc(Oc3ccc(NC(=O)c4cn(C(C)C)c(C)c(-c5ccc(F)cc5)c4=O)nc3)c2cc1OC]

### N-(5-((6,7-dimethoxyquinolin-4yl)oxy)pyridin-2-yl)-5-(4-fluorophenyl)-1-isopropyl-6-methyl-4-oxo-1,4-dihydropyridine-3-carboxamide

Mass spectrum: m/z=569.2 (M+H). ¹H NMR (CDCl₃) δ 13.20 (s, 1H), 8.77 (s, 1H), 8.50 (d, J=5.3 Hz, 1H), 8.40 (d, J=9.0 Hz, 1H), 8.25 (d, J=2.7 Hz, 1H), 7.54 (s, 1H), 7.53 (dd, J=9.0, 2.9 Hz, 1H), 7.43 (s, 1H), 7.24-7.13 (m, 4H), 6.46 (d, J=5.3 Hz, 1H), 4.66 (m, 1H), 4.05 (s, 6H), 2.31 (s, 3H), 1.61 (d, J=6.7 Hz, 6H).

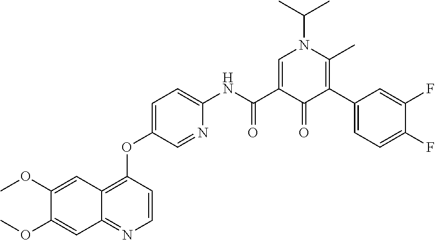 [SMILES: COc1cc2nccc(Oc3ccc(NC(=O)c4cn(C(C)C)c(C)c(-c5ccc(F)c(F)c5)c4=O)nc3)c2cc1OC]

### 5-(3,4-difluorophenyl)-N-(5-((6,7-dimethoxyquinolin-4yl)oxy)pyridin-2-yl)-1-isopropyl-6-methyl-4-oxo-1,4-dihydropyridine-3-carboxamide

Mass spectrum: m/z=587.2 (M+H). ¹H NMR (CDCl₃) δ 13.13 (s, 1H), 8.78 (s, 1H), 8.51 (d, J=5.3 Hz, 1H), 8.40 (dd, J=9.0, 0.6 Hz, 1H), 8.25 (dd, J=2.9, 0.6 Hz, 1H), 7.55-7.51 (m, 2H), 7.43 (s, 1H), 7.25 (m, 1H), 7.09 (m, 1H), 6.96 (m, 1H), 6.46 (d, J=5.3 Hz, 1H), 4.66 (m, 1H), 4.05 (s, 6H), 2.32 (s, 3H), 1.61 (d, J=6.7 Hz, 6H).

### Example 29

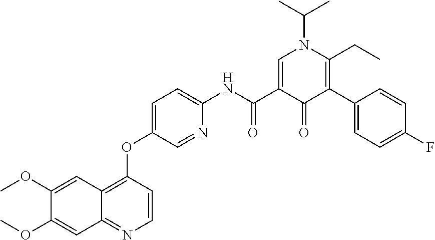 [SMILES: CCc1c(-c2ccc(F)cc2)c(=O)c(C(=O)Nc2ccc(Oc3ccnc4cc(OC)c(OC)cc34)cn2)cn1C(C)C]

### N-(5-((6,7-dimethoxyquinolin-4yl)oxy)pyridin-2-yl)-6-ethyl-5-(4-fluorophenyl)-1-isopropyl-4-oxo-1,4-dihydropyridine-3-carboxamide

Mass spectrum: m/z=583.3 (M+H). ¹H NMR (CDCl₃) δ 13.18 (s, 1H), 8.76 (s, 1H), 8.50 (d, J=5.3 Hz, 1H), 8.39 (d, J=9.0 Hz, 1H), 8.25 (d, J=2.7 Hz, 1H), 7.55-7.50 (m, 2H), 7.43 (s, 1H), 7.24-7.13 (m, 4H), 6.46 (d, J=5.3 Hz, 1H), 4.64 (m, 1H), 4.05 (s, 6H), 2.60 (q, J=7.6 Hz, 2H), 1.63 (d, J=6.7 Hz, 6H), 1.17 (t, J=7.4 Hz, 3H).

### Example 30

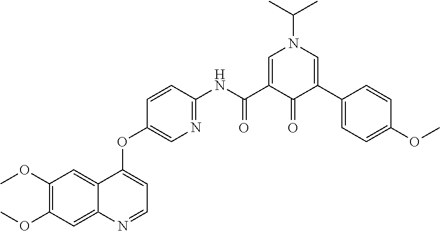

### N-(5-((6,7-dimethoxyquinolin-4yl)oxy)pyridin-2-yl)-1-isopropyl-5-(4-methoxyphenyl)-4-oxo-1,4-dihydropyridine-3-carboxamide

Mass spectrum: m/z=567.2 (M+H). ¹H NMR (CDCl₃) δ 13.39 (s, 1H), 8.66 (d, J=2.5 Hz, 1H), 8.52 (d, J=5.3 Hz, 1H), 8.44 (dd, J=9.0, 0.6 Hz, 1H), 8.30 (dd, J=2.9, 0.6 Hz, 1H), 7.60 (m, 2H), 7.58-7.53 (m, 3H), 7.44 (s, 1H), 6.98 (m, 2H), 6.48 (d, J=5.3 Hz, 1H), 4.31 (m, 1H), 4.06 (s, 3H), 4.05 (s, 3H), 3.86 (s, 3H), 1.61 (d, J=6.8 Hz, 6H).

### Example 31

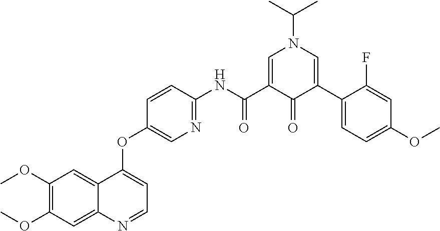

### N-(5-((6,7-dimethoxyquinolin-4yl)oxy)pyridin-2-yl)-5-(2-fluoro-4-methoxyphenyl)-1-isopropyl-4-oxo-1,4-dihydropyridine-3-carboxamide

Mass spectrum: m/z=585.2 (M+H). ¹H NMR (CDCl₃) δ 13.27 (s, 1H), 8.67 (d, J=2.5 Hz, 1H), 8.51 (d, J=5.3 Hz, 1H), 8.43 (dd, J=9.0, 0.6 Hz, 1H), 8.29 (dd, J=2.9, 0.6 Hz, 1H), 7.65 (dd, J=2.5, 1.8 Hz, 1H), 7.62 (t, J=8.6 Hz, 1H), 7.57-7.52 (m, 2H), 7.43 (s, 1H), 6.79 (dd, j=8.6, 2.5 Hz, 1H), 6.73 (dd, J=12.1, 2.3 Hz, 1H), 6.48 (d, J=5.3 Hz, 1H), 4.31 (m, 1H), 4.06 (s, 3H), 4.05 (s, 3H), 3.84 (s, 3H), 1.60 (d, J=6.8 Hz, 6H).

### Example 32

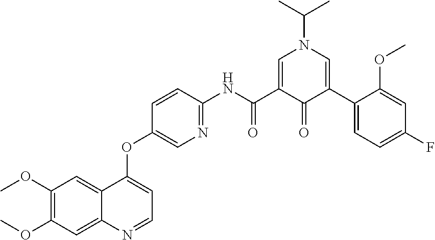 [SMILES: COc1cc2nccc(Oc3ccc(NC(=O)c4cn(C(C)C)cc(-c5ccc(F)cc5OC)c4=O)nc3)c2cc1OC]

### N-(5-((6,7-dimethoxyquinolin-4yl)oxy)pyridin-2-yl)-5-(4-fluoro-2-methoxyphenyl)-1-isopropyl-4-oxo-1,4-dihydropyridine-3-carboxamide

Mass spectrum: m/z=585.2 (M+H). ¹H NMR (CDCl₃) δ 13.30 (s, 1H), 8.67 (d, J=2.5 Hz, 1H), 8.51 (d, J=5.3 Hz, 1H), 8.43 (dd, J=9.0, 0.6 Hz, 1H), 8.28 (dd, J=2.9, 0.6 Hz, 1H), 7.62 (d, J=2.5 Hz, 1H), 7.56-7.51 (m, 2H), 7.46-7.40 (m, 2H), 6.78-6.70 (m, 2H), 6.47 (d, J=5.3 Hz, 1H), 4.29 (m, 1H), 4.06 (s, 3H), 4.05 (s, 3H), 3.80 (s, 3H), 1.59 (d, J=6.8 Hz, 6H).

### Example 33

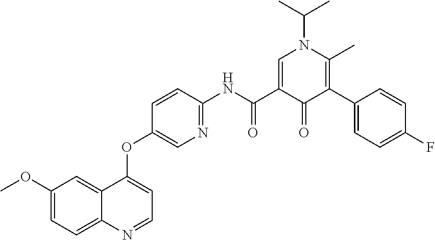 [SMILES: COc1ccc2nccc(Oc3ccc(NC(=O)c4cn(C(C)C)c(C)c(-c5ccc(F)cc5)c4=O)nc3)c2c1]

### 5-(4-fluorophenyl)-1-isopropyl-N-(5-((6-methoxyquinolin-4-yl)oxy)pyridin-2-yl)-6-methyl-4-oxo-1,4-dihydropyridine-3-carboxamide

Mass spectrum: m/z=539.2 (M+H). ¹H NMR (CDCl₃) δ 13.21 (s, 1H), 8.77 (s, 1H), 8.55 (d, J=5.1 Hz, 1H), 8.41 (d, J=9.0 Hz, 1H), 8.26 (d, J=2.9 Hz, 1H), 8.00 (d, J=9.2 Hz, 1H), 7.57 (d, J=2.7 Hz, 1H), 7.53 (dd, J=9.0, 2.9 Hz, 1H), 7.41 (dd, J=9.4, 2.9 Hz, 1H), 7.24-7.13 (m, 4H), 6.54 (d, J=5.1 Hz, 1H), 4.66 (m, 1H), 3.97 (s, 3H), 2.31 (s, 3H), 1.61 (d, J=6.7 Hz, 6H).

### Example 34

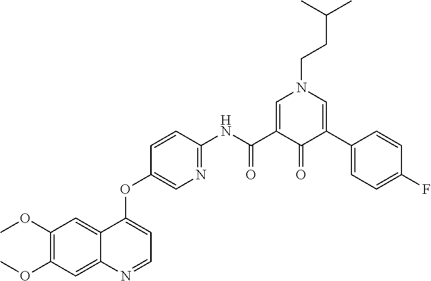 [SMILES: COc1cc2nccc(Oc3ccc(NC(=O)c4cn(CCC(C)C)cc(-c5ccc(F)cc5)c4=O)nc3)c2cc1OC]

### N-(5-((6,7-dimethoxyquinolin-4yl)oxy)pyridin-2-yl)-5-(4-fluorophenyl)-1-isopentyl-4-oxo-1,4-dihydropyridine-3-carboxamide

Mass spectrum: m/z=583.2 (M+H). ¹H NMR (CDCl₃) δ 13.26 (s, 1H), 8.59 (d, J=2.5 Hz, 1H), 8.51 (d, J=5.3 Hz, 1H), 8.43 (dd, J=9.0, 0.6 Hz, 1H), 8.30 (dd, J=2.9, 0.6 Hz, 1H), 7.62 (m, 2H), 7.55 (m, 2H), 7.51 (d, J=2.5 Hz, 1H), 7.44 (s, 1H), 7.13 (m, 2H), 6.47 (d, J=5.3 Hz, 1H), 4.06 (s, 6H), 4.01 (m, 2H), 1.82 (m, 2H), 1.70 (m, 1H), 1.02 (d, J=6.7 Hz, 6H).

### Example 35

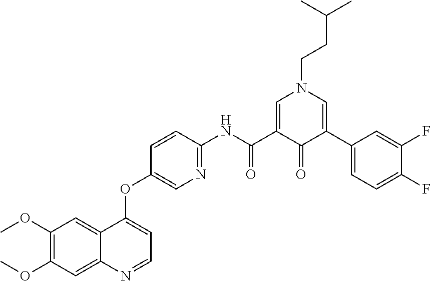 [SMILES: COc1cc2nccc(Oc3ccc(NC(=O)c4cn(CCC(C)C)cc(-c5ccc(F)c(F)c5)c4=O)nc3)c2cc1OC]

### 5-(3,4-difluorophenyl)-N-(5-((6,7-dimethoxyquinolin-4yl)oxy)pyridin-2-yl)-1-isopentyl-4-oxo-1,4-dihydropyridine-3-carboxamide

Mass spectrum: m/z=601.2 (M+H). ¹H NMR (CDCl₃) δ 13.20 (s, 1H), 8.60 (d, J=2.5 Hz, 1H), 8.52 (d, J=5.3 Hz, 1H), 8.43 (dd, J=9.0, 0.6 Hz, 1H), 8.32 (dd, J=2.9, 0.6 Hz, 1H), 7.71-7.64 (m, 1H), 7.62-7.51 (m, 4H), 7.46 (m, 1H), 7.44 (s, 1H), 7.35 (m, 1H), 7.22 (m, 1H), 6.48 (d, J=5.3 Hz, 1H), 4.06 (s, 3H), 4.05 (s, 3H), 4.02 (m, 2H), 1.82 (m, 2H), 1.70 (m, 1H), 1.03 (d, J=6.7 Hz, 6H).

### Example 36

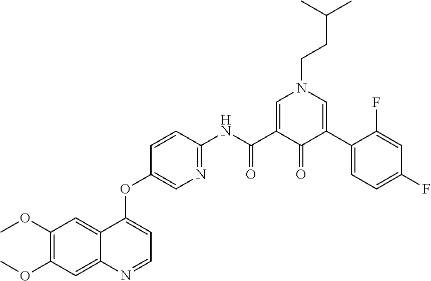 [SMILES: COc1cc2nccc(Oc3ccc(NC(=O)c4cn(CCC(C)C)cc(-c5ccc(F)cc5F)c4=O)nc3)c2cc1OC]

### 5-(2,4-difluorophenyl)-N-(5-((6,7-dimethoxyquinolin-4yl)oxy)pyridin-2-yl)-1-isopentyl-4-oxo-1,4-dihydropyridine-3-carboxamide

Mass spectrum: m/z=601.2 (M+H). ¹H NMR (CDCl₃) δ 13.14 (s, 1H), 8.61 (d, J=2.5 Hz, 1H), 8.51 (d, J=5.3 Hz, 1H), 8.42 (d, J=9.0 Hz, 1H), 8.30 (d, J=2.9 Hz, 1H), 7.67 (td, J=8.6, 6.7 Hz, 1H), 7.58 (dd, J−2.1, 1.8 Hz, 1H), 7.57-7.53 (m, 2H), 7.44 (s, 1H), 6.95 (m, 2H), 6.47 (d, J=5.3 Hz, 1H), 4.06 (s, 6H), 4.01 (m, 2H), 1.81 (m, 2H), 1.70 (m, 1H), 1.02 (d, J=6.7 Hz, 6H).

### Example 37

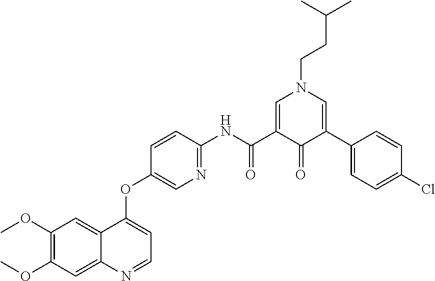 [SMILES: COc1cc2nccc(Oc3ccc(NC(=O)c4cn(CCC(C)C)cc(-c5ccc(Cl)cc5)c4=O)nc3)c2cc1OC]

### 5-(4-chloropheny0-N-(5-((6,7-dimethoxyquinolin-4-yl)oxy)pyridin-2-yI)-1-isopentyl-4-oxo-1,4-dihydropyridine-3-carboxamide

Mass spectrum: m/z=599.2 (M+H). ¹H NMR (CDCl₃) δ 13.24 (s, 1H), 8.59 (d, J=2.5 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.67 (m, 1H), 7.59 (m, 2H), 7.57-7.51 (m, 3H), 7.47 (m, 1H), 7.44 (s, 1H), 7.42 (m, 2H), 6.48 (d, J=5.3 Hz, 1H), 4.06 (s, 3H), 4.05 (s, 3H), 4.01 (m, 2H), 1.82 (m, 2H), 1.70 (m, 1H), 1.02 (d, J=6.7 Hz, 6H).

### Example 38

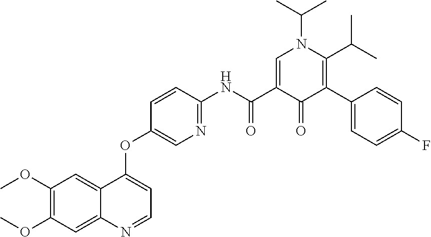 [SMILES: COc1cc2nccc(Oc3ccc(NC(=O)c4cn(C(C)C)c(C(C)C)c(-c5ccc(F)cc5)c4=O)nc3)c2cc1OC]

### N-(5-((6,7-dimethoxyquinolin-4yl)oxy)pyridin-2-yl)-5-(4-fluorophenyl)-1,6-diisopropyl-4-oxo-1,4-dihydropyridine-3-carboxamide

Mass spectrum: m/z=597.2 (M+H). ¹H NMR (CDCl₃) δ 13.15 (s, 1H), 8.72 (s, 1H), 8.51 (d, J=5.3 Hz, 1H), 8.39 (d, J=9.0 Hz, 1H), 8.24 (d, J=2.7 Hz, 1H), 7.54 (s, 1H), 7.52 (dd, J=9.0, 2.9 Hz, 1H), 7.45 (s, 1H), 7.16 (d, J=3H), 6.47 (d, J=5.3 Hz, 1H), 4.82 (m, 1H), 4.06 (s, 3H), 4.05 (s, 3H), 3.36 (m, 1H), 1.63 (d, J=6.7 Hz, 6H), 1.32 (d, J=7.4 Hz, 6H).

### Example 39

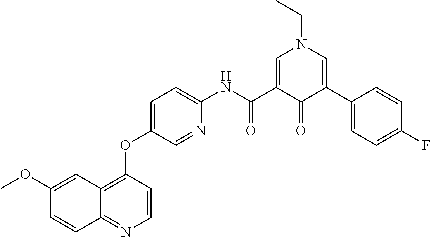 [SMILES: CCn1cc(C(=O)Nc2ccc(Oc3ccnc4ccc(OC)cc34)cn2)c(=O)c(-c2ccc(F)cc2)c1]

### 1-ethyl-5-(4-fluorophenyl)-N-(5-((6-methoxyquinolin-4-yl)oxy)pyridin-2-yl)-4-oxo-1,4-dihydropyridine-3-carboxamide

Mass spectrum: m/z=511.2 (M+H). ¹H NMR (CDCl₃) δ 13.27 (s, 1H), 8.62 (d, J=2.5 Hz, 1H), 8.57 (d, J=5.1 Hz, 1H), 8.44 (dd, J=9.0, 0.6 Hz, 1H), 8.31 (dd, J=2.9, 0.6 Hz, 1H), 8.01 (d, J=9.2 Hz, 1H), 7.65-.752 (m, 5H), 7.42 (dd, J=9.2, 2.7 Hz, 1H), 7.14 (m, 2H), 6.55 (d, J=5.1 Hz, 1H), 4.09 (q, J=7.2 Hz, 2H), 3.98 (s, 3H), 1.60 (t, J=7.2 Hz, 3H).

### Example 40

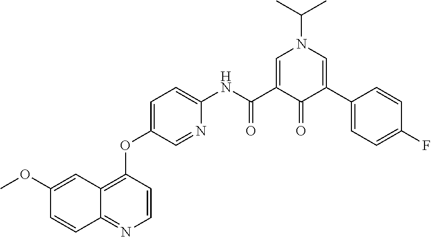 [SMILES: COc1ccc2nccc(Oc3ccc(NC(=O)c4cn(C(C)C)cc(-c5ccc(F)cc5)c4=O)nc3)c2c1]

### 5-(4-fluorophenyl)-1-isopropyl-N-(5-((6-methoxyquinolin-4-yl)oxy)pyridin-2-yl)-4-oxo-1,4-dihydropyridine-3-carboxamide

Mass spectrum: m/z=525.2 (M+H). ¹H NMR (CDCl₃) δ 13.31 (s, 1H), 8.69 (d, J=2.5 Hz, 1H), 8.56 (d, J=5.1 Hz, 1H), 8.44 (dd, J=9.0, 0.6 Hz, 1H), 8.31 (dd, J=2.9, 0.6 Hz, 1H), 8.00 (d, J=9.2 Hz, 1H), 7.63 (m, 2H), 7.59 (m, 2H), 7.56 (dd, J=9.0, 2.9 Hz, 1H), 7.42 (dd, J=9.2, 2.7 Hz, 1H), 7.14 (m, 2H), 6.56 (d, J=5.1 Hz, 1H), 4.33 (m, 1H), 3.98 (s, 3H), 1.62 (d, J=6.7 Hz, 6H).

### Example 41

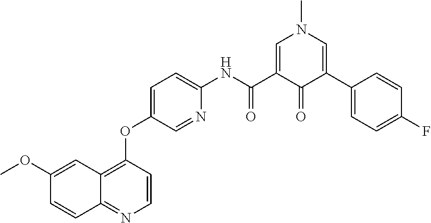 [SMILES: COc1ccc2nccc(Oc3ccc(NC(=O)c4cn(C)cc(-c5ccc(F)cc5)c4=O)nc3)c2c1]

### 5-(4-fluorophenyl)-N-(5-((6-methoxyquinolin-4yl)oxy)pyridin-2-yl)-1-methyl-4-oxo-1,4-dihydropyridine-3-carboxamide

Mass spectrum: m/z=497.2 (M+H). ¹H NMR (CDCl₃) δ 13.23 (s, 1H), 8.58 (d, J=2.5 Hz, 1H), 8.56 (d, J=5.1 Hz, 1H), 8.44 (dd, J=9.0, 0.6 Hz, 1H), 8.31 (dd, J=2.9, 0.6 Hz, 1H), 8.00 (d, J=9.2 Hz, 1H), 7.61 (m, 2H), 7.58 (d, J=2.7 Hz, 1H), 7.56 (dd, J=8.8, 2.7 Hz, 1H), 7.50 (d, J=2.3, 2.7 Hz, 1H), 7.42 (dd, J=9.4, 2.7 Hz, 1H), 7.13 (m, 2H), 6.55 (d, J=5.1 Hz, 1H), 3.98 (s, 3H), 3.90 (s, 3H).

### Example 42

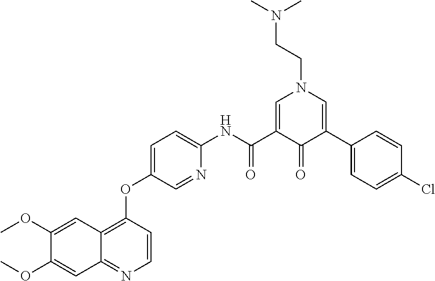 [SMILES: COc1cc2nccc(Oc3ccc(NC(=O)c4cn(CCN(C)C)cc(-c5ccc(Cl)cc5)c4=O)nc3)c2cc1OC]

### 5-(4-chlorophenyl)-N-(5-((6,7-dimethoxyquinolin-4-yl)pyridin-2-yl)-1-(2-(dimethylamino)ethyl)-4-oxo-1,4-dihydropyridine-3-carboxamide

Mass spectrum: m/z=600.2 (M+H). ¹H NMR (CDCl₃) δ 13.26 (s, 1H), 8.60 (d, J=2.5 Hz, 1H), 8.51 (d, J=5.3 Hz, 1H), 8.43 (dd, J=9.0, 0.6 Hz, 1H), 8.31 (dd, J=2.9, 0.6 Hz, 1H), 7.61-7.52 (m, 5H), 7.45 (m, 1H), 7.41 (m, 2H), 6.47 (d, J=5.3 Hz, 1H), 4.07-4.01 (m, 8H), 2.75 (m, 2H), 2.32 (s, 6H).

### Example 43

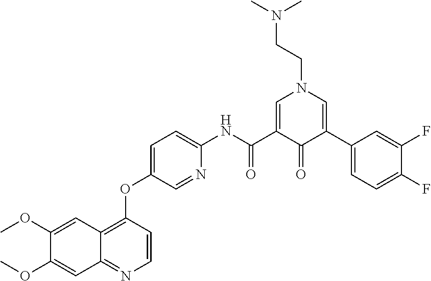 [SMILES: COc1cc2nccc(Oc3ccc(NC(=O)c4cn(CCN(C)C)cc(-c5ccc(F)c(F)c5)c4=O)nc3)c2cc1OC]

### 5-(3,4-difluorophenyl)-N-(5-((6,7-dimethoxyquinolin-4-yl)oxy)pyridin-2-yl)-1-

Mass spectrum: m/z=602.2 (M+H). ¹H NMR (CDCl₃) δ 13.22 (s, 1H), 8.60 (d, J=2.5 Hz, 1H), 8.52 (d, J=5.3 Hz, 1H), 8.43 (dd, J=9.0, 0.6 Hz, 1H), 8.32 (dd, J=2.9, 0.6 Hz, 1H), 7.62 (d, J=2.3 Hz, 1H), 7.57-7.53 (m, 2H), 7.44 (s, 1H), 7.35 (m, 1H), 7.22 (m, 1H), 6.48 (d, J=5.3 Hz, 1H), 4.08-4.01 (m, 8H), 2.75 (m, 2H), 2.32 (s, 6H).

### Example 44

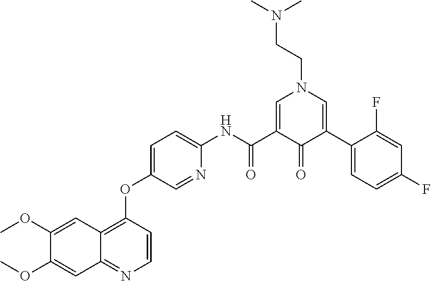 [SMILES: COc1cc2nccc(Oc3ccc(NC(=O)c4cn(CCN(C)C)cc(-c5ccc(F)cc5F)c4=O)nc3)c2cc1OC]

### 5-(2,4-difluorophenyl)-N-(5-((6,7-dimethoxyquinolin-4-yl)oxy)pyridin-2-yl)-1-(2-(dimethylamino)ethyl)-4-oxo-1,4-dihydropyridine-3-carboxamide

Mass spectrum: m/z=602.2 (M+H). ¹H NMR (CDCl₃) δ 13.16 (s, 1H), 8.62 (d, J=2.5 Hz, 1H), 8.51 (d, J=5.3 Hz, 1H), 8.42 (dd, J=9.0, 0.6 Hz, 1H), 8.29 (dd, J=2.9, 0.6 Hz, 1H), 7.70-7.63 (m, 2H), 7.56-7.52 (m, 2H), 7.43 (s, 1H), 6.95 (m, 2H), 6.47 (d, J=5.3 Hz, 1H), 4.07-4.00 (m, 8H), 2.75 (m, 2H), 2.31 (s, 6H).

### Example 45

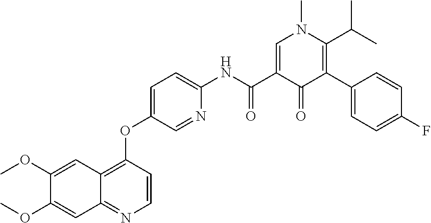 [SMILES: COc1cc2nccc(Oc3ccc(NC(=O)c4cn(C)c(C(C)C)c(-c5ccc(F)cc5)c4=O)nc3)c2cc1OC]

### N-(5-((6,7-dimethoxyquinolin-4-yl)oxy)pyridin-2-yl)-5-(4-fluorophenyl)-6-isopropyl-1-methyl-4-oxo-1,4-dihydropyridine-3-carboxamide

Mass spectrum: m/z=569.2 (M+H). ¹H NMR (CDCl₃) δ 13.07 (s, 1H), 8.55 (s, 1H), 8.50 (d, J=5.3 Hz, 1H), 8.38 (dd, J=9.0, 0.6 Hz, 1H), 8.24 (dd, J=2.9, 0.6 Hz, 1H), 7.54 (s, 1H), 7.52 (dd, J=8.8, 2.7 Hz, 1H), 7.45 (m, 1H), 7.15 (d, J=7.0 Hz, 4H), 6.46 (d, J=5.3 Hz, 1H), 4.06, (s, 3H), 4.05 (s, 3H), 3.95 (s, 3H), 3.29 (m, 1H), 1.26 (d, J=7.0 Hz, 6H).

### Example 46

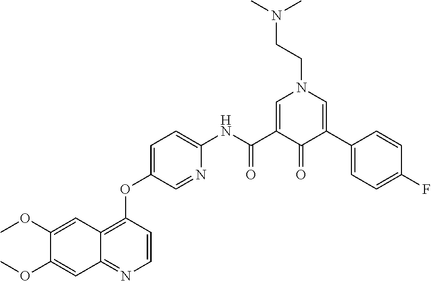 [SMILES: COc1cc2nccc(Oc3ccc(NC(=O)c4cn(CCN(C)C)cc(-c5ccc(F)cc5)c4=O)nc3)c2cc1OC]

### N-(5-((6,7-dimethoxyquinolin-4-ypoxy)pyridin-2-yl)-1-(2-(dimethylamino)ethy0-5-(4-fluorophenyl)-4-oxo-1,4-dihydropyridine-3-carboxamide

Mass spectrum: m/z=584.2 (M+H). ¹H NMR (CDCl₃) δ 13.30 (s, 1H), 8.60 (d, J=2.5 Hz, 1H), 8.51 (d, J=5.3 Hz, 1H), 8.44 (dd, J=9.0, 0.6 Hz, 1H), 8.31 (dd, J=2.9, 0.6 Hz, 1H), 7.63 (m, 2H), 7.60 (d, J=2.3 Hz, 1H), 7.57-7.53 (m, 2H), 7.47 (m, 1H), 7.14 (m, 2H), 6.49 (d, J=5.3 Hz, 1H), 4.08-4.02 (m, 8H), 2.76 (m, 2H), 2.32 (s, 6H).

### Example 47

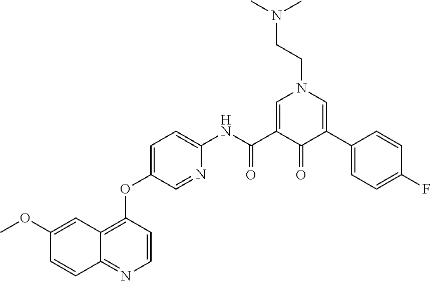 [SMILES: COc1ccc2nccc(Oc3ccc(NC(=O)c4cn(CCN(C)C)cc(-c5ccc(F)cc5)c4=O)nc3)c2c1]

### 1-(2-(dimethylamino)ethy0-5-(4-fluorophenyl)-N-(5-((6-methoxyquinolin-4-yl)oxy)pyridin-2-yl)-4-oxo-1,4-dihydropyridine-3-carboxamide

Mass spectrum: m/z=554.2 (M+H). ¹H NMR (CDCl₃) δ 13.30 (s, 1H), 8.60 (d, J=2.5 Hz, 1H), 8.56 (d, J=5.3 Hz, 1H), 8.44 (dd, J=9.0, 0.6 Hz, 1H), 8.31 (dd, J=2.9, 0.6 Hz, 1H), 8.00 (d, J=9.2 Hz, 1H), 7.63 (m, 2H), 7.59 (m, 2H), 7.55 (dd, J=9.0, 2.9 Hz, 1H), 7.42 (dd, J=9.2, 2.7 Hz, 1H), 7.13 (m, 2H), 6.55 (d, J=5.3 Hz, 1H), 4.04 (m, 2H), 3.98 (s, 3H), 2.75 (m, 2H), 2.32 (s, 6H).

### Example 48

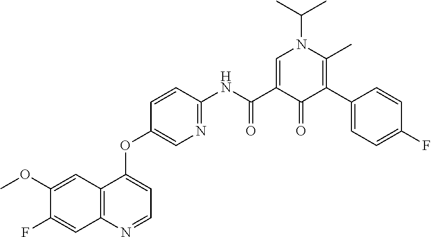 [SMILES: COc1cc2c(Oc3ccc(NC(=O)c4cn(C(C)C)c(C)c(-c5ccc(F)cc5)c4=O)nc3)ccnc2cc1F]

### N-(5-((7-fluoro-6-methoxyquinolin-4yl)oxy)pyridin-2-yl)-5-(4-fluorophenyl)-1-isopropyl-6-methyl-4-oxo-1,4-dihydropyridine-3-carboxamide

Mass spectrum: m/z=557.2 (M+H). ¹H NMR (CDCl₃) δ 13.23 (s, 1H), 8.77 (s, 1H), 8.56 (d, J=5.3 Hz, 1H), 8.42 (dd, J=9.0, 0.6 Hz, 1H), 8.25 (dd, J=2.9, 0.6 Hz, 1H), 7.73 (d, J=11.9 Hz, 1H), 7.67 (d, J=9.0 Hz, 1H), 7.53 (dd, J=9.0, 2.9 Hz, 1H), 7.24-7.13 (m, 4H), 6.52 (d, J=5.3 Hz, 1H), 4.66 (m, 1H), 4.05 (s, 3H), 2.31 (s, 3H), 1.61 (d, J=6.8 Hz, 6H).

### Example 49

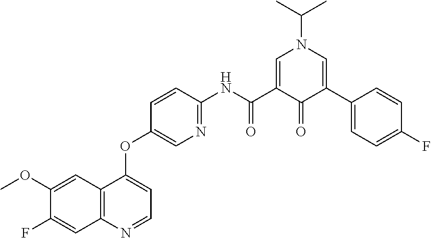 [SMILES: COc1cc2c(Oc3ccc(NC(=O)c4cn(C(C)C)cc(-c5ccc(F)cc5)c4=O)nc3)ccnc2cc1F]

### N-(5-((7-fluoro-6-methoxyquinolin-4yl)oxy)pyridin-2-yl)-5-(4-fluorophenyl)-1-isopropyl-4-oxo-1,4-dihydropyridine-3-carboxamide

Mass spectrum: m/z=543.2 (M+H). ¹H NMR (CDCl₃) δ 13.32 (s, 1H), 8.69 (d, J=2.5 Hz, 1H), 8.57 (d, J=5.3 Hz, 1H), 8.45 (dd, J=9.0, 0.6 Hz, 1H), 8.31 (dd, J=2.9, 0.6 Hz, 1H), 7.74 (d, J=12.1 Hz, 1H), 7.68 (d, J=9.0 Hz, 1H), 7.63 (m, 2H), 7.58 (d, J=2.5 Hz, 1H), 7.56 (dd, J=8.6, 2.9 Hz, 1H), 7.14 (m, 2H), 6.53 (d, J=5.3 Hz, 1H), 4.33 (m, 1H), 4.06 (s, 3H), 1.62 (d, J=6.8 Hz, 6H).

### Example 50

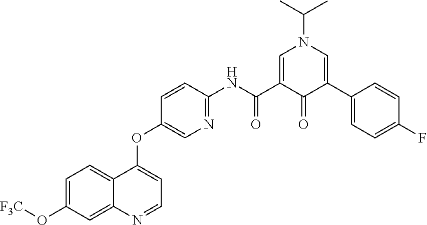 [SMILES: CC(C)n1cc(C(=O)Nc2ccc(Oc3ccnc4cc(OC(F)(F)F)ccc34)cn2)c(=O)c(-c2ccc(F)cc2)c1]

### 5-(4-fluorophenyl)-1-isopropyl-4-oxo-N-(5-((7-(trifluoromethoxy)quinolin-4-yl)oxy)pyridin-2-yl)-1,4-dihydropyridine-3-carboxamide

Mass spectrum: m/z=579.2 (M+H). ¹H NMR (CDCl₃) δ 13.33 (s, 1H), 8.73 (d, J=2.5 Hz, 1H), 8.69 (d, J=5.3 Hz, 1H), 8.46 (dd, J=9.0, 0.6 Hz, 1H), 8.42 (d, J=9.2 Hz, 1H), 8.30 (dd, J=2.9, 0.6 Hz, 1H), 7.94 (m, 1H), 7.63 (m, 2H), 7.58 (d, J=2.5 Hz, 1H), 7.56 (dd, J=9.0, 2.8 Hz, 1H), 7.46 (m, 1H), 7.14 (m, 2H), 6.58 (d, J=5.3 Hz, 1H), 4.33 (m, 1H), 1.62 (d, J=6.8 Hz, 6H).

### Example 51

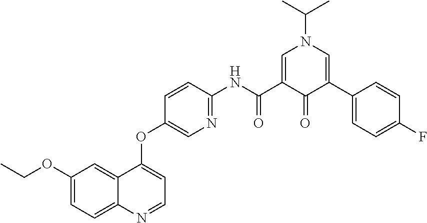 [SMILES: CCOc1ccc2nccc(Oc3ccc(NC(=O)c4cn(C(C)C)cc(-c5ccc(F)cc5)c4=O)nc3)c2c1]

### N-(5-((6-ethoxyquinolin-4-yl)oxy)pyridin-2-yl)-5-(4-fluorophenyl)-1-isopropyl-4-oxo-1,4-dihydropyridine-3-carboxamide

Mass spectrum: m/z=539.2 (M+H). ¹H NMR (CDCl₃) δ 13.30 (s, 1H), 8.69 (d, J=2.5 Hz, 1H), 8.55 (d, J=5.3 Hz, 1H), 8.43 (dd, J=9.0, 0.6 Hz, 1H), 8.30 (dd, J=2.9, 0.6 Hz, 1H), 8.00 (d, J=9.2 Hz, 1H), 7.63 (m, 2H), 7.58 (d, J=2.5 Hz, 1H), 7.57 (d, J=2.7 Hz, 1H), 7.55 (dd, J=9.0, 2.9 Hz, 1H), 7.41 (dd, J=9.2, 2.9 Hz, 1H), 7.14 (m, 2H), 6.55 (d, J=5.3 Hz, 1H), 4.33 (m, 1H), 4.21 (q, J=7.0 Hz, 2H), 1.62 (d, J=6.8 Hz, 6H), 1.50 (t, J=6.8 Hz, 3H).

### Example 52

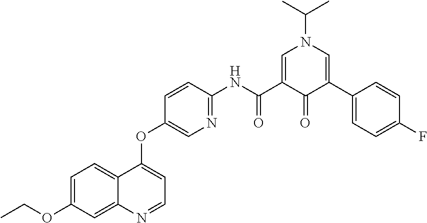 [SMILES: CCOc1ccc2c(Oc3ccc(NC(=O)c4cn(C(C)C)cc(-c5ccc(F)cc5)c4=O)nc3)ccnc2c1]

### N-(5-((7-ethoxyquinolin-4-yl)oxy)pyridin-2-yl)-5-(4-fluorophenyl)-1-isopropyl-4-oxo-1,4-dihydropyridine-3-carboxamide

Mass spectrum: m/z=539.2 (M+H). ¹H NMR (CDCl₃) δ 13.30 (s, 1H), 8.69 (d, J=2.5 Hz, 1H), 8.62 (d, J=5.3 Hz, 1H), 8.43 (dd, J=9.0, 0.6 Hz, 1H), 8.29 (dd, J=2.9, 0.6 Hz, 1H), 8.24 (d, J=9.2 Hz, 1H), 7.63 (m, 2H), 7.58 (d, J=2.5 Hz, 1H), 7.54 (dd, J=9.0, 2.9 Hz, 1H), 7.44 (d, J=2.3 Hz, 1H), 7.24 (dd, J=9.0, 2.3 Hz, 1H), 7.14 (m, 2H), 6.45 (d, J=5.3 Hz, 1H), 4.33 (m, 1H), 4.22 (q, J=7.0 Hz, 2H), 1.62 (d, J=6.7 Hz, 6H), 1.51 (t, J=7.0 Hz, 3H).

### Example 53

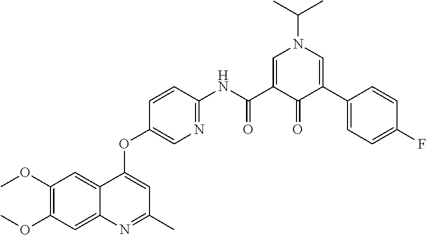 [SMILES: COc1cc2nc(C)cc(Oc3ccc(NC(=O)c4cn(C(C)C)cc(-c5ccc(F)cc5)c4=O)nc3)c2cc1OC]

### N-(5-((6,7-dimethoxy-2-methylquinolin-4-yl)oxy)pyridin-2-yl)-5-(4-fluorophenyl)-1-isopropyl-4-oxo-1,4-dihydropyridine-3-carboxamide

Mass spectrum: m/z=569.2 (M+H). ¹H NMR (d₆-DMSO) δ 13.42 (s, 1H), 8.79 (d, J=2.2 Hz, 1H), 8.43 (d, J=8.8 Hz, 1H), 8.36 (d, J=2.7 Hz, 1H), 8.27 (d, J=1.7 Hz, 1H), 7.84 (dd, J=8.8, 2.4 Hz, 1H), 7.75 (m, 3H), 7.48 (s,1H), 7.34 (s, 1H), 7.29 (m, 2H), 6.47 (s, 1H), 4.65 (m, 1H), 3.93 (s, 3H), 3.92 (s, 3H),2.46 (s, 3H), 1.52 (d, J=6.6 Hz, 6H).

### Example 54

### N-(5-((6,7-dimethoxyquinolin-4-yl)oxy)pyrimidin-2-yl)-5-(4-fluorophenyl)-1-isopropyl-4-oxo-1,4-dihydropyridine-3-carboxamide

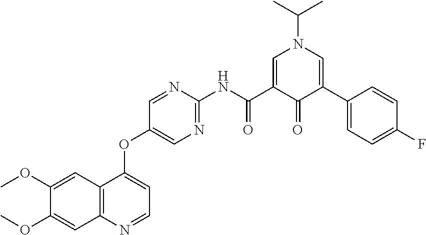 [SMILES: COc1cc2nccc(Oc3cnc(NC(=O)c4cn(C(C)C)cc(-c5ccc(F)cc5)c4=O)nc3)c2cc1OC]

A solution of 5-((6,7-dimethoxyquinolin-4-yl)oxy)pyrimidin-2-amine (Preparation 47; 0.025 g, 0.0838 mmol), 5-(4-fluorophenyl)-1-isopropyl-4-oxo-1,4-dihydropyridine-3-carboxylic acid (Preparation 2; 0.0346 g, 0.126 mmol), DIEA (0.0439 mL, 0.251 mmol) and HATU (0.0637 g, 0.168 mmol) in DMF (0.8 mL) was stirred at room temperature overnight. Additional DIEA (2 equivalents) and HATU (1 equivalent) was added and the reaction mixture was heated to 50° C. for 4 days. The reaction was cooled and poured into water (30 mL) and stirred for 10 min. The resultant solids were filtered and washed with water. The solids were suspended in 3 mL of a solution of 3:2 ACN:water with 2% TFA additive and then purified over a C18 column (5-95% ACN:water gradient with 0.1% TFA additive). Fractions containing the desired product were concentrated in vacuo and dried under high vacuum to provide N-(5-((6,7-dimethoxyquinolin-4-yl)oxy)pyrimidin-2-yl)-5-(4-fluorophenyl)-1-isopropyl-4-oxo-1,4-dihydropyridine-3-carboxamide (0.0134 g, 0.0241 mmol, 28.8% yield) as a slightly yellowish white solid. Mass spectrum: m/z=556.2 (M+H). ¹H NMR (CDCl₃) δ 13.64 (s, 1H), 8.72 (d, J=2.5 Hz, 1H), 8.61 (s, 2H), 8.54 (d, J=5.3 Hz, 1H), 7.65-7.58 (m, 3H), 7.53 (s, 1H), 7.45 (s, 1H), 7.15 (m, 2H), 6.48 (d, J=5.3 Hz, 1H), 4.36 (m, 1H), 4.07 (s, 3H), 4.06 (s, 3H), 1.62 (d, J=6.8 Hz, 6H).

### Example 55

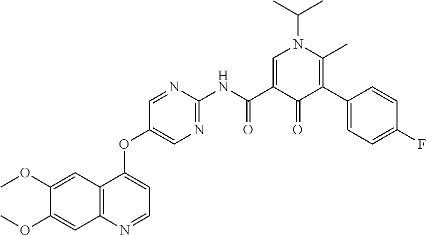 [SMILES: COc1cc2nccc(Oc3cnc(NC(=O)c4cn(C(C)C)c(C)c(-c5ccc(F)cc5)c4=O)nc3)c2cc1OC]

### N-(5-((6,7-dimethoxyquinolin-4-yl)oxy)pyrimidin-2-yl)-5-(4-fluorophenyl)-1-isopropyl-6-methyl-4-oxo-1,4-dihydropyridine-3-carboxamide

Prepared according to the procedure of Example 54. Mass spectrum: m/z=570.2 (M+H). ¹H NMR (CDCl₃) δ 13.64 (s, 1H), 8.72 (d, J=2.5 Hz, 1H), 8.61 (s, 2H), 8.54 (d, J=5.3 Hz, 1H), 7.62 (m, 2H), 7.59 (d, J=2.5 Hz, 1H), 7.53 (s, 1H), 7.45 (s, 1H), 7.15 (m, 2H), 6.48 (d, J=5.3 Hz, 1H), 4.36 (m, 1H), 4.07 (s, 3H), 4.06 (s, 3H), 1.62 (d, J=6.8 Hz, 6H).

### Example 56

### 5-(4-fluorophenyl)-1-isopropyl-N-(5-((6-methoxy-7-(3-morpholinopropoxy)quinolin-4-yl)oxy)pyridin-2-yl)-4-oxo-1,4-dihydropyridine-3-carboxamide

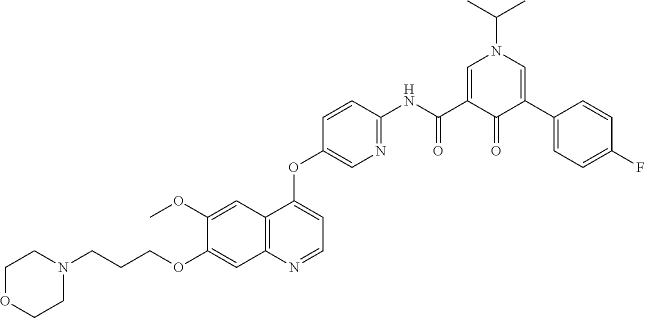 [SMILES: COc1cc2c(Oc3ccc(NC(=O)c4cn(C(C)C)cc(-c5ccc(F)cc5)c4=O)nc3)ccnc2cc1OCCCN1CCOCC1]

A solution of 5-((6-methoxy-7-(3-morpholinopropoxy)quinolin-4-yl)oxy)pyridin-2-amine (Preparation 48; 0.035 g, 0.0853 mmol), 5-(4-fluorophenyl)-1-isopropyl-4-oxo-1,4-dihydropyridine-3-carboxylic acid (Preparation 2; 0.0352 g, 0.128 mmol), DIEA (0.0447 mL, 0.256 mmol) and HATU (0.0648 g, 0.171 mmol) in DMF (0.8 mL) was stirred for 3 hours. The reaction was added to cold water (30 mL) with stirring and a white solid precipitated out of solution. The solids were isolated by filtration and washed with water (15 mL) and then air dried. The solids were suspended in 3 mL of a solution of 3:2 ACN:water with 2% TFA additive and purified using a preparatory HPLC column (5% up to >95% ACN:water gradient with 0.1% TFA additive). Fractions containing the desired product were treated with saturated NaHCO₃ (15 mL) and then extracted with DCM (2×15 mL) to provide the product in free base form. The combined organic layers were washed with brine (15 mL), dried over Na₂SO₄, filtered and concentrated in vacuo to provide 5-(4-fluorophenyl)-1-isopropyl-N-(5-((6-methoxy-7-(3-morpholinopropoxy)quinolin-4-yl)oxy)pryridin-2-yl)-4-oxo-1,4-dihydropyridine-3-carboxamide (0.0111 g, 0.0166 mmol, 19.5 yield) as a white solid. Mass spectrum: m/z=668.3 (M+H). ¹H NMR (d₆-DMSO) δ 13.41 (s, 1H), 8.79 (d, J=1.7 Hz, 1H), 8.49 (d, J=5.1 Hz, 1H), 8.43 (d, J=9.0 Hz, 1H), 8.37 (d, J=2.9 Hz, 1H), 8.27 (d, J=1.7 Hz, 1H), 7.85 (dd, J=9.0, 2.4 Hz, 1H), 7.75 (m, 2H), 7.54 (s, 1H), 7.41 (s, 1H), 7.29 (t, J=8.8 Hz, 2H), 6.56 (d, J=5.1 Hz, 1H), 4.65 (m, 1H), 4.21 (t, J=6.1 Hz, 2H), 3.95 (s, 3H), 3.59 (m, 4H), 2.50-2.35 (m, 6H), 1.98 (m, 2H), 1.52 (d, J=6.6 Hz, 6H).

### Example 57

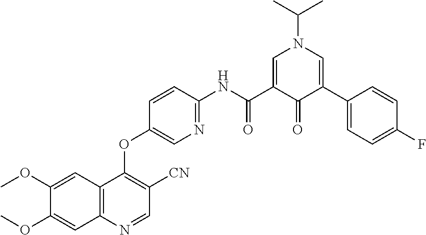 [SMILES: COc1cc2ncc(C#N)c(Oc3ccc(NC(=O)c4cn(C(C)C)cc(-c5ccc(F)cc5)c4=O)nc3)c2cc1OC]

### N-(5-((3-cyano-6,7-dimethoxyquinolin-4-yl)oxy)pyridin-2-yl)-5-(4-fluorophenyl)-1-isopropyl-4-oxo-1,4-dihydropyridine-3-carboxamide

Prepared according to the procedure of Example 56. Mass spectrum: m/z=580.2 (M+H). ¹H NMR (d₆-DMSO) δ 13.36 (s, 1H), 8.88 (s, 1H), 8.77 (d, J=2.0 Hz, 1H), 8.36 (d, J=2.9 Hz, 1H), 8.34 (d, J=9.0 Hz, 1H), 8.26 (d, J=1.7 Hz, 1H), 7.76-7.72 (m, 3H), 7.55 (s, 1H), 7.44 (s, 1H), 7.28 (t, J=8.8 Hz, 2H), 4.63 (m, 1H), 4.01 (s, 3H), 3.89 (s, 3H), 1.51 (d, J=6.3 Hz, 6H)

### Example 58

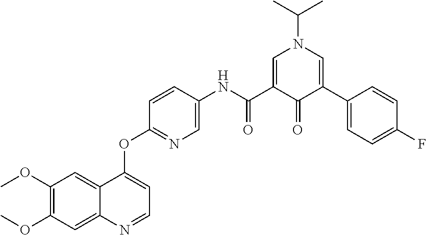 [SMILES: COc1cc2nccc(Oc3ccc(NC(=O)c4cn(C(C)C)cc(-c5ccc(F)cc5)c4=O)cn3)c2cc1OC]

### N-(6-((6,7-dimethoxyquinolin-4-yl)oxy)pyridin-3-yl)-5-(4-fluorophenyl)-1-isopropyl-4-oxo-1,4-dihydropyridine-3-carboxamide

Prepared according to the procedure of Example 56. Mass spectrum: m/z=555.2 (M+H). ¹H NMR (CDCl₃) δ 12.96 (s, 1H), 8.71 (d, J=2.5 Hz, 1H), 8.66 (d, J=2.7 Hz, 1H), 8.60 (d, J=5.3 Hz, 1H), 8.36 (dd, J=8.8, 2.7 Hz, 1H), 7.59 (d, J=2.5 Hz, 1H), 7.56 (m, 2H), 7.48 (s, 1H), 7.44 (s, 1H), 7.17 (m, 2H), 7.12 (d, J=8.6 Hz, 1H), 6.80 (d, J=5.1 Hz, 1H), 4.34 (m, 1H), 4.05 (s, 3H), 4.01 (s, 3H), 1.62 (d, J=6.7 Hz, 6H).

### Abbreviations:

| | |
|---|---|
| DCM | Dichloromethane |
| DIEA | N,N-Diisopropylethylamine |
| DMAP | 4-Dimethylaminopyridine |
| DMF | N,N-Dimethylformamide |
| EtOAc | Ethyl Acetate |
| EtOH | Ethanol |
| eq | equivalent |
| h | hour, hours |
| HATU | (1-[Bis(dimethylamino)methylene]- |
| | 1H-1,2,3-triazolo[4,5-b]pyridinium |
| | 3-oxid hexafluorophosphate; |
| | Hexafluorophosphate Azabenzotriazole |
| | Tetramethyl Uronium) |
| LiHMDS | Lithium bis(trimethylsilyl)amide; |
| | lithium hexamethyldisilamide |
| min | minute, minutes |
| NBM | N-bromosuccinimide |
| Pd(PPh₃)₄ | Tetrakis(triphenylphosphine)palladium(0) |
| THF | tetrahydrofuran |

### Exemplary Embodiments

Embodiment 1. A compound of Formula I, wherein the compound is a compound of Example No. 1, 2, 3, 4, 7, 18, 19, 20, 27, 28, 29, 32, 33, 44, 46, 48, 55, 56, or 58, or a pharmaceutically acceptable salt or solvate thereof.

Embodiment 1A. A compound of Formula I, wherein the compounds is a compound of Example No. 1, 4, 5, 16, 18, 19, 21, 28, 30, 31, 33, 35, 39, 41, 48, 49, 51, 52, 57 or 58, or a pharmaceutically acceptable salt or solvate thereof.

Embodiment 2. A pharmaceutical combination which comprises (a) a compound of Formula I or a pharmaceutically acceptable salt thereof, and (b) an additional therapeutic agent.

Embodiment 3. A pharmaceutical combination which comprises (a) a compound of Formula I or a pharmaceutically acceptable salt thereof, and (b) an additional therapeutic agent, for use in therapy.

Embodiment 4. The pharmaceutical combination of Embodiment 2, or for use of Embodiment 3, wherein the compound of Formula I or the pharmaceutically acceptable salt thereof and the additional therapeutic agent are formulated as separate compositions or dosages for simultaneous, separate or sequential use for use in therapy, wherein the amounts of the compound of Formula I or a pharmaceutically acceptable salt thereof and of the additional therapeutic agent are together therapeutically effective.

Embodiment 5. A pharmaceutical composition, comprising (a) a compound of Formula I or a pharmaceutically acceptable salt thereof, (b) an additional therapeutic agent, and (c) a pharmaceutically acceptable diluent or carrier.

Embodiment 6. The pharmaceutical combination of Embodiment 2, or for use of Embodiment 3, or the pharmaceutical composition of Embodiment 5, wherein the additional therapeutic agent is an anticancer agent.

Embodiment 7. The pharmaceutical combination of Embodiment 2, or for use of Embodiment 3, or the pharmaceutical composition of Embodiment 5, wherein the additional therapeutic agent is binimetinib and encorafenib.

Embodiment 8. The pharmaceutical combination of Embodiment 2, or for use of Embodiment 3, or the pharmaceutical composition of Embodiment 5, wherein the additional therapeutic agent is binimetinib.

Embodiment 9. The pharmaceutical combination of Embodiment 2, or for use of Embodiment 3, or the pharmaceutical composition of Embodiment 5, wherein the additional therapeutic agent is encorafenib.

Embodiment 10. The pharmaceutical combination of Embodiment 2, or for use of Embodiment 3, or the pharmaceutical composition of Embodiment 5, wherein the additional therapeutic agent is selumetinib.

Embodiment 11. The pharmaceutical combination of Embodiment 2, or for use of Embodiment 3, or the pharmaceutical composition of Embodiment 5, wherein the additional therapeutic agent is sorafenib.

Embodiment 12. The pharmaceutical combination of Embodiment 2, or for use of Embodiment 3, or the pharmaceutical composition of Embodiment 5, wherein the additional therapeutic agent is trametinib.

Embodiment 13. The pharmaceutical combination of Embodiment 2, or for use of Embodiment 3, or the pharmaceutical composition of Embodiment 5, wherein the additional therapeutic agent is vemurafenib.

Embodiment 14. The pharmaceutical combination of Embodiment 2, or for use of Embodiment 3, or the pharmaceutical composition of Embodiment 5, wherein the additional therapeutic agent is an EGFR inhibitor.

Embodiment 15. The pharmaceutical combination of Embodiment 2, or for use of Embodiment 3, or the pharmaceutical composition of Embodiment 5, wherein the additional therapeutic agent is cetuximab or a biosimilar thereof.

Embodiment 16. The pharmaceutical combination of Embodiment 2, or for use of Embodiment 3, or the pharmaceutical composition of Embodiment 5, wherein the additional therapeutic agent is panitumumab or a biosimilar thereof.

Embodiment 17. The pharmaceutical combination of Embodiment 2, or for use of Embodiment 3, or the pharmaceutical composition of Embodiment 5, wherein the additional therapeutic agent is erlotinib.

Embodiment 18. The pharmaceutical combination of Embodiment 2, or for use of Embodiment 3, or the pharmaceutical composition of Embodiment 5, wherein the additional therapeutic agent is lapatinib.

Embodiment 19. The pharmaceutical combination of Embodiment 2, or for use of Embodiment 3, or the pharmaceutical composition of Embodiment 5, wherein the additional therapeutic agent is gefitinib.

Embodiment 20. The pharmaceutical combination of Embodiment 2, or for use of Embodiment 3, or the pharmaceutical composition of Embodiment 5, wherein the additional therapeutic agent is a checkpoint inhibitor.

Embodiment 21. The pharmaceutical combination of Embodiment 2, or for use of Embodiment 3, or the pharmaceutical composition of Embodiment 5, wherein the additional therapeutic agent is nivolumab or a biosimilar thereof.

Embodiment 22. The pharmaceutical combination of Embodiment 2, or for use of Embodiment 3, or the pharmaceutical composition of Embodiment 5, wherein the additional therapeutic agent is pembrolizumab or a biosimilar thereof.

Embodiment 23. The pharmaceutical combination of Embodiment 2, or for use of Embodiment 3, or the pharmaceutical composition of Embodiment 5, wherein the additional therapeutic agent is cemiplimab or a biosimilar thereof.

Embodiment 24. The pharmaceutical combination of Embodiment 2, or for use of Embodiment 3, or the pharmaceutical composition of Embodiment 5, wherein the additional therapeutic agent is pidilizumab or a biosimilar thereof.

Embodiment 25. The pharmaceutical combination of Embodiment 2, or for use of Embodiment 3, or the pharmaceutical composition of Embodiment 5, wherein the additional therapeutic agent is atezolizumab or a biosimilar thereof.

Embodiment 26. The pharmaceutical combination of Embodiment 2, or for use of Embodiment 3, or the pharmaceutical composition of Embodiment 5, wherein the additional therapeutic agent is avelumab or a biosimilar thereof.

Embodiment 27. The pharmaceutical combination of Embodiment 2, or for use of Embodiment 3, or the pharmaceutical composition of Embodiment 5, wherein the additional therapeutic agent is durvalumab or a biosimilar thereof.

Embodiment 28. A pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt thereof, and (b) an additional therapeutic agent.

Embodiment 28. A pharmaceutical combination which comprises (a) a compound of Example No. 1, 4, 5, 16, 18, 19, 21, 28, 30, 31, 33, 35, 39, 41, 48, 49, 51, 52, 57 or 58, or a pharmaceutically acceptable salt or solvate thereof.

Embodiment 29. A pharmaceutical combination which comprises (a) a compound of Example No. 1, 2, 3, 4, 7, 18, 19, 20, 27, 28, 29, 32, 33, 44, 46, 48, 55, 56, or 58, or a pharmaceutically acceptable salt thereof, and (b) an additional therapeutic agent, for use in therapy.

Embodiment 29A. A pharmaceutical combination which comprises (a) a compound of Example No. 1, 4, 5, 16, 18, 19, 21, 28, 30, 31, 33, 35, 39, 41, 48, 49, 51, 52, 57 or 58, or a pharmaceutically acceptable salt thereof, for use in therapy.

Embodiment 30. The pharmaceutical combination of Embodiment 28, or for use of Embodiment 29, wherein the compound of Example No. 1, 2, 3, 4, 7, 18, 19, 20, 27, 28, 29, 32, 33, 44, 46, 48, 55, 56, or 58, or the pharmaceutically acceptable salt thereof and the additional therapeutic agent are formulated as separate compositions or dosages for simultaneous, separate or sequential use for use in therapy, wherein the amounts of the compound of Example No. 1, 2, 3, 4, 7, 18, 19, 20, 27, 28, 29, 32, 33, 44, 46, 48, 55, 56, or 58, or a pharmaceutically acceptable salt thereof and of the additional therapeutic agent are together therapeutically effective.

Embodiment 30A. The pharmaceutical combination of Embodiment 28A, or for use of Embodiment 29A, wherein the compound of Example No. 1, 4, 5, 16, 18, 19, 21, 28, 30, 31, 33, 35, 39, 41, 48, 49, 51, 52, 57 or 58, or the pharmaceutically acceptable salt thereof, and the additional therapeutic agent are formulated as separate compositions or dosages for simultaneous, separate or sequential use for use in therapy, wherein the amounts of the compound of Example No. 1, 4, 5, 16, 18, 19, 21, 28, 30, 31, 33, 35, 39, 41, 48, 49, 51, 52, 57 or 58, or the pharmaceutically acceptable salt thereof, and of the additional therapeutic agent are together therapeutically effective.

Embodiment 31. A pharmaceutical composition, comprising (a) a compound of Example No. 1, 2, 3, 4, 7, 18, 19, 20, 27, 28, 29, 32, 33, 44, 46, 48, 55, 56, or 58, or a pharmaceutically acceptable salt thereof, (b) an additional therapeutic agent, and (c) a pharmaceutically acceptable diluent or carrier.

Embodiment 32. The pharmaceutical combination of Embodiment 28 or 28A, or for use of Embodiment 29 or 29B, or the pharmaceutical composition of Embodiment 31, wherein the additional therapeutic agent is an anticancer agent.

Embodiment 33. The pharmaceutical combination of Embodiment 28or 28A, or for use of Embodiment 29 or 29B, or the pharmaceutical composition of Embodiment 31, wherein the additional therapeutic agent is binimetinib and encorafenib.

Embodiment 34. The pharmaceutical combination of Embodiment 28or 28A, or for use of Embodiment 29 or 29B, or the pharmaceutical composition of Embodiment 31, wherein the additional therapeutic agent is binimetinib.

Embodiment 35. The pharmaceutical combination of Embodiment 28or 28A, or for use of Embodiment 29 or 29B, or the pharmaceutical composition of Embodiment 31, wherein the additional therapeutic agent is encorafenib.

Embodiment 36. The pharmaceutical combination of Embodiment 28or 28A, or for use of Embodiment 29 or 29B, or the pharmaceutical composition of Embodiment 31, wherein the additional therapeutic agent is selumetinib.

Embodiment 37. The pharmaceutical combination of Embodiment 28 or 28A, or for use of Embodiment 29 or 29B, or the pharmaceutical composition of Embodiment 31, wherein the additional therapeutic agent is sorafenib.

Embodiment 38. The pharmaceutical combination of Embodiment 28 or 28A, or for use of Embodiment 29 or 29B, or the pharmaceutical composition of Embodiment 31, wherein the additional therapeutic agent is trametinib.

Embodiment 39. The pharmaceutical combination of Embodiment 28 or 28A, or for use of Embodiment 29 or 29B, or the pharmaceutical composition of Embodiment 31, wherein the additional therapeutic agent is vemurafenib.

Embodiment 40. The pharmaceutical combination of Embodiment 28 or 28A, or for use of Embodiment 29 or 29B, or the pharmaceutical composition of Embodiment 31, wherein the additional therapeutic agent is an EGFR inhibitor.

Embodiment 41. The pharmaceutical combination of Embodiment 28 or 28A, or for use of Embodiment 29 or 29B, or the pharmaceutical composition of Embodiment 31, wherein the additional therapeutic agent is cetuximab or a biosimilar thereof.

Embodiment 42. The pharmaceutical combination of Embodiment 28 or 28A, or for use of Embodiment 29 or 29B, or the pharmaceutical composition of Embodiment 31, wherein the additional therapeutic agent is panitumumab or a biosimilar thereof.

Embodiment 43. The pharmaceutical combination of Embodiment 28 or 28A, or for use of Embodiment 29 or 29B, or the pharmaceutical composition of Embodiment 31, wherein the additional therapeutic agent is erlotinib.

Embodiment 44. The pharmaceutical combination of Embodiment 28 or 28A, or for use of Embodiment 29 or 29B, or the pharmaceutical composition of Embodiment 31, wherein the additional therapeutic agent is lapatinib.

Embodiment 45. The pharmaceutical combination of Embodiment 28 or 28A, or for use of Embodiment 29 or 29B, or the pharmaceutical composition of Embodiment 31, wherein the additional therapeutic agent is gefitinib.

Embodiment 46. The pharmaceutical combination of Embodiment 28 or 28A, or for use of Embodiment 29 or 29B, or the pharmaceutical composition of Embodiment 31, wherein the additional therapeutic agent is a checkpoint inhibitor.

Embodiment 47. The pharmaceutical combination of Embodiment 28 or 28A, or for use of Embodiment 29 or 29B, or the pharmaceutical composition of Embodiment 31, wherein the additional therapeutic agent is nivolumab or a biosimilar thereof.

Embodiment 48. The pharmaceutical combination of Embodiment 28 or 28A, or for use of Embodiment 29 or 29B, or the pharmaceutical composition of Embodiment 31, wherein the additional therapeutic agent is pembrolizumab or a biosimilar thereof.

Embodiment 49. The pharmaceutical combination of Embodiment 28 or 28A, or for use of Embodiment 29 or 29B, or the pharmaceutical composition of Embodiment 31, wherein the additional therapeutic agent is cemiplimab or a biosimilar thereof.

Embodiment 50. The pharmaceutical combination of Embodiment 28 or 28A, or for use of Embodiment 29 or 29B, or the pharmaceutical composition of Embodiment 31, wherein the additional therapeutic agent is pidilizumab or a biosimilar thereof.

Embodiment 51. The pharmaceutical combination of Embodiment 28 or 28A, or for use of Embodiment 29 or 29B, or the pharmaceutical composition of Embodiment 31, wherein the additional therapeutic agent is atezolizumab or a biosimilar thereof.

Embodiment 52. The pharmaceutical combination of Embodiment 28 or 28A, or for use of Embodiment 29 or 29B, or the pharmaceutical composition of Embodiment 31, wherein the additional therapeutic agent is avelumab or a biosimilar thereof.

Embodiment 53. The pharmaceutical combination of Embodiment 28 or 28A, or for use of Embodiment 29 or 29B, or the pharmaceutical composition of Embodiment 31, wherein the additional therapeutic agent is durvalumab or a biosimilar thereof.

Embodiment 54. A pharmaceutical combination which comprises (a) a compound of Example No. 1, or a pharmaceutically acceptable salt thereof, and (b) an additional therapeutic agent.

Embodiment 55. A pharmaceutical combination which comprises (a) a compound of Example No. 1, or a pharmaceutically acceptable salt thereof, and (b) an additional therapeutic agent, for use in therapy.

Embodiment 56. The pharmaceutical combination of Embodiment 54, or for use of Embodiment 55, wherein the compound of Example No. 1, or the pharmaceutically acceptable salt thereof and the additional therapeutic agent are formulated as separate compositions or dosages for simultaneous, separate or sequential use for use in therapy, wherein the amounts of the compound of Example No. 1 or a pharmaceutically acceptable salt thereof and of the additional therapeutic agent are together therapeutically effective.

Embodiment 57. A pharmaceutical composition, comprising (a) a compound of Example No. 1, or a pharmaceutically acceptable salt thereof, (b) an additional therapeutic agent, and (c) a pharmaceutically acceptable diluent or carrier.

Embodiment 58. The pharmaceutical combination of Embodiment 54, or for use of Embodiment 55, or the pharmaceutical composition of Embodiment 57, wherein the additional therapeutic agent is an anticancer agent.

Embodiment 59. The pharmaceutical combination of Embodiment 54, or for use of Embodiment 55, or the pharmaceutical composition of Embodiment 57, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 60. A pharmaceutical combination which comprises (a) a compound of Example No. 2, or a pharmaceutically acceptable salt thereof, and (b) an additional therapeutic agent.

Embodiment 61. A pharmaceutical combination which comprises (a) a compound of Example No. 2, or a pharmaceutically acceptable salt thereof, and (b) an additional therapeutic agent, for use in therapy.

Embodiment 62. The pharmaceutical combination of Embodiment 60, or for use of Embodiment 61, wherein the compound of Example No. 2, or the pharmaceutically acceptable salt thereof and the additional therapeutic agent are formulated as separate compositions or dosages for simultaneous, separate or sequential use for use in therapy, wherein the amounts of the compound of Example No. 2, or a pharmaceutically acceptable salt thereof and of the additional therapeutic agent are together therapeutically effective.

Embodiment 63. A pharmaceutical composition, comprising (a) a compound of Example No. 2, or a pharmaceutically acceptable salt thereof, (b) an additional therapeutic agent, and (c) a pharmaceutically acceptable diluent or carrier.

Embodiment 64. The pharmaceutical combination of Embodiment 60, or for use of Embodiment 61, or the pharmaceutical composition of Embodiment 63, wherein the additional therapeutic agent is an anticancer agent.

Embodiment 65. The pharmaceutical combination of Embodiment 60, or for use of Embodiment 61, or the pharmaceutical composition of Embodiment 63, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 66. A pharmaceutical combination which comprises (a) a compound of Example No. 3, or a pharmaceutically acceptable salt thereof, and (b) an additional therapeutic agent.

Embodiment 67. A pharmaceutical combination which comprises (a) a compound of Example No. 3, or a pharmaceutically acceptable salt thereof, and (b) an additional therapeutic agent, for use in therapy.

Embodiment 68. The pharmaceutical combination of Embodiment 66, or for use of Embodiment 67, wherein the compound of Example No. 3, or the pharmaceutically acceptable salt thereof and the additional therapeutic agent are formulated as separate compositions or dosages for simultaneous, separate or sequential use for use in therapy, wherein the amounts of the compound of Example No. 3, or a pharmaceutically acceptable salt thereof and of the additional therapeutic agent are together therapeutically effective.

Embodiment 69. A pharmaceutical composition, comprising (a) a compound of Example No. 3, or a pharmaceutically acceptable salt thereof, (b) an additional therapeutic agent, and (c) a pharmaceutically acceptable diluent or carrier.

Embodiment 70. The pharmaceutical combination of Embodiment 66, or for use of Embodiment 67, or the pharmaceutical composition of Embodiment 69, wherein the additional therapeutic agent is an anticancer agent.

Embodiment 71. The pharmaceutical combination of Embodiment 66, or for use of Embodiment 67, or the pharmaceutical composition of Embodiment 70, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 72. A pharmaceutical combination which comprises (a) a compound of Example No. 4, or a pharmaceutically acceptable salt thereof, and (b) an additional therapeutic agent.

Embodiment 73. A pharmaceutical combination which comprises (a) a compound of Example No. 4, or a pharmaceutically acceptable salt thereof, and (b) an additional therapeutic agent, for use in therapy.

Embodiment 74. The pharmaceutical combination of Embodiment 72, or for use of Embodiment 73, wherein the compound of Example No. 4, or the pharmaceutically acceptable salt thereof and the additional therapeutic agent are formulated as separate compositions or dosages for simultaneous, separate or sequential use for use in therapy, wherein the amounts of the compound of Example No. 4, or a pharmaceutically acceptable salt thereof and of the additional therapeutic agent are together therapeutically effective.

Embodiment 75. A pharmaceutical composition, comprising (a) a compound of Example No. 4, or a pharmaceutically acceptable salt thereof, (b) an additional therapeutic agent, and (c) a pharmaceutically acceptable diluent or carrier.

Embodiment 76. The pharmaceutical combination of Embodiment 73, or for use of Embodiment 74, or the pharmaceutical composition of Embodiment 75, wherein the additional therapeutic agent is an anticancer agent.

Embodiment 77. The pharmaceutical combination of Embodiment 73, or for use of Embodiment 74, or the pharmaceutical composition of Embodiment 76, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 78. A pharmaceutical combination which comprises (a) a compound of Example No. 7, or a pharmaceutically acceptable salt thereof, and (b) an additional therapeutic agent.

Embodiment 79. A pharmaceutical combination which comprises (a) a compound of Example No. 7, or a pharmaceutically acceptable salt thereof, and (b) an additional therapeutic agent, for use in therapy.

Embodiment 80. The pharmaceutical combination of Embodiment 78, or for use of Embodiment 79, wherein the compound of Example No. 7, or the pharmaceutically acceptable salt thereof and the additional therapeutic agent are formulated as separate compositions or dosages for simultaneous, separate or sequential use for use in therapy, wherein the amounts of the compound of Example No. 7, or a pharmaceutically acceptable salt thereof and of the additional therapeutic agent are together therapeutically effective.

Embodiment 81. A pharmaceutical composition, comprising (a) a compound of Example No. 7, or a pharmaceutically acceptable salt thereof, (b) an additional therapeutic agent, and (c) a pharmaceutically acceptable diluent or carrier.

Embodiment 82. The pharmaceutical combination of Embodiment 78, or for use of Embodiment 79, or the pharmaceutical composition of Embodiment 81, wherein the additional therapeutic agent is an anticancer agent.

Embodiment 83. The pharmaceutical combination of Embodiment 78, or for use of Embodiment 79, or the pharmaceutical composition of Embodiment 81, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 84. A pharmaceutical combination which comprises (a) a compound of Example No. 18, or a pharmaceutically acceptable salt thereof, and (b) an additional therapeutic agent . Embodiment 85. A pharmaceutical combination which comprises (a) a compound of Example No. 18, or a pharmaceutically acceptable salt thereof, and (b) an additional therapeutic agent, for use in therapy.

Embodiment 86. The pharmaceutical combination of Embodiment 84, or for use of Embodiment 85, wherein the compound of Example No. 18, or the pharmaceutically acceptable salt thereof and the additional therapeutic agent are formulated as separate compositions or dosages for simultaneous, separate or sequential use for use in therapy, wherein the amounts of the compound of Example No. 18, or a pharmaceutically acceptable salt thereof and of the additional therapeutic agent are together therapeutically effective.

Embodiment 87. A pharmaceutical composition, comprising (a) a compound of Example No. 18, or a pharmaceutically acceptable salt thereof, (b) an additional therapeutic agent, and (c) a pharmaceutically acceptable diluent or carrier.

Embodiment 88. The pharmaceutical combination of Embodiment 84, or for use of Embodiment 85, or the pharmaceutical composition of Embodiment 87, wherein the additional therapeutic agent is an anticancer agent.

Embodiment 89. The pharmaceutical combination of Embodiment 84, or for use of Embodiment 85, or the pharmaceutical composition of Embodiment 87, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 90. A pharmaceutical combination which comprises (a) a compound of Example No. 19, or a pharmaceutically acceptable salt thereof, and (b) an additional therapeutic agent.

Embodiment 91. A pharmaceutical combination which comprises (a) a compound of Example No. 19, or a pharmaceutically acceptable salt thereof, and (b) an additional therapeutic agent, for use in therapy.

Embodiment 92. The pharmaceutical combination of Embodiment 90, or for use of Embodiment 91, wherein the compound of Example No. 19, or the pharmaceutically acceptable salt thereof and the additional therapeutic agent are formulated as separate compositions or dosages for simultaneous, separate or sequential use for use in therapy, wherein the amounts of the compound of Example No. 19, or a pharmaceutically acceptable salt thereof and of the additional therapeutic agent are together therapeutically effective.

Embodiment 93. A pharmaceutical composition, comprising (a) a compound of Example No. 19, or a pharmaceutically acceptable salt thereof, (b) an additional therapeutic agent, and (c) a pharmaceutically acceptable diluent or carrier.

Embodiment 94. The pharmaceutical combination of Embodiment 90, or for use of Embodiment 91, or the pharmaceutical composition of Embodiment 93, wherein the additional therapeutic agent is an anticancer agent.

Embodiment 95. The pharmaceutical combination of Embodiment 90, or for use of Embodiment 91, or the pharmaceutical composition of Embodiment 93, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 96. A pharmaceutical combination which comprises (a) a compound of Example No. 20, or a pharmaceutically acceptable salt thereof, and (b) an additional therapeutic agent .

Embodiment 97. A pharmaceutical combination which comprises (a) a compound of Example No. 20, or a pharmaceutically acceptable salt thereof, and (b) an additional therapeutic agent, for use in therapy.

Embodiment 98. The pharmaceutical combination of Embodiment 96, or for use of Embodiment 97, wherein the compound of Example No. 20, or the pharmaceutically acceptable salt thereof and the additional therapeutic agent are formulated as separate compositions or dosages for simultaneous, separate or sequential use for use in therapy, wherein the amounts of the compound of Example No. 20, or a pharmaceutically acceptable salt thereof and of the additional therapeutic agent are together therapeutically effective.

Embodiment 99. A pharmaceutical composition, comprising (a) a compound of Example No. 20, or a pharmaceutically acceptable salt thereof, (b) an additional therapeutic agent, and (c) a pharmaceutically acceptable diluent or carrier.

Embodiment 100. The pharmaceutical combination of Embodiment 96, or for use of Embodiment 97, or the pharmaceutical composition of Embodiment 99, wherein the additional therapeutic agent is an anticancer agent.

Embodiment 101. The pharmaceutical combination of Embodiment 96, or for use of Embodiment 97, or the pharmaceutical composition of Embodiment 99, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 102. A pharmaceutical combination which comprises (a) a compound of Example No. 27, or a pharmaceutically acceptable salt thereof, and (b) an additional therapeutic agent .

Embodiment 103. A pharmaceutical combination which comprises (a) a compound of Example No. 27, or a pharmaceutically acceptable salt thereof, and (b) an additional therapeutic agent, for use in therapy.

Embodiment 104. The pharmaceutical combination of Embodiment 102, or for use of Embodiment 103, wherein the compound of Example No. 27, or the pharmaceutically acceptable salt thereof and the additional therapeutic agent are formulated as separate compositions or dosages for simultaneous, separate or sequential use for use in therapy, wherein the amounts of the compound of Example No. 27, or a pharmaceutically acceptable salt thereof and of the additional therapeutic agent are together therapeutically effective.

Embodiment 105. A pharmaceutical composition, comprising (a) a compound of Example No. 27, or a pharmaceutically acceptable salt thereof, (b) an additional therapeutic agent, and (c) a pharmaceutically acceptable diluent or carrier.

Embodiment 106. The pharmaceutical combination of Embodiment 102, or for use of Embodiment 103, or the pharmaceutical composition of Embodiment 105, wherein the additional therapeutic agent is an anticancer agent.

Embodiment 107. The pharmaceutical combination of Embodiment 102, or for use of Embodiment 103, or the pharmaceutical composition of Embodiment 105, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 108. A pharmaceutical combination which comprises (a) a compound of Example No. 28, or a pharmaceutically acceptable salt thereof, and (b) an additional therapeutic agent.

Embodiment 109. A pharmaceutical combination which comprises (a) a compound of Example No. 28, or a pharmaceutically acceptable salt thereof, and (b) an additional therapeutic agent, for use in therapy.

Embodiment 110. The pharmaceutical combination of Embodiment 108, or for use of Embodiment 109, wherein the compound of Example No. 28, or the pharmaceutically acceptable salt thereof and the additional therapeutic agent are formulated as separate compositions or dosages for simultaneous, separate or sequential use for use in therapy, wherein the amounts of the compound of Example No. 28, or a pharmaceutically acceptable salt thereof and of the additional therapeutic agent are together therapeutically effective.

Embodiment 111. A pharmaceutical composition, comprising (a) a compound of Example No. 28, or a pharmaceutically acceptable salt thereof, (b) an additional therapeutic agent, and (c) a pharmaceutically acceptable diluent or carrier.

Embodiment 112. The pharmaceutical combination of Embodiment 108, or for use of Embodiment 109, or the pharmaceutical composition of Embodiment 111, wherein the additional therapeutic agent is an anticancer agent.

Embodiment 113. The pharmaceutical combination of Embodiment 108, or for use of Embodiment 109, or the pharmaceutical composition of Embodiment 111, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 114. A pharmaceutical combination which comprises (a) a compound of Example No. 29, or a pharmaceutically acceptable salt thereof, and (b) an additional therapeutic agent.

Embodiment 115. A pharmaceutical combination which comprises (a) a compound of Example No. 29, or a pharmaceutically acceptable salt thereof, and (b) an additional therapeutic agent, for use in therapy.

Embodiment 116. The pharmaceutical combination of Embodiment 114, or for use of Embodiment 115, wherein the compound of Example No. 29, or the pharmaceutically acceptable salt thereof and the additional therapeutic agent are formulated as separate compositions or dosages for simultaneous, separate or sequential use for use in therapy, wherein the amounts of the compound of Example No. 29, or a pharmaceutically acceptable salt thereof and of the additional therapeutic agent are together therapeutically effective.

Embodiment 117. A pharmaceutical composition, comprising (a) a compound of Example No. 29, or a pharmaceutically acceptable salt thereof, (b) an additional therapeutic agent, and (c) a pharmaceutically acceptable diluent or carrier.

Embodiment 118. The pharmaceutical combination of Embodiment 114, or for use of Embodiment 115, or the pharmaceutical composition of Embodiment 117, wherein the additional therapeutic agent is an anticancer agent.

Embodiment 119. The pharmaceutical combination of Embodiment 114, or for use of Embodiment 115, or the pharmaceutical composition of Embodiment 117, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 120. A pharmaceutical combination which comprises (a) a compound of Example No. 32, or a pharmaceutically acceptable salt thereof, and (b) an additional therapeutic agent.

Embodiment 121. A pharmaceutical combination which comprises (a) a compound of Example No. 32, or a pharmaceutically acceptable salt thereof, and (b) an additional therapeutic agent, for use in therapy.

Embodiment 122. The pharmaceutical combination of Embodiment 120, or for use of Embodiment 121, wherein the compound of Example No. 32, or the pharmaceutically acceptable salt thereof and the additional therapeutic agent are formulated as separate compositions or dosages for simultaneous, separate or sequential use for use in therapy, wherein the amounts of the compound of Example No. 32, or a pharmaceutically acceptable salt thereof and of the additional therapeutic agent are together therapeutically effective.

Embodiment 123. A pharmaceutical composition, comprising (a) a compound of Example No. 32, or a pharmaceutically acceptable salt thereof, (b) an additional therapeutic agent, and (c) a pharmaceutically acceptable diluent or carrier.

Embodiment 124. The pharmaceutical combination of Embodiment 120, or for use of Embodiment 121, or the pharmaceutical composition of Embodiment 123, wherein the additional therapeutic agent is an anticancer agent.

Embodiment 125. The pharmaceutical combination of Embodiment 120, or for use of Embodiment 121, or the pharmaceutical composition of Embodiment 123, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 126. A pharmaceutical combination which comprises (a) a compound of Example No. 33, or a pharmaceutically acceptable salt thereof, and (b) an additional therapeutic agent.

Embodiment 127. A pharmaceutical combination which comprises (a) a compound of Example No. 33, or a pharmaceutically acceptable salt thereof, and (b) an additional therapeutic agent, for use in therapy.

Embodiment 128. The pharmaceutical combination of Embodiment 126, or for use of Embodiment 127, wherein the compound of Example No. 33, or the pharmaceutically acceptable salt thereof and the additional therapeutic agent are formulated as separate compositions or dosages for simultaneous, separate or sequential use for use in therapy, wherein the amounts of the compound of Example No. 33, or a pharmaceutically acceptable salt thereof and of the additional therapeutic agent are together therapeutically effective.

Embodiment 129. A pharmaceutical composition, comprising (a) a compound of Example No. 33, or a pharmaceutically acceptable salt thereof, (b) an additional therapeutic agent, and (c) a pharmaceutically acceptable diluent or carrier.

Embodiment 130. The pharmaceutical combination of Embodiment 126, or for use of Embodiment 127, or the pharmaceutical composition of Embodiment 129, wherein the additional therapeutic agent is an anticancer agent.

Embodiment 131. The pharmaceutical combination of Embodiment 126, or for use of Embodiment 127, or the pharmaceutical composition of Embodiment 129, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 132. A pharmaceutical combination which comprises (a) a compound of Example No. 44, or a pharmaceutically acceptable salt thereof, and (b) an additional therapeutic agent.

Embodiment 133. A pharmaceutical combination which comprises (a) a compound of Example No. 44, or a pharmaceutically acceptable salt thereof, and (b) an additional therapeutic agent, for use in therapy.

Embodiment 134. The pharmaceutical combination of Embodiment 132, or for use of Embodiment 133, wherein the compound of Example No. 44, or the pharmaceutically acceptable salt thereof and the additional therapeutic agent are formulated as separate compositions or dosages for simultaneous, separate or sequential use for use in therapy, wherein the amounts of the compound of Example No. 44, or a pharmaceutically acceptable salt thereof and of the additional therapeutic agent are together therapeutically effective.

Embodiment 135. A pharmaceutical composition, comprising (a) a compound of Example No. 44, or a pharmaceutically acceptable salt thereof, (b) an additional therapeutic agent, and (c) a pharmaceutically acceptable diluent or carrier.

Embodiment 136. The pharmaceutical combination of Embodiment 132, or for use of Embodiment 133, or the pharmaceutical composition of Embodiment 135, wherein the additional therapeutic agent is an anticancer agent.

Embodiment 137. The pharmaceutical combination of Embodiment 132, or for use of Embodiment 133, or the pharmaceutical composition of Embodiment 135, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 138. A pharmaceutical combination which comprises (a) a compound of Example No. 46, or a pharmaceutically acceptable salt thereof, and (b) an additional therapeutic agent.

Embodiment 139. A pharmaceutical combination which comprises (a) a compound of Example No. 46, or a pharmaceutically acceptable salt thereof, and (b) an additional therapeutic agent, for use in therapy.

Embodiment 140. The pharmaceutical combination of Embodiment 138, or for use of Embodiment 139, wherein the compound of Example No. 46, or the pharmaceutically acceptable salt thereof and the additional therapeutic agent are formulated as separate compositions or dosages for simultaneous, separate or sequential use for use in therapy, wherein the amounts of the compound of Example No. 46, or a pharmaceutically acceptable salt thereof and of the additional therapeutic agent are together therapeutically effective.

Embodiment 141. A pharmaceutical composition, comprising (a) a compound of Example No. 46, or a pharmaceutically acceptable salt thereof, (b) an additional therapeutic agent, and (c) a pharmaceutically acceptable diluent or carrier.

Embodiment 142. The pharmaceutical combination of Embodiment 138, or for use of Embodiment 139, or the pharmaceutical composition of Embodiment 141, wherein the additional therapeutic agent is an anticancer agent.

Embodiment 143. The pharmaceutical combination of Embodiment 138, or for use of Embodiment 139, or the pharmaceutical composition of Embodiment 141, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 144. A pharmaceutical combination which comprises (a) a compound of Example No. 48, or a pharmaceutically acceptable salt thereof, and (b) an additional therapeutic agent.

Embodiment 145. A pharmaceutical combination which comprises (a) a compound of Example No. 48, or a pharmaceutically acceptable salt thereof, and (b) an additional therapeutic agent, for use in therapy.

Embodiment 146. The pharmaceutical combination of Embodiment 144, or for use of Embodiment 145, wherein the compound of Example No. 48, or the pharmaceutically acceptable salt thereof and the additional therapeutic agent are formulated as separate compositions or dosages for simultaneous, separate or sequential use for use in therapy, wherein the amounts of the compound of Example No. 48, or a pharmaceutically acceptable salt thereof and of the additional therapeutic agent are together therapeutically effective.

Embodiment 147. A pharmaceutical composition, comprising (a) a compound of Example No. 48, or a pharmaceutically acceptable salt thereof, (b) an additional therapeutic agent, and (c) a pharmaceutically acceptable diluent or carrier.

Embodiment 148. The pharmaceutical combination of Embodiment 144, or for use of Embodiment 145, or the pharmaceutical composition of Embodiment 147, wherein the additional therapeutic agent is an anticancer agent.

Embodiment 149. The pharmaceutical combination of Embodiment 144, or for use of Embodiment 145, or the pharmaceutical composition of Embodiment 147, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 150. A pharmaceutical combination which comprises (a) a compound of Example No. 55, or a pharmaceutically acceptable salt thereof, and (b) an additional therapeutic agent.

Embodiment 151. A pharmaceutical combination which comprises (a) a compound of Example No. 55, or a pharmaceutically acceptable salt thereof, and (b) an additional therapeutic agent, for use in therapy.

Embodiment 152. The pharmaceutical combination of Embodiment 150, or for use of Embodiment 151, wherein the compound of Example No. 55, or the pharmaceutically acceptable salt thereof and the additional therapeutic agent are formulated as separate compositions or dosages for simultaneous, separate or sequential use for use in therapy, wherein the amounts of the compound of Example No. 55, or a pharmaceutically acceptable salt thereof and of the additional therapeutic agent are together therapeutically effective.

Embodiment 153. A pharmaceutical composition, comprising (a) a compound of Example No. 55, or a pharmaceutically acceptable salt thereof, (b) an additional therapeutic agent, and (c) a pharmaceutically acceptable diluent or carrier.

Embodiment 154. The pharmaceutical combination of Embodiment 150, or for use of Embodiment 151, or the pharmaceutical composition of Embodiment 153, wherein the additional therapeutic agent is an anticancer agent.

Embodiment 155. The pharmaceutical combination of Embodiment 150, or for use of Embodiment 151, or the pharmaceutical composition of Embodiment 153, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 156. A pharmaceutical combination which comprises (a) a compound of Example No. 56, or a pharmaceutically acceptable salt thereof, and (b) an additional therapeutic agent.

Embodiment 157. A pharmaceutical combination which comprises (a) a compound of Example No. 56, or a pharmaceutically acceptable salt thereof, and (b) an additional therapeutic agent, for use in therapy.

Embodiment 158. The pharmaceutical combination of Embodiment 156, or for use of Embodiment 157, wherein the compound of Example No. 56, or the pharmaceutically acceptable salt thereof and the additional therapeutic agent are formulated as separate compositions or dosages for simultaneous, separate or sequential use for use in therapy, wherein the amounts of the compound of Example No. 56, or a pharmaceutically acceptable salt thereof and of the additional therapeutic agent are together therapeutically effective.

Embodiment 159. A pharmaceutical composition, comprising (a) a compound of Example No. 56, or a pharmaceutically acceptable salt thereof, (b) an additional therapeutic agent, and (c) a pharmaceutically acceptable diluent or carrier.

Embodiment 160. The pharmaceutical combination of Embodiment 156, or for use of Embodiment 157, or the pharmaceutical composition of Embodiment 159, wherein the additional therapeutic agent is an anticancer agent.

Embodiment 161. The pharmaceutical combination of Embodiment 156, or for use of Embodiment 157, or the pharmaceutical composition of Embodiment 159, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

Embodiment 162. A pharmaceutical combination which comprises (a) a compound of Example No. 58, or a pharmaceutically acceptable salt thereof, and (b) an additional therapeutic agent.

Embodiment 163. A pharmaceutical combination which comprises (a) a compound of Example No. 58, or a pharmaceutically acceptable salt thereof, and (b) an additional therapeutic agent, for use in therapy.

Embodiment 164. The pharmaceutical combination of Embodiment 162, or for use of Embodiment 163, wherein the compound of Example No. 58, or the pharmaceutically acceptable salt thereof and the additional therapeutic agent are formulated as separate compositions or dosages for simultaneous, separate or sequential use for use in therapy, wherein the amounts of the compound of Example No. 58, or a pharmaceutically acceptable salt thereof and of the additional therapeutic agent are together therapeutically effective.

Embodiment 165. A pharmaceutical composition, comprising (a) a compound of Example No. 58, or a pharmaceutically acceptable salt thereof, (b) an additional therapeutic agent, and (c) a pharmaceutically acceptable diluent or carrier.

Embodiment 166. The pharmaceutical combination of Embodiment 162, or for use of Embodiment 163, or the pharmaceutical composition of Embodiment 165, wherein the additional therapeutic agent is an anticancer agent.

Embodiment 167. The pharmaceutical combination of Embodiment 162, or for use of Embodiment 163, or the pharmaceutical composition of Embodiment 165, wherein the additional therapeutic agent is selected from the group consisting of binimetinib, encorafenib, selumetinib, sorafenib, trametinib, vemurafenib, cetuximab or a biosimilar thereof, panitumumab or a biosimilar thereof, erlotinib, lapatinib, gefitinib, nivolumab or a biosimilar thereof, pembrolizumab or a biosimilar thereof, cemiplimab or a biosimilar thereof, pidilizumab or a biosimilar thereof, atezolizumab or a biosimilar thereof, avelumab or a biosimilar thereof, and durvalumab or a biosimilar thereof.

What is claimed is:

1. A compound of Formula II 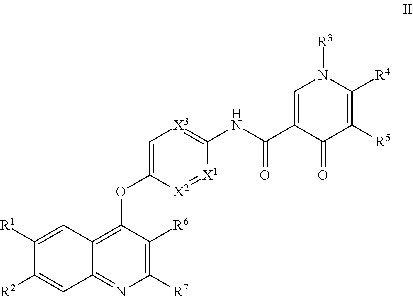
or a pharmaceutically acceptable salt thereof, wherein:
X¹, X² and X³ are independently N or CH, wherein one or two of X¹, X² and X³ are N;
R¹ is hydrogen or C1-C6 alkoxy;
R² is hydrogen, C1-C6 alkoxy, fluoroC1-C6 alkoxy, halogen or (hetCyc¹)C1-C6 alkoxy-;
hetCyc¹ is a 5-6 membered heterocyclic ring having 1-2 ring heteroatoms independently selected from N and O;
R³ is hydrogen, C1-C7 alkyl, (C1-C6 alkoxy)C1-C6 alkyl-, hydroxyC1-C6 alkyl-, R^{a}R^{b}NC(═O)C1-C6 alkyl-, or (R^{c}R^{d}N)C1-C6 alkyl-;
R^{a} and R^{b} are independently hydrogen or C1-C6 alkyl, or
R^{a} and R^{b} together with the nitrogen atom to which they are attached form a 5-6 membered heterocyclic ring having one ring nitrogen atom and optionally having a second ring heteroatom selected from O and N;
R^{c} and R^{d} are independently hydrogen or C1-C6 alkyl;
R⁴ is hydrogen or C1-C6 alkyl;
R⁵ is phenyl optionally substituted with one to five substituents independently selected from halogen, C1-C6 alkyl and C1-C6 alkoxy;
R⁶ is hydrogen or CN; and
R⁷ is hydrogen or C1-C3 alkyl.

2. A compound of Formula III 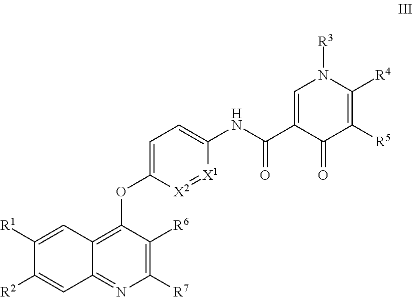
or a pharmaceutically acceptable salt thereof, wherein:
X¹ is N and X² is CH, or X¹ is CH and X² is N;
R¹ is hydrogen or C1-C6 alkoxy;
R² is hydrogen, C1-C6 alkoxy, fluoroC1-C6 alkoxy, halogen or (hetCyc¹)C1-C6 alkoxy-;
hetCyc¹ is a 5-6 membered heterocyclic ring having 1-2 ring heteroatoms independently selected from N and O;
R³ is C1-C7 alkyl;
R⁴ is hydrogen or C1-C6 alkyl;
R⁵ is phenyl optionally substituted with one or two substituents independently selected from halogen, C1-C6 alkyl and C1-C6 alkoxy;
R⁶ is hydrogen or CN; and
R⁷ is C1-C3 alkyl.

3. A compound of Formula IV 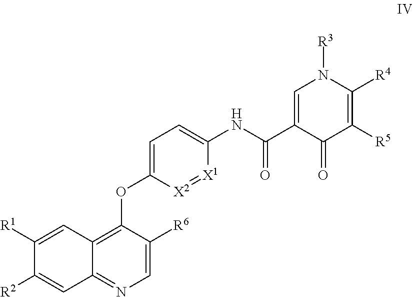
or a pharmaceutically acceptable salt thereof, wherein:
X¹ is N and X² is CH, or X¹ is CH and X² is N;
R¹ is hydrogen or C1-C6 alkoxy;
R² is hydrogen, C1-C6 alkoxy, fluoroC1-C6 alkoxy or halogen;
R³ is C1-C6 alkyl;
R⁴ is hydrogen or methyl;
R⁵ is phenyl optionally substituted with one or two substituents independently selected from halogen, C1-C6 alkyl and C1-C6 alkoxy; and
R⁶ is hydrogen or CN.

4. A compound according to claim 1 or a pharmaceutically acceptable salt thereof, wherein R⁴ is hydrogen.

5. A compound according to claim 1 or a pharmaceutically acceptable salt thereof, wherein R⁵ is phenyl substituted with one or two substituents independently selected from fluoro, chloro, methyl and methoxy.

6. A compound according to claim 1 or a pharmaceutically acceptable salt thereof, wherein R² is C1-C6 alkoxy.

7. A compound according to claim 6 or a pharmaceutically acceptable salt thereof, wherein R² is methoxy.

8. A compound according to claim 1 or a pharmaceutically acceptable salt thereof, wherein R¹ is C1-C6 alkoxy.

9. A compound according to claim 8 or a pharmaceutically acceptable salt thereof, wherein R¹ is methoxy.

10. A compound according to claim 1 or a pharmaceutically acceptable salt thereof, wherein R⁶ is hydrogen.

11. A compound according to claim 1 or a pharmaceutically acceptable salt thereof, wherein X¹ is N, X² is CH and X³ is CH.

12. A compound according to claim 1, which is N-(5-((6,7-dimethoxyquinolin-4-yl)oxy)pyridin-2-yl)-5-(4-fluorophenyl)-1-isopropyl-4-oxo-1,4-dihydropyridine-3-carboxamide or a pharmaceutically acceptable salt thereof.

13. A compound according to claim 1, which is 5-(2,4-difluorophenyI)-N-(5-((6,7-dimethoxyquinolin-4-yl)oxy)pyridin-2-yl)-1-isopropyl-4-oxo-1,4-dihydropyridine-3-carboxamide or a pharmaceutically acceptable salt thereof.

14. A compound according to claim 1, which is 5-(4-chlorophenyl)-N-(5-((6,7-dimethoxyquinolin-4-yl)oxy)pyridin-2-yl)-1-isopropyl-4-oxo-1,4-dihydropyridine-3-carboxamide or a pharmaceutically acceptable salt thereof.

15. A compound according to claim 1, which is 5-(2,4-difluorophenyl)-N-(5-((6,7-dimethoxyquinolin-4-yl)oxy)pyridin-2-yl)-4-oxo-1-(pentan-3-yl)-1,4-dihydropyridine-3-carboxamide or a pharmaceutically acceptable salt thereof.

16. A compound according to claim 1, which is 5-(2-chloro-4-fluorophenyl)-N-(5-((6,7-dimethoxyquinolin-4-yl)oxy)pyridin-2-yl)-1-isopropyl-4-oxo-1,4-dihydropyridine-3-carboxamide or a pharmaceutically acceptable salt thereof.

17. A compound according to claim 1, which is N-(5-((6,7-dimethoxyquinolin-4-yl)oxy)pyridin-2-yl)-5-(4-fluoro-2-methylphenyl)-1-isopropyl-4-oxo-1,4-dihydropyridine-3-carboxamide or a pharmaceutically acceptable salt thereof.

18. A compound according to claim 1, which is 5-(3-chloro-4-fluorophenyl)-N-(5-((6,7-dimethoxyquinolin-4-yl)oxy)pyridin-2-yl)-1-isopropyl-4-oxo-1,4-dihydropyridine-3-carboxamide or a pharmaceutically acceptable salt thereof.

19. A compound according to claim 1, which is 5-(3,4-difluorophenyI)-N-(5-((6,7-dimethoxyquinolin-4-yl)oxy)pyridin-2-yl)-1-isopropyl-6-methyl-4-oxo-1,4-dihydropyridine-3-carboxamide or a pharmaceutically acceptable salt thereof.

20. A compound according to claim 1, which is N-(5-((6,7-dimethoxyquinolin-4-yl)oxy)pyridin-2-yl)-1-isopropyl-5-(4-methoxyphenyl)-4-oxo-1,4-dihydropyridine-3-carboxamide or a pharmaceutically acceptable salt thereof.

21. A compound according to claim 1, which is N-(5-((6,7-dimethoxyquinolin-4-yl)oxy)pyridin-2-yl)-5-(2-fluoro-4-methoxyphenyl)-1-isopropyl-4-oxo-1,4-dihydropyridine-3-carboxamide or a pharmaceutically acceptable salt thereof.

22. A compound according to claim 1, which is 5-(4-fluorophenyl)-1-isopropyl-N-(5((6-methoxyquinolin-4-yl)oxy)pyridin-2-yl)-6-methyl-4-oxo-1,4-dihydropyridine-3-carboxamide or a pharmaceutically acceptable salt thereof.

23. A compound according to claim 1, which is 5-(3,4-difluorophenyI)-N-(5-((6,7-dimethoxyquinolin-4-yl)oxy)pyridin-2-yl)-1-isopentyl-4-oxo-1,4-dihydropyridine-3-carboxamide or a pharmaceutically acceptable salt thereof.

24. A compound according to claim 1, which is 1-ethyl-5-(4-fluorophenyl)-N-(5-((6-methoxyquinolin-4-yl)oxy)pyridin-2-yl)-4-oxo-1,4-dihydropyridine-3-carboxamide or a pharmaceutically acceptable salt thereof.

25. A compound according to claim 1, which is 5-(4-fluorophenyl)-N-(5-((6-methoxyquinolin-4-yl)oxy)pyridin-2-yl)-1-methyl-4-oxo-1,4-dihydropyridine-3-carboxamide or a pharmaceutically acceptable salt thereof.

26. A compound according to claim 1, which is N-(5-((7-fluoro-6-methoxyquinolin-4-yl)oxy)pyridin-2-yl)-5-(4-fluorophenyl)-1-isopropyl-6-methyl-4-oxo-1,4-dihydropyridine-3-carboxamide or a pharmaceutically acceptable salt thereof.

27. A compound according to claim 1, which is N-(5-((7-fluoro-6-methoxyquinolin-4-yl)oxy)pyridin-2-yl)-5-(4-fluorophenyl)-1-isopropyl-4-oxo-1,4-dihydropyridine-3-carboxamide or a pharmaceutically acceptable salt thereof.

28. A compound according to claim 1, which is N-(5-((6-ethoxyquinolin-4-yl)oxy)pyridin-2-yl)-5-(4-fluorophenyl)-1-isopropyl-4-oxo-1,4-dihydropyridine-3-carboxamide or a pharmaceutically acceptable salt thereof.

29. A compound according to claim 1, which is N-(5-((7-ethoxyquinolin-4-yl)oxy)pyridin-2-yl)-5-(4-fluorophenyl)-1-isopropyl-4-oxo-1,4-dihydropyridine-3-carboxamide or a pharmaceutically acceptable salt thereof.

30. A compound according to claim 1, which is N-(5-((3-cyano-6,7-dimethoxyquinolin-4-yl)oxy)pyridin-2-yl)-5-(4-fluorophenyl)-1-isopropyl-4-oxo-1,4-dihydropyridine-3-carboxamide or a pharmaceutically acceptable salt thereof.

31. A compound according to claim 1, which is N-(6((6,7-dimethoxyquinolin-4-yl)oxy)pyridin-3-yl)-5-(4-fluorophenyl)-1-isopropyl-4-oxo-1,4-dihydropyridine-3-carboxamide or a pharmaceutically acceptable salt thereof.

32. A pharmaceutical composition, comprising a compound as defined in any one of claims 1 to 36 or a pharmaceutically acceptable salt thereof, and a pharmaceutically acceptable diluent or carrier.

33. A process for preparing a compound according to claim 1, comprising:
reacting a compound having the formula: 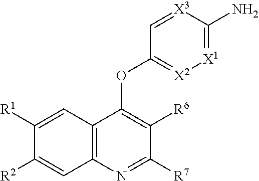
wherein R¹, R², R⁶, R⁷, X¹, X² and X³ are as defined for claim 1, with a compound having the formula 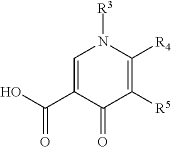
wherein R³, R⁴ and R⁵ are as defined for claim 1, in the presence of 1-[bis(dimethylamino)methylene]-1H-1,2,3-triazolo[4,5-b]pyridinium 3-oxide hexafluorophosphate and an amine base.

34. A method for treating cancer in a patient in need thereof, the method comprising administering to the patient an effective amount of a compound of claim 1 or a pharmaceutically acceptable salt thereof.

35. A method of treating a patient identified or diagnosed as having a TAM-associated cancer, a c-Met-associated cancer, or both, the method comprising administering to a patient identified or diagnosed as having a TAM-associated cancer, a c-Met-associated cancer, or both, a therapeutically effective amount of a compound of claim 1 or a pharmaceutically acceptable salt thereof.

36. The method of claim 34, wherein the method further includes administering to the patient at least one additional anticancer agent.

37. The method of claim 36, wherein the at least one additional anticancer agent or therapy is selected from the group consisting of: an immune checkpoint inhibitor, a kinase inhibitor, a chemotherapy, radiation and surgery.

38. The method of claim 37, wherein the at least one additional anticancer agent is selected from the group consisting of: a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor, a RAF inhibitor, an Akt inhibitor, a FTL-3 inhibitor, a MAP kinase pathway inhibitor, a PD-1 inhibitor and a PD-L1 inhibitor.

39. The method according to claim 35, wherein the TAM-associated cancer is selected from the group consisting of: gastrointestinal stromal tumor (GIST), acute myeloid leukemia (AML), acute lymphocytic leukemia (ALL), chronic myeloid leukemia (CML), B-cell chronic myeloid leukemia (B-CLL), lung cancer, glioblastoma, breast cancer, colorectal cancer, gastric cancer, glioma, pancreatic cancer, esophageal cancer, mantle cell lymphoma, melanoma, squamous cell skin cancer, prostate cancer, endometrial cancer, ovarian cancer, oral squamous cell carcinoma, thyroid cancer, bladder cancer, renal cancer, schwannoma, mesothelioma, Kaposi's sarcoma, osteosarcoma, rhabdomyosarcoma, erythroid leukemia, colon cancer, liver cancer, renal cell carcinoma, pituitary adenoma, urinary tract cancer, kidney cancer, colon

40. The method according to claim 35, wherein the TAM-associated cancer is selected from the group consisting of: acute myeloid leukemia (AML), multiple myeloma, lung cancer, melanoma, prostate cancer, endometrial cancer, thyroid cancer, schwannoma, pancreatic cancer, and brain cancer.

41. The method according to claim 35, wherein the TAM-associated cancer is selected from the group consisting of: gastrointestinal stromal tumor (GIST), acute myeloid leukemia (AML), acute lymphocytic leukemia (ALL), chronic myeloid leukemia (CML), B-cell chronic myeloid leukemia (B-CLL), lung cancer, glioblastoma, breast cancer, colorectal cancer, gastric cancer, pancreatic cancer, prostate cancer, esophageal cancer, melanoma, squamous cell skin cancer, endometrial cancer, ovarian cancer, oral squamous cell carcinoma, thyroid cancer, bladder cancer, renal cancer, schwannoma, mesothelioma, osteosarcoma, erythroid leukemia, colon cancer, liver cancer, renal cell carcinoma, kidney cancer, non-small cell lung cancer, and triple-negative metastatic breast cancer.

42. The method according to claim 35, wherein the TAM-associated cancer is selected from the group consisting of: acute myeloid leukemia (AML), acute lymphocytic leukemia (ALL), B-cell chronic myeloid leukemia (B-CLL), T-cell acute lymphoblastic leukemia (T-ALL), lung cancer, glioma, melanoma, prostate cancer, schwannoma, mantle cell lymphoma, rhabdomyosarcoma, pancreatic cancer, breast cancer, gastric cancer, pituitary adenoma, urinary tract cancer, kidney cancer, liver cancer, colon cancer, and breast cancer.

43. The method according to claim 35, wherein the c-Met-associated cancer is selected from the group of gastrointestinal cancer (Cl), gastric cancer, colorectal adenocarcinoma, colorectal carcinoma (CRC), non-small cell lung cancer (NSCLC), hepatocellular carcinoma (HCC), hereditary papillary renal carcinoma (HPRC), papillary renal carcinoma, melanoma, gastric adenocarcinoma, appendiceal adenocarcinoma, duodenal adenocarcinoma, pancreatic adenocarcinoma, lung adenocarcinoma, thyroid papillary carcinoma, thyroid medullary carcinoma, Ewing sarcoma, prostate adenocarcinoma, squamous cell carcinoma of the head and neck and cervix, renal cell carcinoma, pheochromocytoma and composite pheochromocytoma, ovarian serous carcinoma, ovarian clear cell carcinoma, ovarian mixed carcinoma, peritoneal serous carcinoma, breast ductal adenocarcinoma, uterine leiomyosarcoma, uterine endometrioid adenocarcinoma, uterine malignant mixed Mullerian tumor, glioblastoma, anaplastic glioma, oligodendroglioma, desmoplastic small round cell tumor, squamous cell carcinoma of rectum, salivary gland carcinoma, heart angiosarcoma, gastrointestinal stromal tumor, invasive thymoma, and spindle sarcoma.
